(19) United States
(12) Patent Application Publication
(10) Pub. No.: US 2008/0312435 A1
(43) Pub. Date: Dec. 18, 2008

(54) Imine Compound

(75) Inventors: Shiuji Saito, Tokyo (JP); Hiroshi Ohta, Tokyo (JP); Tomoko Ishizaki, Tokyo (JP); Mitsukane Yoshinaga, Tokyo (JP); Makoto Tatsuzuki, Tokyo (JP); Yuji Yokobori, Tokyo (JP); Yasumitsu Tomishima, Tokyo (JP); Aki Morita, Tokyo (JP); Yoshihisa Toda, Tokyo (JP); Kimiko Tokugawa, Tokyo (JP); Ayaka Kaku, Tokyo (JP); Tomomi Murakami, Tokyo (JP); Hiromitsu Yoshimura, Tokyo (JP); Shingo Sekine, Tokyo (JP); Takao Yoshimizu, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: TAISHO PHARMACEUTICAL CO., LTD., Tokyo (JP)

(21) Appl. No.: 11/667,728

(22) PCT Filed: Oct. 31, 2005
(86) PCT No.: PCT/JP05/19977

§ 371 Date: May 15, 2007

(30) Foreign Application Priority Data
Nov. 15, 2004 (JP) 2004-330079
Nov. 15, 2004 (JP) 2004-330080
Jun. 2, 2005 (JP) 2005-162163
Jul. 20, 2005 (JP) 2005-209774

(51) Int. Cl. C07D 417/12; C07D 213/75; C07D 277/46; C07D 231/40; C07D 263/48

(52) U.S. Cl. 544/133; 546/309; 548/195; 548/372.5; 548/233

(57) ABSTRACT

An imine compound represented by the formula:

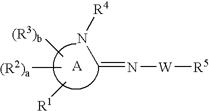

wherein A represents a heterocyclic group; R¹, R², an R³ each represent a hydrogen atom, a halogen atom, a C₁₋₁₀ alkyl group optionally substituted with an aryl group(s) substituted with a halogen atom(s), a C₃₋₁₀ cycloalkyl group, a C₁₋₆ haloalkyl group, a C₁₋₁₀ alkoxy group, etc.; R⁴ represents an optionally substituted C₁₋₁₀ alkyl, C₂₋₆ alkenyl, or aryl group; R⁵ represents a hydrogen atom, a C₁₋₁₀ alkoxy group, a C₁₋₆ haloalkyl group, an optionally substituted C₁₋₁₀ alkyl or C₂₋₆ alkenyl group, an optionally substituted aryl or heterocyclic group, etc.; W represents —CO—, —CO—CO—, —CO—NH—, —CS—NH—, or —SO₂—,
or a cannabinoid-receptor agonist comprising said imine compound as an active ingredient.

The imine compound of the present invention has a cannabinoid-receptor agonist effect, and is useful as a therapeutic or prophylactic drug for pains and autoimmune diseases.

### TECHNICAL FIELD

The present invention relates to an imine compound having a cannabinoid-receptor agonist effect.

### BACKGROUND ART

Cannabinoids are substances isolated as a physiologically active component of marihuana in 1960, and having effects such as an analgesic, anti-anxiety, sedation or euphoriant effect. Then, its receptor was found. By virtue of this, endogenous ligands having a cannabinoid-like physiological activity, such as anandamide was discovered.

As the cannabinoid receptor, a cannabinoid type 1 (CB1) receptor was discovered in 1990. It was found that CB1 is distributed over a central nervous system such as brain, and its agonist has an analgesic effect. In 1993, a cannabinoid type 2 (CB2) receptor was discovered. It was found that CB2 is distributed over the tissue and cells of an immune system including blood-system cells such as spleen, lymph node and leucocytes, B cells, T cells, macrophages, and mast cells, and that its agonist has effects such as an immune suppressive effect, an anti-inflammatory effect, and an analgesic effect.

Compounds having a CB1 receptor agonist effect and those having a CB2 receptor agonist effect are disclosed, for example, in Non-Patent Documents 1 and 2, etc.

Imine compounds having analogous structures to those of the compounds according to the present invention are described, for example, in Non-Patent Documents 3 to 8 and Patent Documents 1 to 20, etc. It has been reported that the imine compounds are applied to various usages such as agricultural germicides, herbicides, platelet aggregation inhibitors, therapeutic drugs having a leukocyte infiltration inhibitory effect for various types of inflammations, anti-allergic drugs/anti-inflammatory agents/immunomodulators, and analgesics. However, no reports have been made on a cannabinoid receptor agonist effect caused by an imine compound as an active ingredient.
Non-Patent Document 1: Exp. Opin. Ther. Patent (2002) 12 (10): 1475-1489
Non-Patent Document 2: Exp. Opin. Ther. Patent (2004) 14 (10): 1435-1452
Non-Patent Document 3: European Journal of Medicinal Chemistry (1994) 29 (11): 841-854
Non-Patent Document 4: Journal of Medicinal Chemistry (1966)9(1):151-153
Non-Patent Document 5: IzVestiya Akademii Nauk SSSR, Seriya Kimicheskaya (1953): 154-162
Non-Patent Document 6: Farmaco, Edizione Scientifica (1985) 40 (3): 178-189
Non-Patent Document 7: Journal of Heterocyclic Chemistry (1983) 20 (5): 1153-1154
Non-Patent Document 8: Journal of Heterocyclic Chemistry (1981) 18 (4): 745-750
Patent Document 1: WO9215564
Patent Document 2: EP432600
Patent Document 3: DE1036326
Patent Document 4: WO2001055139
Patent Document 5: WO2000063207
Patent Document 6: JP2003292485
Patent Document 7: WO2002002542
Patent Document 8: WO2003097605
Patent Document 9: JP2003192591
Patent Document 10: WO2000017196
Patent Document 11: WO9842703
Patent Document 12: WO2002002542
Patent Document 13: JP02250874
Patent Document 14: JP62004277
Patent Document 15: EP40573
Patent Document 16: JP63203672
Patent Document 17: JP08081449
Patent Document 18: WO9703058
Patent Document 19: WO9404516
Patent Document 20: JP02229164

### DISCLOSURE OF THE INVENTION

An object of the present invention is to provide a novel imine compound having a cannabinoid receptor agonist effect.

The present inventors conducted intensive studies on imine compounds, and found a novel imine compound having a cannabinoid receptor agonist effect. Based on the finding, the present invention was accomplished.

The present invention will be explained below.

According to the present invention, there is provided a cannabinoid-receptor agonist comprising an imine compound represented by Formula (I)

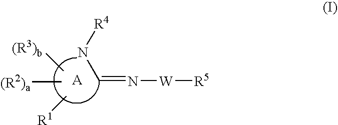

[where A represents any one of the rings represented by the following formulas (where X represents an oxygen atom or a sulfur atom, and X′ represents CH or a nitrogen atom):

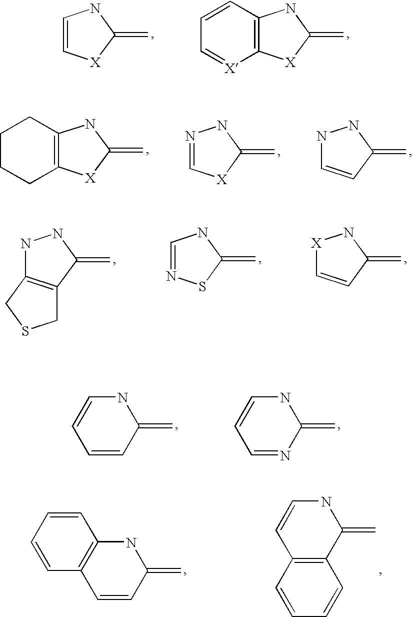

R¹ represents

a hydrogen atom;
a halogen atom;
a C₁₋₁₀ alkyl group that may be substituted with an aryl group(s) substituted with a halogen atom(s);
a C₃₋₁₀ cycloalkyl group;
a C₂₋₆ alkenyl group;
a C₁₋₆ haloalkyl group;
a C₁₋₁₀ alkoxy group;
a carboxyl group;
a C₂₋₆ alkoxycarbonyl group;
a hydroxy-C₁₋₆ alkyl group;
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group);
a group represented by Formula —CON(R⁶¹)R⁷¹ (where R⁶¹ and R⁷¹ each represent a hydrogen atom, or a C₁₋₆ alkyl group that may be substituted with a cyclic amino group(s), or R⁶¹ and R⁷¹, in combination with the adjacent nitrogen atom, form a cyclic amino group); or
an aryl group that may be substituted with 1 to 3 groups selected from the group consisting of: a C₁₋₆ alkyl group, a C₁₋₆ haloalkyl group and a halogen atom,

R² and R³ each represent

a hydrogen atom;
a halogen atom;
a C₁₋₆ alkyl group;
a C₁₋₆ haloalkyl group; or
an aryl group that may be substituted with 1 to 3 groups selected from the group consisting of: a C₁₋₆ alkyl group, a C₁₋₆ haloalkyl group and a halogen atom,

R⁴ represents

a C₁₋₁₀ alkyl group;
a C₁₋₆ haloalkyl group;
a C₁₋₁₀ alkyl group or C₂₋₆ alkenyl group substituted with: a C₃₋₁₀ cycloalkyl group(s), a C₁₋₆ alkoxy group(s), a hydroxyl group(s), an amino group(s), a phthalimide group(s), a cyano group(s), an arylthio group(s), a C₂₋₆ alkoxycarbonyl group(s), a carboxyl group(s), a group(s) represented by
Formula —CON(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group), or an aryl group(s) that may be substituted with a C₁₋₆ haloalkyl group(s), a C₂₋₆ alkoxycarbonyl group(s), a carboxyl group(s), or an N-piperidinocarbamoyl group(s);
a C₂₋₆ haloalkenyl group;
a C₂₋₆ alkynyl group;
a 1,1-dioxothiolanyl group; or
an aryl group,

R⁵ represents

a hydrogen atom;
a C₁₋₁₀ alkoxy group;
a C₁₋₆ alkoxy-C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, a C₂₋₆ alkoxycarbonyl group, a C₁₋₆ haloalkyl group, a C₁₋₆ alkoxy group that may be substituted with a C₁₋₆ alkoxy group(s) or an aryl group(s), a C₃₋₁₀ cycloalkoxy group that may be substituted with 1 to 2 C₁₋₆ alkyl groups, an aryl group or aryloxy group that may be substituted with 1 to 5 groups selected from the group consisting of a C₁₋₆ alkyl group, a C₁₋₆ alkoxy group, a C₁₋₆ haloalkyl group, a C₁₋₆ haloalkoxy group, an aralkyloxy group, a nitro group and a halogen atom, a heterocyclic group, a phthalimide group, a C₁₋₆ alkanoyloxy group, an aralkyloxy group, a C₁₋₆ alkylthio group, an arylthio group and a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with: a C₁₋₆ alkyl group(s), a C₁₋₆ alkoxy group(s), a C₂₋₆ alkoxycarbonyl group(s), a C₁₋₆ haloalkyl group(s) or a C₁₋₆ haloalkoxy group(s);
an aralkyloxy group; or
a group represented by Formula (II)

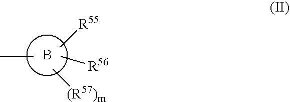

{where

B represents

a C₃₋₁₀ cycloalkyl group;
an aryl group;
a heterocyclic group;
a C₂₋₆ cyclic amino group;
a fluorenyl group;
a phthalimide group;
a 2-oxopyrrolidinyl group;
a group represented by Formula (III)

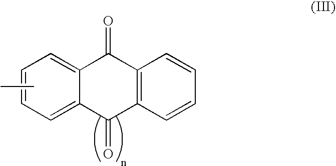

(where n represents 0 or 1); or
a group represented by Formula (IV)

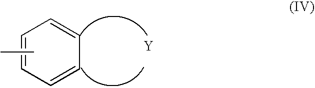

(where Y represents —(CH₂)p-, —CO—CH₂—CH₂—, —CO—CH₂—CH₂—CH₂—, —O—CH₂—CH₂—, —O—CH₂—CH═CH—, or —O—(CH₂)_{q}—O—, in which p represents an integer of 2 to 4, and q represents an integer of 1 to 3);

R⁵⁵ represents

a hydrogen atom;
a halogen atom;
a C₁₋₁₀ alkyl group that may be substituted with: an aryl group(s), a heterocyclic group(s) or an aryloxy group(s), each of which may be substituted with a halogen atom(s); or with a group(s) represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ alkylsulfonyl group;
an arylsulfonyl group that may be substituted with a C₁₋₆ alkyl group(s) or a halogen atom(s);
a C₁₋₆ haloalkoxy group;
a C₁₋₆ haloalkylthio group;
a C₃₋₁₀ cycloalkyl group;
a C₂₋₆ alkenyl group;
a C₂₋₆ alkenyloxy group;
a C₂₋₆ alkenylthio group;
a C₁₋₆ alkoxy-C₁₋₆ alkoxy group;
an aryl group that may be substituted with 1 to 3 groups selected from the group consisting of: a C₁₋₆ alkyl group, a C₁₋₆ haloalkyl group, a halogen atom, a C₁₋₆ alkoxy group, a cyano group and a nitro group;
a heterocyclic group that may be substituted with a C₁₋₆ alkyl group(s) or a C₁₋₆ haloalkyl group(s);
an aryloxy group or arylthio group that may be substituted with a halogen atom(s) or a C₁₋₆ alkyl group(s);
a group represented by Formula —N(R⁶³)R⁷³ (where R⁶³ and R⁷³ each represent a hydrogen atom, a C₁₋₆ alkyl group, a C₁₋₆ hydroxyalkyl group, a C₁₋₆ alkoxy-C₁₋₆ alkyl group, an aryl group, a C₁₋₆ alkanoyl group, a di-C₁₋₆ alkylamino-C₂₋₆ alkanoyl group, a benzoyl group, or a heterocyclic group that may be substituted with a C₁₋₆ alkyl group(s), or R⁶³ and R⁷³, in combination with the adjacent nitrogen atom, form a cyclic amino group);
a hydroxyl group;
a cyano group;
a nitro group;
a C₁₋₆ alkanoyl group;
a C₁₋₆ alkanoyloxy group;
a C₁₋₆ alkanoyloxy-C₁₋₆ alkyl group;
a C₂₋₆ haloalkanoyl group;
a carboxyl group;
a C₂₋₆ alkoxycarbonyl group;
a C₂₋₆ cyclic amino group that may be substituted with an aryl group(s);
a group represented by Formula —CON(R⁶⁴)R⁷⁴ (where R⁶⁴ and R⁷⁴ each represent a hydrogen atom, a C₁₋₆ alkyl group, a C₁₋₆ alkoxy-C₁₋₆ alkyl group, or a heterocyclic group that may be substituted with a C₁₋₆ alkyl group(s), or R⁶⁴ and R⁷⁴, in combination with the adjacent nitrogen atom, form a cyclic amino group);
a group represented by Formula —SO₂N(R⁶²)R⁷² (where R⁶² and R⁷² each represent a hydrogen atom, a C₁₋₆ alkyl group, or R⁶² and R⁷², in combination with the adjacent nitrogen atom, form a cyclic amino group);
a C₁₋₆ alkylsulfenyl group;
a C₁₋₆ alkylsulfonyl group that may be substituted with a halogen atom(s); or
an arylsulfonyl group that may be substituted with a halogen atom(s),

R⁵⁶ represents

a hydrogen atom;
a halogen atom;
a C₁₋₁₀ alkyl group that may be substituted with an aryl group(s), a pyridyl group(s), a thienyl group(s) or a heterocyclic group(s), each of which may be substituted with a C₁₋₆ alkyl group(s) or a halogen atom(s);
a C₁₋₆ haloalkyl group;
a C₃₋₁₀ cycloalkyl group;
a C₁₋₁₀ alkoxy group;
a C₂₋₆ alkenyl group;
an aryl group that may be substituted with 1 to 3 groups selected from the group consisting of: a C₁₋₆ alkyl group, a halogen atom, a C₁₋₆ alkoxy group and a nitro group;
a heterocyclic group that may be substituted with a C₁₋₆ alkyl group(s);
a C₁₋₆ alkanoyl group;
a C₁₋₆ alkylsulfenyl group;
a C₁₋₆ alkylsulfonyl group;
an arylsulfonyl group that may be substituted with a halogen atom(s);
a hydroxyl group;
a cyano group; or
a nitro group,

R⁵⁷ represents

a hydrogen atom;
a C₁₋₁₀ alkyl group that may be substituted with a pyridyl group(s) or a thienyl group(s);
a C₁₋₆ haloalkyl group;
a C₃₋₁₀ cycloalkyl group;
a halogen atom;
a C₂₋₆ alkenyl group;
an aryl group that may be substituted with a halogen atom(s);
a C₁₋₁₀ alkoxy group;
a C₁₋₆ alkanoyl group; or
a C₁₋₆ alkylsulfenyl group, and

m represents an integer of 1 to 3),

a and b each represent 0 or 1, and

W represents —CO—, —CO—CO—, —CO—NH—, —CS—NH—, or —SO₂—],

or a pharmaceutically acceptable salt thereof, as an active ingredient.

According to another aspect of the present invention, there is provided a cannabinoid-receptor agonist comprising an imine compound represented by the following Formula (I-1)

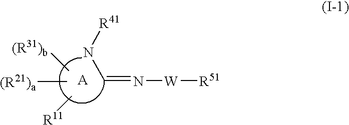

[where A¹ represents any one of the rings represented by the following formulas (where X represents an oxygen atom or a sulfur atom):

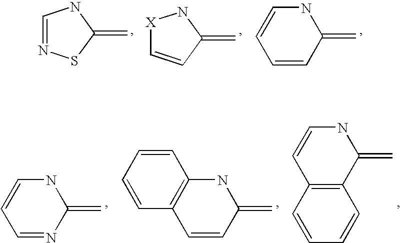

R¹¹ represents

a hydrogen atom;
a halogen atom;
a C₁₋₆ alkyl group
a C₂₋₆ alkenyl group;
a C₁₋₆ haloalkyl group;
a C₁₋₆ alkoxy group; or
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group),

R²¹ and R³¹ each represent

a hydrogen atom;
a halogen atom; or
a C₁₋₆ alkyl group,

R⁴¹ represents

a C₁₋₁₀ alkyl group or C₂₋₆ alkenyl group that may be substituted with: a halogen atom(s), a C₃₋₁₀ cycloalkyl group(s), an aryl group (s) or a C₁₋₆ alkoxy group,

R⁵¹ represents

a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, an aryl group or aryloxy group that may be substituted with 1 to 5 groups selected from the group consisting of a C₁₋₆ alkyl group, a C₁₋₆ alkoxy group, a C₁₋₆ haloalkyl group and a halogen atom, and a heterocyclic group;
a group represented by Formula (II-1)

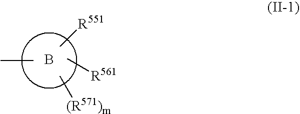

{where

B represents

a C₃₋₁₀ cycloalkyl group;
an aryl group;
a heterocyclic group;
a fluorenyl group; or
a group represented by Formula (IV-1)

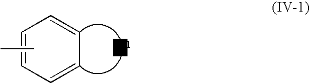

(where Y¹ represents —(CH₂)p—, or —O—(CH₂)_{q}—O—, in which p represents an integer of 2 to 4, and q represents an integer of 1 to 3),

R⁵⁵¹ and R⁵⁶¹ each represent

a hydrogen atom;
a halogen atom;
a C₁₋₁₀ alkyl group that may be substituted with: an aryl group(s), a heterocyclic group(s) or an aryloxy group(s), each of which may be substituted with a halogen atom(s); or with a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ haloalkoxy group;
a C₃₋₁₀ cycloalkyl group;
a C₂₋₆ alkenyl group;
an aryl group that may be substituted with 1 to 3 groups selected from the group consisting of: a C₁₋₆ alkyl group, a C₁₋₆ haloalkyl group, a halogen atom, a C₁₋₆ alkoxy group, and a nitro group;
a heterocyclic group that may be substituted with a C₁₋₆ alkyl group(s);
an aryloxy group that may be substituted with a halogen atom(s);
a group represented by Formula —N(R⁶³⁴)R⁷³⁴ (where R⁶³⁴ and R⁷³⁴ each represent a hydrogen atom, a C₁₋₆ alkyl group, an aryl group, or a C₁₋₆ alkanoyl group, or R⁶³⁴ and R⁷³⁴, in combination with the adjacent nitrogen atom, form a cyclic amino group);
a hydroxyl group;
a cyano group;
a nitro group;
a C₁₋₆ alkanoyl group;
a C₂₋₆ haloalkanoyl group;
a C₁₋₆ alkylsulfonyl group; or
an arylsulfonyl group that may be substituted with a halogen atom(s),

R⁵⁷¹ represents

a hydrogen atom;
C₁₋₁₀ alkyl group;
a C₁₋₁₀ alkoxy group; or
a halogen atom, and

m represents an integer of 1 to 3)

a and b each represent 0 or 1, and

W represents CO or SO₂],

or a pharmaceutically acceptable salt thereof, as an active ingredient.

According to another aspect of the present invention, there is provided an imine compound represented by the following Formula (I-1)

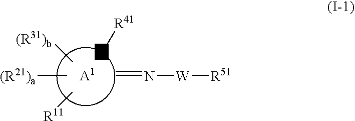

[where A¹ represents any one of the rings represented by the following formulas (where X represents an oxygen atom or a sulfur atom):

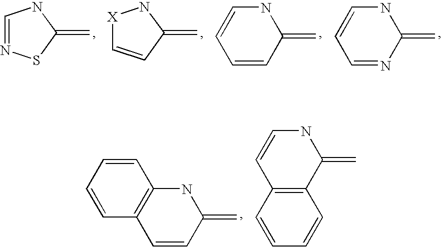

R¹¹ represents

a hydrogen atom;
a halogen atom;
a C₁₋₆ alkyl group
a C₂₋₆ alkenyl group;
a C₁₋₆ haloalkyl group;
a C₁₋₆ alkoxy group; or
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group),

R²¹ and R³¹ each represent

a hydrogen atom;
a halogen atom; or
a C₁₋₆ alkyl group,

R⁴¹ represents

a C₁₋₁₀ alkyl group or C₂₋₆ alkenyl group that may be substituted with: a halogen atom(s), a C₃₋₁₀ cycloalkyl group(s), an aryl group(s) or a C₁₋₆ alkoxy group(s),

R⁵¹ represents

a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, an aryl group or aryloxy group that may be substituted with 1 to groups selected from the group consisting of a C₁₋₆ alkyl group, a C₁₋₆ alkoxy group, a C₁₋₆ haloalkyl group and a halogen atom, and a heterocyclic group;
a group represented by Formula (II-1)

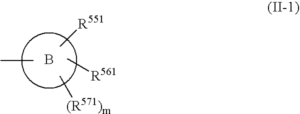

(where

B represents

a C₃₋₁₀ cycloalkyl group;
an aryl group;
a heterocyclic group;
a fluorenyl group; or
a group represented by Formula (IV-1)

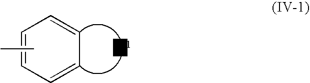

(where Y¹ represents —(CH₂)p—, or —O—(CH₂)_{q}—O—, in which p represents an integer of 2 to 4, and q represents an integer of 1 to 3),

R⁵⁵¹ and R⁵⁶¹ each represent

a hydrogen atom;
a halogen atom;
a C₁₋₁₀ alkyl group that may be substituted with: an aryl group(s), a heterocyclic group(s) or an aryloxy group(s), each of which may be substituted with a halogen atom(s); or with a group(s) represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ haloalkoxy group
a C₃₋₁₀ cycloalkyl group;
a C₂₋₆ alkenyl group;
an aryl group that may be substituted with 1 to 3 groups selected from the group consisting of: a C₁₋₆ alkyl group, a C₁₋₆ haloalkyl group, a halogen atom, a C₁₋₆ alkoxy group and a nitro group;
a heterocyclic group that may be substituted with a C₁₋₆ alkyl group(s);
an aryloxy group that may be substituted with a halogen atom(s);
a group represented by Formula —N(R⁶³⁴)R⁷³⁴ (where R⁶³⁴ and R⁷³⁴ each represent a hydrogen atom, a C₁₋₆ alkyl group, an aryl group, or a C₁₋₆ alkanoyl group, or R⁶³⁴ and R⁷³⁴, in combination with the adjacent nitrogen atom, form a cyclic amino group);
a hydroxyl group;
a cyano group;
a nitro group;
a C₁₋₆ alkanoyl group;
a C₂₋₆ haloalkanoyl group;
a C₁₋₆ alkylsulfonyl group; or
an arylsulfonyl group that may be substituted with a halogen atom(s),

R⁵⁷¹ represents

a hydrogen atom;
a halogen atom;
a C₁₋₁₀ alkyl group; or
a C₁₋₁₀ alkoxy group, and

m represents an integer of 1 to 3)

a and b each represent 0 or 1, and

W represents CO or SO₂],

or a pharmaceutically acceptable salt thereof.

According to another aspect of the present invention, there is provided a cannabinoid-receptor agonist comprising an imine compound represented by the Formula (I-2)

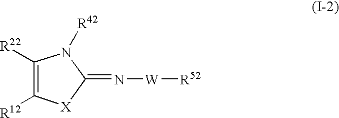

[where

R¹² and R²² each represent

a hydrogen atom;
a halogen atom;
a C₁₋₁₀ alkyl group;
a C₁₋₆ haloalkyl group;
a C₁₋₆ alkoxy group;
a carboxyl group;
a C₂₋₆ alkoxycarbonyl group;
a hydroxy-C₁₋₆ alkyl group;
an aryl group that may be substituted with 1 to 3 halogen atoms; or
a group represented by Formula —CON(R⁶¹)R⁷¹ (where R⁶¹ and R⁷¹ each represent a hydrogen atom or a C₁₋₆ alkyl group that may be substituted with a cyclic amino group(s), or R⁶¹ and R⁷¹, in combination with the adjacent nitrogen atom, form a cyclic amino group), or,

R¹¹ and R²², in combination with the adjacent carbon atom, form a benzene ring, a pyridine ring or a cyclohexenyl ring, each of which may be substituted with a C₁₋₆ alkyl group(s) or a halogen atom(s);

R⁴² represents

a C₁₋₁₀ alkyl group or C₂₋₆ alkenyl group that may be substituted with: a halogen atom (s), a cyano group(s), a carboxyl group(s), a C₂₋₆ alkoxycarbonyl group(s), a C₃₋₁₀ cycloalkyl group (s), an aryl group(s) that may be substituted with a C₁₋₆ haloalkyl group(s), a C₁₋₆ haloalkoxy group(s), a C₁₋₆ haloalkylthio group(s), a carboxyl group(s), a C₂₋₆ alkoxycarbonyl group(s) or a piperidinocarbamoyl group(s), an arylthio group(s), a C₁₋₆ alkoxy group(s), or a group(s) represented by Formula —CON(R⁶²)R⁷² (where R⁶² and R⁷² each represent a hydrogen atom or a C₁₋₆ alkyl group, or R⁶² and R⁷² in combination with the adjacent nitrogen atom, form a cyclic amino group); or
a C₂₋₆ alkynyl group,

R⁵² represents

a hydrogen atom;
a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, a C₂₋₆ alkoxycarbonyl group, a C₁₋₆ alkoxy group that may be substituted with a C₁₋₆ alkoxy group or an aryl group, a C₃₋₁₀ cycloalkoxy group that may be substituted with a C₁₋₆ alkyl group(s), an aryl group or aryloxy group that may be substituted with 1 to 5 groups selected from the group consisting of a C₁₋₆ alkyl group, a C₁₋₆ haloalkyl group, a C₁₋₆ alkoxy group, an aralkyloxy group, a nitro group and a halogen atom, a heterocyclic group, a phthalimide group, a C₁₋₆ alkanoyloxy group, an aralkyloxy group, a C₁₋₆ alkylthio group, an arylthio group and a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with: a C₁₋₆ alkyl group(s), a C₁₋₆ alkoxy group(s), C₂₋₆ alkoxycarbonyl group(s), or a C₁₋₆ haloalkyl group(s);
an aralkyloxy group;
a group represented by Formula (II-2)

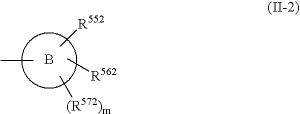

(where

B represents

a C₃₋₁₀ cycloalkyl group;
an aryl group;
a heterocyclic group;
a C₂₋₆ cyclic amino group;
a fluorenyl group;
a 2-oxopyrrolidinyl group
a group represented by Formula (III)

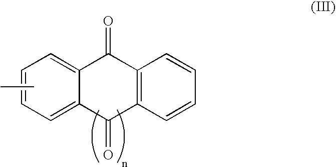

(where n represents 0 or 1); or
a group represented by Formula (IV-2)

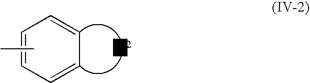

(where Y² represents —(CH₂)ₚ—, —CO—CH₂—CH₂—, —O—CH₂—CH═CH—, or —O—(CH₂)_{q}—O—, in which p represents an integer of 2 to 4, and q represents an integer of 1 to 3),

R⁵⁵² represents

a hydrogen atom;
a halogen atom;
a C₁₋₁₀ alkyl group that may be substituted with: an aryl group(s) that may be substituted with a halogen atom(s); with an aryloxy group(s); or with a group(s) represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ alkylsulfonyl group
an arylthio group that may be substituted with a C₁₋₆ alkyl group(s) or a halogen atom(s);
an arylsulfonyl group that may be substituted with a C₁₋₆ alkyl group(s) or a halogen atom(s);
a C₁₋₆ haloalkoxy group;
a C₁₋₆ haloalkylthio group;
a C₃₋₁₀ cycloalkyl group;
a C₂₋₆ alkenyl group;
a C₁₋₆ alkoxy-C₁₋₆ alkoxy group;
an aryl group that may be substituted with 1 to 3 groups selected from the group consisting of: a C₁₋₆ alkyl group, a C₁₋₆ haloalkyl group, a halogen atom, a C₁₋₆ alkoxy group and a nitro group;
a heterocyclic group that may be substituted with a C₁₋₆ alkyl group(s) or a C₁₋₆ haloalkyl group(s);
an aryloxy group that may be substituted with a halogen atom(s);
a group represented by Formula —N(R⁶³)R⁷³ (where R⁶³ and R⁷³ each represent a hydrogen atom, a C₁₋₆ alkyl group, a C₁₋₆ hydroxyalkyl group, a C₁₋₆ alkoxy-C₁₋₆ alkyl group, an aryl group, a C₁₋₆ alkanoyl group, or a benzoyl group, or R⁶³ and R⁷³, in combination with the adjacent nitrogen atom, form a cyclic amino group);
a hydroxyl group;
a cyano group;
a nitro group;
a carboxyl group;
a C₂₋₆ alkoxycarbonyl group;
a C₂₋₆ cyclic amino group that may be substituted with an aralkyl group(s) or an aryl group(s);
a group represented by Formula —CON(R⁶⁴)R⁷⁴ (where R⁶⁴ and R⁷⁴ each represent a hydrogen atom, a C₁₋₆ alkyl group, a C₁₋₆ alkoxy-C₁₋₆ alkyl group, or a heterocyclic group that may be substituted with a C₁₋₆ alkyl group(s), or R⁶⁴ and R⁷⁴, in combination with the adjacent nitrogen atom, form a cyclic amino group);
a group represented by Formula —SO₂N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ alkylsulfonyl group that may be substituted with a halogen atom(s);
an arylsulfonyl group that may be substituted with a halogen atom(s); or
a 2-oxa-3-oxobicyclo[2.2.1]heptyl group,

R⁵⁶² represents

a hydrogen atom;
a halogen atom;
a C₁₋₁₀ alkyl group;
a C₁₋₆ haloalkyl group; or
a C₁₋₆ alkoxy group,

R⁵⁷² represents

a hydrogen atom;
a C₁₋₁₀ alkyl group
a C₁₋₆ haloalkyl group;
a halogen atom; or
a C₁₋₆ alkoxy group;

m represents an integer of 1 to 3)

X represents an oxygen atom or a sulfur atom;

W represents CO or SO₂],

or a pharmaceutically acceptable salt thereof, as an active ingredient.

According to another aspect of the present invention, there is provided an imine compound represented by the following Formula (I-2)

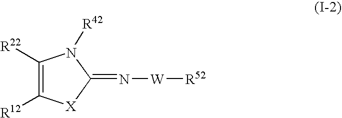

where

W is CO,

R¹² represents
a halogen atom;
a C₁₋₆ alkyl group;
a C₁₋₆ haloalkyl group;
a C₁₋₆ alkoxy group;
a carboxyl group;
a C₂₋₆ alkoxycarbonyl group;
a hydroxy-C₁₋₆ alkyl group; or
an aryl group that may be substituted with 1 to 3 halogen atoms; or
a group represented by Formula —CON(R⁶¹)R⁷¹ (where R⁶¹ and R⁷¹ each represent a hydrogen atom(s) or a C₁₋₆ alkyl group that may be substituted with a cyclic amino group(s), or R⁶¹ and R⁷¹, in combination with the adjacent nitrogen atom, form a cyclic amino group),

R²² represents

a hydrogen atom:
a halogen atom;
a C₁₋₁₀ alkyl group;
a C₁₋₆ haloalkyl group; or
an aryl group; or

R¹² and R²², in combination with the adjacent carbon atom, form a benzene ring, a pyridine ring or a cyclohexenyl ring, each of which may be substituted with a C₁₋₆ alkyl group(s) or a halogen atom(s);

R⁴² represents

a C₁₋₁₀ alkyl group or C₂₋₆ alkenyl group substituted with a C₃₋₁₀ cycloalkyl group(s) or a C₁₋₆ alkoxy group(s), and

X and R⁵² are as defined above,

or a pharmaceutically acceptable salt thereof.

According to another aspect of the present invention, there is provided a cannabinoid-receptor agonist comprising an imine compound represented by the Formula (I-3)

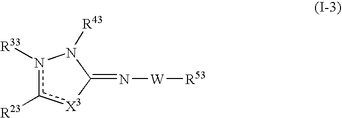

[where the broken line indicates that one of the bonds is a double bond,

X³ represents C(R¹³), S or O, R¹³, R²³ and R³³ each represent

a hydrogen atom;
a C₁₋₁₀ alkyl group that may be substituted with an aryl group(s) substituted with a halogen atom(s);
a C₁₋₆ haloalkyl group;
a C₃₋₁₀ cycloalkyl group; or
an aryl group or aralkyl group that may be substituted with 1 to 3 halogen atoms, or

in the case where X³ is C(R¹³) R¹³ and R²³ together represent a group represented by —CH₂—S—CH₂— (with the proviso that, R³³ is not substituted in the case where X³ is S or O),

R⁴³ represents

a 1,1-dioxothiolanyl group; or
a C₁₋₁₀ alkyl group or C₂₋₆ alkenyl group that may be substituted with a group(s) selected from the group consisting of: a C₃₋₁₀ cycloalkyl group, a C₁₋₆ haloalkyl group and a C₁₋₆ alkoxy group; or
an aryl group,

R⁵³ represents

a hydrogen atom;
a C₁₋₁₀ alkoxy group;
a C₁₋₆ alkoxy-C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a C₁₋₆ alkoxy group, a C₃₋₁₀ cycloalkyl group, a C₂₋₆ alkoxycarbonyl group, an aryl group or aryloxy group that may be substituted with 1 to 5 groups selected from the group consisting of a C₁₋₆ alkoxy group and a halogen atom, a heterocyclic group, a C₁₋₆ alkanoyloxy group, an aralkyloxy group, and a C₁₋₆ alkylthio group;
a group represented by Formula (II-3)

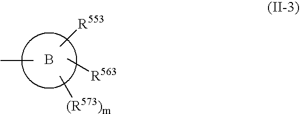

(where B represents
a C₃₋₁₀ cycloalkyl group;
an aryl group;
a heterocyclic group;
a C₂₋₆ cyclic amino group;
a group represented by Formula (III)

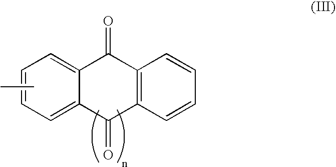

(where n represents 0 or 1); or
a group represented by Formula (IV-3)

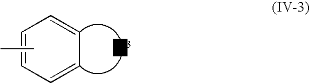

(where Y³ represents —O—CH₂—CH═CH— or —O—(CH₂)_{q}—O—, in which q represents an integer of 1 to 3),

R⁵⁵³ represents

a hydrogen atom;
a halogen atom;
an aryl group;
a C₁₋₁₀ alkyl group;
a C₁₋₆ alkanoyloxy-C₁₋₆ alkyl group;
a C₁₋₆ haloalkyl group;
a C₁₋₁₀ alkoxy group;
a C₁₋₆ alkylthio group;
a C₂₋₆ alkenyloxy group;
a C₂₋₆ alkenylthio group;
a C₁₋₆ haloalkoxy group;
a C₁₋₆ haloalkylthio group;
an aryl group that may be substituted with 1 to 3 halogen atoms or cyano groups;
a heterocyclic group;
an aryloxy group or arylthio group that may be substituted with a halogen atom(s) or a C₁₋₆ alkyl group(s);
a group represented by Formula —N(R⁶³³)R⁷³³ (where R⁶³³ and R⁷³³ each represent a hydrogen atom, a C₁₋₆ alkyl group, a C₁₋₆ alkanoyl group, a di-C₁₋₆ alkylamino-C₂₋₆ alkanoyl group, or a heterocyclic group that may be substituted with a C₁₋₆ alkyl group(s), or R⁶³³ and R⁷³³, in combination with the adjacent nitrogen atom, form a cyclic amino group);
a cyano group;
a nitro group; or
a C₂₋₆ alkoxycarbonyl group,

R⁵⁶³ represents

a hydrogen atom;
a halogen atom;
a C₁₋₁₀ alkyl group; or
a C₁₋₆ haloalkyl group,

R⁵⁷³ represents

a hydrogen atom;
a C₁₋₁₀ alkyl group;
a halogen atom; or
a C₁₋₁₀ alkoxy group,

m represents an integer of 1 to 3), and

W represents —CO—, —CO—CO—, —CO—NH—, —CS—NH— or —SO₂—],

or a pharmaceutically acceptable salt thereof, as an active ingredient.

According to another aspect of the present invention, there is provided an imine compound represented by Formula (I-3)

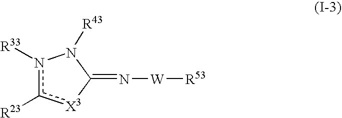

[where the broken line indicates that one of the bonds is a double bond,
X³ represents C(R¹³), S or O,

R¹³, R¹³ and R³³ each represent

a hydrogen atom;
a C₁₋₁₀ alkyl group that may be substituted with an aryl group(s) substituted with a halogen atom(s);
a C₁₋₆ haloalkyl group;
a C₃₋₁₀ cycloalkyl group; or
an aryl group or aralkyl group that may be substituted with 1 to 3 halogen atoms, or

in the case where X³ is C(R¹³) R¹³ and R²³ together represent a group represented by —CH₂—S—CH₂— (with the proviso that, R³³ is not substituted in the case where X³ is S or O),

R⁴³ represents

a 1,1-dioxothiolanyl group;
a C₁₋₁₀ alkyl group or C₂₋₆ alkenyl group that may be substituted with a group(s) selected from the group consisting of: a C₃₋₁₀ cycloalkyl group, a C₁₋₆ haloalkyl group and a C₁₋₆ alkoxy group; or
an aryl group,

R⁵³ represents

a hydrogen atom;
a C₁₋₁₀ alkoxy group;
a C₁₋₆ alkoxy-C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a C₁₋₆ alkoxy group, a C₃₋₁₀ cycloalkyl group, a C₂₋₆ alkoxycarbonyl group, an aryl group or aryloxy group that may be substituted with 1 to 5 groups selected from the group consisting of a C₁₋₆ alkoxy group and a halogen atom, a heterocyclic group, a C₁₋₆ alkanoyloxy group, an aralkyloxy group, and a C₁₋₆ alkylthio group;
a group represented by Formula (II-3)

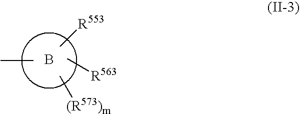

(where B represents
a C₃₋₁₀ cycloalkyl group;
an aryl group;
a heterocyclic group;
a C₂₋₆ cyclic amino group;
a group represented by Formula (III)

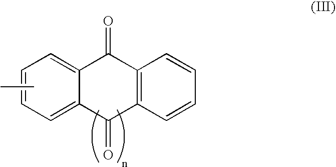

(where n represents 0 or 1); or
a group represented by Formula (IV-3)

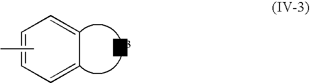

(where Y³ represents —O—CH₂—CH═CH— or —O—(CH₂)_{q}—O—, where q represents an integer of 1 to 3),

R⁵⁵³ represents

a hydrogen atom;
a halogen atom;
an aryl group;
a C₁₋₁₀ alkyl group;
a C₁₋₆ alkanoyloxy-C₁₋₆ alkyl group;
a C₁₋₆ haloalkyl group;
a C₁₋₁₀ alkoxy group;
a C₁₋₆ alkylthio group;
a C₂₋₆ alkenyloxy group;
a C₂₋₆ alkenylthio group;
a C₁₋₆ haloalkoxy group;
a C₁₋₆ haloalkylthio group,
an aryl group that may be substituted with 1 to 3 halogen atoms or cyano groups;
a heterocyclic group;
an aryloxy group or arylthio group that may be substituted with a halogen atom(s) or a C₁₋₆ alkyl group(s);
a group represented by Formula —N(R⁶³³)R⁷³³ (where R⁶³³ and R⁷³³ each represent a hydrogen atom, a C₁₋₆ alkyl group, a C₁₋₆ alkanoyl group, a di-C₁₋₆ alkylamino-C₂₋₆ alkanoyl group, or a heterocyclic group that may be substituted with a C₁₋₆ alkyl group(s), or R⁶³³ and R⁷³³, in combination with the adjacent nitrogen atom, form a cyclic amino group);
a cyano group;
a nitro group; or
a C₂₋₆ alkoxycarbonyl group;

R⁵⁶³ represents

a hydrogen atom;
a halogen atom;
a C₁₋₁₀ alkyl group; or
a C₁₋₆ haloalkyl group,

R⁵⁷³ represents

a hydrogen atom;
a C₁₋₁₀ alkyl group;
a halogen atom; or
a C₁₋₁₀ alkoxy group, and

m represents an integer of 1 to 3), and

W represents —CO—, —CO—CO—, —CO—NH—, —CS—NH— or —SO₂—],

or a pharmaceutically acceptable salt thereof.

In the present invention, R⁴, R″ R⁴² or R⁴³ is preferably a C₂₋₆ alkenyl group, or a C₁₋₁₀ alkyl group substituted with a C₃₋₁₀ cycloalkyl group(s) or a C₁₋₁₀ alkoxy group(s), and further preferably, a C₁₋₁₀ alkyl group substituted with a C₃₋₁₀ cycloalkyl group(s).

In the present invention, a preferred compound is one where R⁵, R⁵¹ R⁵² or R⁵³ is a C₁₋₁₀ alkyl group or C₂₋₆ alkenyl group that may be substituted with 1 to 3 groups selected from the group consisting of: a C₃₋₁₀ cycloalkyl group, an aryl group that may be substituted with a C₁₋₆ haloalkoxy group(s) or a halogen atom (s), a thienyl group, a halogen atom and an aryloxy group; or is a group represented by Formula (II), (II-1), (II-2) or (II-3);

R⁵⁵, R⁵⁵¹, R⁵⁵² or R⁵⁵³ is a hydrogen atom; a halogen atom; a C₁₋₁₀ alkyl group; a C₁₋₆ haloalkyl group; a C₁₋₁₀ alkoxy group; a C₁₋₆ haloalkoxy group; a C₃₋₁₀ cycloalkyl group; an aryl group; a heterocyclic group that may be substituted with a C₁₋₆ alkyl group(s); an aryloxy group; a morpholino group; an arylamino group; a cyano group; a C₁₋₆ alkanoyl group; a C₁₋₂₆ haloalkanoyl group; or a C₁₋₆ alkylsulfonyl group, R⁵⁶, R⁵⁶¹ R⁵⁶² or R⁵⁶³ is a hydrogen atom; a halogen atom; a C₁₋₆ haloalkyl group; or C₁₋₆ alkoxy group, and R⁵⁷, R⁵⁷¹ R⁵⁷² or R⁵⁷³ is a hydrogen atom; a halogen atom; a C₁₋₁₀ alkyl group; or a C₁₋₁₀ alkoxy group.

Furthermore, a preferable compound is one where each of R⁵, R⁵¹, R⁵² or R⁵³ is a group represented by Formula (II), (II-1) (II-2) or (II-3), B is a phenyl group, R⁵⁵, R⁵⁵¹ R⁵⁵² or R⁵⁵³ is a halogen atom; a C₁₋₁₀ alkyl group; a C₁₋₆ haloalkyl group; a C₁₋₆ alkoxy group; a C₁₋₆ haloalkoxy group; a C₃₋₈ cycloalkyl group; an aryl group; an aryloxy group; a morpholino group; an arylamino group; a cyano group; a C₁₋₆ alkanoyl group; a C₂₋₆ haloalkanoyl group; or a C₁₋₆ alkylsulfonyl group, R⁵⁶, R⁵⁶¹, R⁵⁶² or R⁵⁶³ is a hydrogen atom; a halogen atom; a C₁₋₆ haloalkyl group; or C₁₋₆ alkoxy group, and R⁵⁷, R⁵⁷¹ R⁵⁷² or R⁵⁷³ is a hydrogen atom; a halogen atom; a C₁₋₆ alkyl group; or C₁₋₆ alkoxy group; and m is 1. Moreover, the most preferable compound is one where R⁵, R⁵¹, R⁵² or R⁴³ is a phenyl group substituted with 1 to 3 groups selected from the group consisting of a halogen atom, a C₁₋₆ alkyl group, a C₁₋₆ haloalkyl group, a C₁₋₆ alkoxy group, a cyano group and a C₁₋₆ haloalkoxy group.

In formulas (I), (I-1), (I-2) and (I-3), the double bond made of the carbon atom and the nitrogen atom contained in the group represented by >C═N—CO— is preferably in (Z) configuration.

The imine compounds of the present invention include their prodrugs, hydrates and solvates.

The meaning of the terms used in the present specification will be explained below.

In the present invention, the term “C_{X-Y}” means that the group following the term has X to Y carbon atoms.

The term “halogen atom” refers to fluorine, chlorine, bromine or iodine.

The term “C₁₋₆ alkyl group” refers to a straight or branched alkyl group having 1 to 6 carbon atoms, and includes, for example, methyl group, ethyl group, n-propyl group, isopropyl group, n-butyl group, 2-butyl group, t-butyl group, 1,1-dimethylethyl group, n-pentyl group, isopentyl group, 1,1-dimethylpropyl group and n-hexyl group.

The term “C₁₋₁₀ alkyl group” refers to a straight or branched alkyl group having 1 to 10 carbon atoms, and includes, in addition to the specific examples mentioned above regarding the “C₁₋₆ alkyl group”, 1,1,3,3-tetramethylbutyl group, n-nonanyl group and n-decyl group.

The term “C₁₋₆ haloalkyl group” refers to an alkyl group wherein the “C₁₋₆ alkyl group” as defined above is substituted with one or more halogen atoms, and includes, for example, fluoromethyl group, difluoromethyl group, trifluoromethyl group, 2,2,2-trifluoroethyl group, 2,2,2-trichloroethyl group, pentafluoroethyl group, 3,3,3-trifluoropropyl group, perfluoropropyl group, 4-fluorobutyl group, 4-chlorobutyl group, 4-bromobutyl group and perfluorohexyl group.

The term “C₁₋₆ alkoxy group” refers to a straight or branched alkoxy group having 1 to 6 carbon atoms, and includes, for example, methoxy group, ethoxy group, 1-propoxy group, isopropoxy group 1-butoxy group, 1-methyl-1-propoxy group, t-butoxy group and 1-pentyloxy group.

The term “C₁₋₁₀ alkoxy group” refers to a straight or branched alkoxy group having 1 to 10 carbon atoms, and includes, in addition to the specific examples mentioned above regarding the “C₁₋₆ alkoxy group”, 1,1,3,3-tetramethylbutoxy group and n-decyloxy group.

The term “aryl group” refers to a mono- to tetracyclic aromatic carbocyclic group having 6 to 18 carbon atoms, and includes, for example, phenyl group, naphthyl group, anthoryl group, phenanthrenyl group, tetracenyl group and pyrenyl group. A phenyl group is preferred.

The term “C₃₋₁₀ cycloalkyl group” refers to a cycloalkyl group having 3 to 10 carbon atoms, and includes, for example, cyclopropyl group, cyclobutyl group, cyclopentyl group, cyclohexyl group, cycloheptyl group and adamantyl group.

The term “C₂₋₆ alkenyl group” refers to a straight or branched alkyl group having 2 to 6 carbon atoms and one or more double bonds at an arbitrary position of the “alkyl group” as defined above, and includes, for example, vinyl group, 1-propenyl group, 2-propenyl group, 2-butenyl group, 1,3-butadienyl group, 2-pentenyl group, 3-pentel group and 2-hexenyl group.

The term “C₂₋₆ alkynyl group” refers to a straight or branched alkynyl group having 2 to 6 carbon atoms, and includes, for example, ethynyl group. 1-propynyl group and 2-propynyl group.

The term “C₂₋₆ alkoxycarbonyl group” refers to a group wherein the alkoxyl group as defined above is attached to a carbonyl group, and includes, for example, methoxycarbonyl group, ethoxycarbonyl group and t-butoxycarbonyl group.

The term “hydroxy-C₁₋₆ alkyl group” refers to a group wherein the C₁₋₆ alkyl group as defined above is substituted with 1 to 2 hydroxyl groups and includes, for example, hydroxymethyl group, 2-hydroxyethyl group and 4-hydroxybutyl group.

The term “cyclic amino group” refers to a cyclic amino group having 2 to 6 carbon atoms, and includes, for example, pyrrolidino group, piperidino group, piperazino group, morpholino group and thiomorpholino group. In the present invention, the term thiomorpholino group includes its sulfur-dioxide form.

The term “C₁₋₆ haloalkoxy group” refers to an alkoxy group wherein the “C₁₋₆ alkoxyl group” as defined above is substituted with one or more halogen atoms, and includes, for example, fluoromethoxy group difluoromethoxy group, trifluoromethoxy group, 2,2,2-trifluoroethoxy group, 2,2,2-trichloroethoxy group, pentafluoroethoxy group, perfluoropropoxy group, 4-fluorobutoxy group, 4-chlorobutoxy group, 4-bromobutoxy group and perfluorohexyloxy group.

The term “C₁₋₆ alkylthio group” refers to a straight or branched alkylthio group having 1 to 6 carbon atoms, and includes, for example, methylthio group, ethylthio group, n-propylthio group, isopropylthio group, n-butylthio group, 2-butylthio group, t-butylthio group, 1,1-dimethylethylthio group, n-pentylthio group, isopentylthio group 1,1-dimethylpropylthio group and n-hexylthio group.

The term “C₁₋₆ haloalkylthio group” refers to an alkylthio group wherein the C₁₋₆ alkylthio group as defined above is substituted with one or more halogen atoms, and includes, for example, fluoromethylthio group, difluoromethylthio group, trifluoromethylthio group, 2,2,2-trifluoroethylthio group, 2,2,2-trichloroethylthio group, pentafluoroethylthio group, 4-fluorobutylthio group, 4-chlorobutylthio group, 4-bromobutylthio group and perfluorohexylthio group.

Example of “arylthio group” may include phenylthio group and naphthylthio group.

The term “C₁₋₆ alkenylthio group” refers to a straight or branched alkenylthio group having 2 to 6 carbon atoms, and includes, for example, vinylthio group, 1-propenylthio group, 2-propenylthio group, 2-butenylthio group, 1,3-butadienylthio group, 2-pentenylthio group, 3-pentenylthio group and 2-hexenylthio group.

The term “C₁₋₆ alkanoyl group” refers to a straight or branched alkanoyl group having 1 to 6 carbon atoms, and includes, for example, formyl group, acetyl group, propionyl group, isopropionyl group, butyryl group and pivaloyl group.

The term “C₁₋₆ alkanoyloxy group” refers to a group wherein the C₁₋₆ alkanoyl group as defined above is attached to an oxy group, and includes, for example, acetyloxy group, propionyloxy group and pivaloyloxy group.

The term “C₁₋₆ alkanoyloxy-C₁₋₆ alkyl group” refers to a group wherein the C₁₋₆ alkanoyloxy as defined above is attached to a C₁₋₆ alkyl group, and includes, for example, acetyloxyethyl group, propionyloxymethyl group and pivaloyloxymethyl group.

The term “C₂₋₆ haloalkanoyl group” refers to an alkanoyl group wherein the “C₂₋₆ alkanoyl group” is substituted with a halogen atom(s), and includes, for example, fluoroacetyl group, trifluoroacetyl group, 2,2,2-trifluoropropionyl group, 2,2,2-trichloropropionyl group, 4-fluorobutryl group, 4-chlorobutyryl group and 4-bromobutyryl group.

The term “C₁₋₆ alkoxy-C₁₋₆ alkoxy group” refers to a group formed by binding two C₁₋₆ alkoxy groups, and includes, for example, methoxymethoxy group, methoxypropoxy group, ethoxypropoxy group and heptyloxyethoxy group.

The term “C₁₋₆ alkoxy-C₁₋₆ alkyl group” refers to a group formed by binding a C₁₋₆ alkoxy group and a C₁₋₆ alkyl group, and includes, for example, methoxymethyl group, methoxypropyl group, ethoxypropyl group and heptyloxyethyl group.

The term “aryloxy group” refers to a group having an oxygen atom and the aryl group as defined above to be attached via said oxygen atom to another group, and includes, for example, phenoxy group and naphthoxy group.

The term “aralkyl group” refers to a group formed by binding an aryl group and an alkyl group, and includes, for example, a benzyl group, phenethyl group and naphthylmethyl group.

The term “aralkyloxy group” refers to a group formed by binding an aralkyl group and an oxy group, and includes, for example, benzyloxy group, phenethyloxy group and naphthylmethoxy group.

The term “heterocyclic group” refers to a heteromonocyclic group or a fused heterocyclic group containing 1 to 3 atoms selected from the group consisting of a nitrogen atom, oxygen atom and sulfur atom as a ring constituent atom, and includes a saturated heterocyclic group, an aromatic heterocyclic group, and a fused heterocyclic group having a partially saturated aromatic heteromonocyclic group. Furthermore, the fused heterocyclic group having a partially saturated aromatic heteromonocyclic group can be substituted with ═O. As the heterocyclic group, a heterocyclic group having 5 to 10 atoms in the cycle is preferred.

Examples of the saturated heterocyclic group may include aziridinyl group, azetidinyl group, pyrrolidinyl group, imidazolidyl group, pyrazolidinyl group, oxolanyl group, thiolanyl group, piperidinyl group, piperazinyl group and morpholinyl group

Examples of the aromatic heterocyclic group may include pyridyl group, pyridazinyl group, pyrimidinyl group, pyrazinyl group, quinolyl group, isoquinolyl group, thienyl group (for example, 2-thienyl group, 3-thienyl group), pyrrolyl group (for example, 1-pyrrolyl group, 2-pyrrolyl group, 3-pyrrolyl group), thiazolyl group (for example, 2-thiazolyl group, 4-thiazolyl group, 5-thiazolyl group), isothiazolyl group (for example, 3-isothiazolyl group, 4-isothiazolyl group, 5-isothiazolyl group), pyrazolyl group (for example, 1-pyrazolyl group, 3-pyrazolyl group, 4-pyrazolyl group), imidazolyl group (for example, 1-imidazolyl group, 2-imidazolyl group, 3-imidazolyl group), furyl group (for example, 2-furyl group, 3-furyl group), oxazolyl group (for example, 2-oxazolyl group, 4-oxazolyl group, 5-oxazolyl group), isoxazolyl group (for example, 3-isoxazolyl group, 4-isoxazolyl group, 5-isoxazolyl group), oxadiazolyl group (for example, 1,2,3-oxadiazolyl group, 1,3,4-oxadiazolyl group), thiadiazolyl group (for example, 1,2,3-thiadiazolyl bases, 1,3,4-thiadiazolyl group), triazolyl group (for example, 1,2,4-triazolyl group), benzofuranyl group (for example, 2-benzofuranyl group, 3-benzofuranyl group, 4-benzofuranyl group, 5-benzofuranyl group), benzothienyl group (for example, 2-benzothienyl group, 3-benzothienyl group, 4-benzothienyl group, 5-benzothienyl group), indolyl group (for example, 2-indolyl group, 3-indolyl group, 4-indolyl group, 5-indolyl group), benzoxazolyl group (for example, 2-benzoxazolyl group, 4-benzoxazolyl group, 5-benzoxazolyl group, 6-benzoxazolyl group), benzisoxazolyl group (for example, 3-benzo[c]isoxazolyl group, 4-benzo[c]isoxazolyl group, 5-benzo[c]isoxazolyl group, 6-benzo[c]isoxazolyl group, 3-benzo[d]isoxazolyl group, 4-benzo[d]isoxazolyl group, 5-benzo[d]isoxazolyl group, 6-benzo[d] isoxazolyl group), indazolyl group (for example, 3-indazolyl group, 4-indazolyl group, 5-indazolyl group, 6-indazolyl group), benzimidazolyl group (for example, 2-benzimidazolyl group, 4-benzimidazolyl group, 5-benzimidazolyl group, 6-benzimidazolyl group), benzoxadiazolyl group (for example, 4-benzo[1,2,5]oxadiazolyl group, 5-benzo[1,2,5]oxadiazolyl group, 4-benzo[1,2,3]oxadiazolyl group, 5-benzo[1,2,3]oxadiazolyl group), benzothiadiazolyl group (for example, 4-benzo[1,2,5]thiadiazolyl group, 5-benzo[1,2,5]thiadiazolyl group, 4-benzo[1,2,3]thiadiazolyl group, 5-benzo[1,2,3]thiadiazolyl group), indolidinyl group (for example, 1-indolidinyl group, 2-indolidinyl group, 3-indolidinyl group, 5-indolidinyl group), thienopyridyl group (for example, 2-thieno[2,3-b]pyridyl group, 3-thieno[2,3-b]pyridyl group, 5-thieno[2,3-b]pyridyl group, 6-thieno[2,3-b]pyridyl group, 2-thieno[3,2-b]pyridyl group, 3-thieno[3,2-b]pyridyl group, 5-thieno[3,2-b]pyridyl group, 6-thieno[3,2-b]pyridyl group), pyrazolopyridyl group (for example, 2-pyrazolopyridyl group, 3-pyrazolopyridyl group, 5-pyrazolopyridyl group, 6-pyrazolo pyridyl group), imidazopyridyl group (for example, 1-imidazo[1,5-a]pyridyl group, 3-imidazo[1,5-a]pyridyl group, 5-imidazo[1,5-a]pyridyl group, 7-imidazo[1,5-a]pyridyl group, 2-imidazo[1,2-a]pyridyl group, 3-imidazo[1,2-a]pyridyl group, 5-imidazo[1,2-a]pyridyl group, 7-imidazo[1,2-a]pyridyl group), imidazopyrazyl group (for example, 1-imidazo[1,5-a]pyrazyl group, 3-imidazo[1,5-a]pyrazyl group, 5-imidazo[1,5-a]pyrazyl group, 8-imidazo[1,5-a]pyrazyl group, 2-imidazo[1,2-a]pyrazyl group, 3-imidazo[1,2-a]pyrazyl group, 5-imidazo[1,2-a]pyrazyl group, 8-imidazo[1,2-a]pyrazyl group), pyrazolopyrimidyl group (for example, 2-pyrazolo[1,5-a]pyrimidyl, 3-pyrazolo[1,5-a]pyrimidyl group, 5-pyrazolo[1,5-a]pyrimidyl group, 6-pyrazolo[1,5-a]pyrimidyl group, 2-pyrazolo[1,5-c]pyrimidyl group, 3-pyrazolo[1,5-c]pyrimidyl group, 4-pyrazolo[1,5-c]pyrimidyl group, 5-pyrazolo[1,5-c]pyrimidyl group), triazolopyrimidyl group (for example, 3-[1,2,3]triazolo[1,5-a]pyrimidyl group, 5-[1,2,3]triazolo[1,5-a]pyrimidyl group, 6-[1,2,3]triazolo[1,5-a]pyrimidyl group, 3-[1,2,3]triazolo[1,5-c]pyrimidyl group, 4-[1,2,3]triazolo[1,5-c]pyrimidyl group, 5-[1,2,3]triazolo[1,5-c]pyrimidyl group, 2-[1,2,4]triazolo[1,5-a]pyrimidyl group, 5-[1,2,4]triazolo[1,5-a]pyrimidyl group, 6-[1,2,4]triazolo[1,5-a]pyrimidyl group, 7-[1,2,4]triazolo[1,5-a]pyrimidyl group, 2-[1,2,4]triazolo[1,5-c]pyrimidyl group, 5-[1,2,4]triazolo[1,5-c]pyrimidyl group, 7-[1,2,4]triazolo[1,5-c]pyrimidyl group, 8-[1,2,4]triazolo[1,5-c]pyrimidyl group), thienothienyl group (for example, 2-thieno[2,3-b]thienyl group, 3-thieno[2,3-b]thienyl group, 2-thieno[3,2-b]thienyl group, 3-thieno[3,2-b]thienyl group), imidazothiazolyl group (for example, 2-imidazo[2,1-b]thiazolyl group, 3-imidazo[2,1-b]thiazolyl group, 5-imidazo[2,1-b]thiazolyl group, 2-imidazo[5,1-b]thiazolyl group, 3-imidazo[5,1-b]thiazolyl group, and 5-imidazo[5,1-b]thiazolyl group).

Examples of the fused heterocyclic group having a partially saturated aromatic heteromonocyclic group may include tetrahydrobenzofuranyl group, tetrahydrobenzothienyl group, tetrahydrobenzopyrrolyl group, 2,3-dihydro-1H-benzofuranyl group, 2,3-dihydro-1H-benzothienyl group, 2,3-dihydro-1H-indolyl group, 2,3-dihydro-1H-indazolyl group, 2,3-dihydro-1H-benzotriazolyl group, 2,3-dihydro-1H-benzoxazolyl group, 2,3-dihydro-1H-benzothiazolyl group, benzo[1,3]oxathiolyl group, benzo[1,3]dioxolyl group, 2H-chromenyl group, chromanyl group, indolynyl group and isoindolynyl group.

Examples of the fused heterocyclic group having the partially saturated monocycle and substituted with ═O may include

2-oxo-1,3-dihydro-1H-indolyl ring, 3-oxo-1,2-dihydro-1H-indazolyl ring, 2-oxo-3H-benzoxazolyl ring, 2-oxo-3H-benzothiazolyl ring, 2-oxo-benz[1,3]oxathiolyl ring, 2-oxo-benz[1,3]dioxolyl ring and 2-oxo-chlomenyl ring.

Preferable examples of the heterocyclic ring of B ring may include pyridyl group, pyridazinyl group, pyrimidinyl group, pyrazinyl group, quinolyl group and isoquinolyl group.

The term “C₁₋₆ alkylsulfenyl group” refers to a group having SO and the “C₁₋₆ alkyl group(s)” as defined above to be attached via said SO to another group, and includes, for example, methylsulfenyl group, ethylsulfenyl group, n-propylsulfenyl group, n-butylsulfenyl group, t-butylsulfenyl group and n-pentylsulfenyl group.

The term “C₁₋₆ alkylsulfonyl group that may be substituted with a halogen atom(s)” refers to a group having a sulfonyl group and the “C₁₋₆ alkyl group(s)” as defined above or the “C₁₋₆ haloalkyl group(s)” as defined above to be attached via said sulfonyl group to another group. Examples thereof may include methylsulfonyl group, ethylsulfonyl group, n-propylsulfonyl group, n-butylsulfonyl group, t-butylsulfonyl group, n-pentylsulfonyl group, fluoromethylsulfonyl group, difluoromethylsulfonyl group, trifluoromethylsulfonyl group, 2,2,2-trifluoroethylsulfonyl group, 2,2,2-trichloroethylsulfonyl group, pentafluoroethylsulfonyl group, 4-fluorobutylsulfonyl group, 4-chlorobutylsulfonyl group and 4-bromobutylsulfonyl group.

The term “arylsulfonyl group that may be substituted with a halogen atom(s)” refers to an arylsulfonyl group wherein the aryl may be substituted with a halogen atom(s). Examples thereof may include phenylsulfonyl group, 4-chlorophenylsulfonyl group, 4-fluorophenylsulfonyl group, 2,4-dibromophenylsulfonyl group, 2,4-difluorophenylsulfonyl group, naphthylsulfonyl group and 6-bromonaphthylsulfonyl group.

The term “prodrug” refers to a compound that is hydrolyzed in vivo to regenerate an imine compound having a cannabinoid receptor agonist effect.

The term “pharmaceutically acceptable salt” refers to an acid addition salt or a base addition salt. Examples of the acid addition salt may include inorganic salts such as hydrochloride, hydrobromate and sulfate, and organic salts such as citrate, oxalate, malate, tartrate, fumarate, maleate, methanesulfonate, ethanesulfonate, benzenesulfonate, para-toluenesulfonate, benzoate, aspartate and glutamate. Examples of the base addition salt may include inorganic base salts such as a sodium salt, calcium salt, magnesium salt, calcium salt and aluminum salt, and organic salts such as an ethanolamine salt, lysine salt, ornithine salt, meglumine salt and trishydroxymethylaminomethane salt, and ammonium salts.

### BEST MODE FOR CARRYING OUT THE INVENTION

A compound according to the present invention can be produced by the following (1) to (4) steps.

(1) A compound (Ia) according to the present invention can be produced from an amine compound (V) by a process represented by the following reaction scheme.

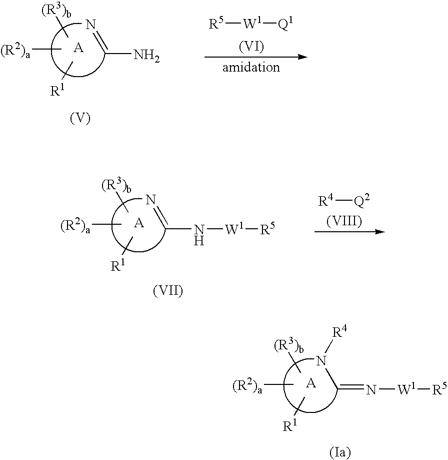

[where A, R¹, R², R³, R⁴, R⁵, a and b are the same as defined above; Q¹ represents a hydroxyl group or a halogen atom such as chlorine atom and bromine atom; Q² represents a leaving group such as a chlorine atom, bromine atom, iodine atom, methanesulfonyloxy group, trifluoromethanesulfonyloxy group or para-toluenesulfonyloxy group; and W¹ represents —CO—, —CO—CO— or —SO₂—].

Step 1: Production of Amido Compound (VII)

(i) An amino compound (VII) can be produced by an amidation reaction using an amine compound (V) and a compound (VI).

When Q¹ of a compound (VI) is a halogen atom, the reaction is preferably performed in the presence of a base. Examples of the base to be used may include alkali metal hydroxides (such as lithium hydroxide, sodium hydroxide, potassium hydroxide), alkali metal carbonates (such as lithium carbonate, sodium carbonate, potassium carbonate), alkali metal bicarbonates (such as sodium hydrogencarbonate, potassium hydrogencarbonate), and organic bases (such as triethylamine, diisopropylethylamine, tri-n-butylamine, 1,5-diazabicyclo[4.3.0]-5-nonene, 1,8-diazabicyclo[5.4.0]-7-undecene, pyridine, N,N-dimethylaminopyridine).

As the reaction temperature, a cooling temperature to the boiling point of the solvent or reagent to be used, in particular, a temperature from −20° C. to room temperature is preferable.

The reaction can be performed in the presence or absence of a solvent. Examples of the solvent to be used may include dioxane, tetrahydrofuran, diethyl ether, petroleum ether, n-hexane, cyclohexane, benzene, toluene, xylene, chlorobenzene, pyridine, acetonitrile, ethyl acetate, ethylmethylketone, N,N-dimethylformamide, dimethylsulfoxide, dichloromethane, chloroform, carbon tetrachloride and water.

In the reaction, it is preferable that the type of solvent and reagent to be used and amounts thereof may be appropriately selected, depending on the substrate to be used in the reaction and reaction conditions.

When Q¹ of the compound (VI) is a hydroxyl group, a condensing agent is preferably used. Examples of the condensing agent may include acid halogenating agents such as thionyl chloride and oxalyl chloride, alkyl chlorocarbonates such as ethyl chlorocarbonate, carbodiimide compounds such as dicyclohexylcarbodiimide, 1-ethyl-3-(3-dimethylamino)propylcarbodiimide, sulfonyl chloride compounds such as methanesulfonyl chloride, phosphorus compounds such as diphenyl phosphite, diphenylphosphoryl chloride, triphenylphosphine-diethylazodicarboxylate, and N,N′-carbodiimidazole.

The reaction can be performed in the presence or absence of a solvent. Examples of the solvent to be used include methanol, ethanol, n-propanol, isopropanol, n-butanol, t-butanol, dioxane, tetrahydrofuran, diethyl ether, petroleum ether, n-hexane, cyclohexane, benzene, toluene, xylene, chlorobenzene, pyridine, ethyl acetate, N,N-dimethylformamide, dimethylsulfoxide, dichloromethane, chloroform, carbon tetrachloride and water.

In the reaction, it is preferable that the type of solvent and reagent to be used and amounts thereof may be appropriately selected depending on the substrate to be used in the reaction and reaction conditions.

(ii) When W¹ of the compound (VI) is —CO— or —CO—CO—, the amide compound (VII) can be produced by using an acid anhydride of the compound (VI) or mixed acid anhydride in place of the compound (VI).

The reaction is preferably performed in the presence of a base. Examples of the base to be used may include alkali metal hydroxides (such as lithium hydroxide, sodium hydroxide, potassium hydroxide), alkali metal carbonates (such as lithium carbonate, sodium carbonate, potassium carbonate), alkali metal bicarbonates (such as sodium hydrogencarbonate, potassium hydrogencarbonate), and organic bases (such as triethylamine, diisopropylethylamine, tri-n-butylamine, 1,5-diazabicyclo[4.3.0]-5-nonene, 1,8-diazabicyclo[5.4.0]-7-undecene, pyridine, N,N-dimethylaminopyridine).

As the reaction temperature, a cooling temperature to the boiling point of the solvent or reagent to be used is preferable.

The reaction can be performed in the presence or absence of a solvent. Examples of the solvent to be used may include methanol, ethanol, n-propanol, isopropanol, n-butanol, t-butanol, dioxane, tetrahydrofuran, diethyl ether, petroleum ether, n-hexane, cyclohexane, benzene, toluene, xylene, chlorobenzene, pyridine, acetonitrile, ethyl acetate, ethylmethylketone, N,N-dimethylformamide, dimethylsulfoxide, dichloromethane, chloroform, carbon tetrachloride, and water.

In the reaction, it is preferable that the type of solvent and reagent to be used and amounts thereof may be appropriately selected depending on the substrate to be used in the reaction and reaction conditions.

Step 2: Production of the Compound (Ia) of the Present Invention

The compound (Ia) of the present invention can be produced by reacting an amide compound (VII) and a compound (VIII).

The reaction is preferably performed in the presence of a base. Examples of the base to be used may include alkali metal hydroxides (such as lithium hydroxide, sodium hydroxide, potassium hydroxide), alkali metal carbonates (such as lithium carbonate, sodium carbonate, potassium carbonate), alkali metal bicarbonates (such as sodium hydrogencarbonate, potassium hydrogencarbonate), alkali metal hydrides (such as sodium hydride, potassium hydride), alkali metals (such as metallic sodium, metallic potassium), organic bases (such as triethylamine, diisopropylethylamine, tri-n-butylamine, 1,5-diazabicyclo[4.3.0]-5-nonene, 1,8-diazabicyclo[5.4.0]-7-undecene, pyridine, N,N-dimethylaminopyridine), alkali metal amides (such as sodium amide), alkali metal alkoxides (such as sodium methoxide, sodium ethoxide, t-butoxy potassium), organometallic compounds (such as n-butyllithium, s-butyllithium, t-butyllithium, lithium diisopropylamide, sodium bis(trimethylsilyl)amide).

As the reaction temperature, a cooling temperature to the boiling point of the solvent or reagent to be used is preferable.

The reaction can be performed in the presence or absence of a solvent. Examples of the solvent to be used may include methanol, ethanol, n-propanol, isopropanol, n-butanol, t-butanol, dioxane, tetrahydrofuran, diethyl ether, petroleum ether, n-hexane, cyclohexane, benzene, toluene, xylene, chlorobenzene, pyridine, ethyl acetate, N,N-dimethylformamide, dimethylsulfoxide, dichloromethane, chloroform, and carbon tetrachloride.

In the reaction, it is preferable that the type of solvent and reagent to be used and amounts thereof may be appropriately selected depending on the substrate to be used in the reaction and reaction conditions.

(2) A compound according to the present invention where W is —CO—NH— or —CS—NH— can be produced by use of an amide compound (VII) where W¹ is —CO—NH— or —CS—NH— in a similar manner to the process shown in Step 2 of Section (1).

A compound where W¹ of an amide compound (VII) is —CO—NH— or —CS—NH— can be produced by use of an amine compound (V) and a compound: R⁵—NCO and a compound: R⁵—NCS.

The reaction can be performed in the presence or absence of a solvent.

As the reaction temperature, a cooling temperature to the boiling point of the solvent or reagent to be used is preferable. Examples of the solvent to be used may include dioxane, tetrahydrofuran, diethyl ether, petroleum ether, n-hexane, cyclohexane, benzene, toluene, xylene, chlorobenzene, pyridine, acetonitrile, ethyl acetate, ethylmethylketone, N,N-dimethylformamide, dimethylsulfoxide, dichloromethane, chloroform, and carbon tetrachloride.

In the reaction, it is preferable that the type of solvent and reagent to be used and amounts thereof may be appropriately selected depending on the substrate to be used in the reaction and reaction conditions.

(3) A compound (I) according to the present invention can be produced by using an imine compound represented by the following formula (IX)

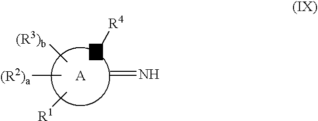

(where R¹, R², R³, R⁴, a and b are the same as defined above) and a compound (VI), a compound: R⁵—N═C═O or a compound: R⁵—N═C═S, in the same manner as the process shown in Step 1 of Section (1) or the process shown in Section (2).

An imine compound (IX) can be produced by hydrolyzing a compound (I) according to the present invention. In particular, R⁵ is preferably a hydrogen atom, a C₁₋₁₀ alkyl group, or a C₁₋₆ haloalkyl group. Examples of the hydrolysis reaction may include acid hydrolysis using hydrochloric acid, sulfuric acid, acetic acid, trifluoroacetic acid, methanesulfonic acid, phosphoric acid, and polyphosphoric acid, etc., singly or in any combination; and alkali hydrolysis using lithium hydroxide, sodium hydroxide, potassium hydroxide, lithium carbonate, sodium carbonate, potassium carbonate, sodium hydrogencarbonate, potassium hydrogencarbonate and ammonia, etc.

The reaction can be performed in the presence or absence of a solvent. Examples of the solvent to be used may include methanol, ethanol, n-propanol, isopropanol, n-butanol, t-butanol, dioxane, tetrahydrofuran, diethyl ether, petroleum ether, n-hexane, cyclohexane, benzene, toluene, xylene, chlorobenzene, pyridine, N,N-dimethylformamide, dimethylsulfoxide, dichloromethane, chloroform, carbon tetrachloride and water.

As the reaction temperature, a cooling temperature to the boiling point of the solvent or reagent to be used, particularly 0° C. to 100° C. is preferable.

In the hydrolytic reaction, it is preferable that the type of solvent and reagent to be used and amounts thereof may be appropriately selected depending on the substrate to be used in the reaction and reaction conditions.

(4) A compound according to the present invention represented by the following formula (Ib)

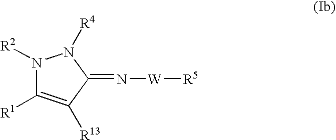

(where R¹, R², R³, R⁴, R⁵, R¹³ and W are the same as defined above) can be produced by the processes shown in the following reaction schemes (i) and (ii).

(i) Pyrazole compound (X)

A compound represented by

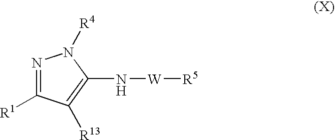

(where R¹, R⁴, R⁵, R¹³ and W are the same as defined above) is reacted with a compound: R²-Q³ (where R² is the same as defined above, Q³ is a leaving group such as a chlorine atom, bromine atom, iodine atom, methanesulfonyloxy group, trifluoromethanesulfonyloxy group or para-toluenesulfonyloxy group), in the presence or absence of a base, to obtain a compound (Ib) according to the present invention.

Examples of the base to be used may include alkali metal hydroxides (such as lithium hydroxide, sodium hydroxide, potassium hydroxide), alkali metal carbonates (such as lithium carbonate, sodium carbonate, potassium carbonate), alkali metal bicarbonates (such as sodium hydrogencarbonate, potassium hydrogencarbonate), alkali metal hydrides (such as sodium hydride, potassium hydride), alkali metals (such as metallic sodium, metallic potassium), organic bases (such as triethylamine, diisopropylethylamine, tri-n-butylamine, 1,5-diazabicyclo[4.3.0]-5-nonene, 1,8-diazabicyclo[5.4.0]-7-undecene, pyridine, N,N-dimethylaminopyridine), alkali metal amides (such as sodium amide), alkali metal alkoxides (such as sodium methoxide, sodium ethoxide, t-butoxy potassium), organometallic compounds (such as n-butyllithium, s-butyllithium, t-butyllithium, lithium diisopropylamide, sodium bis(trimethylsilyl)amide).

As the reaction temperature, a cooling temperature to the boiling point of the solvent or reagent to be used is preferable.

The reaction can be performed in the presence or absence of a solvent. Examples of the solvent to be used may include dioxane, tetrahydrofuran, diethyl ether, petroleum ether, n-hexane, cyclohexane, benzene, toluene, xylene, chlorobenzene, pyridine, ethyl acetate, N,N-dimethylformamide, dimethylsulfoxide, dichloromethane, chloroform, and carbon tetrachloride.

In the reaction, it is preferable that the type of solvent and reagent to be used and amounts thereof may be appropriately selected depending on the substrate to be used in the reaction and reaction conditions.

(ii) The compound: R²-Q³ used in the process shown in reaction scheme (i) of the Step (4) is replaced by a compound: (R²O)₂SO₂ (where R² is the same as defined above) and the same reaction is performed to obtain a compound (Ib) according to the present invention.

Furthermore, when a compound according to the present invention is produced, depending upon the type of functional group, it is sometimes effective to protect the functional group of a raw material or an intermediate in a production process, or to convert it into a group that can be easily converted into the functional group. Examples of such a functional group include amino group, hydroxyl group and carboxyl group. Examples of the protecting group include a general protecting group for an amino group, a hydroxyl group, and a carboxyl group. It is preferable that the reaction temperature of protecting and de-protecting procedures, the types of solvent and reagent to be used and amounts thereof are appropriately selected depending upon a substrate to be used in the reaction and reaction conditions thereof.

A compound according to the present invention can be administered orally or parenterally. Examples of dosage form may include tablet, encapsulation, granular, pulvis, powdery, troche, ointment, cream, emulsion, suspension, suppository, and injection forms. These dosage forms can be prepared by a customary preparation technique (for example, a method defined in the 14th revision of the Japanese Pharmacopeia). The dosage form can be appropriately selected depending on the symptom and age of a patient and the therapeutic purpose. When such various types of dosage forms are prepared, conventional excipients (for example, crystalline cellulose, starch, lactose, mannitol), binders (for example, hydroxypropylcellulose, polyvinylpyrrolidone), lubricants (for example, magnesium stearate, talc), and disintegrators (for example, calcium carboxymethylcellulose) can be used.

The dose of a compound according to the present invention is 1 to 2000 mg per day per adult. This is administered once per day or by dividing it several portions. The dosage may be appropriately increased or decreased depending on the age, weight and symptom of a patient.

### EXAMPLES

The present invention will be more specifically described by way of Examples and Experimental examples below, which should not be construed as limiting the invention.

### Example 1

### Production of 1-cyclopropylmethyl-2-(trifluoroacetylimino)-1,2-dihydropyridine (Compound No. 1)

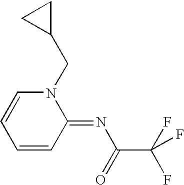

To a solution of 2-aminopyridine (2.0 g) and pyridine ml) in chloroform (20 ml), trifluoroacetic anhydride (3.3 ml) was added under ice cooling. The mixture was stirred at room temperature for three days. The reaction solution was washed sequentially with water and saturated brine and dried over anhydrous sodium sulfate. After filtration, the filtrate was concentrated under vacuum. The residue obtained was purified by silica gel column chromatography (developing solvent: n-hexane:ethyl acetate=3:1) to obtain colorless liquid of 2-(trifluoroacetylamino)pyridine (2.3 g).

The obtained 2-(trifluoroacetylamino)pyridine (1.3 g) was dissolved in N,N-dimethylformamide (13 ml). To the solution, sodium iodide (0.01 g), 60% sodium hydride (0.27 g) and cyclopropylmethyl bromide (1.1 g) were added at room temperature. The reaction solution was stirred at 50° C. for 5 hours and returned to room temperature. After water was added, the reaction solution was extracted with ethyl acetate, washed with saturated brine, dried over anhydrous sodium sulfate, and filtrated. Thereafter the filtrate was concentrated under vacuum. The residue obtained was purified by silica gel column chromatography (developing solvent: chloroform:methanol=18:1) to obtain colorless crystals of 1-cyclopropylmethyl-2-(trifluoroacetylimino)-1,2-dihydropyridine (1.4 g).

¹H-NMR, MS (ESI) and the melting point are shown in Table 8.

### Example 2

### Production of 1-cyclopropylmethyl-2-(3-(trifluoromethyl)benzoylimino)-1,2-dihydropyridine (Compound No. 2)

### (1) 1-cyclopropylmethyl-2-imino-1,2-dihydropyridine

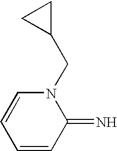

1-cyclopropylmethyl-2-(trifluoroacetylimino)-1,2-dihydropyridine (1.1 g) produced by the process shown in Example 1 was dissolved in methanol (25 ml). To this, an aqueous solution having anhydrous potassium carbonate (1.2 g) dissolved in water (12.5 ml) was added at room temperature and stirred for 2 hours. The reaction solution was extracted with ethyl acetate, dried over anhydrous sodium sulfate, and filtrated. Thereafter, the filtrate was concentrated under vacuum. The residue obtained was purified by silica gel column chromatography (developing solvent: chloroform:methanol=18:1) to obtain a yellow solution of 1-cyclopropylmethyl-2-imino-1,2-dihydropyridine (0.5 g).

¹H-NMR (200 MHz, CHLOROFORM-d) d ppm; 0.31-0.37 (m, 2H), 0.58-0.67 (m, 2H), 1.23-1.43 (m, 1H), 3.69 (d, J=6.0 Hz, 2H), 5.70 (m, 1H), 6.29 (d, J=8.0, 1H), 6.76 (m, 1H), 7.04 (m, 1H)

MS (ESI) (Positive) m/z; 149 (M+H)⁺

### (2) 1-cyclopropylmethyl-2-(3-(trifluoromethyl)benzoylimino)-1,2-dihydropyridine (Compound No. 2)

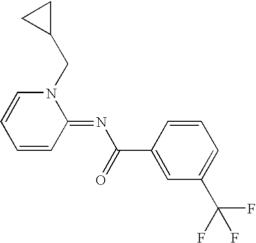

To a solution of 1-cyclopropylmethyl-2-imino-1,2-dihydropyridine (0.20 g) and triethylamine (0.19 ml) in chloroform (2 ml), 3-(trifluoromethyl)benzoyl chloride (0.24 ml) was added under ice cooling. The reaction solution was stirred at room temperature for 17 hours and water was added thereto. The reaction solution was extracted with chloroform, dried over anhydrous sodium sulfate, and filtrated. Thereafter, the filtrate was concentrated under vacuum and the residue obtained was purified by silica gel column chromatography (developing solvent: n-hexane:ethyl acetate=3:1) to obtain a colorless solid substance of 1-cyclopropylmethyl-2-(3-(trifluoromethyl)benzoylimino}-1,2-dihydropyridine (0.44 g).

¹H-NMR, MS (ESI) and the melting point are shown in Table 8.

### Example 3

### Production of 1-cyclopropylmethyl-2-(3-(trifluoromethyl)phenylsulfonylimino}-1,2-dihydropyridine (Compound No. 50)

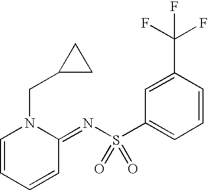

A process in line with the process shown in Example 2 was performed using 3-(trifluoromethyl)phenylsulfonyl chloride in place of 3-(trifluoromethyl)benzoyl chloride to obtain a colorless solid substance of 1-cyclopropylmethyl-2-{3-(trifluoromethyl)phenylsulfonylimino)-1,2-dihydropyridine.

¹H-NMR, Mass and the melting point are shown in Table 8.

Compounds Nos. 3-49 and 51 to 59 shown in Table 1; compounds Nos. 60, 71 to 107, 116 to 174, 178 to 213, 215 to 342, 343 to 351, and 369 to 372; and compounds Nos. 504 to 515 shown in Table 3 were obtained in line with the methods shown in Examples 1 and 2.

¹H-NMR, Mass and the melting points of these compounds are shown in Tables 8, 9 and 11.

### Example 4

### Production of 5-t-butyl-3-(2-ethoxyethyl)-4-methyl-2-(3-trifluoromethylbenzoyl)imino-2,3-dihydrothiazole (Compound No. 352)

### (1) 5-t-butyl-4-methyl-2-(3-trifluoromethylbenzoyl)aminothiazole

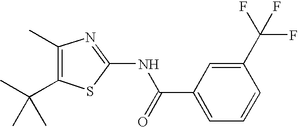

A solution of 2-amino-5-t-butyl-4-methylthiazole (1.0 g), 3-(trifluoromethyl)benzoic acid (1.2 g), 1-ethyl-3-(3-dimethylaminopropyl)carbodiimide monohydrochloride (1.2 g) and 1-hydroxybenzotriazole monohydrate (1.0 g) in N,N,-dimethyl formamide (10 ml) was stirred at room temperature for 48 hours. To the reaction solution, ethyl acetate was added and the reaction solution was washed sequentially with 2M hydrochloric acid, an aqueous 2M sodium hydroxide solution, and saturated brine, dried over anhydrous sodium sulfate and filtrated. Thereafter, the filtrate was concentrated under vacuum to obtain a yellow amorphous substance of 5-t-butyl-4-methyl-2-(3-trifluoromethylbenzoyl)aminothiazole (1.7 g).

¹H-NMR (200 MHz, CHLOROFORM-D) d ppm; 1.42 (s, 9H), 2.20 (s, 3H), 7.55 (t, J=7.5 Hz, 1H), 7.78 (d, J=7.5 Hz, 1H), 8.05 (d, J=7.5 Hz, 1H), 8.16 (s, 1H),

MS (ESI) (Positive) m/z, 343 (M+H)

### (2) 5-t-butyl-3-(2-ethoxyethyl)-4-methyl-2-(3-trifluoromethylbenzoyl)imino-2,3-dihydrothiazole (Compound No. 352)

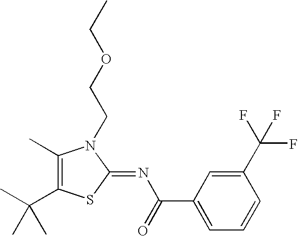

A solution of 5-t-butyl-4-methyl-2-(3-trifluoromethylbenzoyl)aminothiazole (0.15 g), sodium iodide g), 60% sodium hydride (0.02 g) and 2-ethoxyethylbromide (0.11 g) in N,N-dimethylformamide (1.5 ml) was stirred with heating at 50° C. for 5 hours. The reaction solution was returned to room temperature. To the reaction solution, water was added and the reaction solution was extracted with ethyl acetate, washed with saturated brine, dried over anhydrous sodium sulfate, and filtrated. Thereafter, the filtrate was concentrated under vacuum. The residue obtained was purified by silica gel column chromatography (developing solvent: n-hexane:ethyl acetate=5:1) to obtain colorless crystals of 5-t-butyl-3-(2-ethoxyethyl)-4-methyl-2-(3-trifluoromethylbenzoyl)imino-2,3-dihydrothiazole (0.03 g).

¹H-NMR, Mass and the melting point are shown in Table 9.

Compounds Nos. 384 to 430, 433, 434 and 438-447, shown in Table 2, were produced in line with the process shown in Example 3.

¹H-NMR, Mass and the melting point are shown in Table 10.

### Example 5

### Production of N-(5-t-butyl-3-cyclopropylmethyl-4-methyl-3H-thiazol-2-ylidene)-1-naphthylsulfonamide (Compound No. 383)

### (1) N-(5-t-butyl-4-methylthiazol-2-yl)-1-naphthylsulfonamide

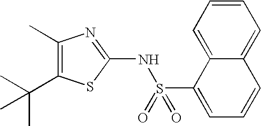

To a solution of 2-amino-5-t-butyl-4-methylthiazole (0.12 g) and N,N-dimethylaminopyridine (catalyst quantity) in pyridine (1.5 ml), 1-naphtylsulfonyl chloride (0.19 g) was added under ice cooling and stirred at room temperature overnight. To the reaction solution, water was added, and a precipitate was obtained by filtration and dried. The crude crystals obtained were recrystallized from chloroform/n-hexane to obtain colorless crystals of N-(5-t-butyl-4-methylthiazol-2-yl)-1-naphthylsulfonamide (0.22 g).

¹H-NMR (200 MHz, CHLOROFORM-D) d ppm; 1.43 (s, 9H), 2.36 (s, 3H)

MS (ESI) (Negative) m/z; 265 (M−H)⁻⁽2)

### N-(5-t-butyl-3-cyclopropylmethyl-4-methyl-3H-thiazol-2-ylidene)-1-naphthylsulfonamide (compound No. 383)

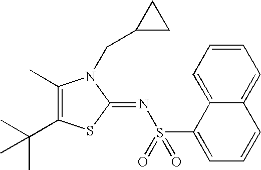

A solution of N-(5-t-butyl-4-methyl-thiazol-2-yl)-1-naphthylsulfonamide (0.20 g), 55% sodium hydride (0.03 g), sodium iodide (catalyst quantity) and (bromomethyl)cyclopropane (0.11 g) in N,N-dimethylformamide ml) was stirred at room temperature overnight. To the reaction solution, water was added and the reaction solution was extracted with ethyl acetate, washed sequentially with water and saturated brine, dried over anhydrous magnesium sulfate and filtrated. Thereafter the filtrate was concentrated under vacuum. The residue obtained was purified by silica gel column chromatography (developing solvent: n-hexane:ethyl acetate=4:1) and recrystallized from chloroform/n-hexane to obtain colorless crystals of N-(5-t-butyl-3-cyclopropylmethyl-4-methyl-3H-thiazol-2-ylidene)-1-naphthylsulfonamide (0.12 g).

¹H-NMR, Mass and the melting point are shown in Table 10.

### Example 6

### Production of 3-(2-aminoethyl)-5-t-butyl-4-methyl-2-(3-trifluoromethylbenzoyl)imino-2,3-dihydrothiazole (Compound No. 354)

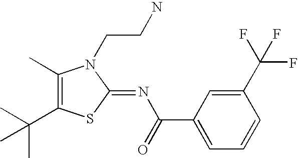

A solution of 3-(2-phthaliminoethyl)-5-t-butyl-4-methyl-2-(3-trifluoromethylbenzoyl)imino-2,3-dihydrothiazole (Compound No. 355) produced line the process shown in Example 4 and hydrazine monohydrate (0.2 ml) in ethanol (6.3 ml) was refluxed under heating for one hour. The reaction solution was returned to room temperature and a precipitate was removed by filtration. To the filtrate, chloroform was added and the resultant solution was washed sequentially with an aqueous 2M sodium hydroxide solution and saturated brine, dried over anhydrous magnesium sulfate, and filtrated. Thereafter, the filtrate was concentrated under vacuum to obtain light yellow crystals of 3-(2-aminoethyl)-5-t-butyl-4-methyl-2-(3-trifluoromethylbenzoyl)imino-2,3-dihydrothiazole (0.12 g).

¹H-NMR, Mass and the melting point are shown in Table 9.

### Example 7

### Production of 5-carboxy-3-cyclopropylmethyl-4-methyl-2-(3-trifluoromethylbenzoyl)imino-2,3-dihydrothiazole (Compound No. 357)

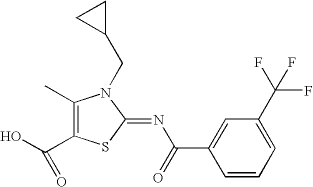

A solution (8 ml) of tetrahydrofuran/ethanol (1:1) containing 5-ethoxycarbonyl-3-cyclopropylmethyl-4-methyl-2-(3-trifluoromethylbenzoyl)imino-2,3-dihydrothiazole (Compound No. 356) (0.23 g) produced in line with the process shown in Example 4 and a 20% aqueous sodium hydroxide solution (1.25 ml) was stirred at room temperature for one hour. To the reaction solution, 3M hydrochloric acid was added to make the solution acidic and a precipitate was obtained by filtration. The solid substance thus obtained was dissolved in tetrahydrofuran/chloroform, dried over anhydrous magnesium sulfate and filtrated. Thereafter, the filtrate was concentrated under vacuum to obtain a colorless amorphous substance of 5-carboxy-3-cyclopropylmethyl-4-methyl-2-(3-trifluoromethylbenzoyl)imino-2,3-dihydrothiazole (0.20 g).

¹H-NMR, Mass and the melting point are shown in Table 9.

Compound No. 214 shown in Table 2 and compounds Nos. 431 and 435 shown in Table 3 were produced in line with the process shown in Example 7.

¹H-NMR, Mass and the melting points of these compounds are shown in Tables 9, 10 and 11.

### Example 8

### Production of 3-cyclopropylmethyl-5-isopropylaminocarbonyl-4-methyl-2-(3-trifluoromethylbenzoyl)imino-2,3-dihydrothiazole (Compound No. 358)

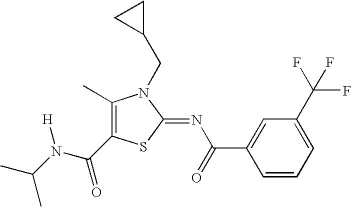

A colorless powdery substance of 3-cyclopropylmethyl-5-isopropylaminocarbonyl-4-methyl-2-(3-trifluoromethylbenzoyl)imino-2,3-dihydrothiazole was obtained in line with the process shown in Example 3 (1) by using 5-carboxy-3-cyclopropylmethyl-4-methyl-2-(3-trifluoromethylbenzoyl)imino-2,3-dihydrothiazole (Compound No. 357) produced in line with the process shown in Example 7.

¹H-NMR, Mass and the melting point are shown in Table 9.

Compounds Nos. 359 to 361 were produced in line with the production example shown in Example 8 by using Compound No. 356.

Compound No. 432 was produced similarly, by using compound No. 431.

Compounds Nos. 436 and 437 were produced similarly, by using compound No. 435.

¹H-NMR, Mass and the melting points of these compounds are shown in Tables 9 and 10.

### Example 9

### Production of N-(3-allyl-4,5-diphenyl-3H-thiazol-2-ylidene)benzamide (Compound No. 368)

### (1) 1-allyl-3-benzoylthiourea

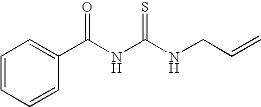

A solution of benzoylisothiocyanate (1.4 g) and allylamine (0.7 ml) in benzene (9 ml) was stirred at room temperature overnight. The reaction solution was concentrated under vacuum and the residue obtained was purified by silica gel column chromatography (developing solvent: n-hexane:ethyl acetate=10:1 to 5:1) to obtain colorless solid substance of 1-allyl-3-benzoylthiourea (1.9 g).

¹H-NMR (200 MHz, CHLOROFORM-D) d ppm; 4.30-4.43 (m, 2H), 5.21-5.42 (m, 2H), 5.87-6.09 (m, 1H), 7.45-7.69 (m, 3H), 7.78-7.90 (m, 2H), 9.00 (s, 1H), 10.80 (s, 1H),

MS (ESI) (Negative) m/z; 219 (M−H)⁻

The melting point was 68 to 69° C.

### (2) N-(3-allyl-4,5-diphenyl-3H-thiazol-2-ylidene)benzamide (Compound No. 368)

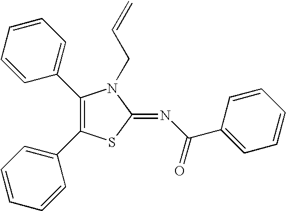

A solution of 1-allyl-3-benzoylthiourea (0.20 g) and 2-bromo-1,2-diphenylethane (0.24 g) in toluene (4.5 ml) was refluxed under heating for 4.5 hours. The solvent was concentrated under vacuum and the residue obtained was purified by silica gel column chromatography (developing solvent: n-hexane:ethyl acetate=30:1 to 20:1) and recrystallized from n-hexane/ethyl acetate to obtain colorless crystals of N-(3-allyl-4,5-diphenyl-3H-thiazol-2-ylidene)benzamide (0.13 g).

¹H-NMR, Mass and the melting point are shown in Table 9.

### Example 10

### Production of N-[1,2-dihydro-1,5-dimethyl-2-(2-ethoxyethyl)pyrazol-3-ylidene]-2-fluoro-3-(trifluoromethyl)benzamide (Compound No. 452)

### (1) N-1,5-dimethylpyrazol-3-yl]-2-fluoro-3-(trifluoromethyl)benzamide

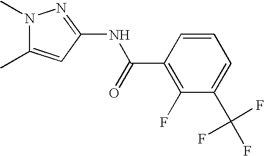

To a solution of 3-amino-1,5-dimethylpyrazole (0.50 g) and triethylamine (0.63 ml) in chloroform (5 ml), 2-fluoro-3-(trifluoromethyl)benzoyl chloride (0.65 ml) was added under ice cooling. The reaction solution was stirred at room temperature for 0.5 hours, washed with an aqueous 2M sodium hydroxide solution, dried over anhydrous sodium sulfate and filtrated. Thereafter, the filtrate was concentrated under vacuum. The residue obtained was washed with n-hexane to obtain a colorless solid substance of N-(1,5-dimethylpyrazol-3-yl)-2-fluoro-3-(trifluoromethyl)benzamide (1.25 g).

¹H-NMR (200 MHz, CHLOROFORM-D) d ppm; 2.30 (s, 39H), 3.72 (s, 3H), 6.59 (s, 1H), 7.40 (t, J=7.5 Hz, 1H), 7.78 (t, J=7.5 Hz, 1H), 8.34 (td, J=7.5 Hz, 1H), 8.60-8.89. (br. s, 1H)

MS (ESI) (Positive) m/z; 302 (M+H)⁺

### (2) N-[1,2-dihydro-1,5-dimethyl-2-(2-ethoxyethyl)pyrazol-3-ylidene]-2-fluoro-3-(trifluoromethyl)benzamide

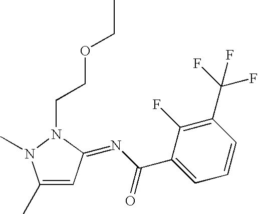

A suspension solution of N-(1,5-dimethylpyrazol-3-yl)-2-fluoro-3-(trifluoromethyl)benzamide (0.50 g), 55% sodium hydride (0.07 g) in N,N-dimethylformamide (5 ml) was stirred at room temperature for 5 minutes. To this mixture, 2-ethoxyethyl bromide (0.38 g) and sodium iodide (catalytic quantity) were added and stirred for 17 hours. To the reaction solution, water was added and the reaction solution was extracted with ethyl acetate and then concentrated under vacuum. The residue obtained was purified by silica gel column chromatography (developing solvent: chloroform:methanol=18:1) to obtain a colorless solid substance of N-[1,2-dihydro-1,5-dimethyl-2-(2-ethoxyethyl)pyrazol-3-ylidene]-2-fluoro-3-(trifluoromethyl)benzamide (0.01 g).

¹H-NMR, Mass and the melting points of these compounds are shown in Table 11.

Compounds Nos. 448 to 451, 453, 454 and 456 shown in Table 3 were produced in line with the process shown in Example 10.

¹H-NMR, Mass and the melting points of these compounds are shown in Table 11.

### Example 11

### Production of N-{1,2-dihydro-1,5-dimethyl-2-(1,1-dioxotetrahydrothiophen-3-yl)pyrazol-3-ylidene}-2-fluoro-3-(trifluoromethyl)benzamide (Compound No. 455)

### (1) N-(2-(1,1-dioxotetrahydrothiophen-3-yl)-5-methylpyrazol-3-yl)-2-fluoro-3-(trifluoromethyl)benzamide

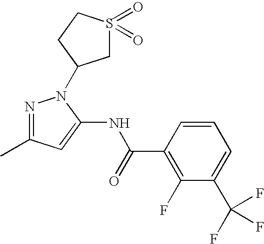

A colorless solid substance of N-(2-(1,1-dioxotetrahydrothiophen-3-yl)-5-methylpyrazol-3-yl)-2-fluoro-3-(trifluoromethyl)benzamide was obtained in line with the process shown in Example 8(1) by using 3-amino-2-(1,1-dioxotetrahydrothiophen-3-yl)-5-methylpyrazole.

¹H-NMR (600 MHz, CHLOROFORM-D) d ppm; 2.63-2.69 (m, 1H), 2.75-2.81 (m, 1H), 3.12 (m, 1H), 3.50-3.63 (m, 3H), 4.89 (m, 1H), 6.00 (s, 1H), 7.46 (m, 1H), 7.87 (m, 1H), 8.13 (d, J=13.3 Hz, 1H), 8.33 (m, 1H)

MS (ESI) (Positive) m/z; 406 (M+H)⁺

### (2) N-{1,2-dihydro-1,5-dimethyl-2-(1,1-dioxotetrahydrothiophen-3-yl)pyrazol-3-ylidene)-2-fluoro-3-(trifluoromethyl)benzamide (Compound No. 455)

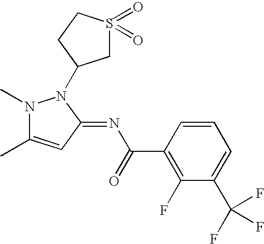

A solution of N-{2-(1,1-dioxotetrahydrothiophen-3-yl)-5-methylpyrazol-3-yl)-2-fluoro-3-(trifluoromethyl)benzamide (0.40 g) and dimethyl sulfate (0.11 ml) in toluene (1.2 ml) was stirred at 80° C. while heating for 47 hours. The reaction solution was returned to room temperature and purified by silica gel column chromatography (developing solvent: chloroform:methanol=20:1) to obtain a colorless amorphous substance of N-(1,2-dihydro-1,5-dimethyl-2-(1,1-dioxotetrahydrothiophen-3-yl)pyrazol-3-ylidene}-2-fluoro-3-(trifluoromethyl)benzamide (0.11 g).

¹H-NMR, Mass and the melting point are shown in Table 11.

Compounds No. 497 to 499 shown in Table 3 were obtained in accordance with the process shown in Example 11.

¹H-NMR, Mass and the melting points thereof are shown in Table 11.

### Example 12

### Production of N-(5-t-butyl-2-cyclopropylmethyl-1,2-dihydro-1-methylpyrazol-3-ylidene)formamide (Compound No. 457)

### (1) 3-amino-5-t-butyl-2-(cyclopropylmethyl)pyrazole

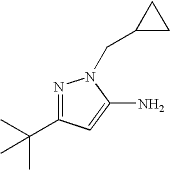

To a solution of cyclopropylmethanol (125 g) and triethylamine (315 ml) in chloroform (500 ml), methanesulfonyl chloride (175 ml) was added under ice cool for 1.5 hours. The mixture was stirred at room temperature for 2 hours. To the reaction solution, water was added and the reaction solution was extracted with chloroform, washed sequentially with water and saturated brine, dried over anhydrous magnesium sulfate, and filtrated. Thereafter, the filtrate was concentrated under vacuum. The residue obtained (232 g) was dissolved in ethanol (500 ml). To this, hydrazine monohydrate (500 g) was added at room temperature and stirred for 17 hours. After the solvent was distilled off under vacuum, the residue was extracted with chloroform, dried over anhydrous magnesium sulfate, and filtrated. The filtrate was concentrated under vacuum. The residue of a yellow oily substance (68 g) obtained was dissolved in ethanol (610 ml). To this, 4,4-dimethyl-3-oxopentanenitrile (99 g) was added at room temperature and then refluxed for 4 hours. After the solvent was removed under vacuum, the residue was purified by silica gel column chromatography (developing solvent: n-hexane:ethyl acetate=5:1) and recrystallized from ethyl acetate/n-hexane to obtain a colorless solid substance of 3-amino-5-t-butyl-2-cyclopropylmethyl)pyrazole (80 g).

¹H-NMR (200 MHz, CHLOROFORM-d) d ppm; 0.26-0.41 (m, 2H), 0.49-0.62 (m, 2H), 1.11-1.27 (m, 1H), 1.26 (s, 9H) 3.42 (br. s, 2H), 3.86 (d, J=6.2 Hz, 2H), 5.42 (s, 1H)

MS (ESI) (Positive) m/z; 194 (M+H)⁺

Melting point: 69.5-70.5° C.

### (2) N-(5-t-butyl-2-cyclopropylmethylpyrazol-3-yl)trifluoroacetamide

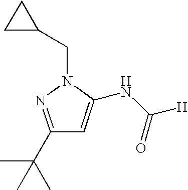

Formic acid (3.9 ml) and acetic anhydride (7.4 ml) were stirred at room temperature for one hour. To this, 3-amino-5-t-butyl-2-(cyclopropylmethyl)pyrazole (5.0 g) was added under ice cooling and stirred at room temperature for one hour. To the reaction solution, an aqueous 2M sodium hydroxide solution was added and the reaction solution was extracted with sodium acetate, washed with water, dried over anhydrous sodium sulfate, and filtrated. Thereafter, the filtrate was concentrated under vacuum to obtain a colorless oily substance of N-(5-t-butyl-2-cyclopropylmethylpyrazol-3-yl)formamide (4.4 g).

¹H-NMR (200 MHz, CHLOROFORM-d) d ppm; 0.27-0.40 (m, 2H), 0.50-0.66 (m, 2H), 1.13-1.27 (m, 1H), 1.29 (s, 9H), 3.91 (d, J=6.6 Hz, 2H), 5.98 (s, 3H), 6.26 (s, 3H), 8.26-8.31 (m, 3H), 8.31-8.39 (m, 3H)

MS (ESI) (Positive) m/z; 222 (M+H)⁺

### (3) N-(5-t-butyl-2-cyclopropylmethyl-1,2-dihydro-1-methylpyrazol-3-ylidene)formamide (Compound No. 457)

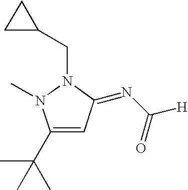

To a solution of N-(5-t-butyl-2-cyclopropylmethylpyrazol-3-yl)formamide (2.0 g) in toluene (6 ml), dimethyl sulfate ml) was added and stirred with heating at 50° C. for 48 hours. The reaction solution was returned to room temperature. To this, an aqueous saturated sodium hydrogencarbonate solution was added and the reaction solution was extracted with ethyl acetate, washed sequentially with water and saturated brine, dried over anhydrous magnesium sulfate, and filtrated. Thereafter, the filtrate was concentrated under vacuum. The residue obtained was purified by silica gel column chromatography (developing solvent: chloroform:methanol=18:1) to obtain a colorless solid substance of N-(5-t-butyl-2-cyclopropylmethyl-1,2-dihydro-1-methylpyrazol-3-ylidene)formamide (0.35 g).

¹H-NMR, Mass and the melting point are shown in Table 11.

### Example 13

### Production of N-(5-t-butyl-2-cyclopropylmethyl-1-methylpyrazol-3-yl)trifluoroacetamide (Compound No. 458)

### (1) N-(5-t-butyl-2-cyclopropylmethylpyrazol-3-yl)trifluoroacetamide

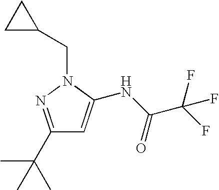

N-(5-t-butyl-2-cyclopropylmethylpyrazol-3-yl) trifluoroacetamide was obtained in line with the process shown in Example 1 by using 3-amino-5-t-butyl-2-(cyclopropylmethyl)pyrazole produced in the process shown in Example 12 (1).

¹H-NMR (200 MHz, CHLOROFORM-d) d ppm; 0.32-0.42 (m, 2H), 0.65-0.74 (m, 2H), 1.10-1.36 (m, 1H), 1.30 (s, 9H), 3.99 (d, J=6.0 Hz, 2H), 6.31 (s, 1H), 8.11-8.23 (brs, 1H)

MS (ESI) (Negative) m/z; 290 (M+H)⁺

### (2) N-(5-t-butyl-2-cyclopropylmethyl-1-methylpyrazol-3-yl)trifluoroacetamide (Compound No. 458)

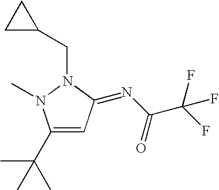

N-(5-t-butyl-2-cyclopropylmethyl-1-methylpyrazol-3-yl)trifluoroacetamide was obtained from N-(5-t-butyl-2-cyclopropylmethylpyrazol-3-yl)trifluoroacetamide in line with the process shown in Example 12 (3).

¹H-NMR, Mass and the melting point are shown in Table 11.

### Example 14

### Production of N-(5-t-butyl-2-cyclopropylmethyl-1,2-dihydro-1-methylpyrazol-3-ylidene)-2-chloro-3-(trifluoromethyl)benzamide (Compound No. 476)

### (1) 5-t-butyl-2-cyclopropylmethyl-1,2-dihydro-1-methylpyrazol-3-ylideneamine

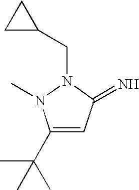

A solution of N-(5-t-butyl-2-cyclopropylmethyl-1,2-dihydro-1-methylpyrazol-3-ylidene)formamide (0.20 g) produced by the process of Example 12 and 12 M hydrochloric acid (0.3 ml) in methanol (3 ml) was stirred at room temperature for 2 hours. To the reaction solution, a 2M aqueous sodium hydroxide solution was added. After the reaction solution was made basic, it was extracted with chloroform, dried over anhydrous sodium sulfate, and filtrated. Thereafter, the filtrate was concentrated under vacuum to obtain a yellow amorphous solid substance of 5-t-butyl-2-cyclopropylmethyl-1,2-dihydro-1-methylpyrazol-3-ylideneamine (0.17 g)

¹H-NMR (200 MHz, CHLOROFORM-d) d ppm; 0.28-0.41 (m, 2H), 0.43-0.55 (m, 2H), 0.95-1.17 (m, 1H), 1.29 (s, 9H), 3.16 (s, 3H), 3.62 (d, J=6.6 Hz, 2H), 5.31 (s, 1H)

MS (ESI) (Negative) m/z; 208 (M+H)⁺

### (2) N-(5-t-butyl-2-cyclopropylmethyl-1,2-dihydro-1-methylpyrazol-3-ylidene)-2-chloro-3-(trifluoromethyl)benzamide (Compound No. 476)

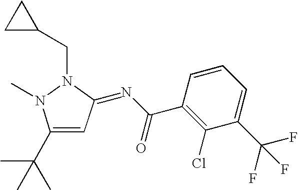

A solution of 2-chloro-3-trifluoromethyl benzoic acid (0.13 g) and thionyl chloride (0.07 ml) and N,N-dimethylformamide (0.01 ml) in tetrahydrofuran (2 ml) was refluxed under heating for 0.5 hours. The solvent was distilled off under vacuum. To the residue, chloroform (1 ml) was added to form a chloroform solution. The chloroform solution was added to a solution of 5-t-butyl-2-cyclopropylmethyl-1-methyl-1,2-dihydropyrazol-3-ylideneamine (0.10 g) and triethylamine (0.10 ml) in chloroform (2 ml) at room temperature and stirred for 17 hours. The reaction solution was washed sequentially with a saturated aqueous sodium hydrogencarbonate solution and a saturated aqueous sodium chloride solution, dried over anhydrous magnesium sulfate, and filtrated. Thereafter, the filtrate was concentrated under vacuum. The residue obtained was purified by silica gel column chromatography (developing solvent: ethyl acetate:methanol=5:1) to obtain a colorless solid substance of N-(5-t-butyl-2-cyclopropylmethyl-1,2-dihydro-1-methylpyrazol-3-ylidene)-2-chloro-3-(trifluoromethyl)benzamide (0.05 g).

¹H-NMR, Mass and the melting point are shown in Table 11.

Compounds Nos. 459 to 473, 475, 477 to 496 and 503 shown in Table 3 were produced in line with the process shown in Table 14.

¹H-NMR, Mass and the melting points of these compounds are shown in Table 11.

### Example 15

### Production of N-(5-t-butyl-2-cyclopropylmethyl-1,2-dihydro-1-methylpyrazol-3-ylidene)-2-fluoro-3-(trifluoromethyl)benzamide (Compound No. 474)

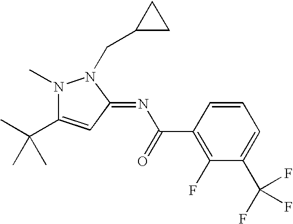

To a solution of 3-amino-5-t-butyl-2-(cyclopropylmethyl)pyrazole (12.8 g) and triethylamine (9.2 ml) in chloroform (120 ml), 2-fluoro-3-(trifluoromethyl)benzoyl chloride (15.0 g) was added under ice cooling and stirred at room temperature for one hour. The reaction solution was washed with an aqueous 2M sodium hydroxide solution, dried over anhydrous sodium sulfate, and filtrated. Thereafter, the filtrate was concentrated under vacuum. The residue obtained was purified by silica gel column chromatography (developing solvent: n-hexane:ethyl acetate=3:1) to obtain a colorless solid substance (18.3 g). The obtained solid substance (4.4 g) was dissolved in toluene (90 ml). To this, dimethyl sulfate (3.2 ml) was added and stirred with heating at 80° C. for 17 hours. The reaction solution was returned at room temperature. An aqueous saturated sodium hydrogencarbonate solution was added to this, the reaction solution was extracted with ethyl acetate, washed sequentially with water and saturated brine, dried over anhydrous sodium sulfate and filtrated. Thereafter, the filtrate was concentrated under vacuum. The residue obtained was purified by silica gel column chromatography (developing solvent: chloroform:methanol=18:1) to obtain a colorless solid substance of N-(5-t-butyl-2-cyclopropylmethyl-1,2-dihydro-1-methylpyrazol-3-ylidene)-2-fluoro-3-(trifluoromethyl)benzamide (0.3 g).

¹H-NMR, Mass and the melting point are shown in Table 11.

### Example 16

### Production of N-(5-t-butyl-2-cyclobutylmethyl-1,2-dihydro-1-methylpyrazol-3-ylidene)-2-fluoro-3-(trifluoromethyl)benzamide (Compound No. 482)

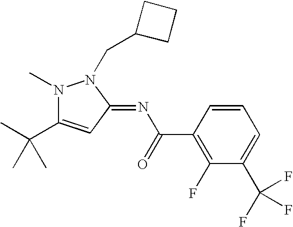

N-(5-t-butyl-2-cyclobutylmethyl-1,2-dihydro-1-methylpyrazol-3-ylidene)-2-fluoro-3-(trifluoromethyl)benzamide was obtained in line with the process shown in Example 15 by using 3-amino-5-t-butyl-2-(cyclobutylmethyl)pyrazole produced in accordance with the process shown in Example 12 (1).

¹H-NMR, Mass and the melting point are shown in Table 11.

The compounds Nos. 481 and 483 to 496 shown in Table 3 were produced in line with the process shown in Example 16.

¹H-NMR, Mass and the melting points of these compounds are shown in Table 11.

### Example 17

### Production of N-(5-t-butyl-2-cyclopropylmethyl-1,2-dihydro-1-methylpyrazol-3-ylidene)-3,5-difluorophenyloxoacetamide (Compound No. 500)

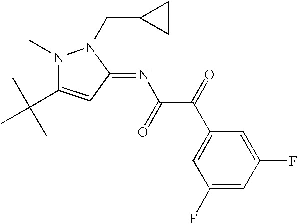

A mixture of 5-t-butyl-2-cyclopropylmethyl-1,2-dihydro-1-methylpyrazol-3-ylideneamine (0.080 g) produced by the process shown in Example 14(1) and ethyl 3,5-difluorophenyloxoacetate m1) was stirred with heating at 110° C. for 7 hours. The mixture was returned to room temperature and separated and purified by silica gel column chromatography (developing solvent: chloroform:methanol=30:1) and sequentially by thin-layer chromatography (developing solvent: n-hexane:ethyl acetate=1:10) to obtain a light brown amorphous substance of N-(5-t-butyl-2-cyclopropylmethyl-1,2′-dihydro-1-methylpyrazol-3-ylidene)-3,5-difluorophenyloxoacetamide (0.5 mg).

¹H-NMR, Mass and the melting point are shown in Table 11.

### Example 18

### Production of 1-(5-t-butyl-2-cyclopropylmethyl-1,2-dihydro-1-methylpyrazol-3-ylidene)-3-(4-chloro-2-(trifluoromethyl)phenyl}urea (Compound No. 501)

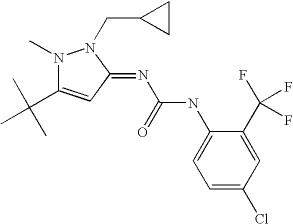

A solution of 5-t-butyl-2-cyclopropylmethyl-1,2-dihydro-1-methylpyrazol-3-ylideneamine (0.050 g) produced by the process shown in Example 14(1) and 4-chloro-2-(trifluoromethyl)phenylisocyanate (0.064 g) in tetrahydrofuran ml) was stirred at room temperature for 20 hours. To the reaction solution, water was added and the reaction solution was extracted with ethyl acetate, washed sequentially with water and saturated brine, dried over anhydrous magnesium sulfate, and filtrated. Thereafter, the filtrate was concentrated under vacuum. The residue obtained was separated and purified by thin-layer column chromatography (developing solvent: chloroform:methanol=25:1) to obtain a light yellow powdery substance of 1-(5-t-butyl-2-cyclopropylmethyl-1,2-dihydro-1-methylpyrazol-3-ylidene)-3-(4-chloro-2-(trifluoromethyl)phenyl)urea (0.001 g).

¹H-NMR, Mass and the melting point are shown in Table 11.

### Example 19

### Production of 1-(5-t-butyl-2-cyclopropylmethyl-1,2-dihydro-1-methylpyrazol-3-ylidene)-3-(4-fluorophenyl)thiourea (Compound No. 502)

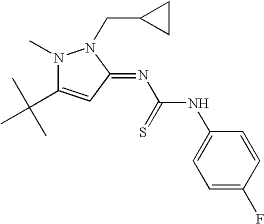

1-(5-t-butyl-2-cyclopropylmethyl-1,2-dihydro-1-methylpyrazol-3-ylidene)-3-(4-fluorophenyl)thiourea was obtained in accordance with the process shown in Example 18.

¹H-NMR, Mass and the melting point are shown in Table 11.

Compounds Nos. 1001 to 1431, 2001 to 2678, 3001 to 3158 and 3159 to 3327 shown in Tables 4 to 7 were produced in accordance with the process shown in Example 2 (2) or Example 14(2) by using imine compounds (1-cyclopropylmethyl-2-imino-1,2-dihydropyridine, 3-cyclopropylmethyl-2,3-dihydro-4,5-dimethylthiazol-2-ylideneamine, 5-t-butyl-2,3-dihydro-3,4-dimethylthiazol-2-ylideneamine, 5-t-butyl-2,3-dihydro-3-ethyl-4-methylthiazol-2-ylideneamine, 5-t-butyl-2,3-dihydro-3-(2-methoxyethyl)-4-methylthiazol-2-ylideneamine, 5-t-butyl-3-cyclopropylmethyl-2,3-dihydro-4-methylthiazol-2-ylideneamine, 5-t-butyl-2-cyclopropylmethyl-1,2-dihydro-1-methylpyrazol-3-ylideneamine, and 5-t-butyl-3-cyclopropylmethyl-2,3-dihydro-1,3,4-thiadiazol-2-ylideneamine).

The Mass of the compounds obtained is shown in Tables 12-15.

TABLE 1

| Compound | |
|---|---|
| No. | chemical structure |
| 1 | 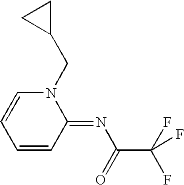 |
| 2 | 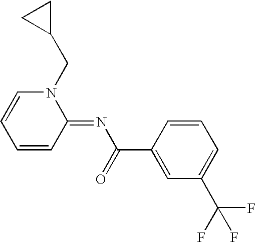 |
| 3 | 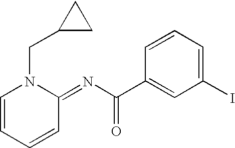 |
| 4 | 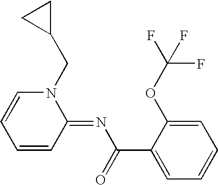 |
| 5 | 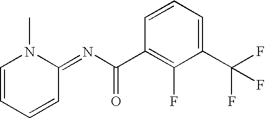 |
| 6 | 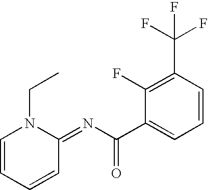 |
| 7 | 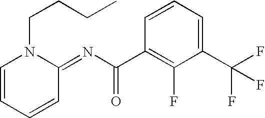 |
| 8 | 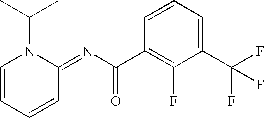 |
| 9 | 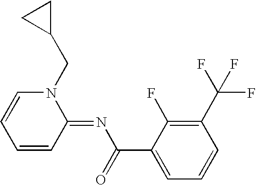 |
| 10 | 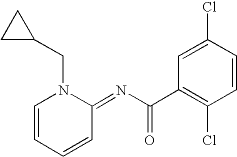 |
| 11 | 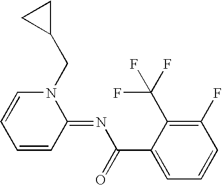 |
| 12 | 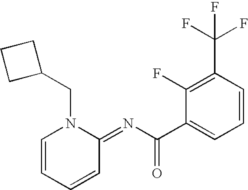 |
| 13 | 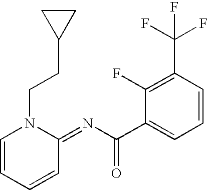 |
| 14 | 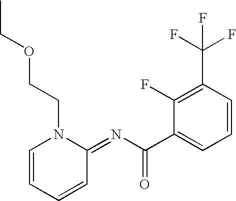 |
| 15 | 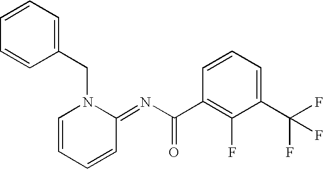 |
| 16 | 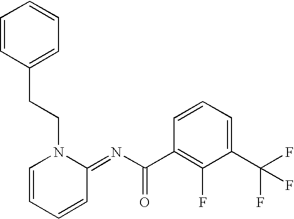 |
| 17 | 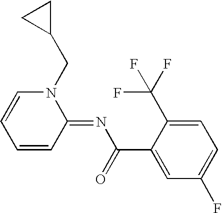 |
| 18 | 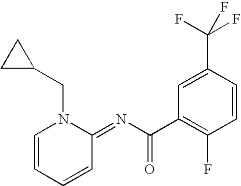 |
| 19 | 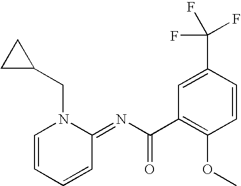 |
| 20 | 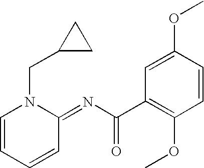 |
| 21 | 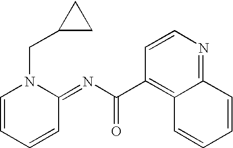 |
| 22 | 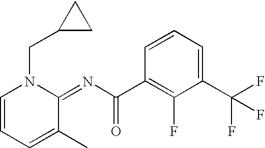 |
| 23 | 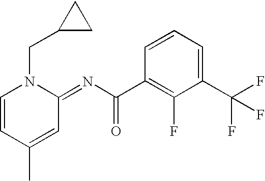 |
| 24 | 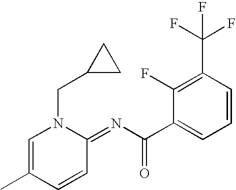 |
| 25 | 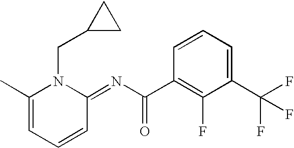 |
| 26 | 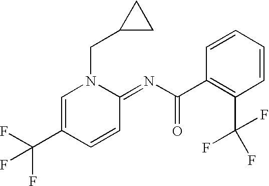 |
| 27 | 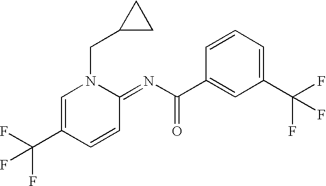 |
| 28 | 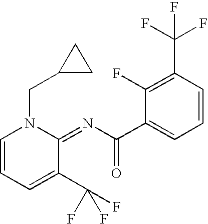 |
| 29 | 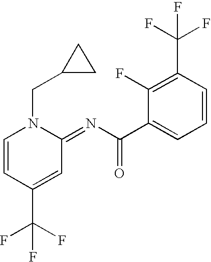 |
| 30 | 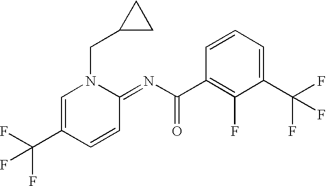 |
| 31 | 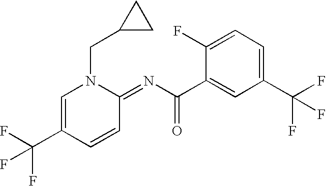 |
| 32 | 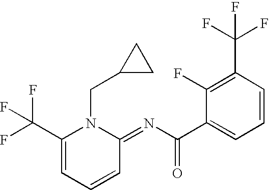 |
| 33 | 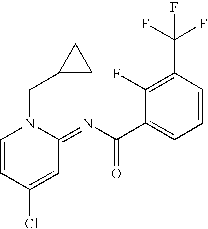 |
| 34 | 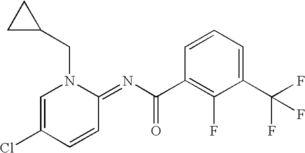 |
| 35 | 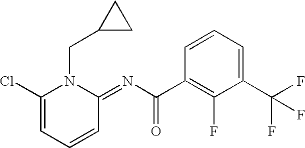 |
| 36 | 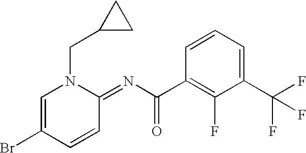 |
| 37 | 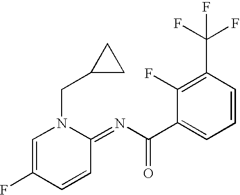 |
| 38 | 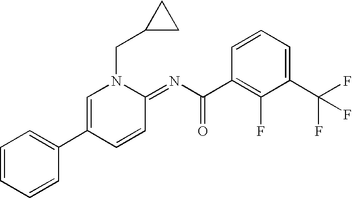 |
| 39 | 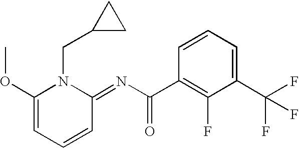 |
| 40 | 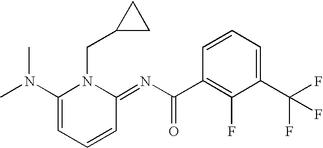 |
| 41 | 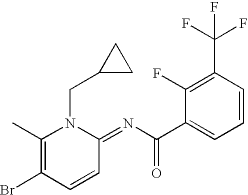 |
| 42 | 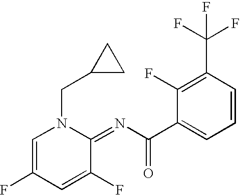 |
| 43 | 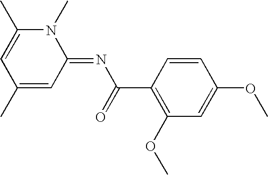 |
| 44 | 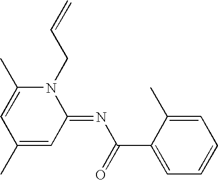 |
| 45 | 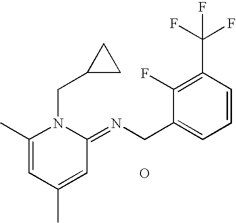 |
| 46 | 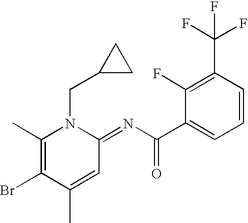 |
| 47 | 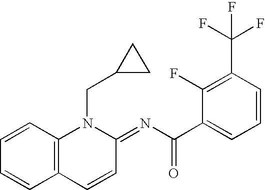 |
| 48 | 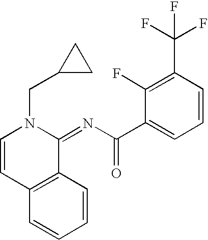 |
| 49 | 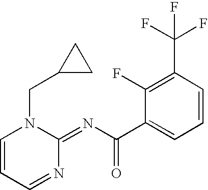 |
| 50 | 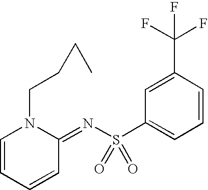 |
| 51 | 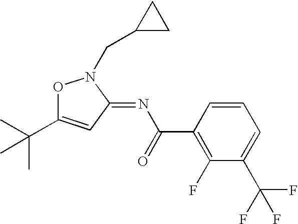 |
| 52 | 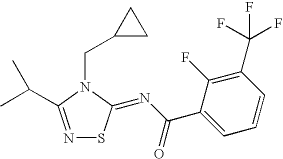 |
| 53 | 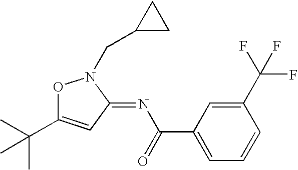 |
| 54 | 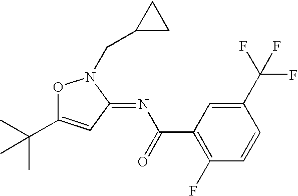 |
| 55 | 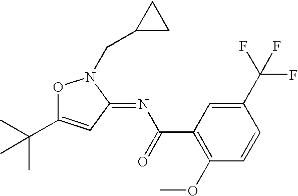 |
| 56 | 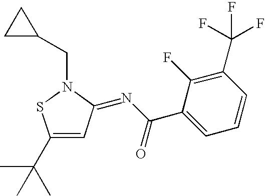 |
| 57 | 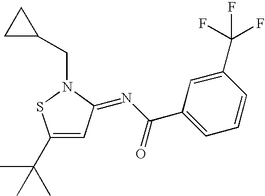 |
| 58 | 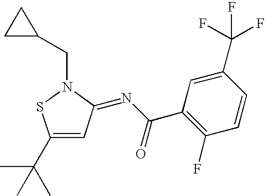 |
| 59 | 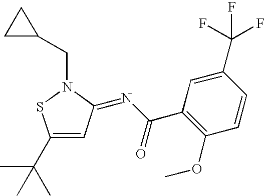 |

TABLE 2

| Example | |
|---|---|
| No. | chemical structure |
| 60 | 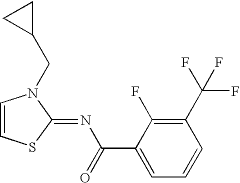 |
| 61 | 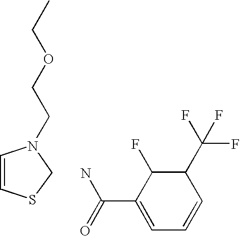 |
| 62 | 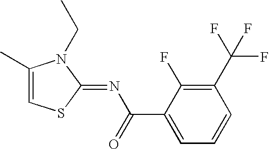 |
| 63 | 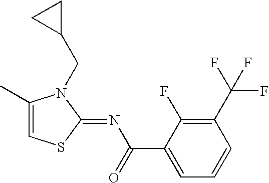 |
| 64 | 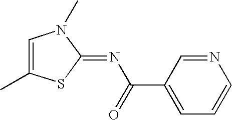 |
| 65 | 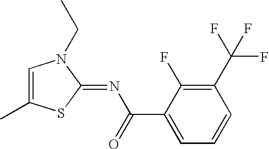 |
| 66 | 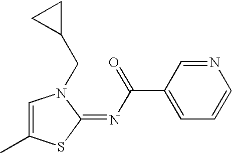 |
| 67 | 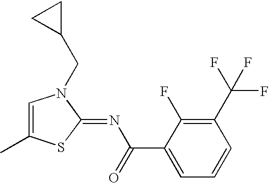 |
| 68 | 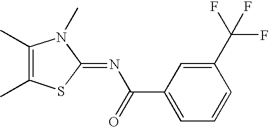 |
| 69 | 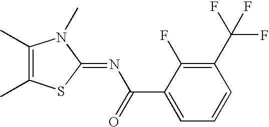 |
| 70 | 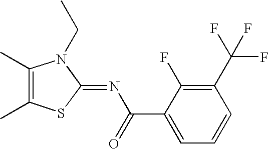 |
| 71 | 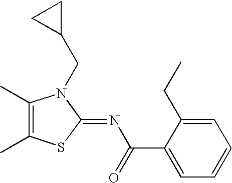 |
| 72 | 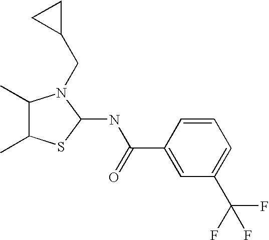 |
| 73 | 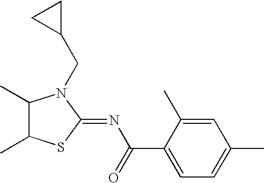 |
| 74 | 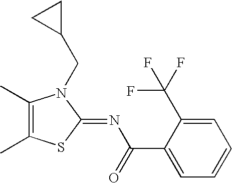 |
| 75 | 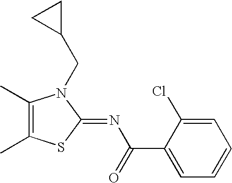 |
| 76 | 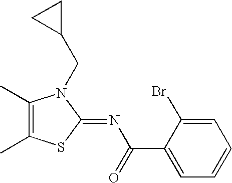 |
| 77 | 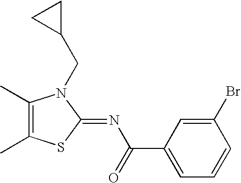 |
| 78 | 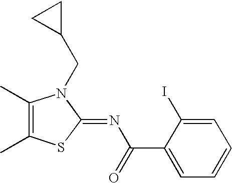 |
| 79 | 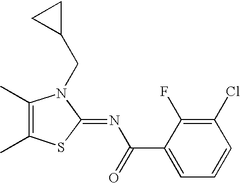 |
| 80 | 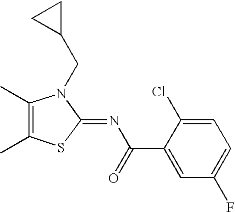 |
| 81 | 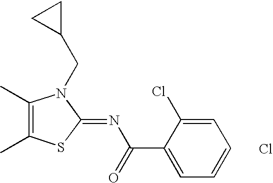 |
| 82 | 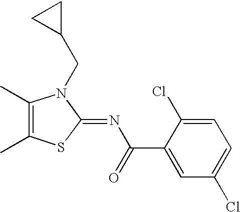 |
| 83 | 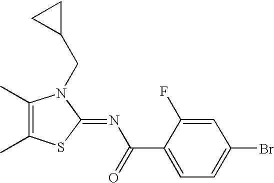 |
| 84 | 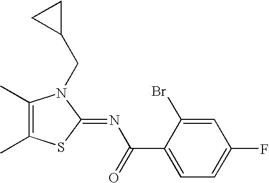 |
| 85 | 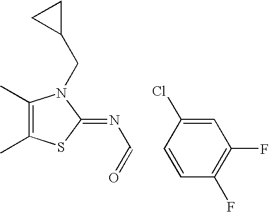 |
| 86 | 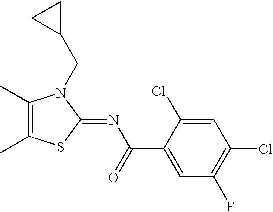 |
| 87 | 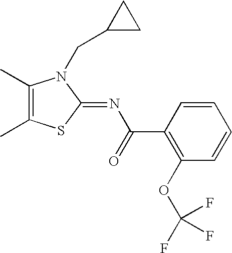 |
| 88 | 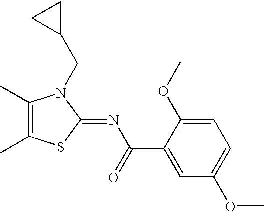 |
| 89 | 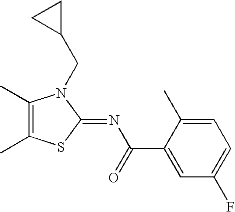 |
| 90 | 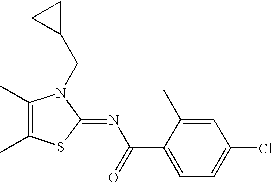 |
| 91 | 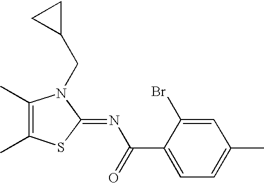 |
| 92 | 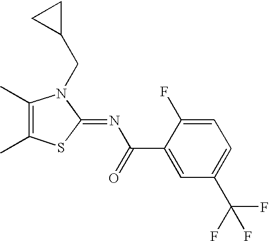 |
| 93 | 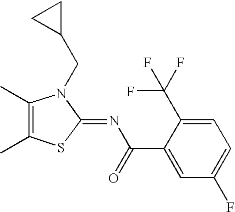 |
| 94 | 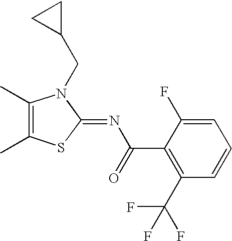 |
| 95 | 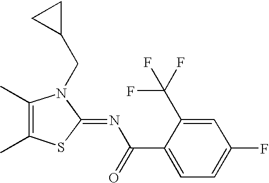 |
| 96 | 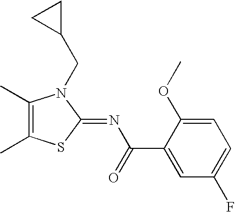 |
| 97 | 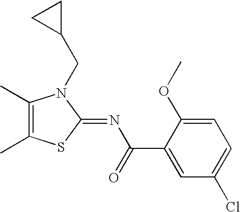 |
| 98 | 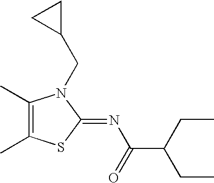 |
| 99 | 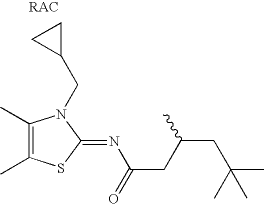 |
| 100 | 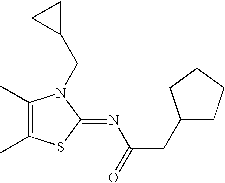 |
| 101 | 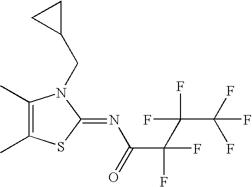 |
| 102 | 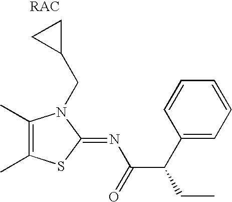 |
| 103 | 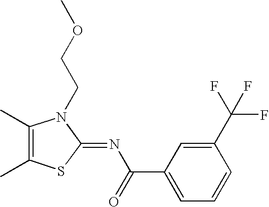 |
| 104 | 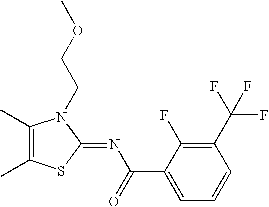 |
| 105 | 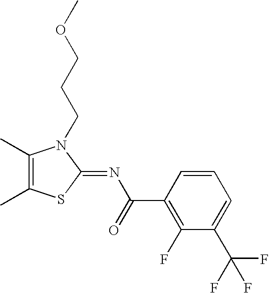 |
| 106 | 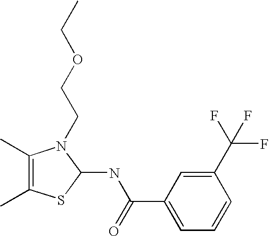 |
| 107 | 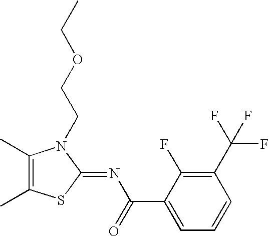 |
| 108 | 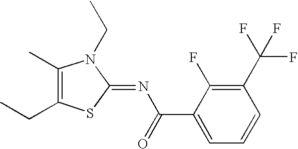 |
| 109 | 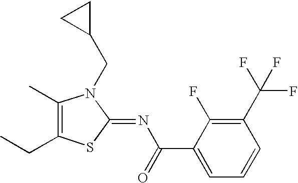 |
| 110 | 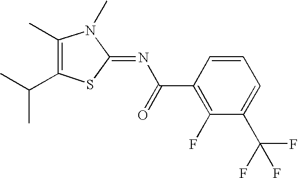 |
| 111 | 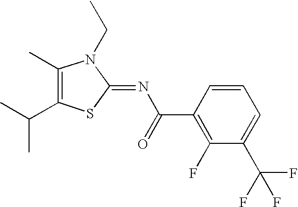 |
| 112 | 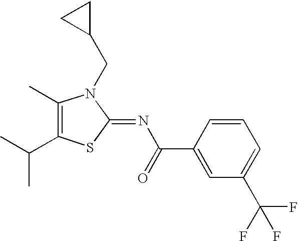 |
| 113 | 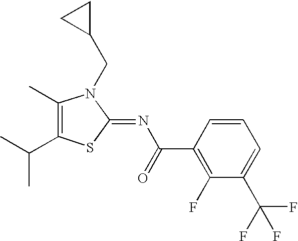 |
| 114 | 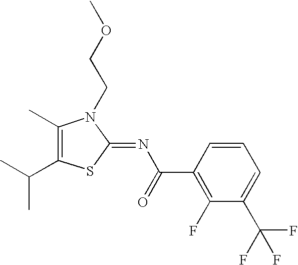 |
| 115 | 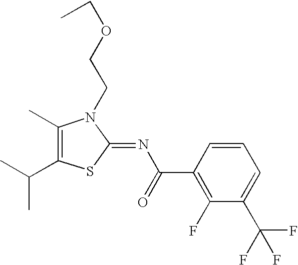 |
| 116 | 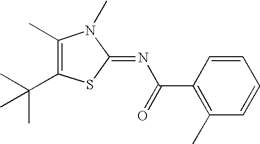 |
| 117 | 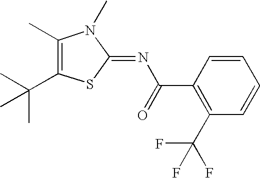 |
| 118 | 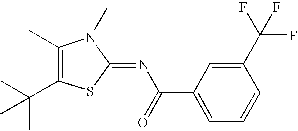 |
| 119 | 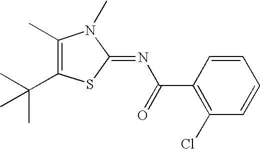 |
| 120 | 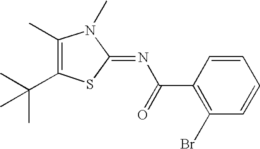 |
| 121 | 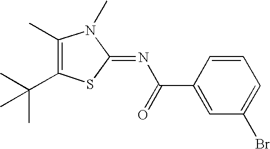 |
| 122 | 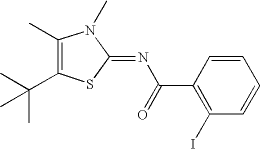 |
| 123 | 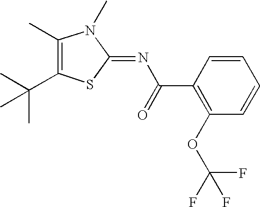 |
| 124 | 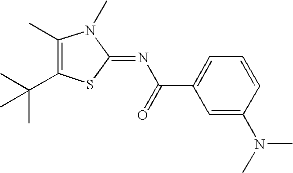 |
| 125 | 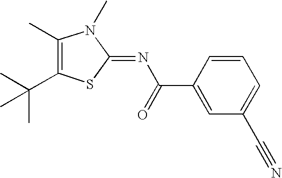 |
| 126 | 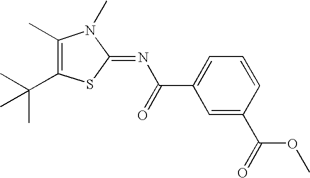 |
| 127 | 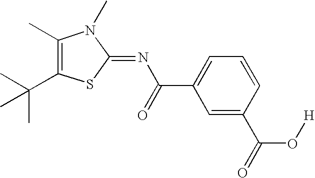 |
| 128 | 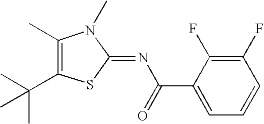 |
| 129 | 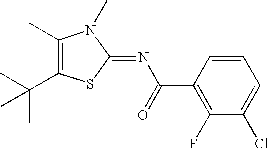 |
| 130 | 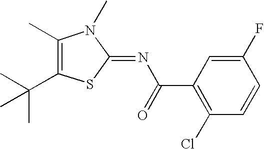 |
| 131 | 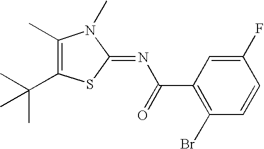 |
| 132 | 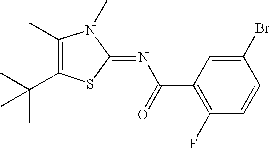 |
| 133 | 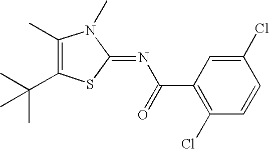 |
| 134 | 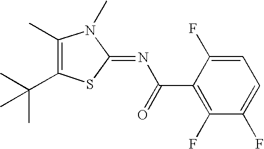 |
| 135 | 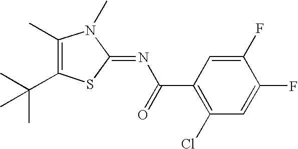 |
| 136 | 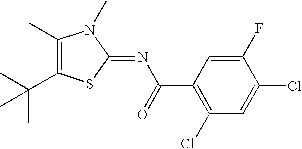 |
| 137 | 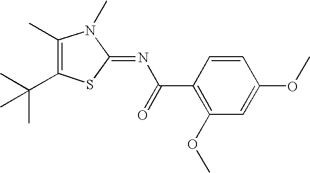 |
| 138 | 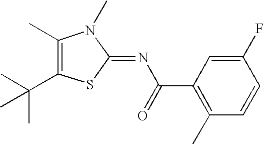 |
| 139 | 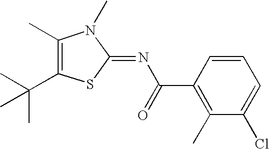 |
| 140 | 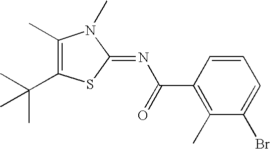 |
| 141 | 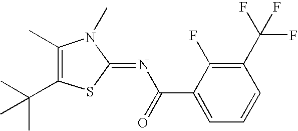 |
| 142 | 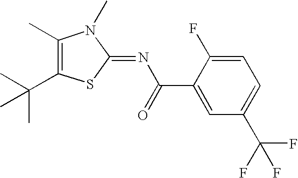 |
| 143 | 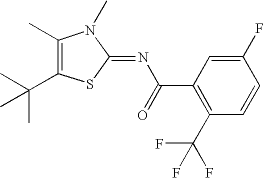 |
| 144 | 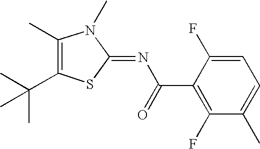 |
| 145 | 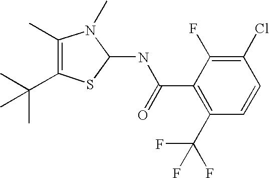 |
| 146 | 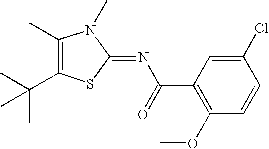 |
| 147 | 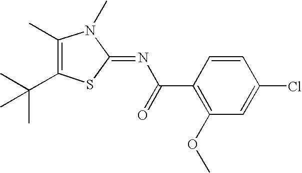 |
| 148 | 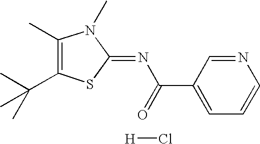 |
| 149 | 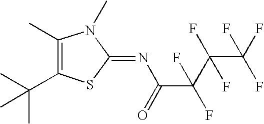 |
| 150 | 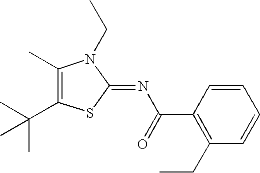 |
| 151 | 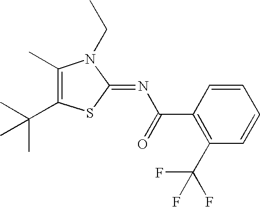 |
| 152 | 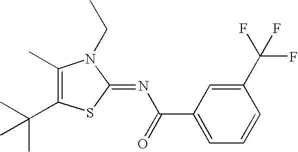 |
| 153 | 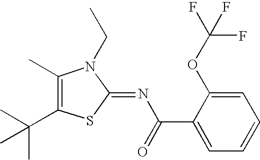 |
| 154 | 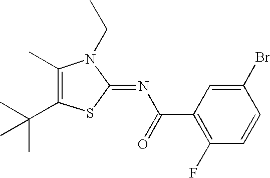 |
| 155 | 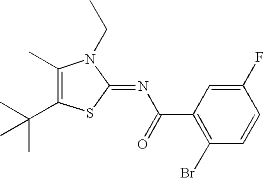 |
| 156 | 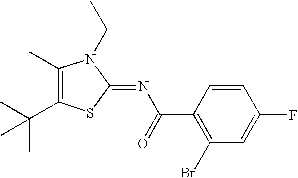 |
| 157 | 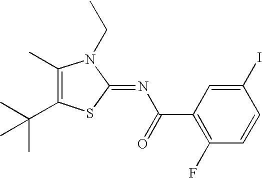 |
| 158 | 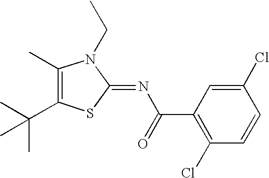 |
| 159 | 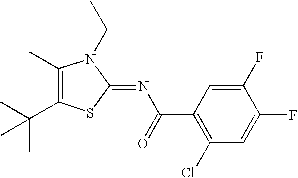 |
| 160 | 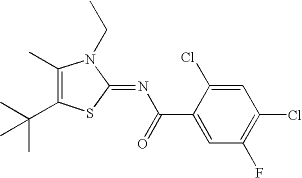 |
| 161 | 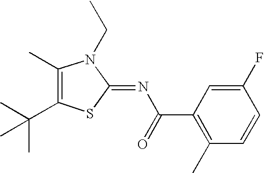 |
| 162 | 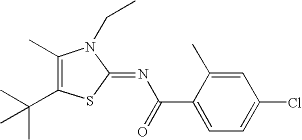 |
| 163 | 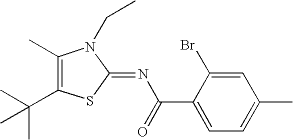 |
| 164 | 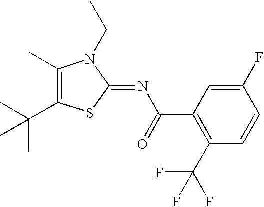 |
| 165 | 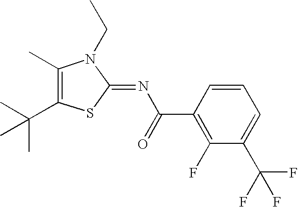 |
| 166 | 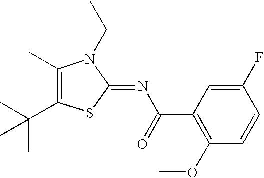 |
| 167 | 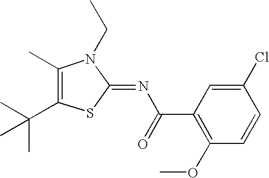 |
| 168 | 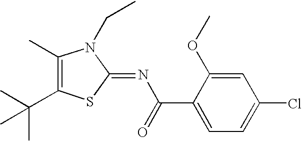 |
| 169 | 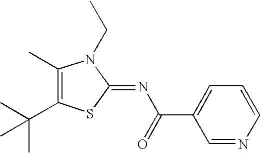 |
| 170 | 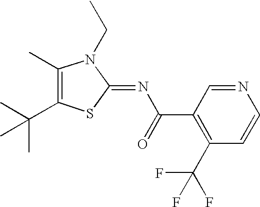 |
| 171 | 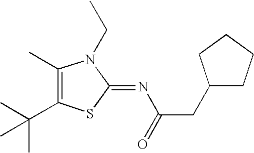 |
| 172 | 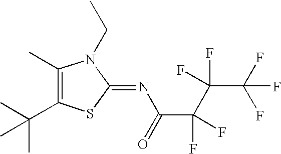 |
| 173 | 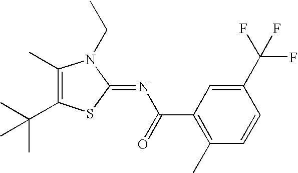 |
| 174 | 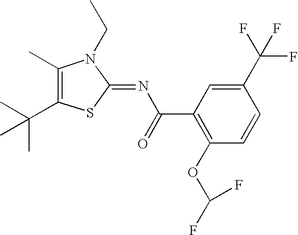 |
| 175 | 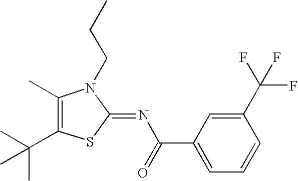 |
| 176 | 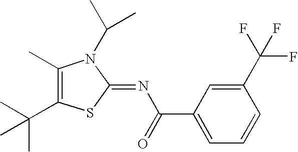 |
| 177 | 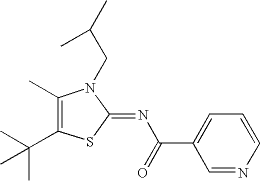 |
| 178 | 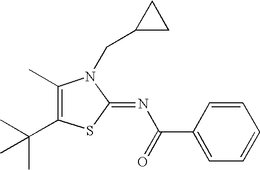 |
| 179 | 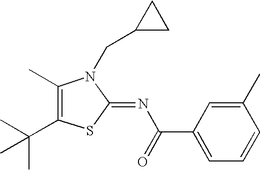 |
| 180 | 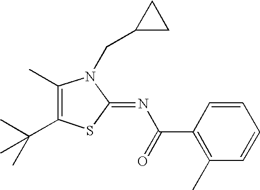 |
| 181 | 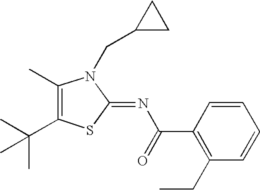 |
| 182 | 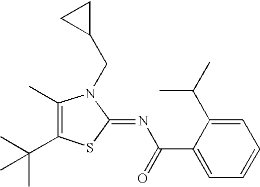 |
| 183 | 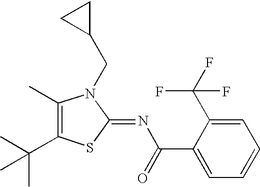 |
| 184 | 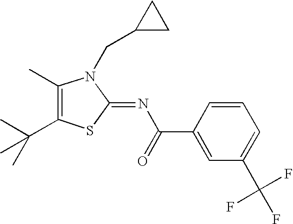 |
| 185 | 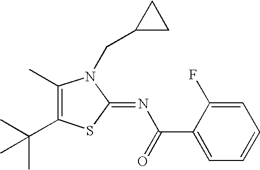 |
| 186 | 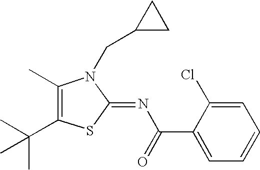 |
| 187 | 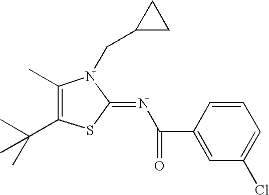 |
| 188 | 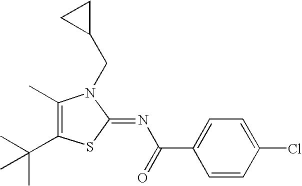 |
| 189 | 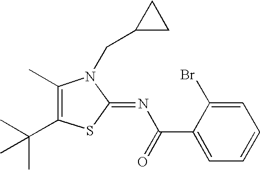 |
| 190 | 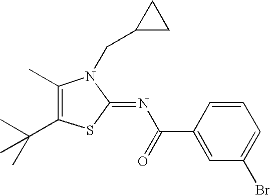 |
| 191 | 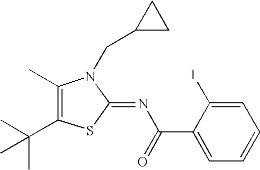 |
| 192 | 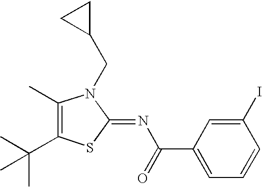 |
| 193 | 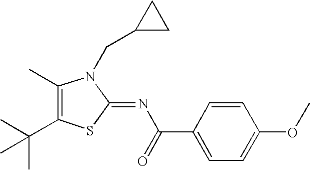 |
| 194 | 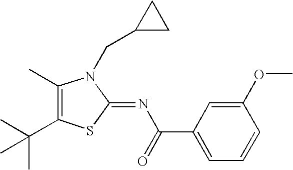 |
| 195 | 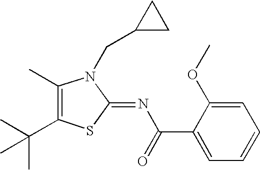 |
| 196 | 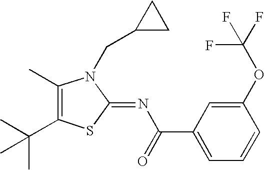 |
| 197 | 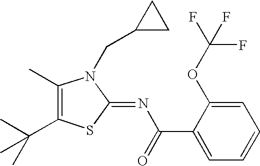 |
| 198 | 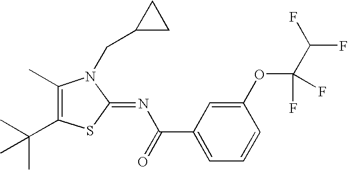 |
| 199 | 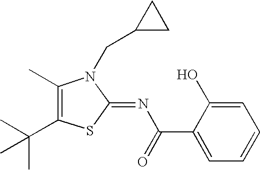 |
| 200 | 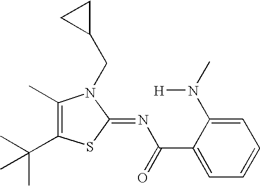 |
| 201 | 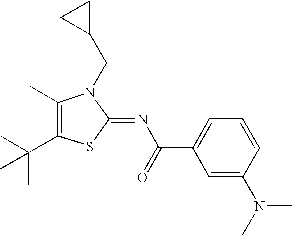 |
| 202 | 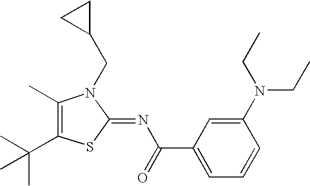 |
| 203 | 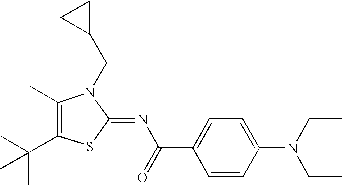 |
| 204 | 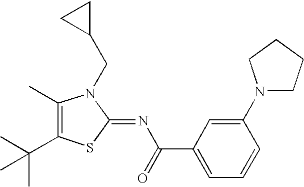 |
| 205 | 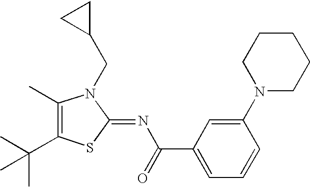 |
| 206 | 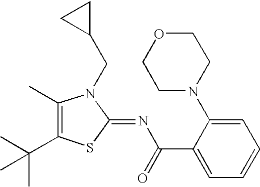 |
| 207 | 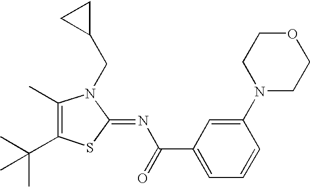 |
| 208 | 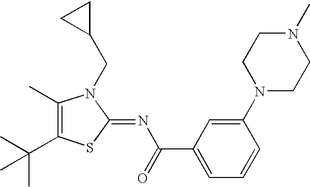 |
| 209 | 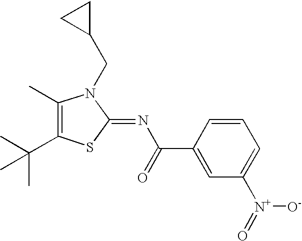 |
| 210 | 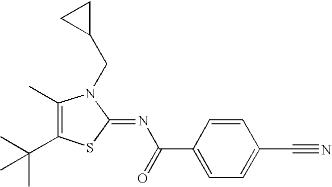 |
| 211 | 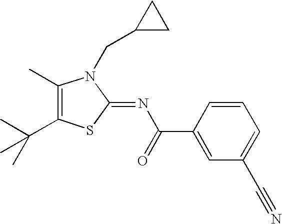 |
| 212 | 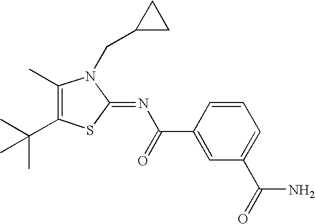 |
| 213 | 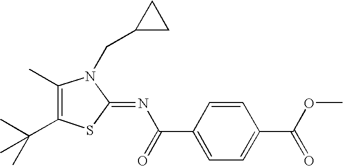 |
| 214 | 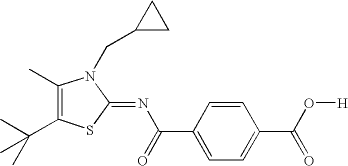 |
| 215 | 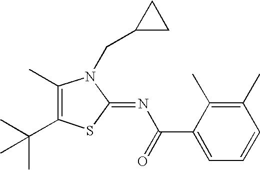 |
| 216 | 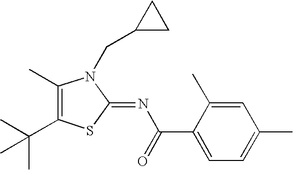 |
| 217 | 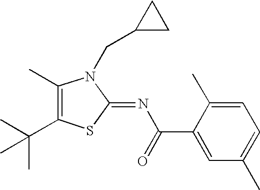 |
| 218 | 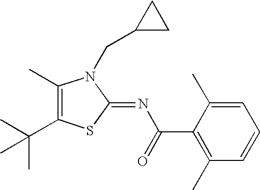 |
| 219 | 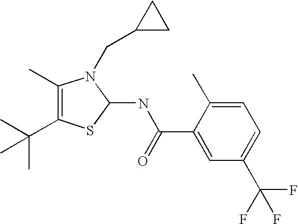 |
| 220 | 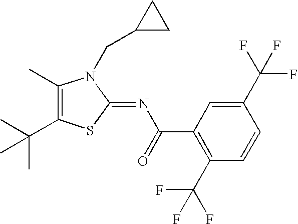 |
| 221 | 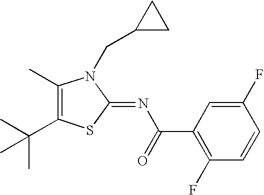 |
| 222 | 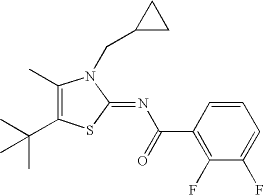 |
| 223 | 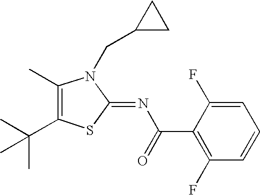 |
| 224 | 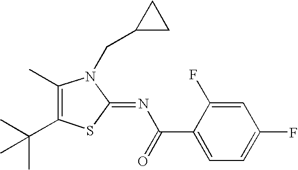 |
| 225 | 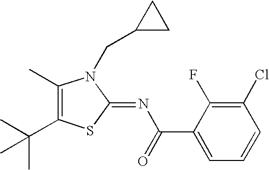 |
| 226 | 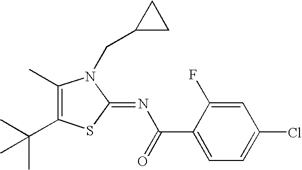 |
| 227 | 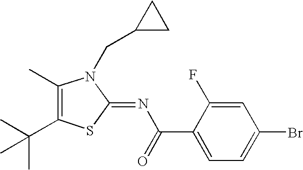 |
| 228 | 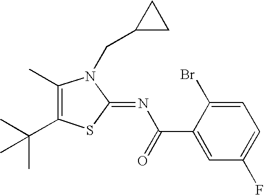 |
| 229 | 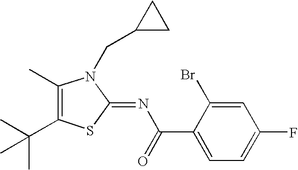 |
| 230 | 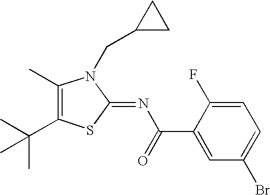 |
| 231 | 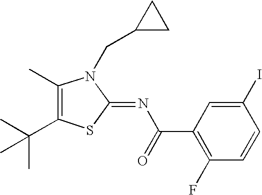 |
| 232 | 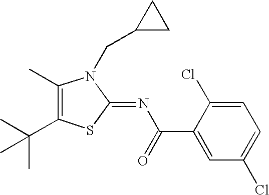 |
| 233 | 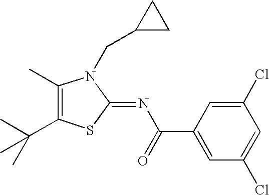 |
| 234 | 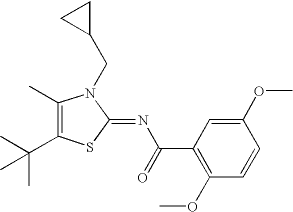 |
| 235 | 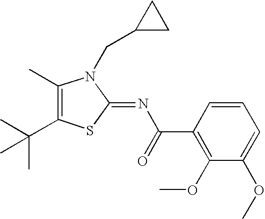 |
| 236 | 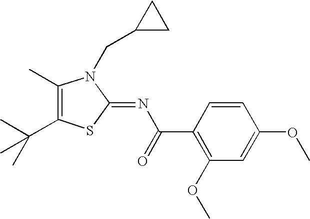 |
| 237 | 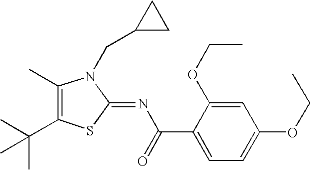 |
| 238 | 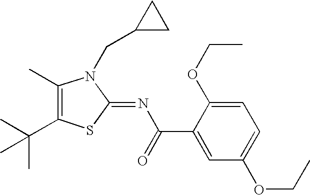 |
| 239 | 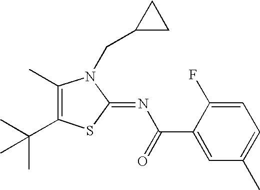 |
| 240 | 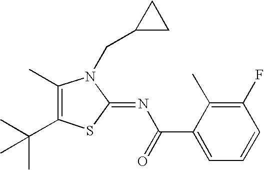 |
| 241 | 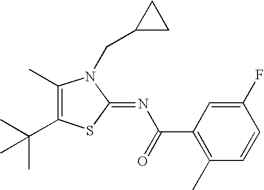 |
| 242 | 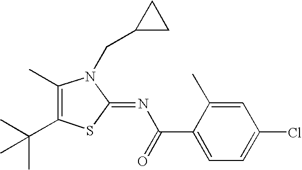 |
| 243 | 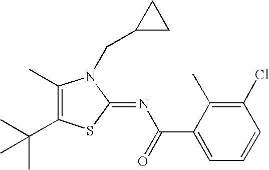 |
| 244 | 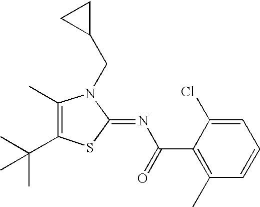 |
| 245 | 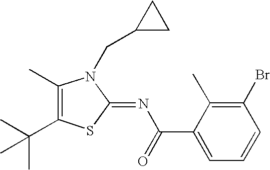 |
| 246 | 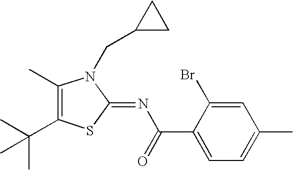 |
| 247 | 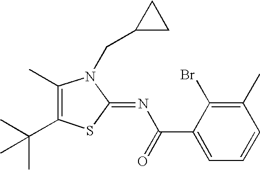 |
| 248 | 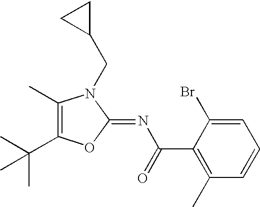 |
| 249 | 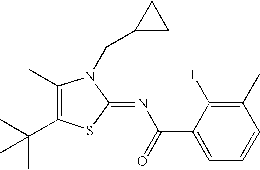 |
| 250 | 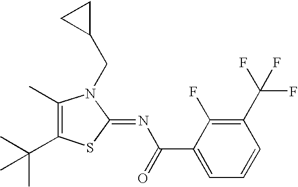 |
| 251 | 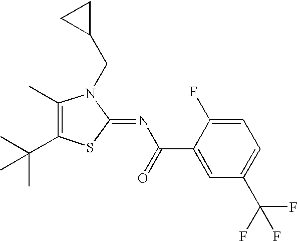 |
| 252 | 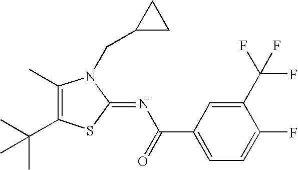 |
| 253 | 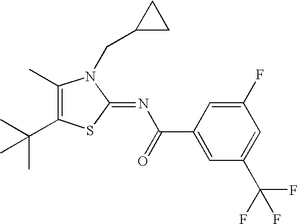 |
| 254 | 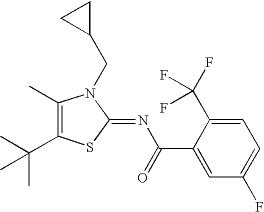 |
| 255 | 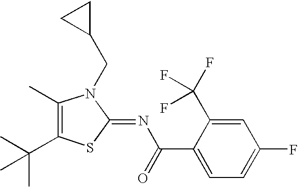 |
| 256 | 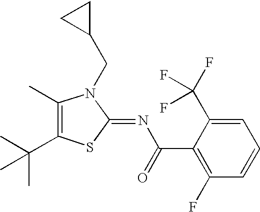 |
| 257 | 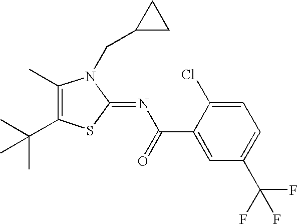 |
| 258 | 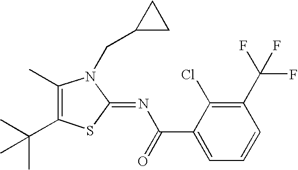 |
| 259 | 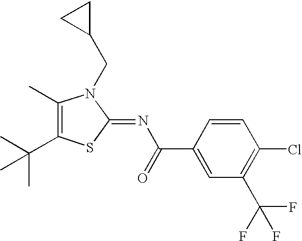 |
| 260 | 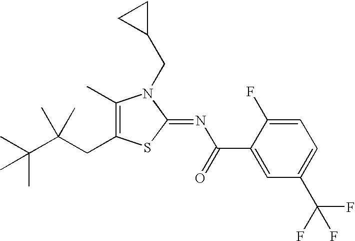 |
| 261 | 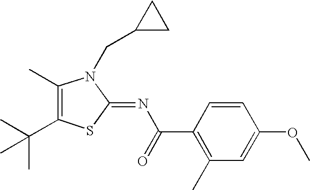 |
| 262 | 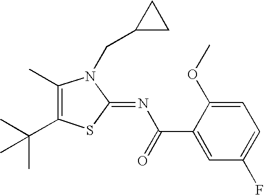 |
| 263 | 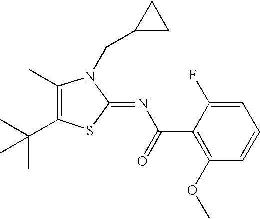 |
| 264 | 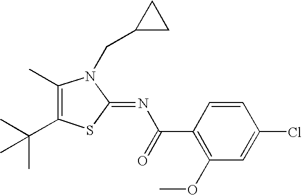 |
| 265 | 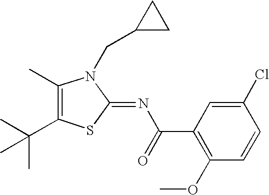 |
| 266 | 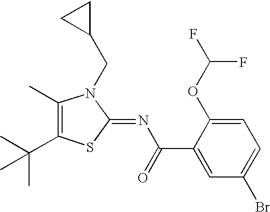 |
| 267 | 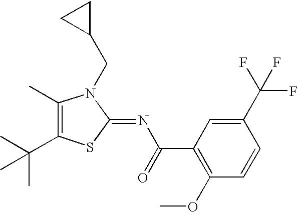 |
| 268 | 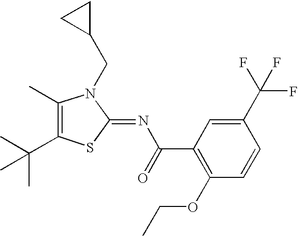 |
| 269 | 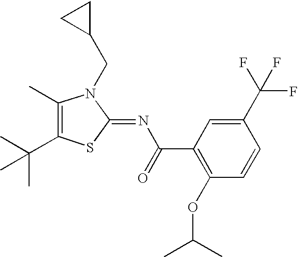 |
| 270 | 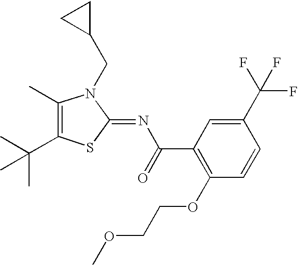 |
| 271 | 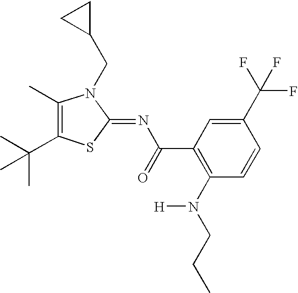 |
| 272 | 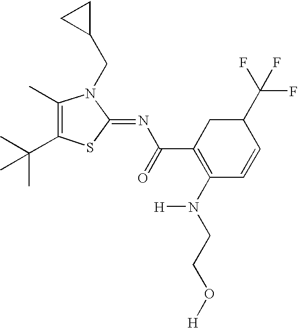 |
| 273 | 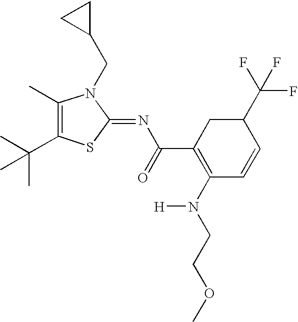 |
| 274 | 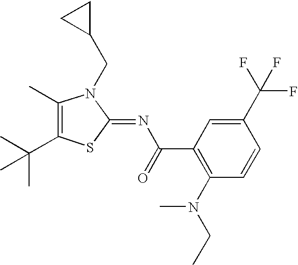 |
| 275 | 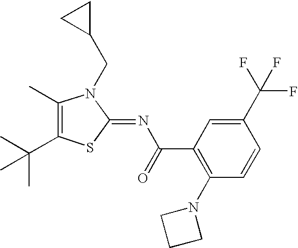 |
| 276 | 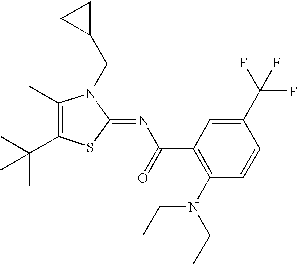 |
| 277 | 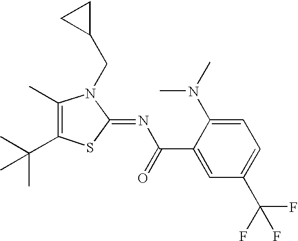 |
| 278 | 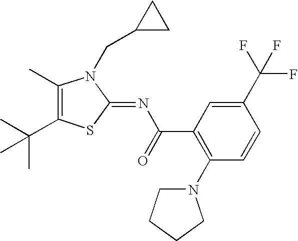 |
| 279 | 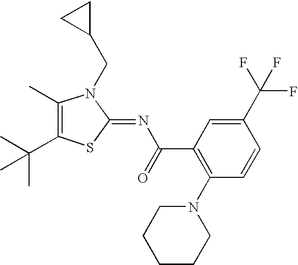 |
| 280 | 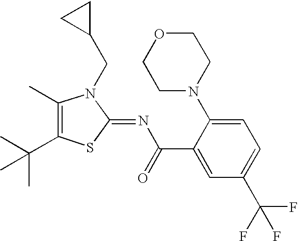 |
| 281 | 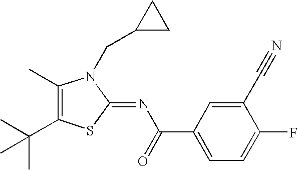 |
| 282 | 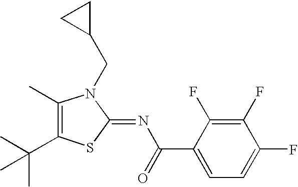 |
| 283 | 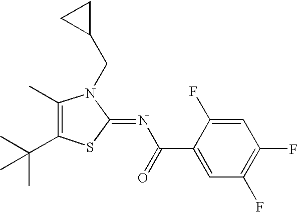 |
| 284 | 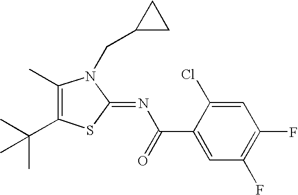 |
| 285 | 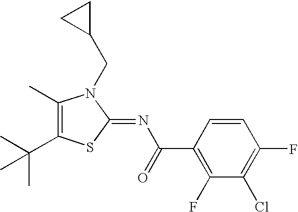 |
| 286 | 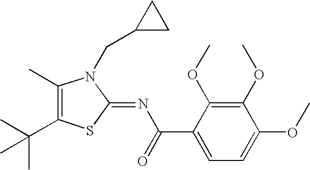 |
| 287 | 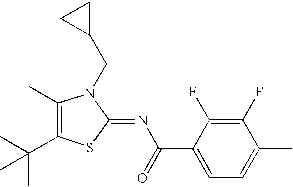 |
| 288 | 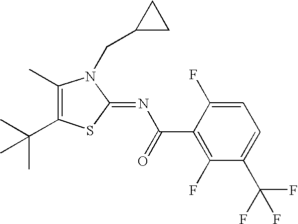 |
| 289 | 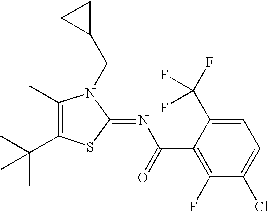 |
| 290 | 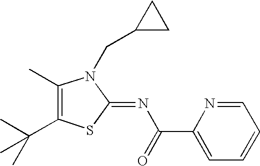 |
| 291 | 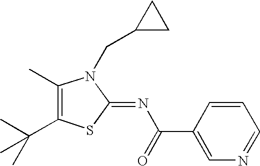 |
| 292 | 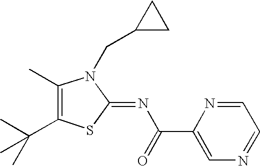 |
| 293 | 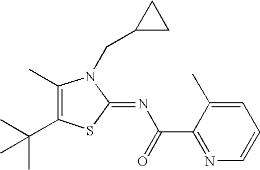 |
| 294 | 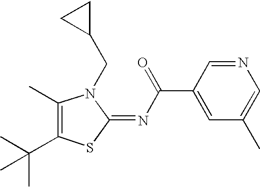 |
| 295 | 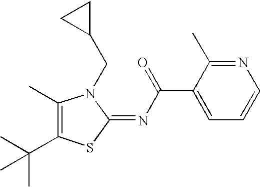 |
| 296 | 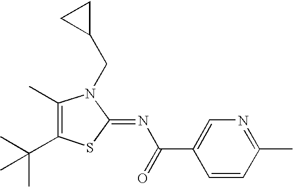 |
| 297 | 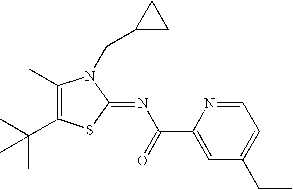 |
| 298 | 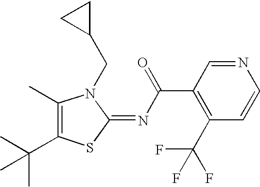 |
| 299 | 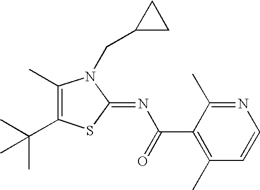 |
| 300 | 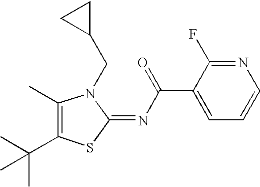 |
| 301 | 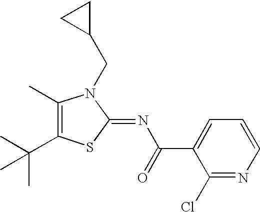 |
| 302 | 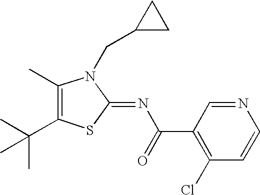 |
| 303 | 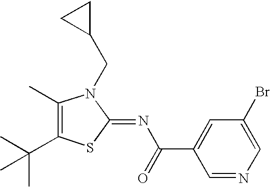 |
| 304 | 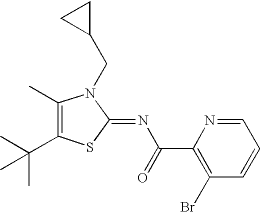 |
| 305 | 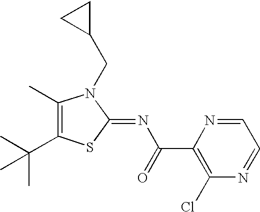 |
| 306 | 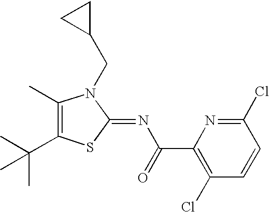 |
| 307 | 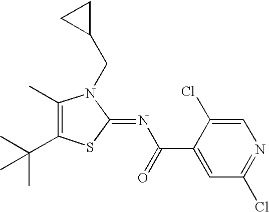 |
| 308 | 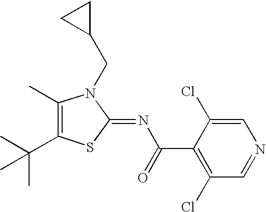 |
| 309 | 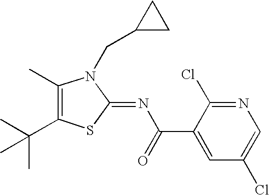 |
| 310 | 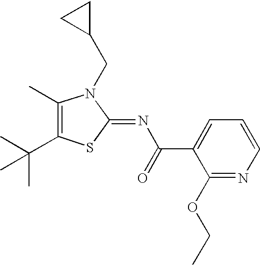 |
| 311 | 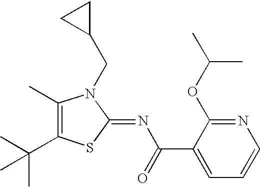 |
| 312 | 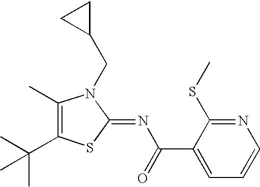 |
| 313 | 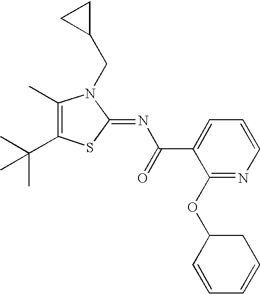 |
| 314 | 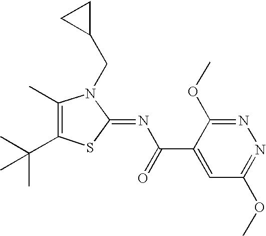 |
| 315 | 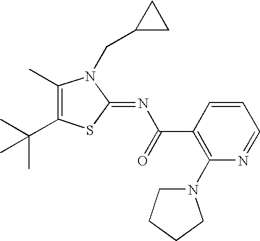 |
| 316 | 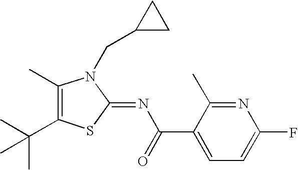 |
| 317 | 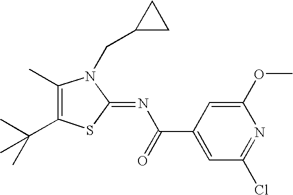 |
| 318 | 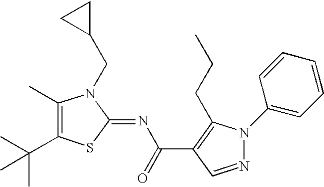 |
| 319 | 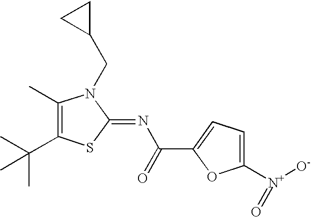 |
| 320 | 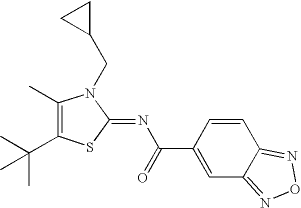 |
| 321 | 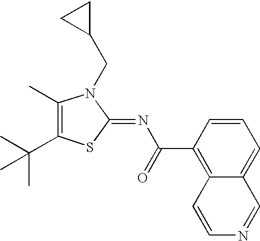 |
| 322 | 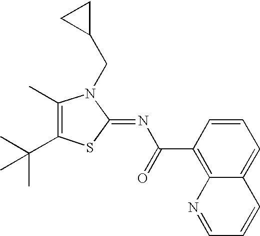 |
| 323 | 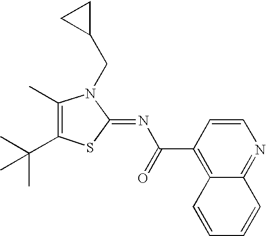 |
| 324 | 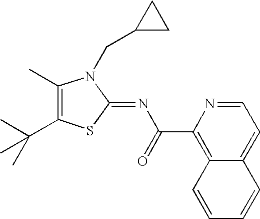 |
| 325 | 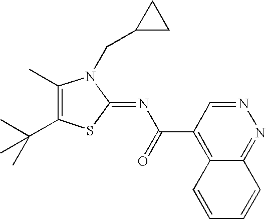 |
| 326 | 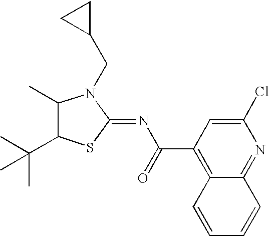 |
| 327 | 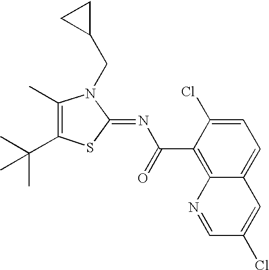 |
| 328 | 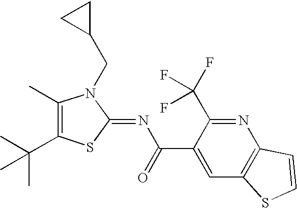 |
| 329 | 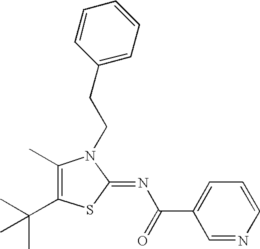 |
| 330 | 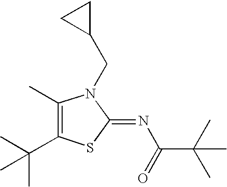 |
| 331 | 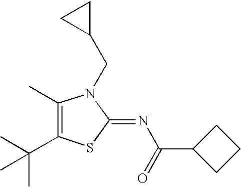 |
| 332 | 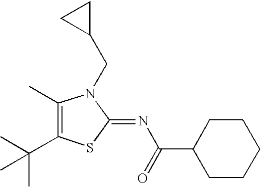 |
| 333 | 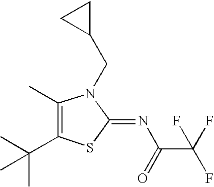 |
| 334 | 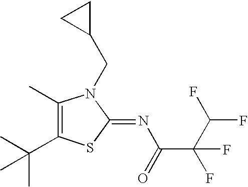 |
| 335 | 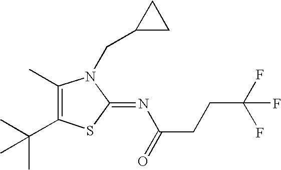 |
| 336 | 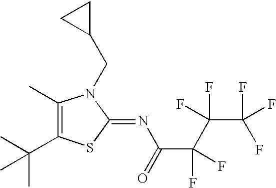 |
| 337 | 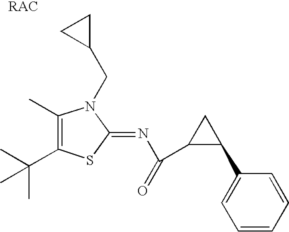 |
| 338 | 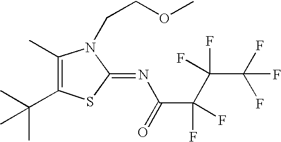 |
| 339 | 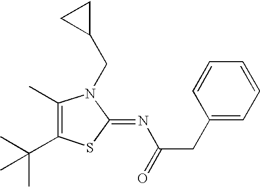 |
| 340 | 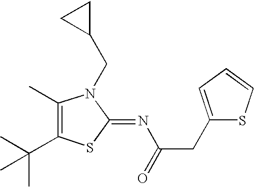 |
| 341 | 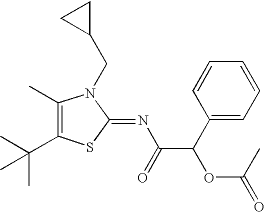 |
| 342 | 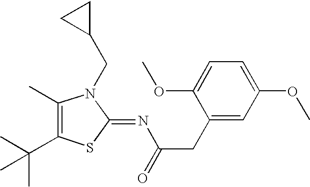 |
| 343 | 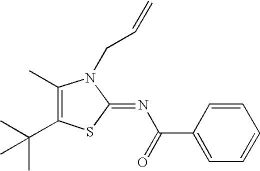 |
| 344 | 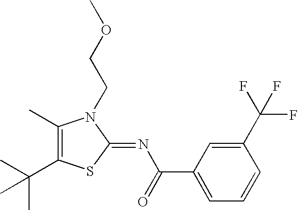 |
| 345 | 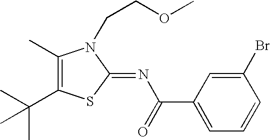 |
| 346 | 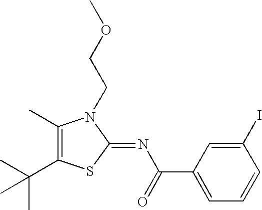 |
| 347 | 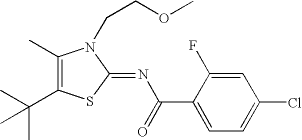 |
| 348 | 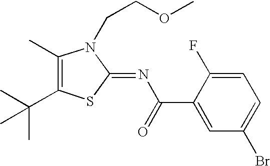 |
| 349 | 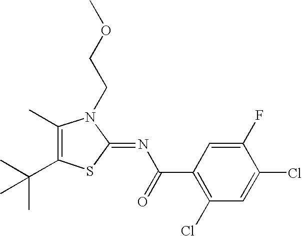 |
| 350 | 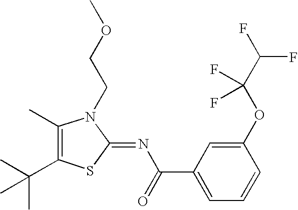 |
| 351 | 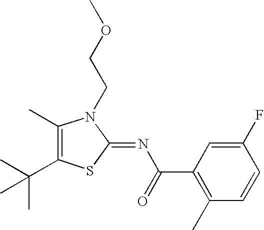 |
| 352 | 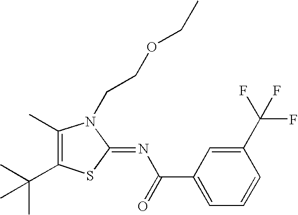 |
| 353 | 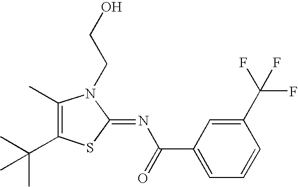 |
| 354 | 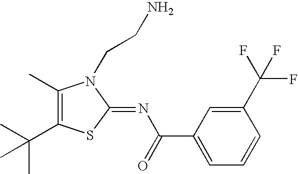 |
| 355 | 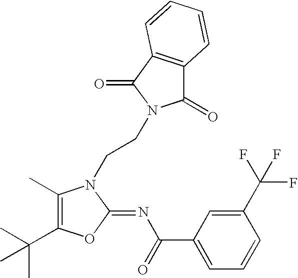 |
| 356 | 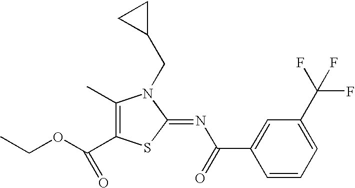 |
| 357 | 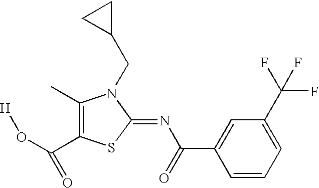 |
| 358 | 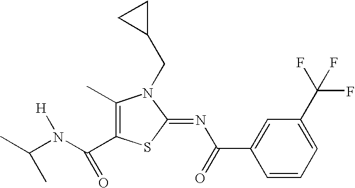 |
| 359 | 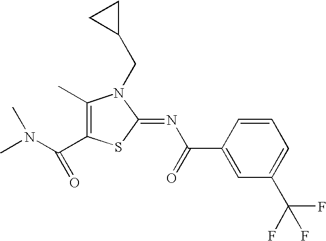 |
| 360 | 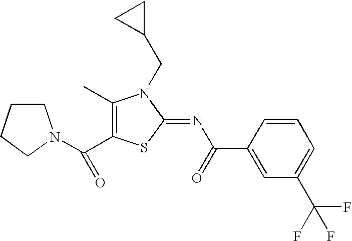 |
| 361 | 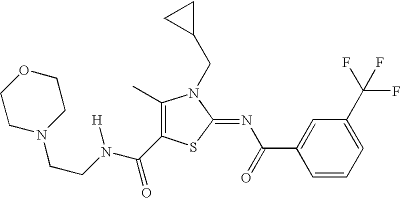 |
| 362 | 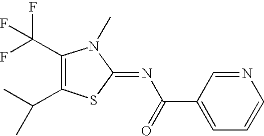 |
| 363 | 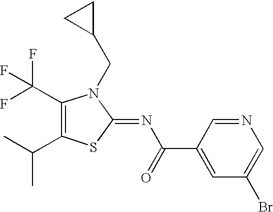 |
| 364 | 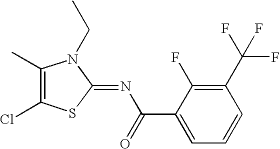 |
| 365 | 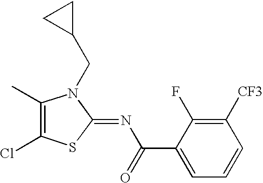 |
| 366 | 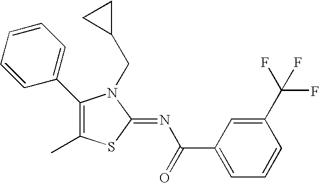 |
| 367 | 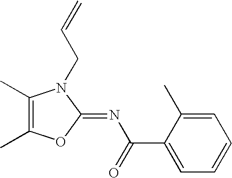 |
| 368 | 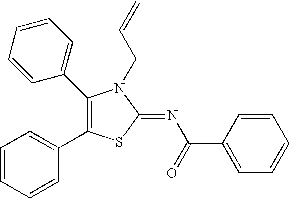 |
| 369 | 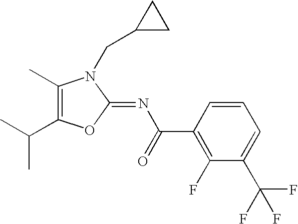 |
| 370 | 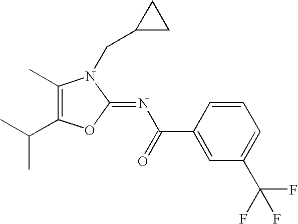 |
| 371 | 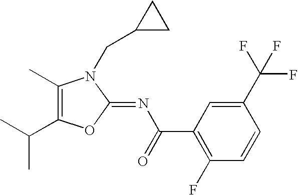 |
| 372 | 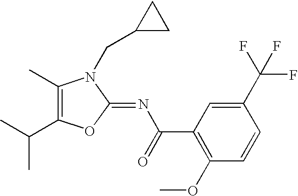 |
| 373 | 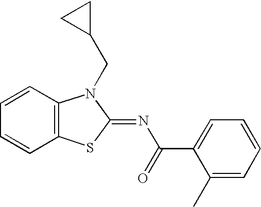 |
| 374 | 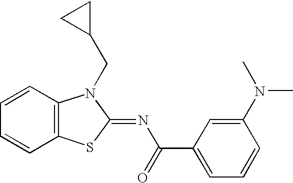 |
| 375 | 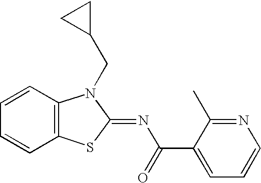 |
| 376 | 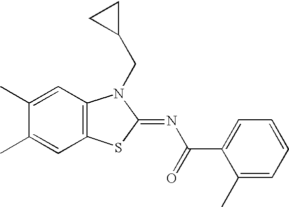 |
| 377 | 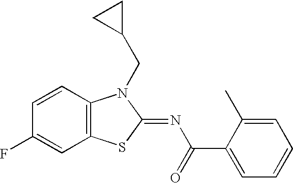 |
| 378 | 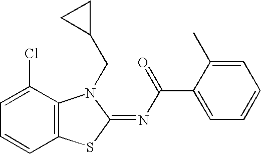 |
| 379 | 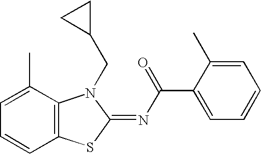 |
| 380 | 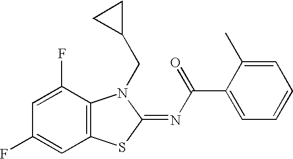 |
| 381 | 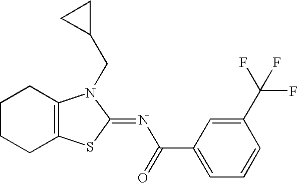 |
| 382 | 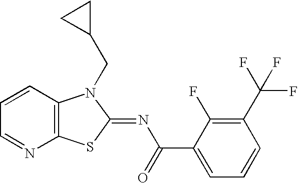 |
| 383 | 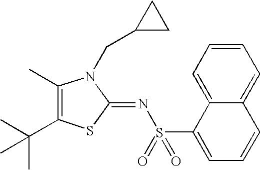 |
| 384 | 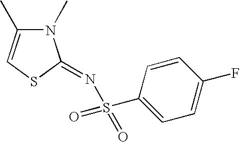 |
| 385 | 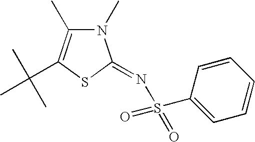 |
| 386 | 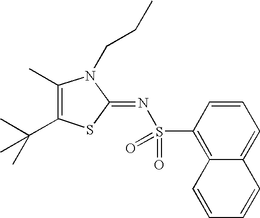 |
| 387 | 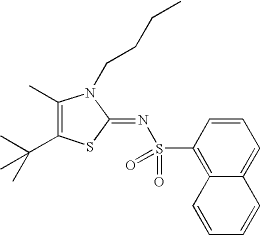 |
| 388 | 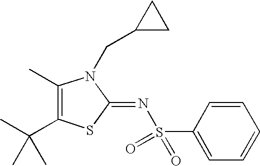 |
| 389 | 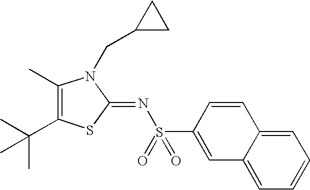 |
| 390 | 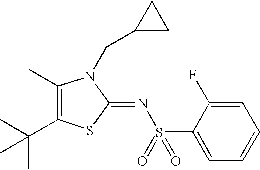 |
| 391 | 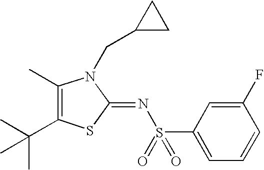 |
| 392 | 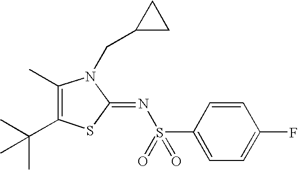 |
| 393 | 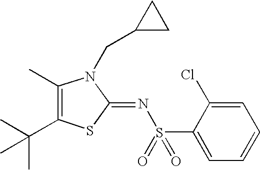 |
| 394 | 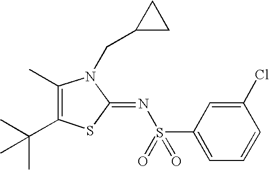 |
| 395 | 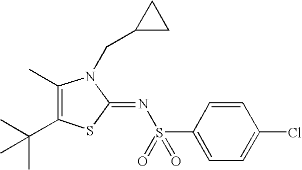 |
| 396 | 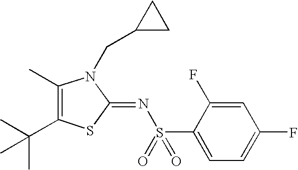 |
| 397 | 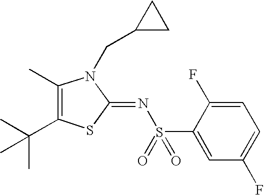 |
| 398 | 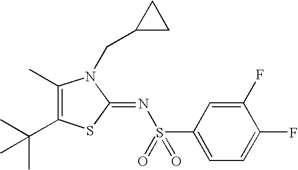 |
| 399 | 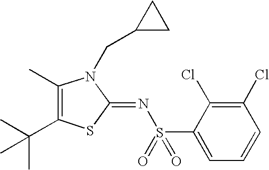 |
| 400 | 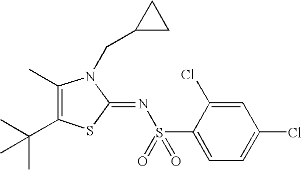 |
| 401 | 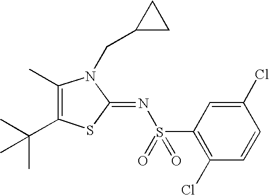 |
| 402 | 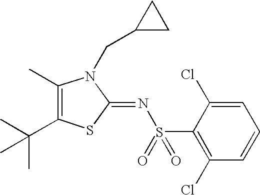 |
| 403 | 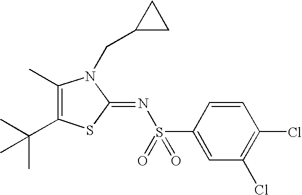 |
| 404 | 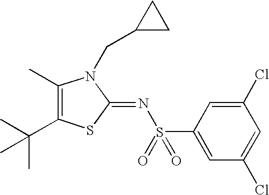 |
| 405 | 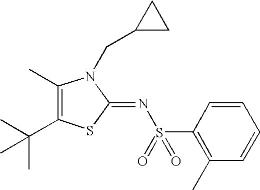 |
| 406 | 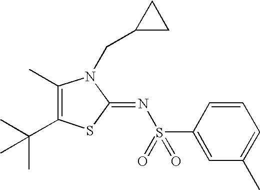 |
| 407 | 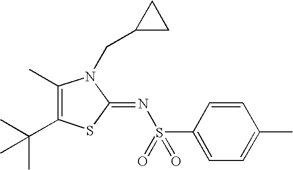 |
| 408 | 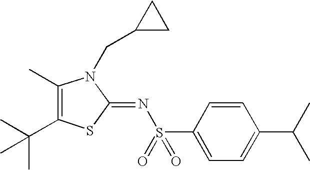 |
| 409 | 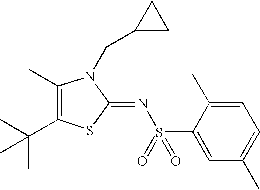 |
| 410 | 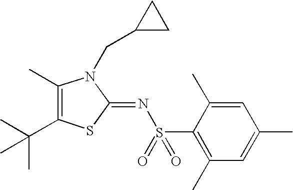 |
| 411 | 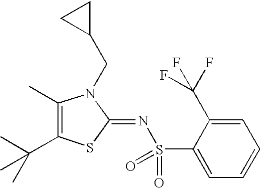 |
| 412 | 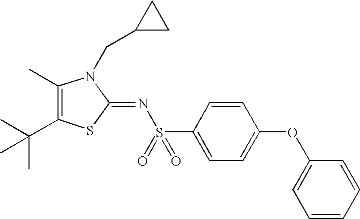 |
| 413 | 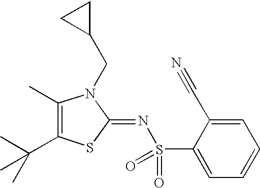 |
| 414 | 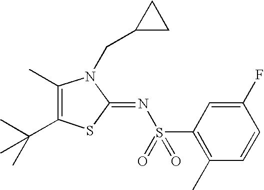 |
| 415 | 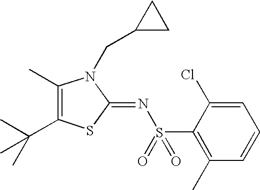 |
| 416 | 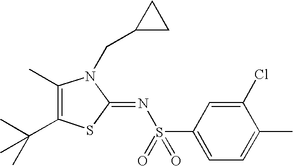 |
| 417 | 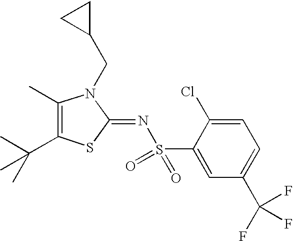 |
| 418 | 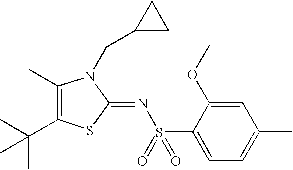 |
| 419 | 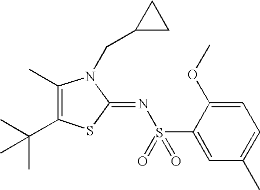 |
| 420 | 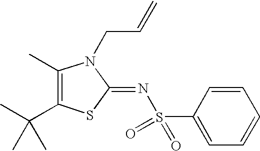 |
| 421 | 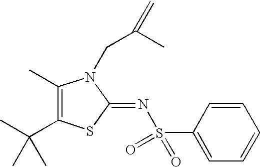 |
| 422 | 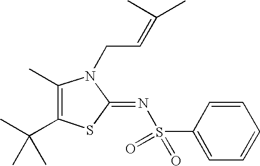 |
| 423 | 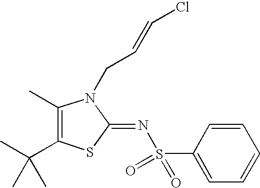 |
| 424 | 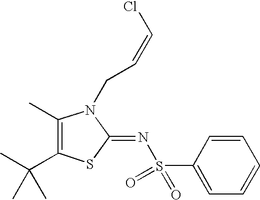 |
| 425 | 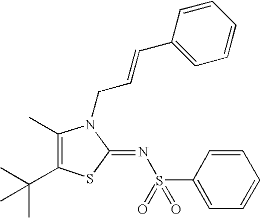 |
| 426 | 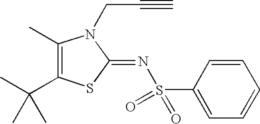 |
| 427 | 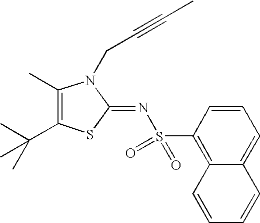 |
| 428 | 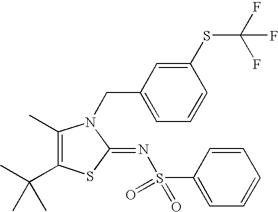 |
| 429 | 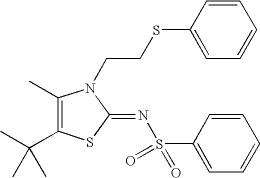 |
| 430 | 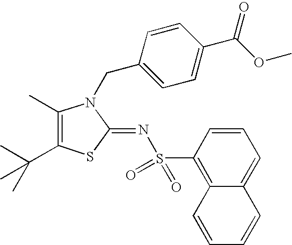 |
| 431 | 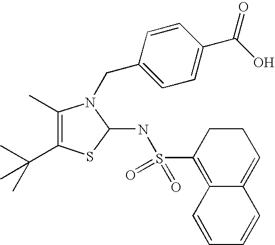 |
| 432 | 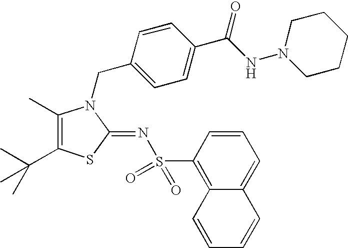 |
| 433 | 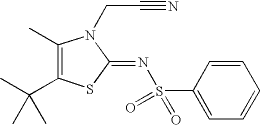 |
| 434 | 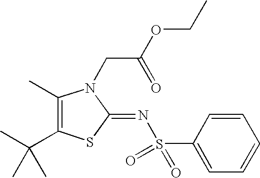 |
| 435 | 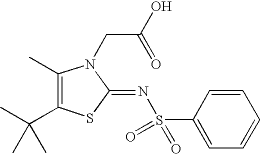 |
| 436 | 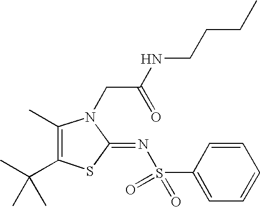 |
| 437 | 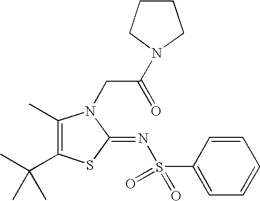 |
| 438 | 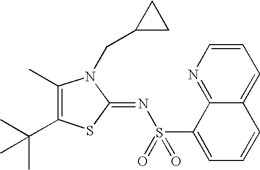 |
| 439 | 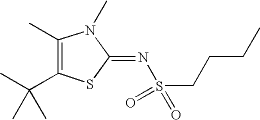 |
| 440 | 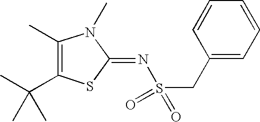 |
| 441 | 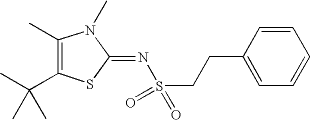 |
| 442 | 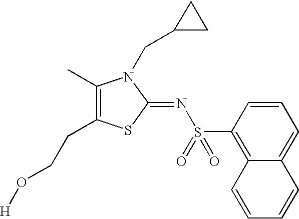 |
| 443 | 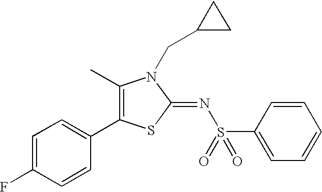 |
| 444 | 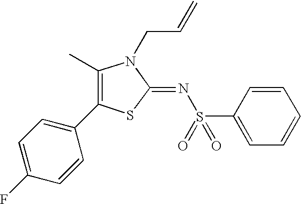 |
| 445 | 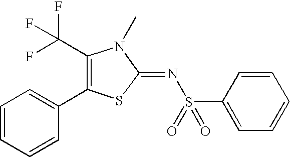 |
| 446 | 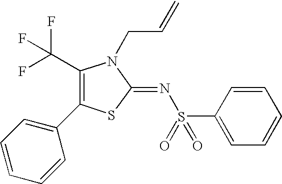 |
| 447 | 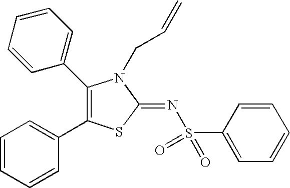 |

TABLE 3

| Example | chemical |
|---|---|
| No. | structure |
| 448 | 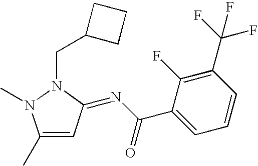 |
| 449 | 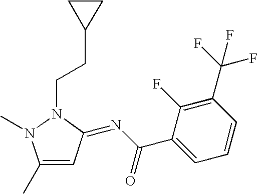 |
| 450 | 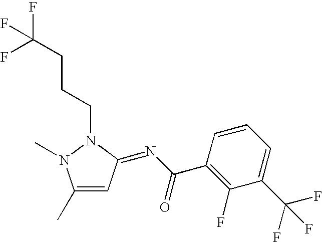 |
| 451 | 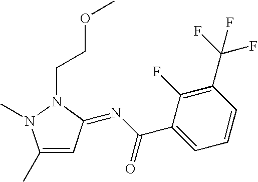 |
| 452 | 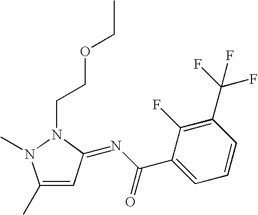 |
| 453 | 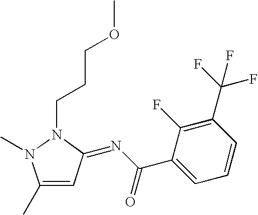 |
| 454 | 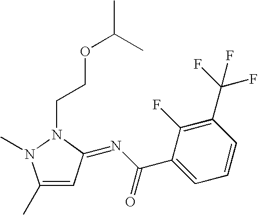 |
| 455 | 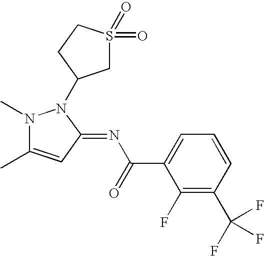 |
| 456 | 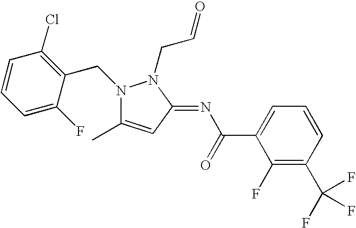 |
| 457 | 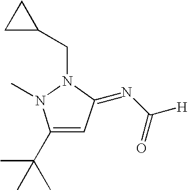 |
| 458 | 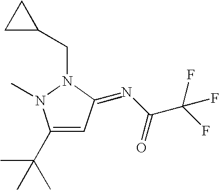 |
| 459 | 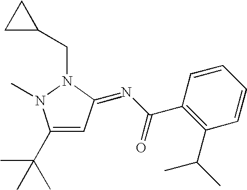 |
| 460 | 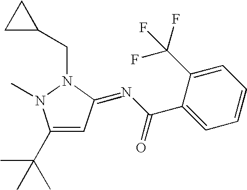 |
| 461 | 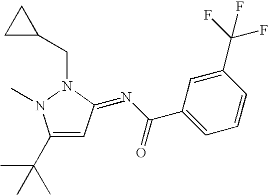 |
| 462 | 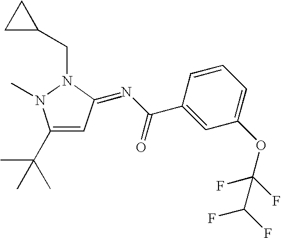 |
| 463 | 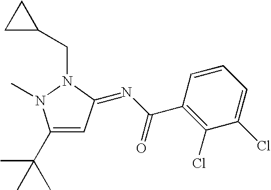 |
| 464 | 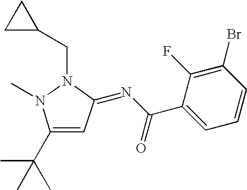 |
| 465 | 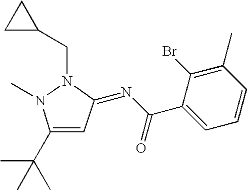 |
| 466 | 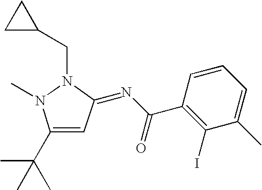 |
| 467 | 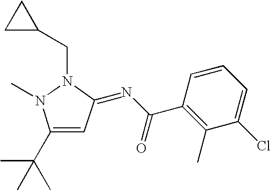 |
| 468 | 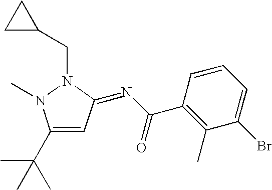 |
| 469 | 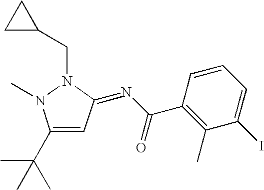 |
| 470 | 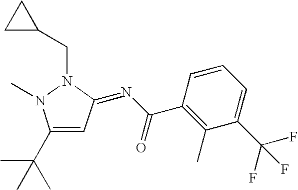 |
| 471 | 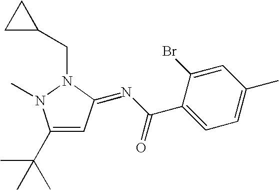 |
| 472 | 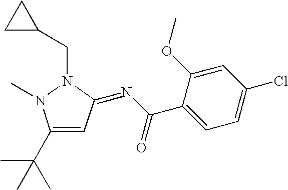 |
| 473 | 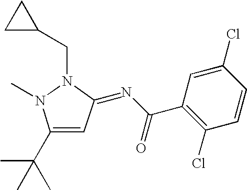 |
| 474 | 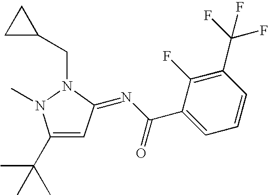 |
| 475 | 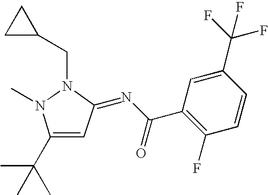 |
| 476 | 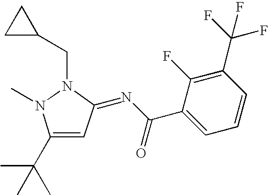 |
| 477 | 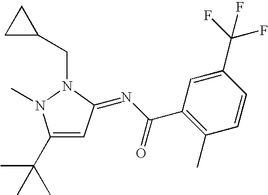 |
| 478 | 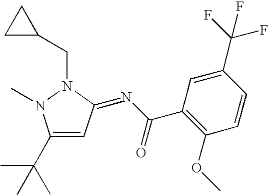 |
| 479 | 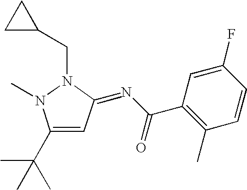 |
| 480 | 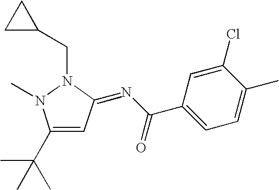 |
| 481 | 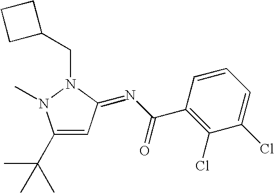 |
| 482 | 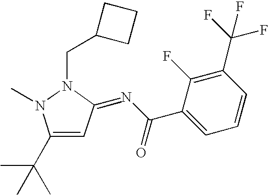 |
| 483 | 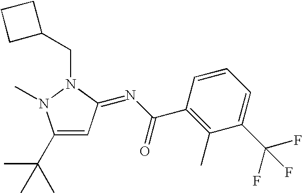 |
| 484 | 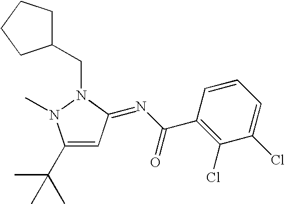 |
| 485 | 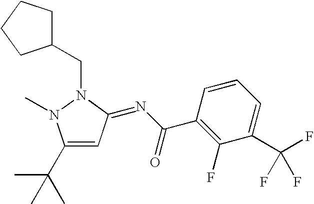 |
| 486 | 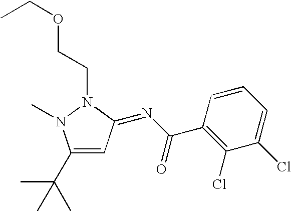 |
| 487 | 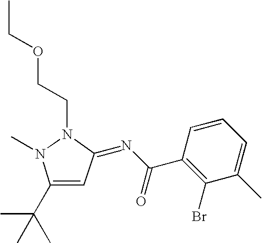 |
| 488 | 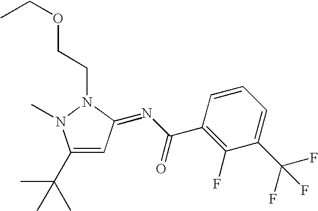 |
| 489 | 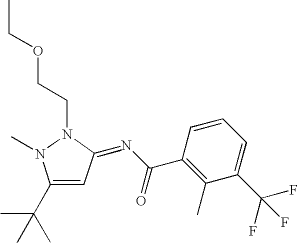 |
| 490 | 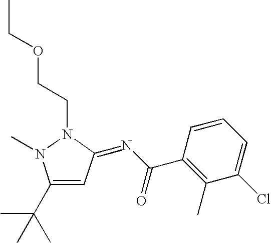 |
| 491 | 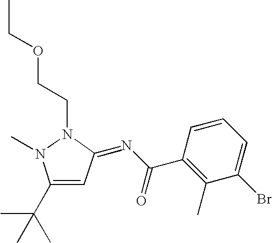 |
| 492 | 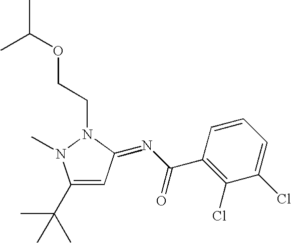 |
| 493 | 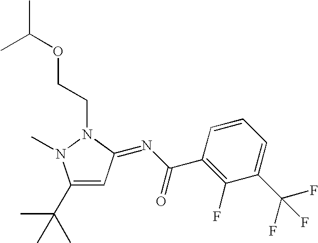 |
| 494 | 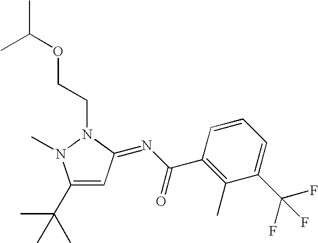 |
| 495 | 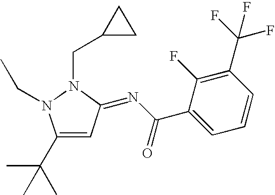 |
| 496 | 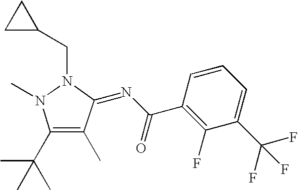 |
| 497 | 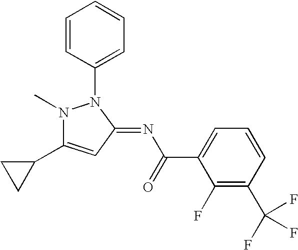 |
| 498 | 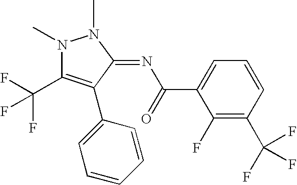 |
| 499 | 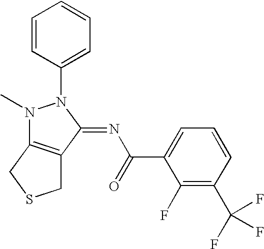 |
| 500 | 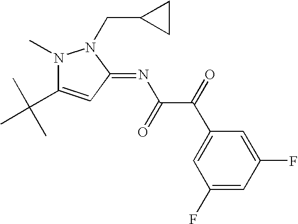 |
| 501 | 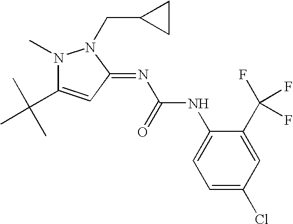 |
| 502 | 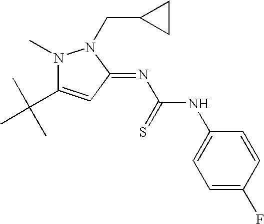 |
| 503 | 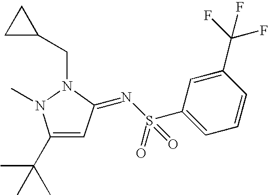 |
| 504 | 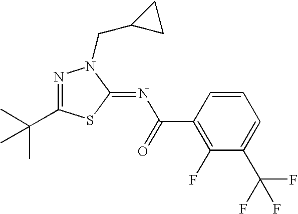 |
| 505 | 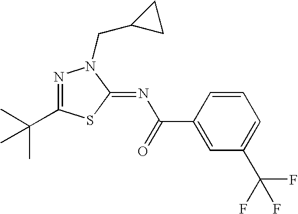 |
| 506 | 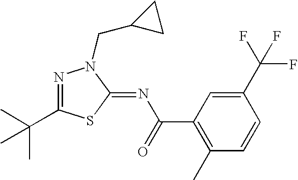 |
| 507 | 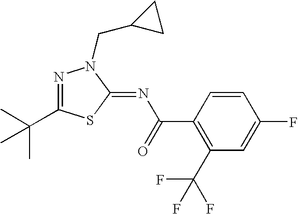 |
| 508 | 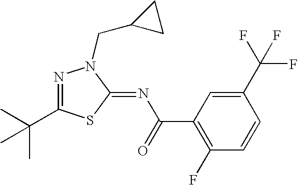 |
| 509 | 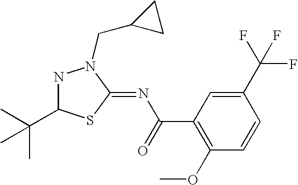 |
| 510 | 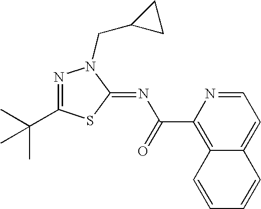 |
| 511 | 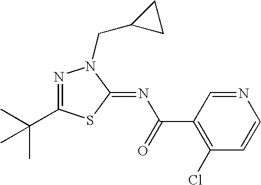 |
| 512 | 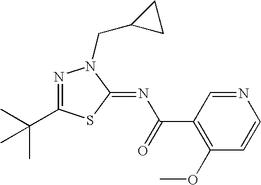 |
| 513 | 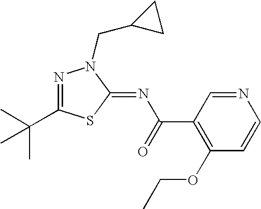 |
| 514 | 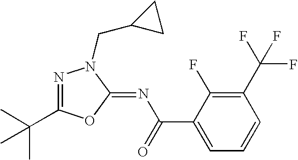 |
| 515 | 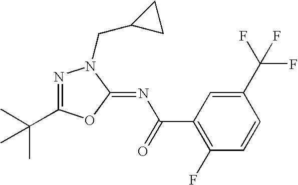 |

TABLE 4

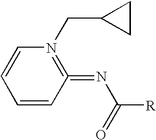

| Compound | |
|---|---|
| No. | R |
| 1001 | 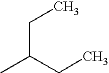 |
| 1002 | 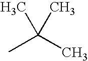 |
| 1003 | 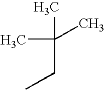 |
| 1004 | 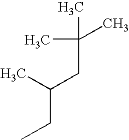 |
| 1005 | 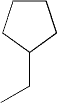 |
| 1006 | 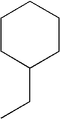 |
| 1007 | 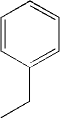 |
| 1008 | 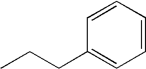 |
| 1009 | 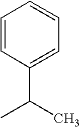 |
| 1010 | 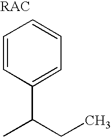 |
| 1011 | 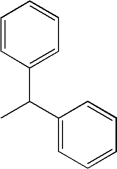 |
| 1012 | 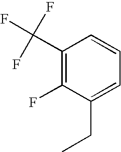 |
| 1013 | 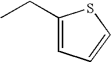 |
| 1014 | 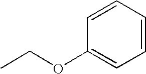 |
| 1015 | 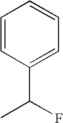 |
| 1016 | 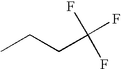 |
| 1017 | 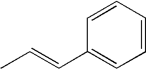 |
| 1018 | 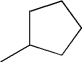 |
| 1019 | 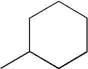 |
| 1020 | 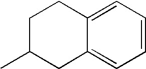 |
| 1021 | 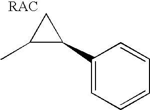 |
| 1022 | 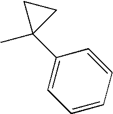 |
| 1023 | 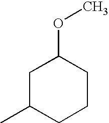 |
| 1024 | 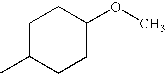 |
| 1025 | 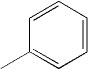 |
| 1026 | 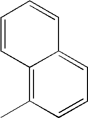 |
| 1027 | 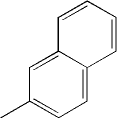 |
| 1028 | 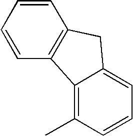 |
| 1029 | 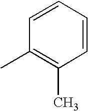 |
| 1030 | 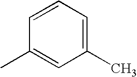 |
| 1031 | 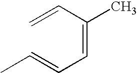 |
| 1032 | 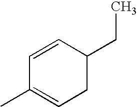 |
| 1033 | 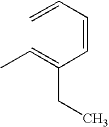 |
| 1034 | 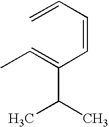 |
| 1035 | 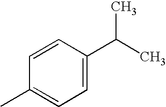 |
| 1036 | 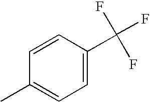 |
| 1037 | 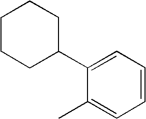 |
| 1038 | 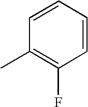 |
| 1039 | 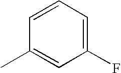 |
| 1040 | 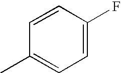 |
| 1041 | 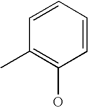 |
| 1042 | 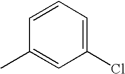 |
| 1043 | 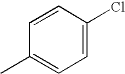 |
| 1044 | 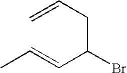 |
| 1045 | 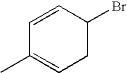 |
| 1046 | 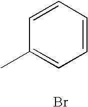 |
| 1047 | 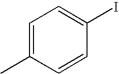 |
| 1048 | 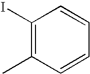 |
| 1049 | 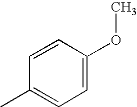 |
| 1050 | 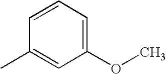 |
| 1051 | 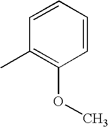 |
| 1052 | 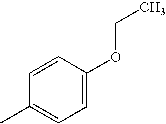 |
| 1053 | 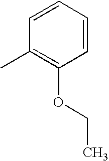 |
| 1054 | 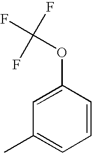 |
| 1055 | 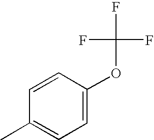 |
| 1056 | 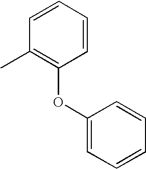 |
| 1057 | 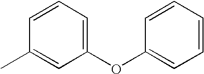 |
| 1058 | 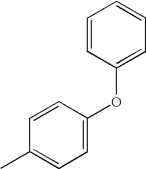 |
| 1059 | 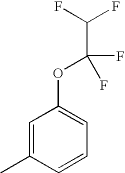 |
| 1060 | 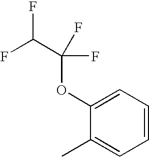 |
| 1061 | 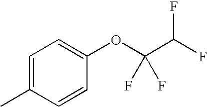 |
| 1062 | 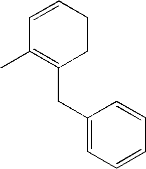 |
| 1063 | 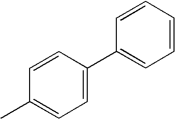 |
| 1064 | 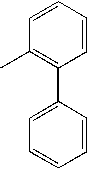 |
| 1065 | 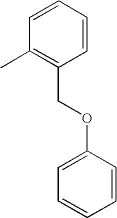 |
| 1066 | 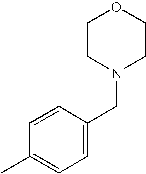 |
| 1067 | 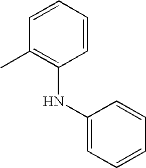 |
| 1068 | 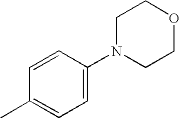 |
| 1069 | 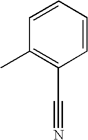 |
| 1070 | 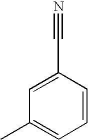 |
| 1071 | 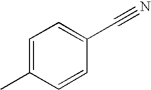 |
| 1072 | 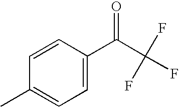 |
| 1073 | 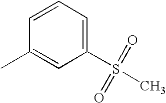 |
| 1074 | 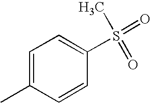 |
| 1075 | 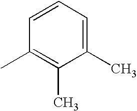 |
| 1076 | 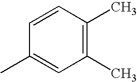 |
| 1077 | 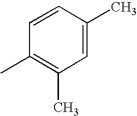 |
| 1078 | 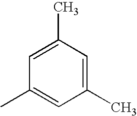 |
| 1079 | 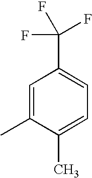 |
| 1080 | 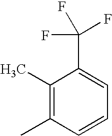 |
| 1081 | 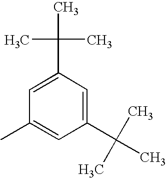 |
| 1082 | 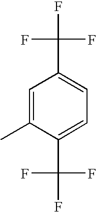 |
| 1083 | 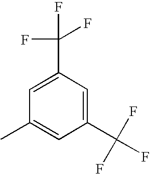 |
| 1084 | 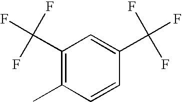 |
| 1085 | 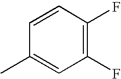 |
| 1086 | 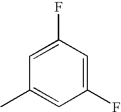 |
| 1087 | 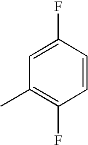 |
| 1088 | 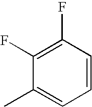 |
| 1089 | 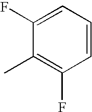 |
| 1090 | 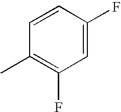 |
| 1091 | 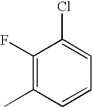 |
| 1092 | 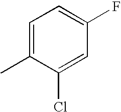 |
| 1093 | 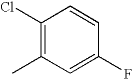 |
| 1094 | 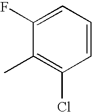 |
| 1095 | 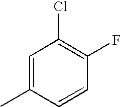 |
| 1096 | 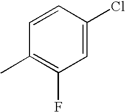 |
| 1097 | 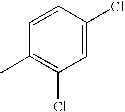 |
| 1098 | 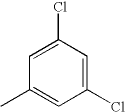 |
| 1099 | 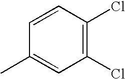 |
| 1100 | 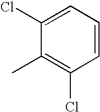 |
| 1101 | 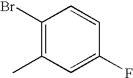 |
| 1102 | 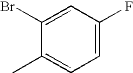 |
| 1103 | 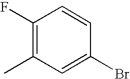 |
| 1104 | 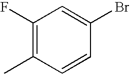 |
| 1105 | 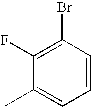 |
| 1106 | 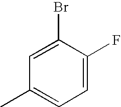 |
| 1107 | 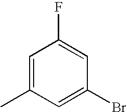 |
| 1108 | 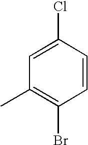 |
| 1109 | 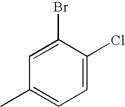 |
| 1110 | 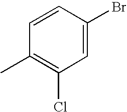 |
| 1111 | 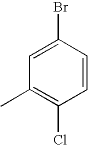 |
| 1112 | 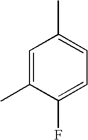 |
| 1113 | 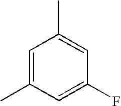 |
| 1114 | 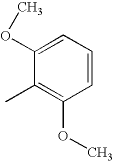 |
| 1115 | 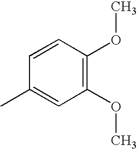 |
| 1116 | 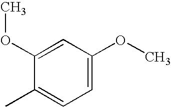 |
| 1117 | 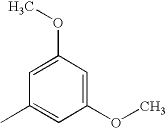 |
| 1118 | 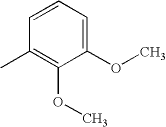 |
| 1119 | 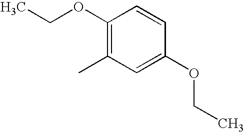 |
| 1120 | 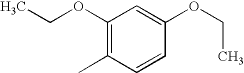 |
| 1121 | 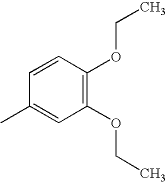 |
| 1122 | 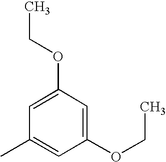 |
| 1123 | 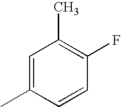 |
| 1124 | 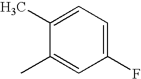 |
| 1125 | 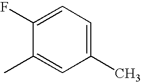 |
| 1126 | 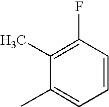 |
| 1127 | 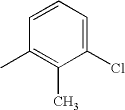 |
| 1128 | 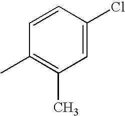 |
| 1129 | 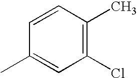 |
| 1130 | 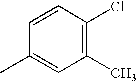 |
| 1131 | 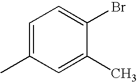 |
| 1132 | 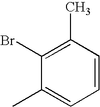 |
| 1133 | 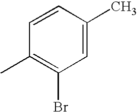 |
| 1134 | 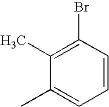 |
| 1135 | 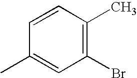 |
| 1136 | 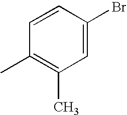 |
| 1137 | 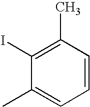 |
| 1138 | 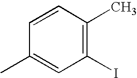 |
| 1139 | 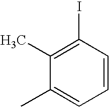 |
| 1140 | 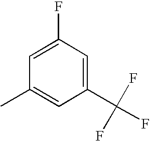 |
| 1141 | 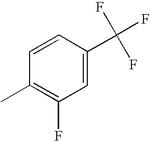 |
| 1142 | 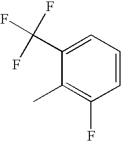 |
| 1143 | 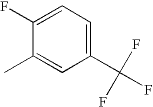 |
| 1144 | 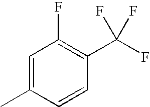 |
| 1145 | 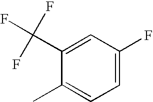 |
| 1146 | 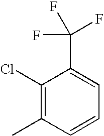 |
| 1147 | 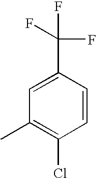 |
| 1148 | 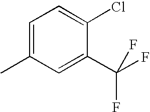 |
| 1149 | 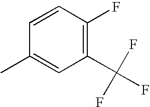 |
| 1150 | 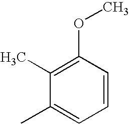 |
| 1151 | 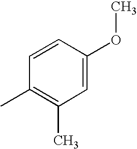 |
| 1152 | 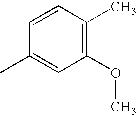 |
| 1153 | 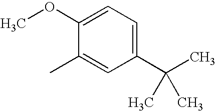 |
| 1154 | 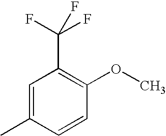 |
| 1155 | 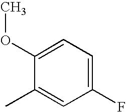 |
| 1156 | 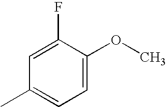 |
| 1157 | 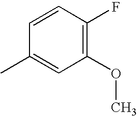 |
| 1158 | 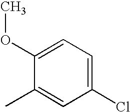 |
| 1159 | 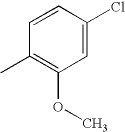 |
| 1160 | 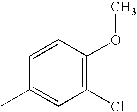 |
| 1161 | 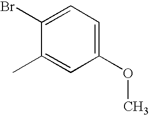 |
| 1162 | 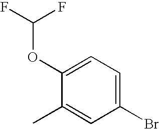 |
| 1163 | 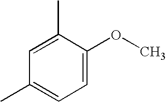 |
| 1164 | 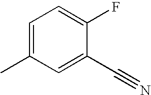 |
| 1165 | 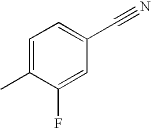 |
| 1166 | 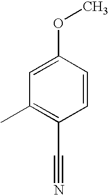 |
| 1167 | 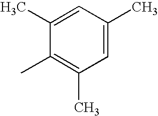 |
| 1168 | 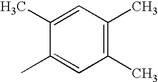 |
| 1169 | 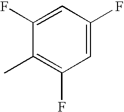 |
| 1170 | 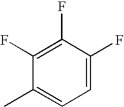 |
| 1171 | 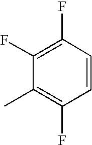 |
| 1172 | 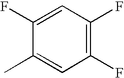 |
| 1173 | 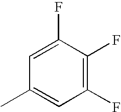 |
| 1174 | 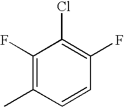 |
| 1175 | 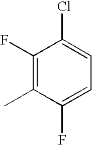 |
| 1176 | 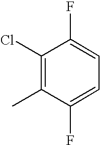 |
| 1177 | 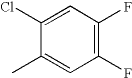 |
| 1178 | 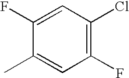 |
| 1179 | 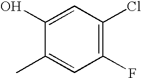 |
| 1180 | 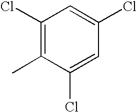 |
| 1181 | 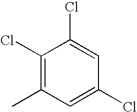 |
| 1182 | 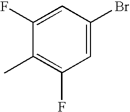 |
| 1183 | 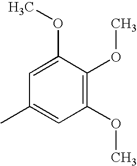 |
| 1184 | 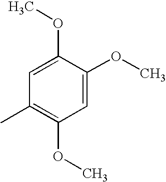 |
| 1185 | 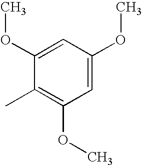 |
| 1186 | 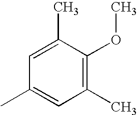 |
| 1187 | 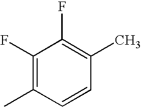 |
| 1188 | 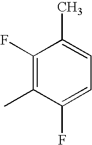 |
| 1189 | 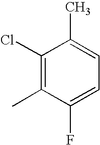 |
| 1190 | 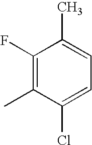 |
| 1191 | 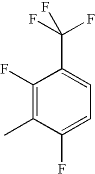 |
| 1192 | 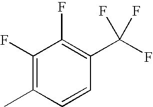 |
| 1193 | 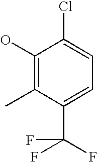 |
| 1194 | 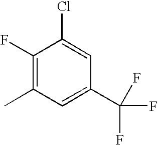 |
| 1195 | 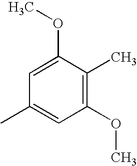 |
| 1196 | 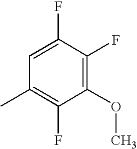 |
| 1197 | 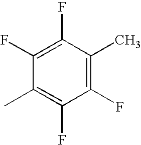 |
| 1198 | 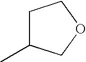 |
| 1199 | 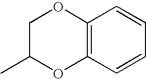 |
| 1200 | 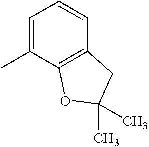 |
| 1201 | 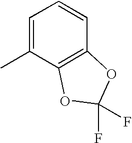 |
| 1202 | 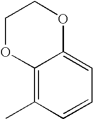 |
| 1203 | 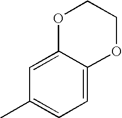 |
| 1204 | 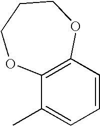 |
| 1205 | 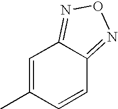 |
| 1206 | 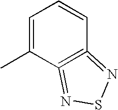 |
| 1207 | 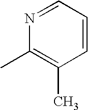 |
| 1208 | 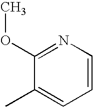 |
| 1209 | 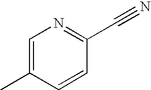 |
| 1210 | 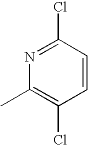 |
| 1211 | 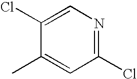 |
| 1212 | 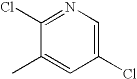 |
| 1213 | 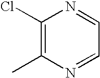 |
| 1214 | 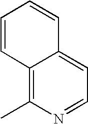 |
| 1215 | 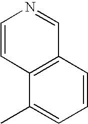 |
| 1216 | 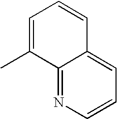 |
| 1217 | 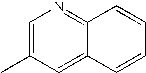 |
| 1218 | 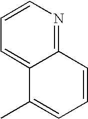 |
| 1219 | 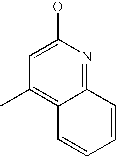 |
| 1220 | 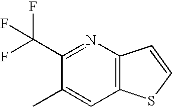 |
| 1221 | 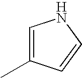 |
| 1222 | 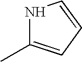 |
| 1223 | 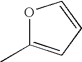 |
| 1224 | 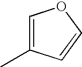 |
| 1225 | 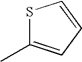 |
| 1226 | 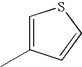 |
| 1227 | 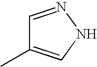 |
| 1228 | 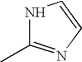 |
| 1229 | 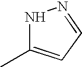 |
| 1230 | 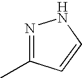 |
| 1231 | 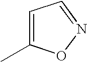 |
| 1232 | 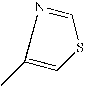 |
| 1233 | 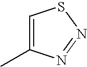 |
| 1234 | 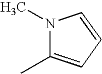 |
| 1235 | 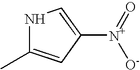 |
| 1236 | 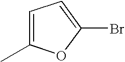 |
| 1237 | 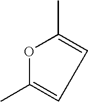 |
| 1238 | 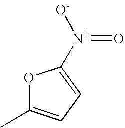 |
| 1239 | 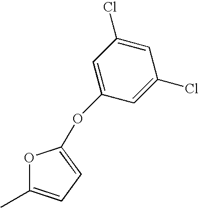 |
| 1240 | 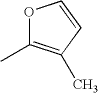 |
| 1241 | 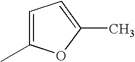 |
| 1242 | 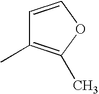 |
| 1243 | 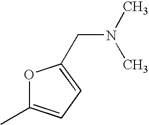 |
| 1244 | 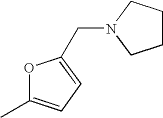 |
| 1245 | 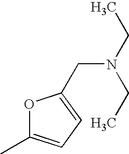 |
| 1246 | 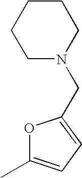 |
| 1247 | 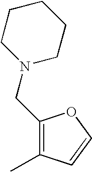 |
| 1248 | 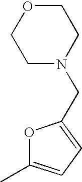 |
| 1249 | 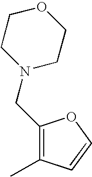 |
| 1250 | 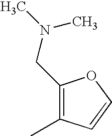 |
| 1251 | 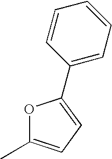 |
| 1252 | 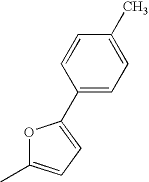 |
| 1253 | 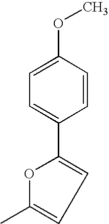 |
| 1254 | 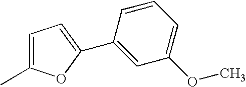 |
| 1255 | 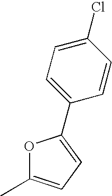 |
| 1256 | 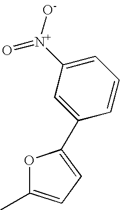 |
| 1257 | 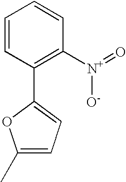 |
| 1258 | 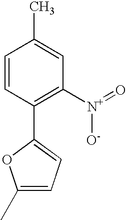 |
| 1259 | 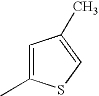 |
| 1260 | 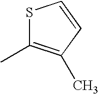 |
| 1261 | 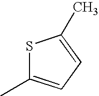 |
| 1262 | 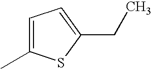 |
| 1263 | 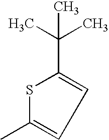 |
| 1264 | 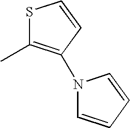 |
| 1265 | 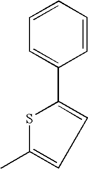 |
| 1266 | 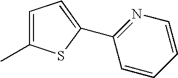 |
| 1267 | 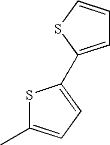 |
| 1268 | 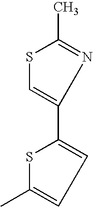 |
| 1269 | 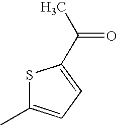 |
| 1270 | 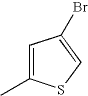 |
| 1271 | 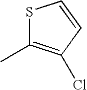 |
| 1272 | 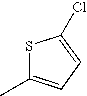 |
| 1273 | 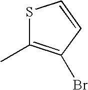 |
| 1274 | 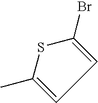 |
| 1275 | 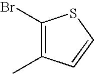 |
| 1276 | 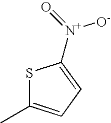 |
| 1277 | 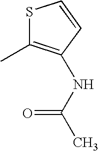 |
| 1278 | 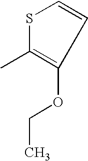 |
| 1279 | 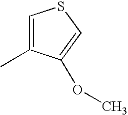 |
| 1280 | 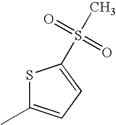 |
| 1281 | 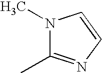 |
| 1282 | 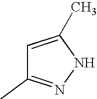 |
| 1283 | 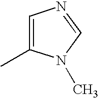 |
| 1284 | 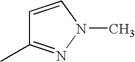 |
| 1285 | 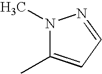 |
| 1286 | 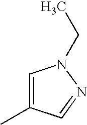 |
| 1287 | 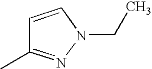 |
| 1288 | 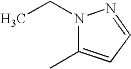 |
| 1289 | 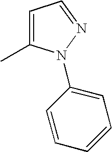 |
| 1290 | 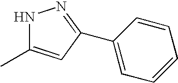 |
| 1291 | 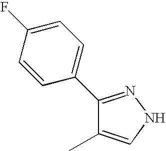 |
| 1292 | 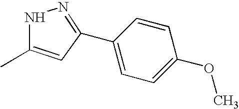 |
| 1293 | 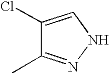 |
| 1294 | 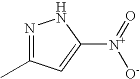 |
| 1295 | 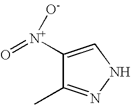 |
| 1296 | 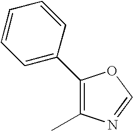 |
| 1297 | 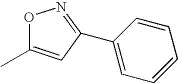 |
| 1298 | 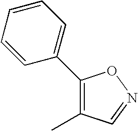 |
| 1299 | 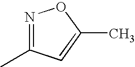 |
| 1300 | 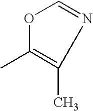 |
| 1301 | 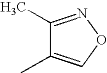 |
| 1302 | 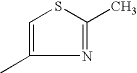 |
| 1303 | 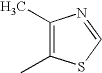 |
| 1304 | 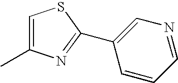 |
| 1305 | 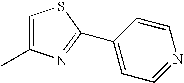 |
| 1306 | 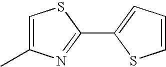 |
| 1307 | 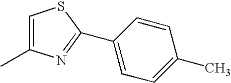 |
| 1308 | 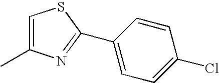 |
| 1309 | 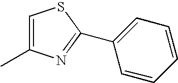 |
| 1310 | 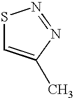 |
| 1311 | 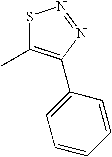 |
| 1312 | 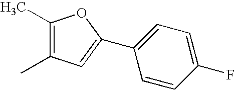 |
| 1313 | 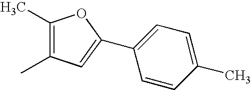 |
| 1314 | 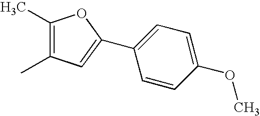 |
| 1315 | 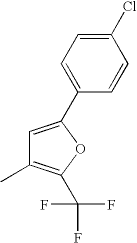 |
| 1316 | 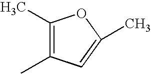 |
| 1317 | 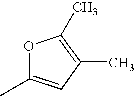 |
| 1318 | 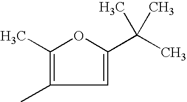 |
| 1319 | 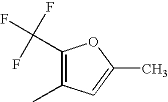 |
| 1320 | 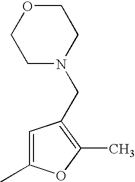 |
| 1321 | 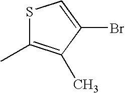 |
| 1322 | 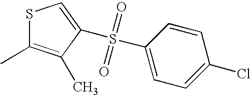 |
| 1323 | 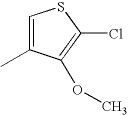 |
| 1324 | 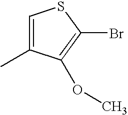 |
| 1325 | 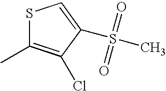 |
| 1326 | 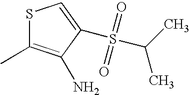 |
| 1327 | 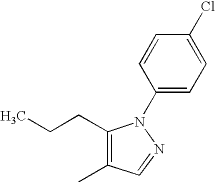 |
| 1328 | 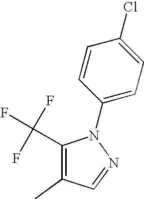 |
| 1329 | 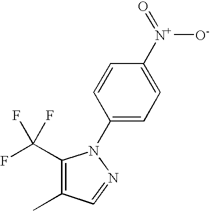 |
| 1330 | 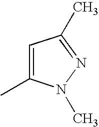 |
| 1331 | 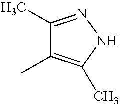 |
| 1332 | 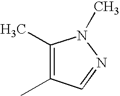 |
| 1333 | 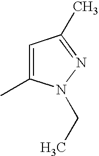 |
| 1334 | 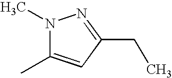 |
| 1335 | 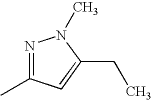 |
| 1336 | 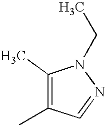 |
| 1337 | 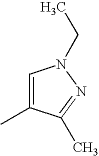 |
| 1338 | 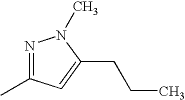 |
| 1339 | 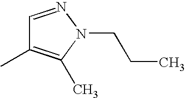 |
| 1340 | 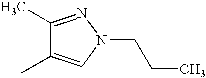 |
| 1341 | 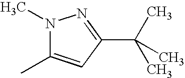 |
| 1342 | 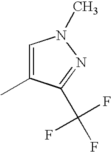 |
| 1343 | 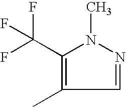 |
| 1344 | 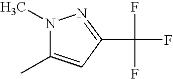 |
| 1345 | 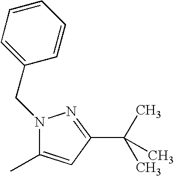 |
| 1346 | 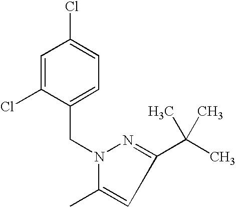 |
| 1347 | 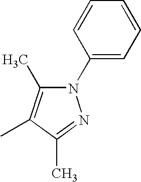 |
| 1348 | 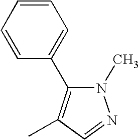 |
| 1349 | 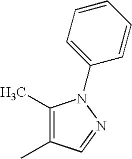 |
| 1350 | 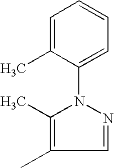 |
| 1351 | 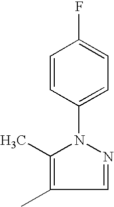 |
| 1352 | 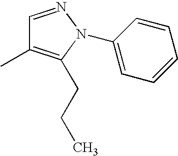 |
| 1353 | 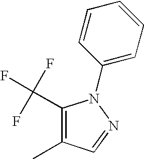 |
| 1354 | 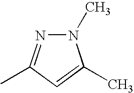 |
| 1355 | 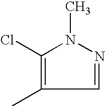 |
| 1356 | 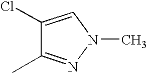 |
| 1357 | 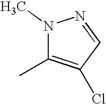 |
| 1358 | 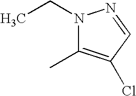 |
| 1359 | 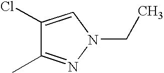 |
| 1360 | 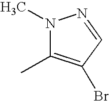 |
| 1361 | 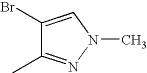 |
| 1362 | 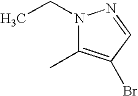 |
| 1363 | 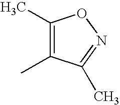 |
| 1364 | 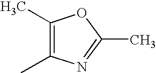 |
| 1365 | 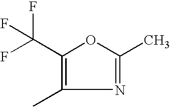 |
| 1366 | 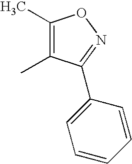 |
| 1367 | 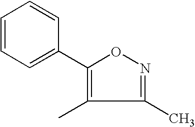 |
| 1368 | 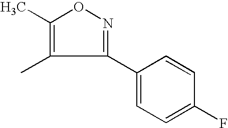 |
| 1369 | 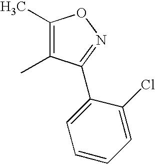 |
| 1370 | 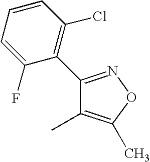 |
| 1371 | 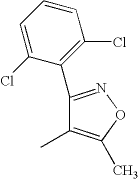 |
| 1372 | 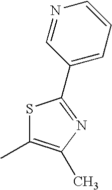 |
| 1373 | 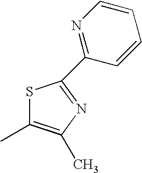 |
| 1374 | 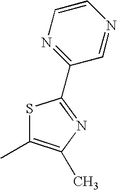 |
| 1375 | 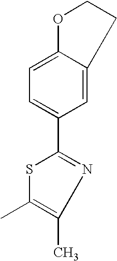 |
| 1376 | 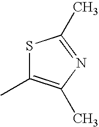 |
| 1377 | 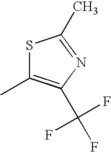 |
| 1378 | 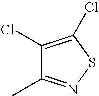 |
| 1379 | 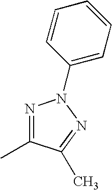 |
| 1380 | 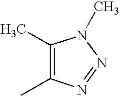 |
| 1381 | 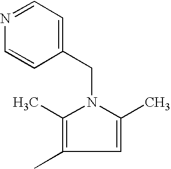 |
| 1382 | 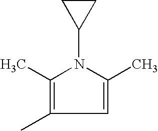 |
| 1383 | 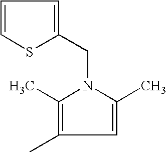 |
| 1384 | 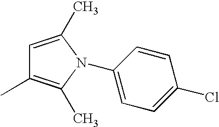 |
| 1385 | 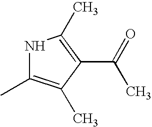 |
| 1386 | 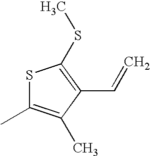 |
| 1387 | 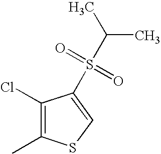 |
| 1388 | 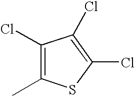 |
| 1389 | 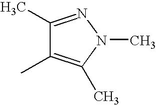 |
| 1390 | 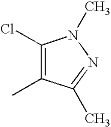 |
| 1391 | 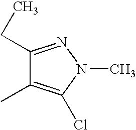 |
| 1392 | 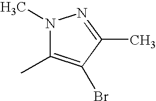 |
| 1393 | 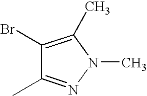 |
| 1394 | 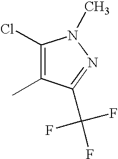 |
| 1395 | 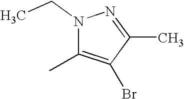 |
| 1396 | 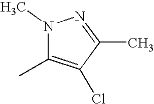 |
| 1397 | 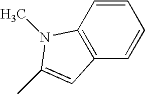 |
| 1398 | 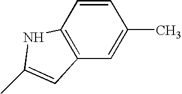 |
| 1399 | 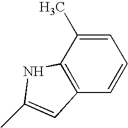 |
| 1400 | 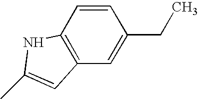 |
| 1401 | 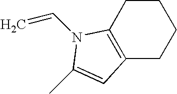 |
| 1402 | 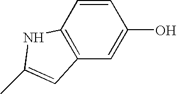 |
| 1403 | 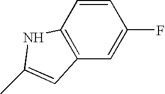 |
| 1404 | 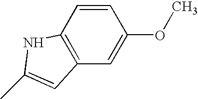 |
| 1405 | 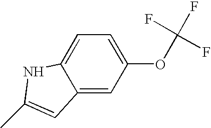 |
| 1406 | 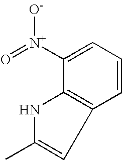 |
| 1407 | 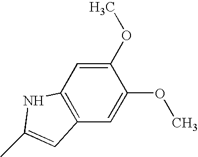 |
| 1408 | 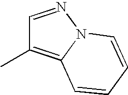 |
| 1409 | 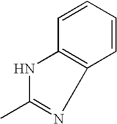 |
| 1410 | 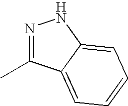 |
| 1411 | 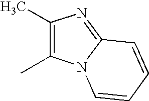 |
| 1412 | 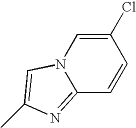 |
| 1413 | 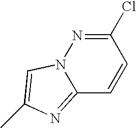 |
| 1414 | 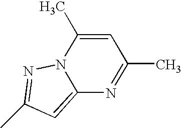 |
| 1415 | 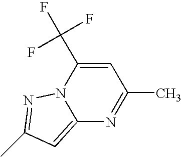 |
| 1416 | 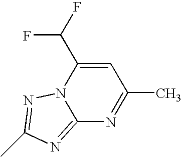 |
| 1417 | 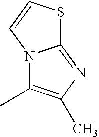 |
| 1418 | 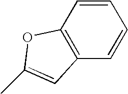 |
| 1419 | 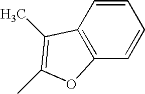 |
| 1420 | 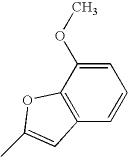 |
| 1421 | 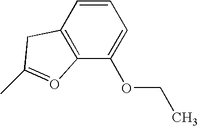 |
| 1422 | 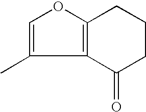 |
| 1423 | 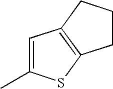 |
| 1424 | 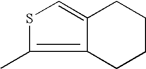 |
| 1425 | 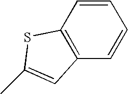 |
| 1426 | 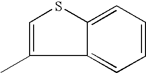 |
| 1427 | 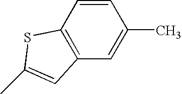 |
| 1428 | 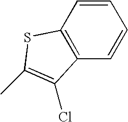 |
| 1429 | 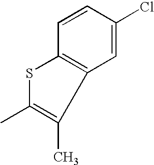 |
| 1430 | 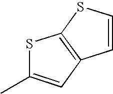 |
| 1431 | 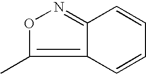 |

TABLE 5-1

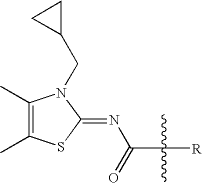

| Compound | |
|---|---|
| No. | R |
| 2001 | 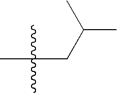 |
| 2002 | 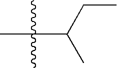 |
| 2003 | 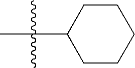 |
| 2004 | 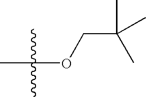 |
| 2005 | 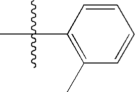 |
| 2006 | 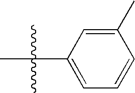 |
| 2007 | 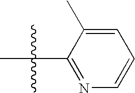 |
| 2008 | 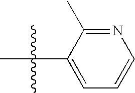 |
| 2009 | 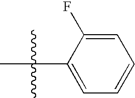 |
| 2010 | 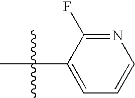 |
| 2011 | 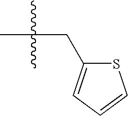 |
| 2012 | 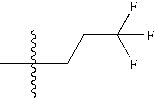 |
| 2013 | 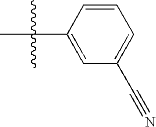 |
| 2014 | 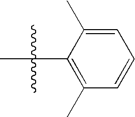 |
| 2015 | 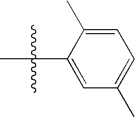 |
| 2016 | 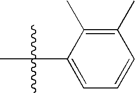 |
| 2017 | 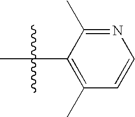 |
| 2018 | 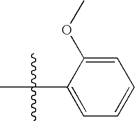 |
| 2019 | 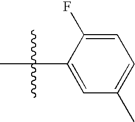 |
| 2020 | 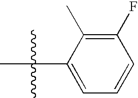 |
| 2021 | 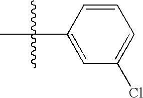 |
| 2022 | 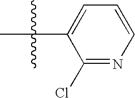 |
| 2023 | 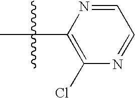 |
| 2024 | 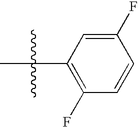 |
| 2025 | 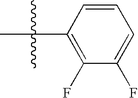 |
| 2026 | 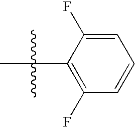 |
| 2027 | 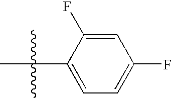 |
| 2028 | 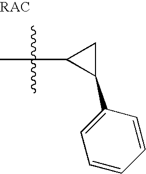 |
| 2029 | 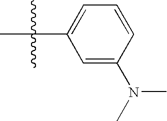 |
| 2030 | 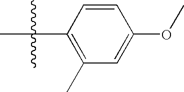 |
| 2031 | 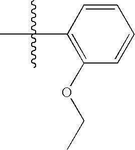 |
| 2032 | 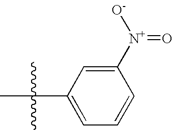 |
| 2033 | 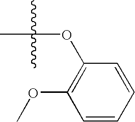 |
| 2034 | 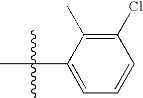 |
| 2035 | 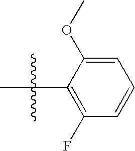 |
| 2036 | 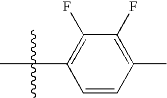 |
| 2037 | 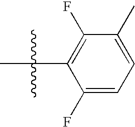 |
| 2038 | 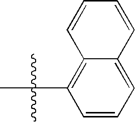 |
| 2039 | 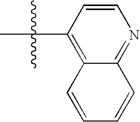 |
| 2040 | 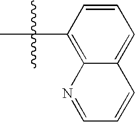 |
| 2041 | 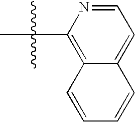 |
| 2042 | 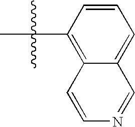 |
| 2043 | 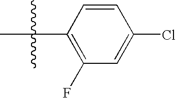 |
| 2044 | 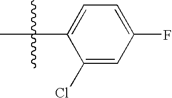 |
| 2045 | 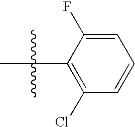 |
| 2046 | 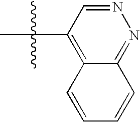 |
| 2047 | 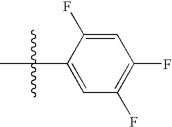 |
| 2048 | 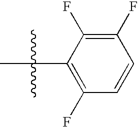 |
| 2049 | 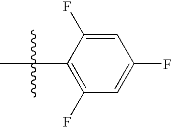 |
| 2050 | 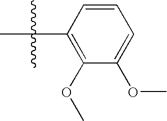 |
| 2051 | 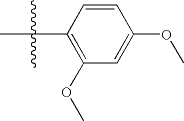 |
| 2052 | 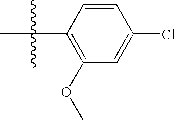 |
| 2053 | 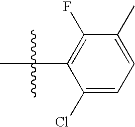 |
| 2054 | 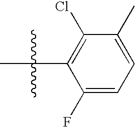 |
| 2055 | 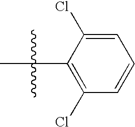 |
| 2056 | 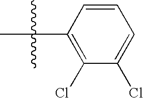 |
| 2057 | 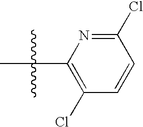 |
| 2058 | 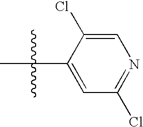 |
| 2059 | 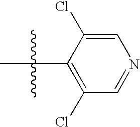 |
| 2060 | 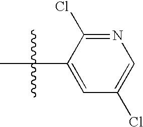 |
| 2061 | 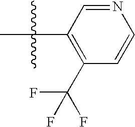 |
| 2062 | 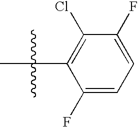 |
| 2063 | 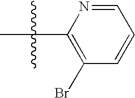 |
| 2064 | 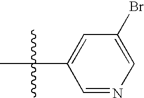 |
| 2065 | 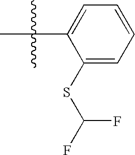 |
| 2066 | 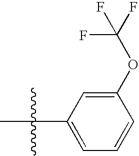 |
| 2067 | 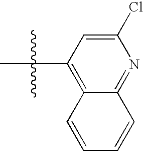 |
| 2068 | 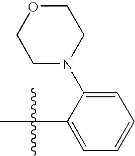 |
| 2069 | 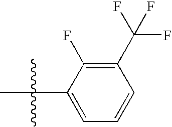 |
| 2070 | 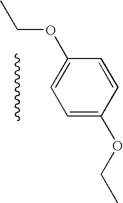 |
| 2071 | 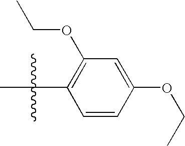 |
| 2072 | 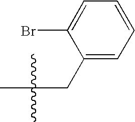 |
| 2073 | 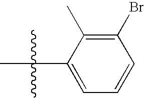 |
| 2074 | 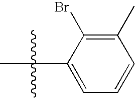 |
| 2075 | 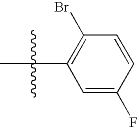 |
| 2076 | 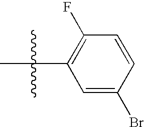 |
| 2077 | 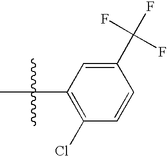 |
| 2078 | 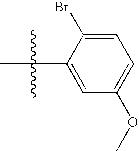 |
| 2079 | 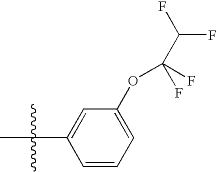 |
| 2080 | 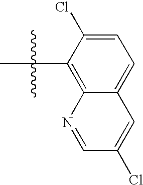 |
| 2081 | 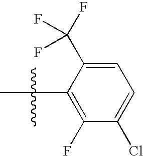 |
| 2082 | 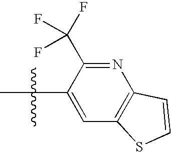 |
| 2083 | 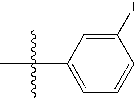 |
| 2084 | 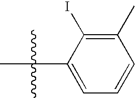 |
| 2085 | 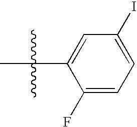 |

TABLE 5-2

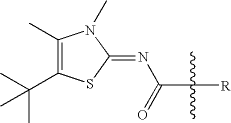

| Compound | |
|---|---|
| No. | R |
| 2086 | 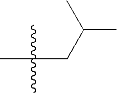 |
| 2087 | 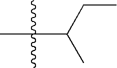 |
| 2088 | 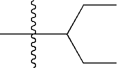 |
| 2089 | 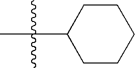 |
| 2090 | 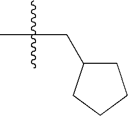 |
| 2091 | 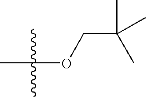 |
| 2092 | 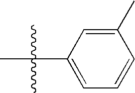 |
| 2093 | 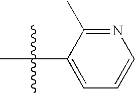 |
| 2094 | 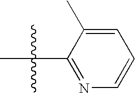 |
| 2095 | 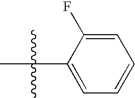 |
| 2096 | 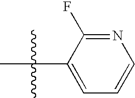 |
| 2097 | 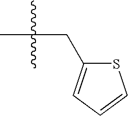 |
| 2098 | 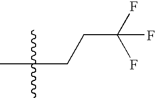 |
| 2099 | 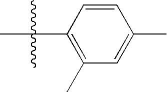 |
| 2100 | 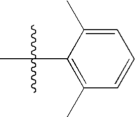 |
| 2101 | 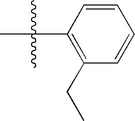 |
| 2102 | 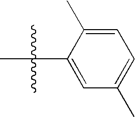 |
| 2103 | 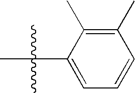 |
| 2104 | 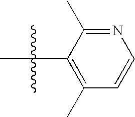 |
| 2105 | 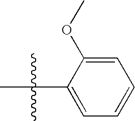 |
| 2106 | 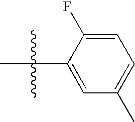 |
| 2107 | 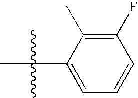 |
| 2108 | 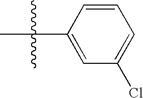 |
| 2109 | 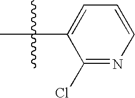 |
| 2110 | 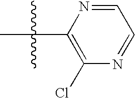 |
| 2111 | 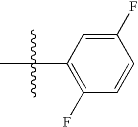 |
| 2112 | 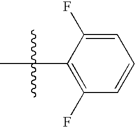 |
| 2113 | 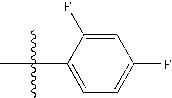 |
| 2114 | 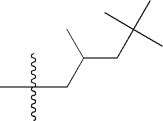 |
| 2115 | 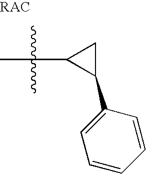 |
| 2116 | 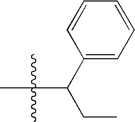 |
| 2117 | 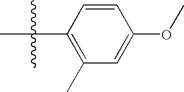 |
| 2118 | 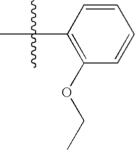 |
| 2119 | 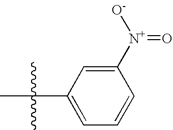 |
| 2120 | 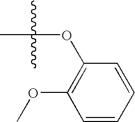 |
| 2121 | 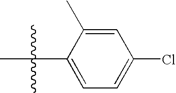 |
| 2122 | 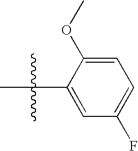 |
| 2123 | 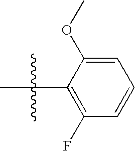 |
| 2124 | 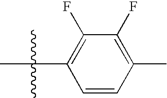 |
| 2125 | 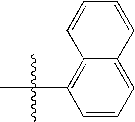 |
| 2126 | 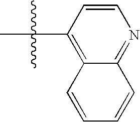 |
| 2127 | 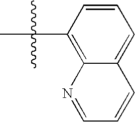 |
| 2128 | 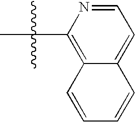 |
| 2129 | 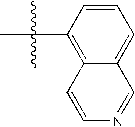 |
| 2130 | 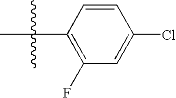 |
| 2131 | 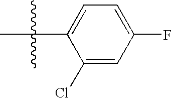 |
| 2132 | 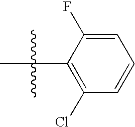 |
| 2133 | 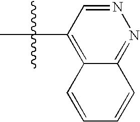 |
| 2134 | 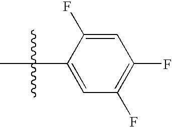 |
| 2135 | 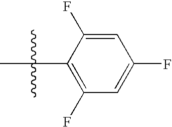 |
| 2136 | 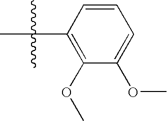 |
| 2137 | 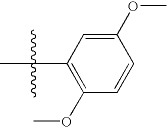 |
| 2138 | 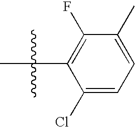 |
| 2139 | 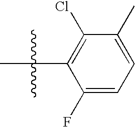 |
| 2140 | 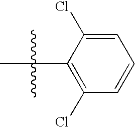 |
| 2141 | 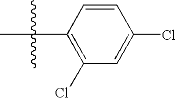 |
| 2142 | 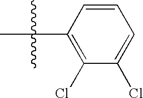 |
| 2143 | 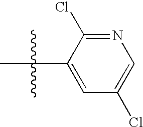 |
| 2144 | 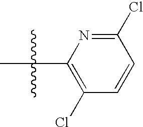 |
| 2145 | 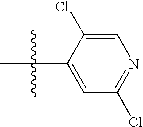 |
| 2146 | 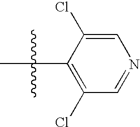 |
| 2147 | 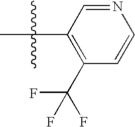 |
| 2148 | 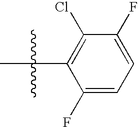 |
| 2149 | 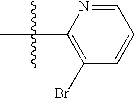 |
| 2150 | 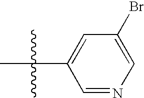 |
| 2151 | 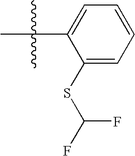 |
| 2152 | 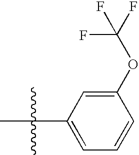 |
| 2153 | 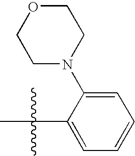 |
| 2154 | 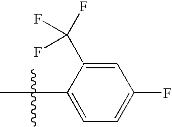 |
| 2155 | 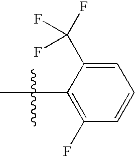 |
| 2156 | 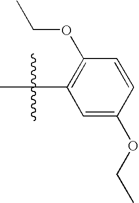 |
| 2157 | 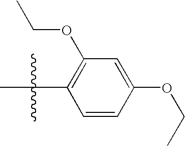 |
| 2158 | 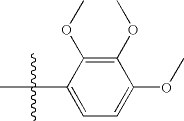 |
| 2159 | 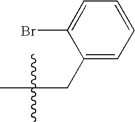 |
| 2160 | 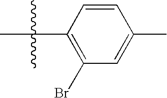 |
| 2161 | 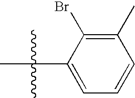 |
| 2162 | 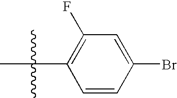 |
| 2163 | 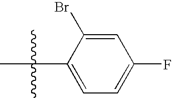 |
| 2164 | 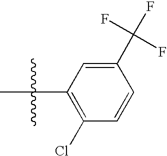 |
| 2165 | 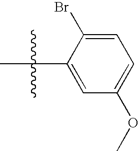 |
| 2166 | 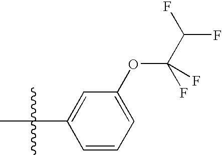 |
| 2167 | 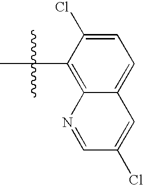 |
| 2168 | 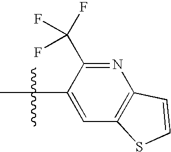 |
| 2169 | 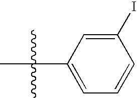 |
| 2170 | 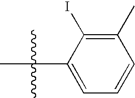 |
| 2171 | 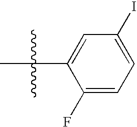 |

TABLE 5-3

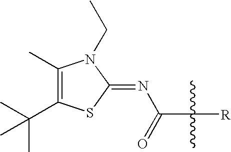

| Compound | |
|---|---|
| No. | R |
| 2172 | 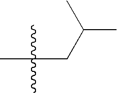 |
| 2173 | 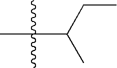 |
| 2174 | 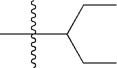 |
| 2175 | 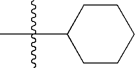 |
| 2176 | 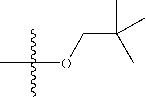 |
| 2177 | 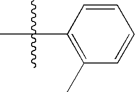 |
| 2178 | 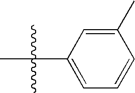 |
| 2179 | 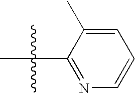 |
| 2180 | 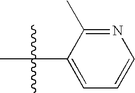 |
| 2181 | 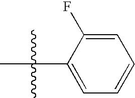 |
| 2182 | 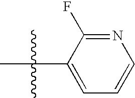 |
| 2183 | 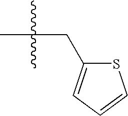 |
| 2184 | 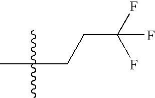 |
| 2185 | 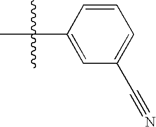 |
| 2186 | 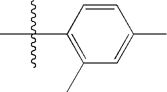 |
| 2187 | 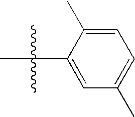 |
| 2188 | 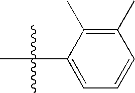 |
| 2189 | 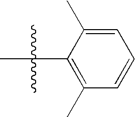 |
| 2190 | 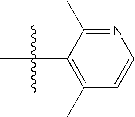 |
| 2191 | 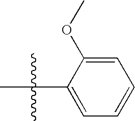 |
| 2192 | 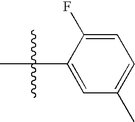 |
| 2193 | 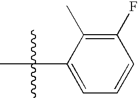 |
| 2194 | 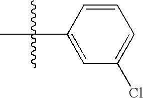 |
| 2195 | 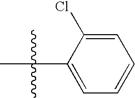 |
| 2196 | 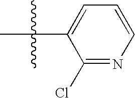 |
| 2197 | 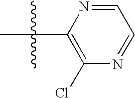 |
| 2198 | 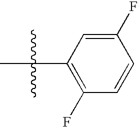 |
| 2199 | 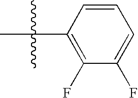 |
| 2200 | 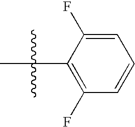 |
| 2201 | 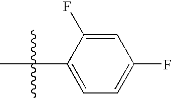 |
| 2202 | 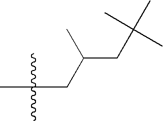 |
| 2203 | 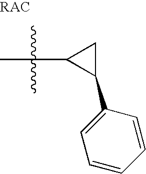 |
| 2204 | 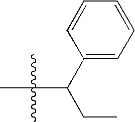 |
| 2205 | 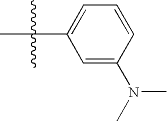 |
| 2206 | 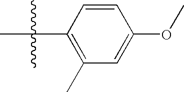 |
| 2207 | 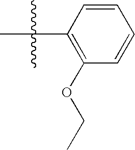 |
| 2208 | 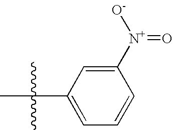 |
| 2209 | 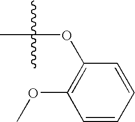 |
| 2210 | 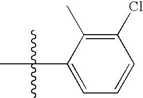 |
| 2211 | 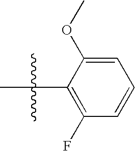 |
| 2212 | 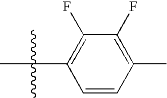 |
| 2213 | 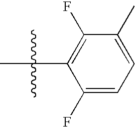 |
| 2214 | 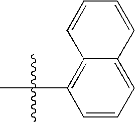 |
| 2215 | 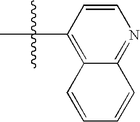 |
| 2216 | 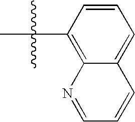 |
| 2217 | 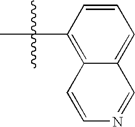 |
| 2218 | 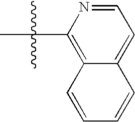 |
| 2219 | 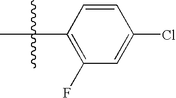 |
| 2220 | 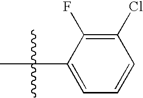 |
| 2221 | 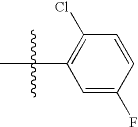 |
| 2222 | 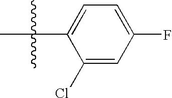 |
| 2223 | 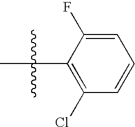 |
| 2224 | 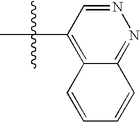 |
| 2225 | 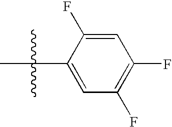 |
| 2226 | 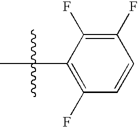 |
| 2227 | 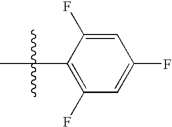 |
| 2228 | 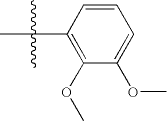 |
| 2229 | 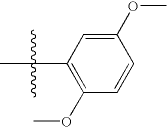 |
| 2230 | 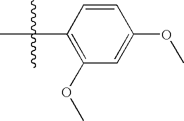 |
| 2231 | 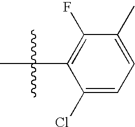 |
| 2232 | 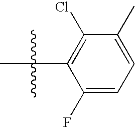 |
| 2233 | 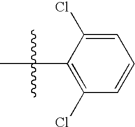 |
| 2234 | 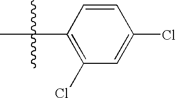 |
| 2235 | 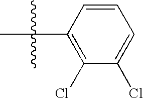 |
| 2236 | 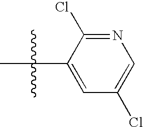 |
| 2237 | 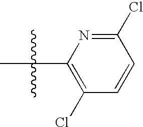 |
| 2238 | 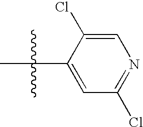 |
| 2239 | 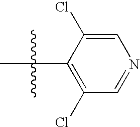 |
| 2240 | 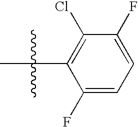 |
| 2241 | 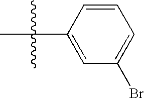 |
| 2242 | 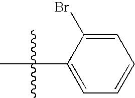 |
| 2243 | 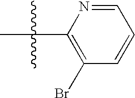 |
| 2244 | 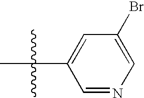 |
| 2245 | 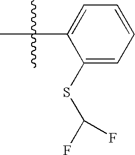 |
| 2246 | 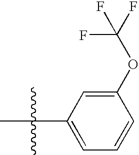 |
| 2247 | 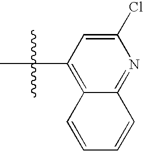 |
| 2248 | 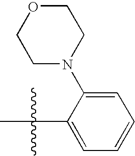 |
| 2249 | 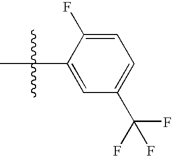 |
| 2250 | 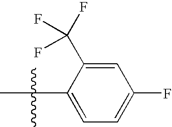 |
| 2251 | 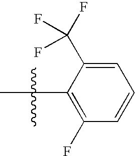 |
| 2252 | 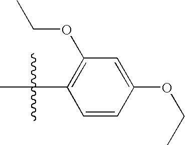 |
| 2253 | 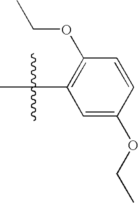 |
| 2254 | 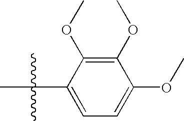 |
| 2255 | 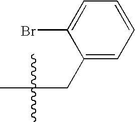 |
| 2256 | 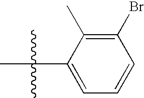 |
| 2257 | 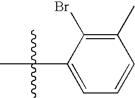 |
| 2258 | 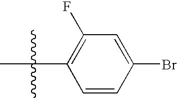 |
| 2259 | 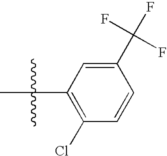 |
| 2260 | 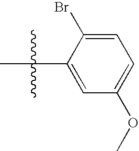 |
| 2261 | 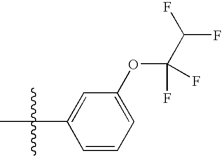 |
| 2262 | 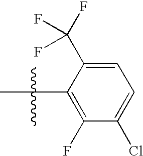 |
| 2263 | 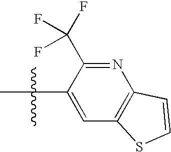 |
| 2264 | 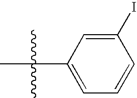 |
| 2265 | 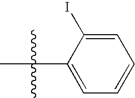 |
| 2266 | 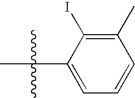 |

TABLE 5-4

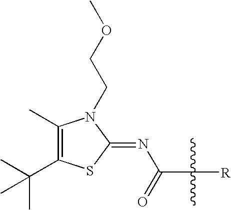

| Compound | |
|---|---|
| No. | R |
| 2267 | 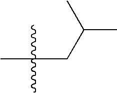 |
| 2268 | 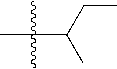 |
| 2269 | 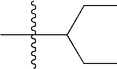 |
| 2270 | 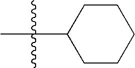 |
| 2271 | 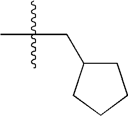 |
| 2272 | 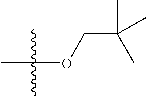 |
| 2273 | 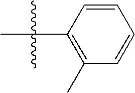 |
| 2274 | 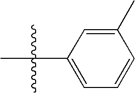 |
| 2275 | 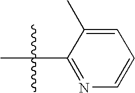 |
| 2276 | 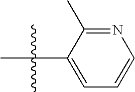 |
| 2277 | 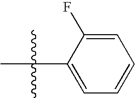 |
| 2278 | 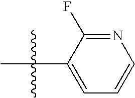 |
| 2279 | 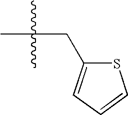 |
| 2280 | 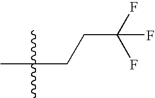 |
| 2281 | 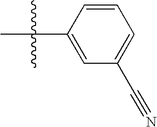 |
| 2282 | 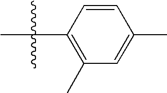 |
| 2283 | 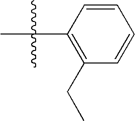 |
| 2284 | 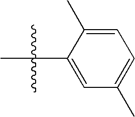 |
| 2285 | 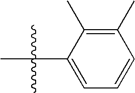 |
| 2286 | 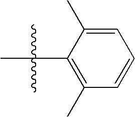 |
| 2287 | 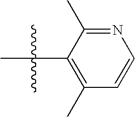 |
| 2288 | 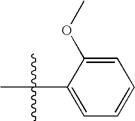 |
| 2289 | 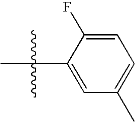 |
| 2290 | 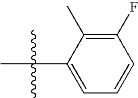 |
| 2291 | 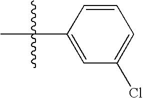 |
| 2292 | 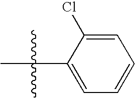 |
| 2293 | 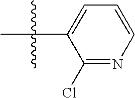 |
| 2294 | 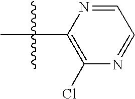 |
| 2295 | 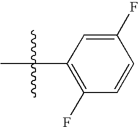 |
| 2296 | 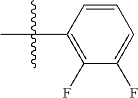 |
| 2297 | 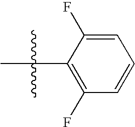 |
| 2298 | 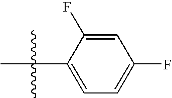 |
| 2299 | 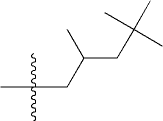 |
| 2300 | 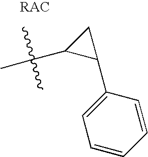 |
| 2301 | 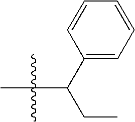 |
| 2302 | 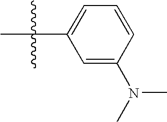 |
| 2303 | 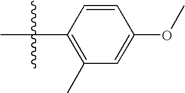 |
| 2304 | 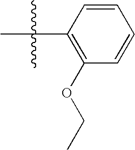 |
| 2305 | 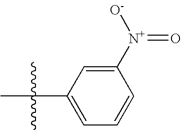 |
| 2306 | 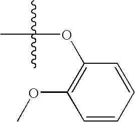 |
| 2307 | 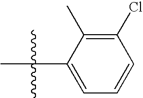 |
| 2308 | 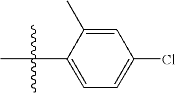 |
| 2309 | 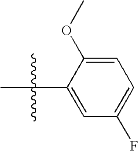 |
| 2310 | 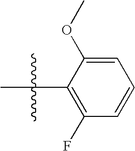 |
| 2311 | 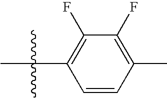 |
| 2312 | 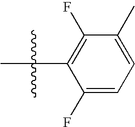 |
| 2313 | 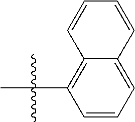 |
| 2314 | 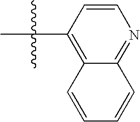 |
| 2315 | 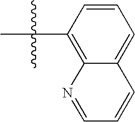 |
| 2316 | 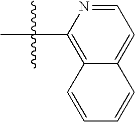 |
| 2317 | 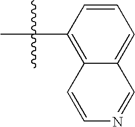 |
| 2318 | 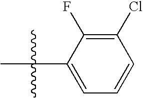 |
| 2319 | 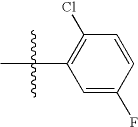 |
| 2320 | 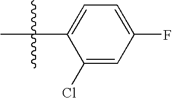 |
| 2321 | 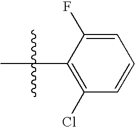 |
| 2322 | 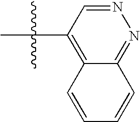 |
| 2323 | 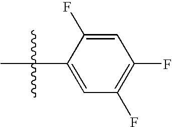 |
| 2324 | 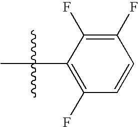 |
| 2325 | 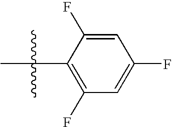 |
| 2326 | 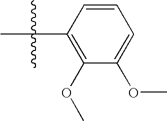 |
| 2327 | 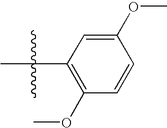 |
| 2328 | 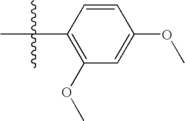 |
| 2329 | 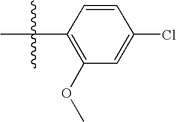 |
| 2330 | 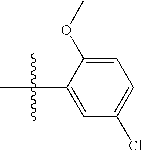 |
| 2331 | 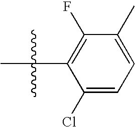 |
| 2332 | 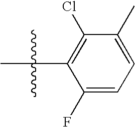 |
| 2333 | 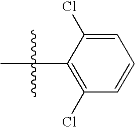 |
| 2334 | 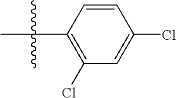 |
| 2335 | 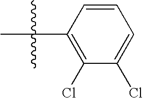 |
| 2336 | 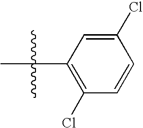 |
| 2337 | 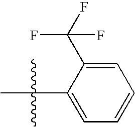 |
| 2338 | 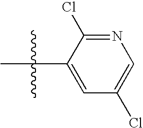 |
| 2339 | 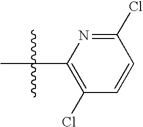 |
| 2340 | 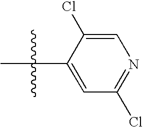 |
| 2341 | 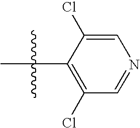 |
| 2342 | 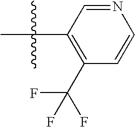 |
| 2343 | 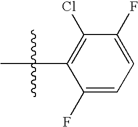 |
| 2344 | 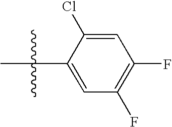 |
| 2345 | 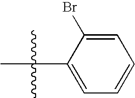 |
| 2346 | 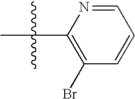 |
| 2347 | 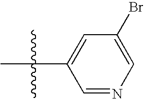 |
| 2348 | 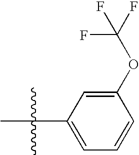 |
| 2349 | 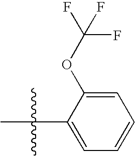 |
| 2350 | 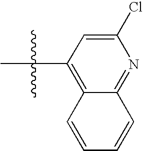 |
| 2351 | 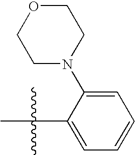 |
| 2352 | 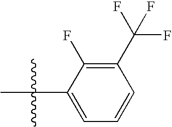 |
| 2353 | 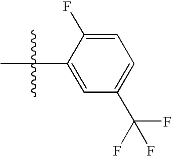 |
| 2354 | 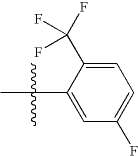 |
| 2355 | 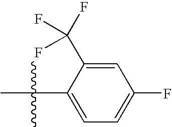 |
| 2356 | 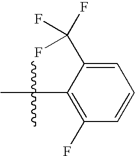 |
| 2357 | 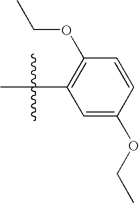 |
| 2358 | 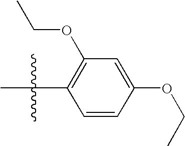 |
| 2359 | 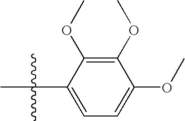 |
| 2360 | 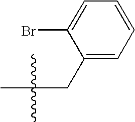 |
| 2361 | 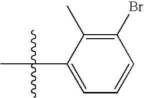 |
| 2362 | 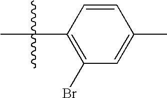 |
| 2363 | 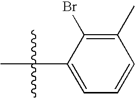 |
| 2364 | 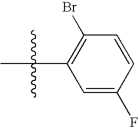 |
| 2365 | 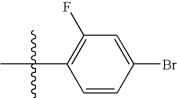 |
| 2366 | 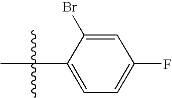 |
| 2367 | 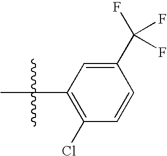 |
| 2368 | 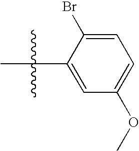 |
| 2369 | 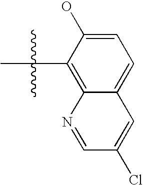 |
| 2370 | 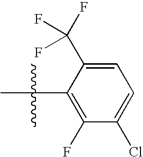 |
| 2371 | 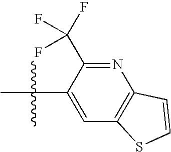 |
| 2372 | 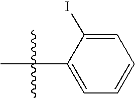 |
| 2373 | 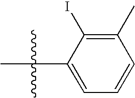 |
| 2374 | 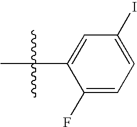 |

TABLE 5-5

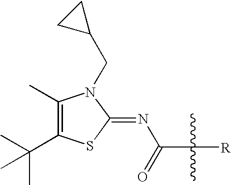

| Com- | |
|---|---|
| pound | |
| No. | R |
| 2375 | 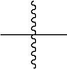 |
| 2376 | 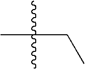 |
| 2377 | 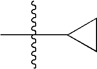 |
| 2378 | 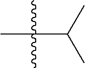 |
| 2379 | 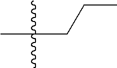 |
| 2380 | 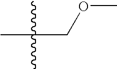 |
| 2381 | 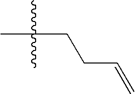 |
| 2382 | 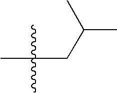 |
| 2383 | 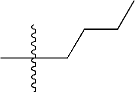 |
| 2384 | 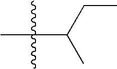 |
| 2385 | 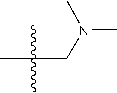 |
| 2386 | 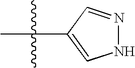 |
| 2387 | 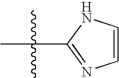 |
| 2388 | 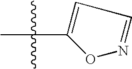 |
| 2389 | 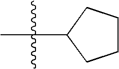 |
| 2390 | 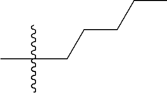 |
| 2391 | 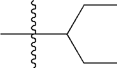 |
| 2392 | 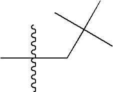 |
| 2393 | 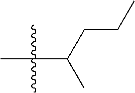 |
| 2394 | 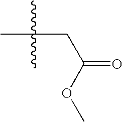 |
| 2395 | 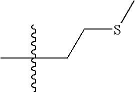 |
| 2396 | 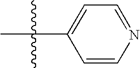 |
| 2397 | 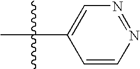 |
| 2398 | 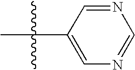 |
| 2399 | 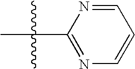 |
| 2400 | 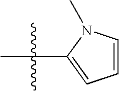 |
| 2401 | 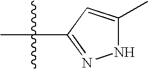 |
| 2402 | 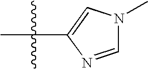 |
| 2403 | 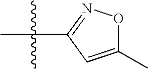 |
| 2404 | 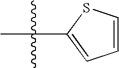 |
| 2405 | 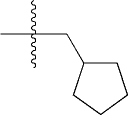 |
| 2406 | 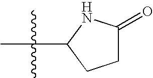 |
| 2407 | 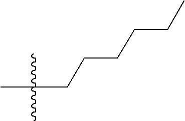 |
| 2408 | 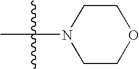 |
| 2409 | 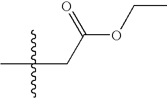 |
| 2410 | 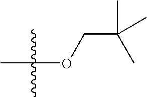 |
| 2411 | 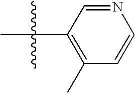 |
| 2412 | 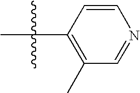 |
| 2413 | 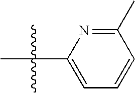 |
| 2414 | 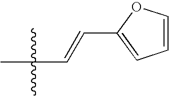 |
| 2415 | 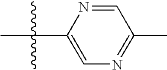 |
| 2416 | 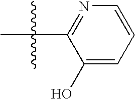 |
| 2417 | 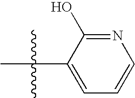 |
| 2418 | 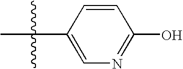 |
| 2419 | 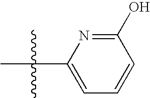 |
| 2420 | 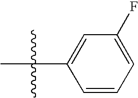 |
| 2421 | 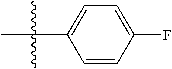 |
| 2422 | 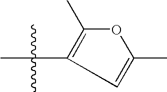 |
| 2423 | 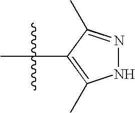 |
| 2424 | 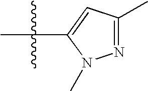 |
| 2425 | 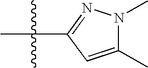 |
| 2426 | 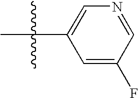 |
| 2427 | 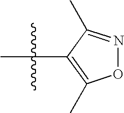 |
| 2428 | 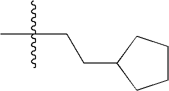 |
| 2429 | 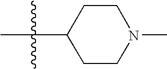 |
| 2430 | 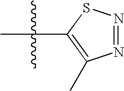 |
| 2431 | 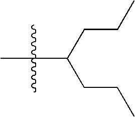 |
| 2432 | 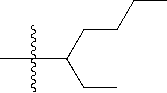 |
| 2433 | 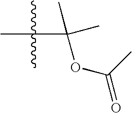 |
| 2434 | 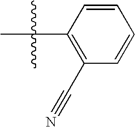 |
| 2435 | 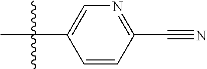 |
| 2436 | 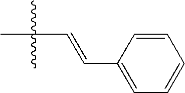 |
| 2437 | 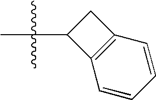 |
| 2438 | 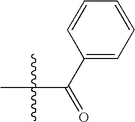 |
| 2439 | 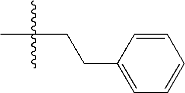 |
| 2440 | 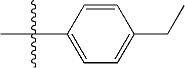 |
| 2441 | 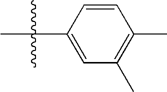 |
| 2442 | 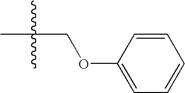 |
| 2443 | 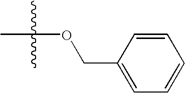 |
| 2444 | 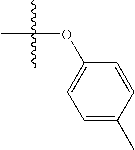 |
| 2445 | 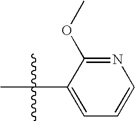 |
| 2446 | 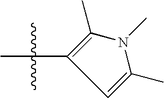 |
| 2447 | 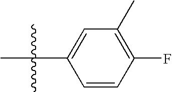 |
| 2448 | 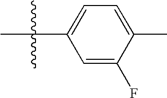 |
| 2449 | 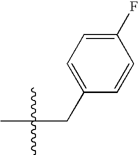 |
| 2450 | 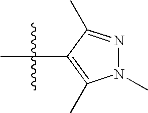 |
| 2451 | 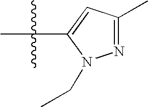 |
| 2452 | 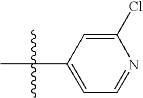 |
| 2453 | 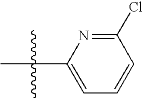 |
| 2454 | 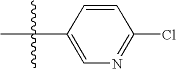 |
| 2455 | 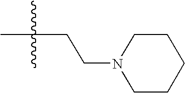 |
| 2456 | 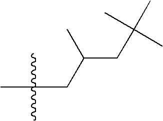 |
| 2457 | 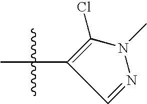 |
| 2458 | 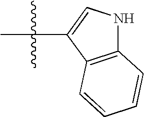 |
| 2459 | 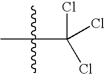 |
| 2460 | 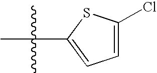 |
| 2461 | 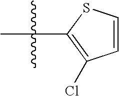 |
| 2462 | 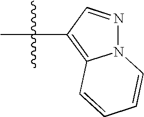 |
| 2463 | 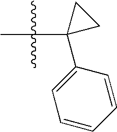 |
| 2464 | 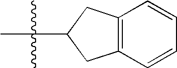 |
| 2465 | 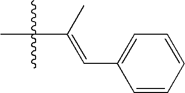 |
| 2466 | 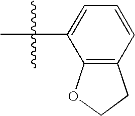 |
| 2467 | 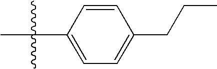 |
| 2468 | 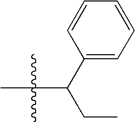 |
| 2469 | 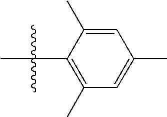 |
| 2470 | 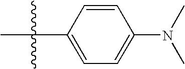 |
| 2471 | 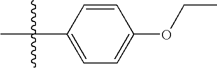 |
| 2472 | 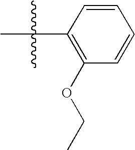 |
| 2473 | 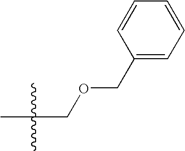 |
| 2474 | 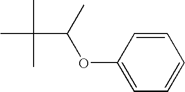 |
| 2475 | 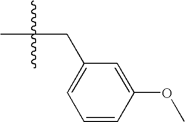 |
| 2476 | 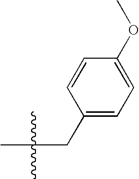 |
| 2477 | 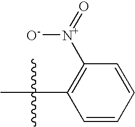 |
| 2478 | 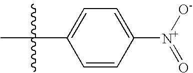 |
| 2479 | 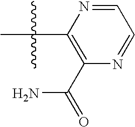 |
| 2480 | 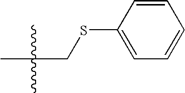 |
| 2481 | 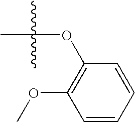 |
| 2482 | 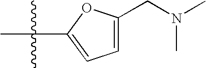 |
| 2483 | 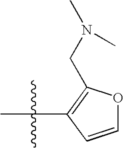 |
| 2484 | 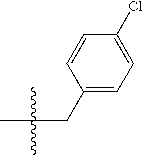 |
| 2485 | 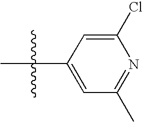 |
| 2486 | 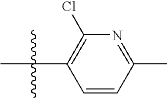 |
| 2487 | 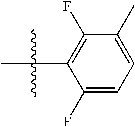 |
| 2488 | 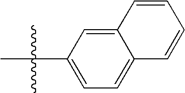 |
| 2489 | 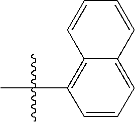 |
| 2490 | 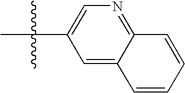 |
| 2491 | 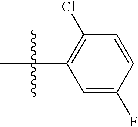 |
| 2492 | 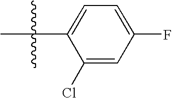 |
| 2493 | 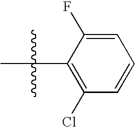 |
| 2494 | 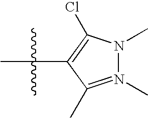 |
| 2495 | 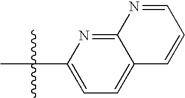 |
| 2496 | 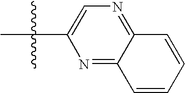 |
| 2497 | 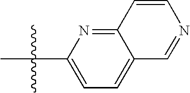 |
| 2498 | 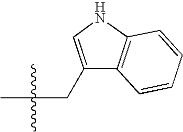 |
| 2499 | 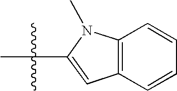 |
| 2500 | 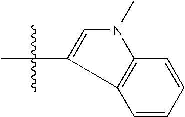 |
| 2501 | 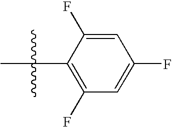 |
| 2502 | 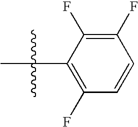 |
| 2503 | 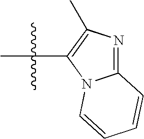 |
| 2504 | 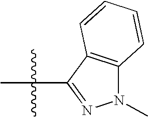 |
| 2505 | 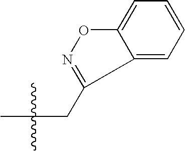 |
| 2506 | 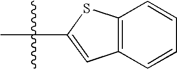 |
| 2507 | 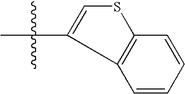 |
| 2508 | 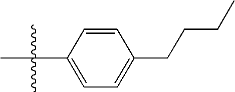 |
| 2509 | 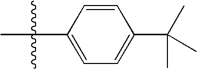 |
| 2510 | 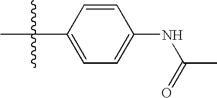 |
| 2511 | 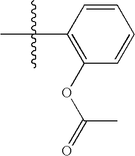 |
| 2512 | 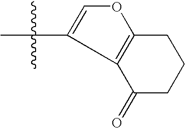 |
| 2513 | 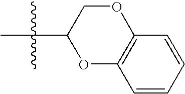 |
| 2514 | 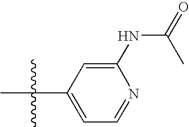 |
| 2515 | 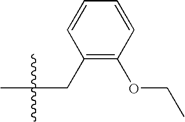 |
| 2516 | 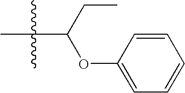 |
| 2517 | 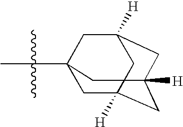 |
| 2518 | 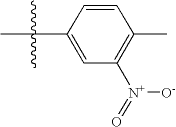 |
| 2519 | 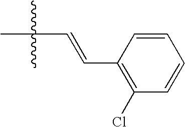 |
| 2520 | 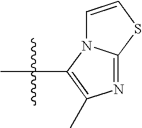 |
| 2521 | 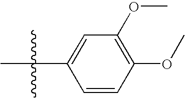 |
| 2522 | 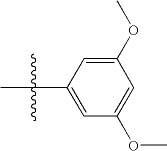 |
| 2523 | 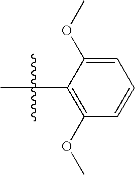 |
| 2524 | 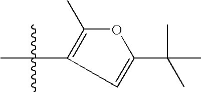 |
| 2525 | 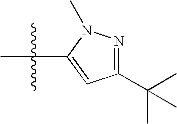 |
| 2526 | 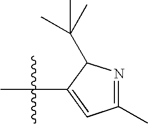 |
| 2527 | 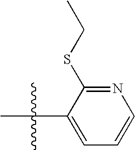 |
| 2528 | 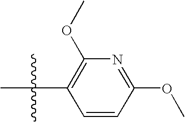 |
| 2529 | 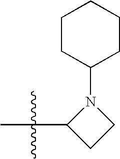 |
| 2530 | 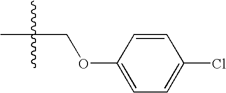 |
| 2531 | 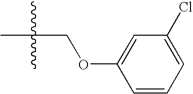 |
| 2532 | 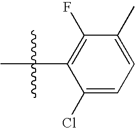 |
| 2533 | 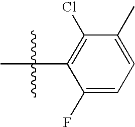 |
| 2534 | 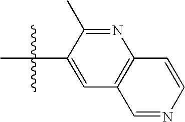 |
| 2535 | 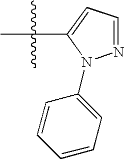 |
| 2536 | 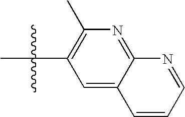 |
| 2537 | 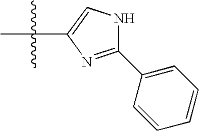 |
| 2538 | 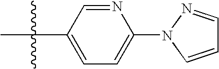 |
| 2539 | 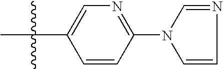 |
| 2540 | 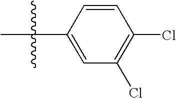 |
| 2541 | 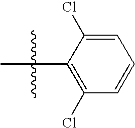 |
| 2542 | 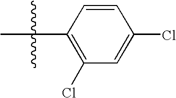 |
| 2543 | 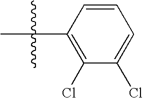 |
| 2544 | 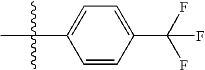 |
| 2545 | 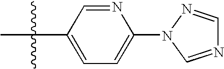 |
| 2546 | 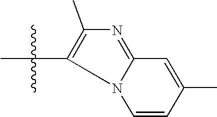 |
| 2547 | 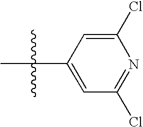 |
| 2548 | 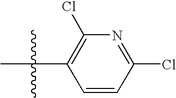 |
| 2549 | 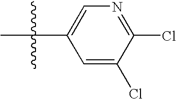 |
| 2550 | 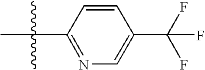 |
| 2551 | 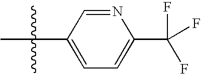 |
| 2552 | 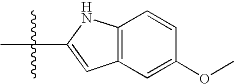 |
| 2553 | 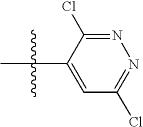 |
| 2554 | 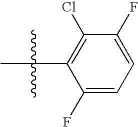 |
| 2555 | 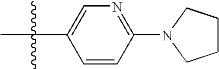 |
| 2556 | 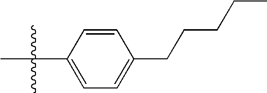 |
| 2557 | 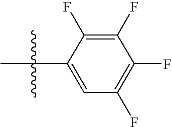 |
| 2558 | 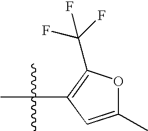 |
| 2559 | 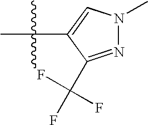 |
| 2560 | 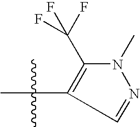 |
| 2561 | 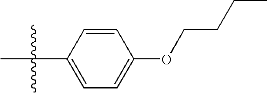 |
| 2562 | 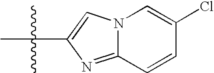 |
| 2563 | 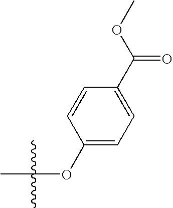 |
| 2564 | 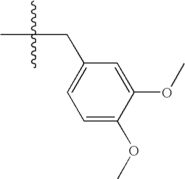 |
| 2565 | 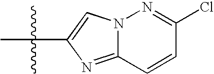 |
| 2566 | 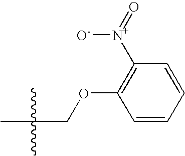 |
| 2567 | 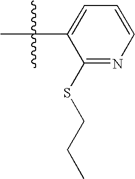 |
| 2568 | 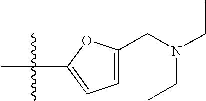 |
| 2569 | 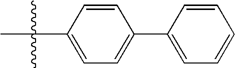 |
| 2570 | 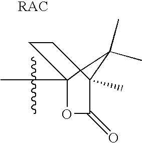 |
| 2571 | 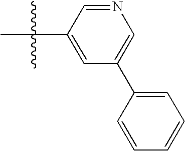 |
| 2572 | 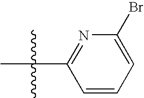 |
| 2573 | 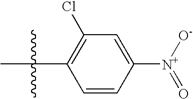 |
| 2574 | 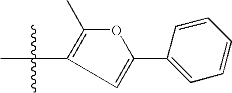 |
| 2575 | 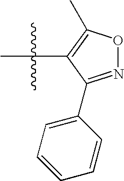 |
| 2576 | 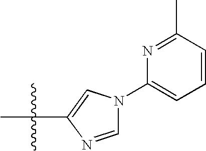 |
| 2577 | 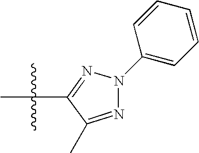 |
| 2578 | 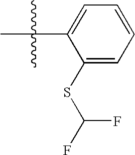 |
| 2579 | 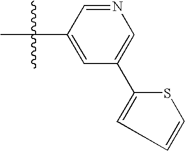 |
| 2580 | 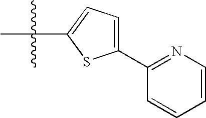 |
| 2581 | 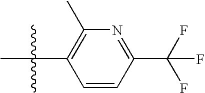 |
| 2582 | 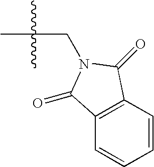 |
| 2583 | 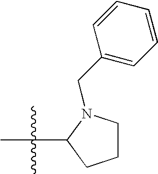 |
| 2584 | 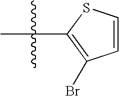 |
| 2585 | 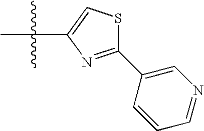 |
| 2586 | 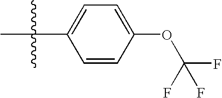 |
| 2587 | 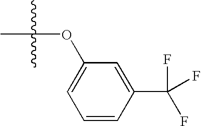 |
| 2588 | 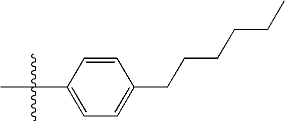 |
| 2589 | 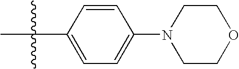 |
| 2590 | 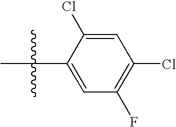 |
| 2591 | 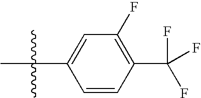 |
| 2592 | 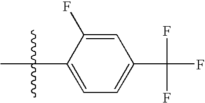 |
| 2593 | 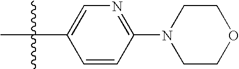 |
| 2594 | 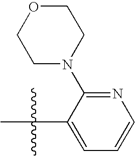 |
| 2595 | 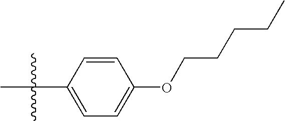 |
| 2596 | 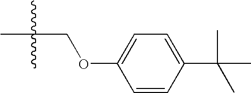 |
| 2597 | 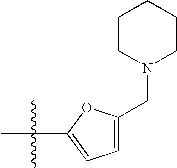 |
| 2598 | 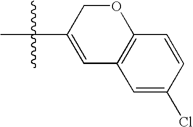 |
| 2599 | 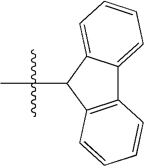 |
| 2600 | 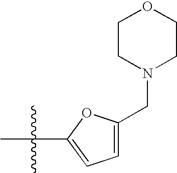 |
| 2601 | 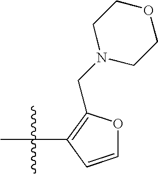 |
| 2602 | 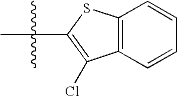 |
| 2603 | 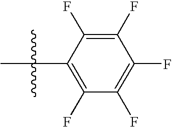 |
| 2604 | 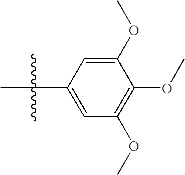 |
| 2605 | 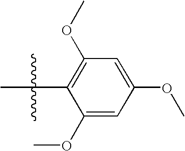 |
| 2606 | 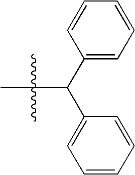 |
| 2607 | 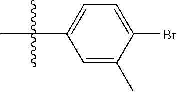 |
| 2608 | 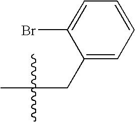 |
| 2609 | 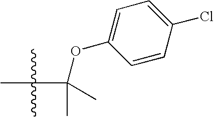 |
| 2610 | 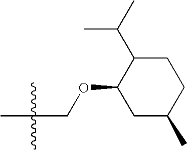 |
| 2611 | 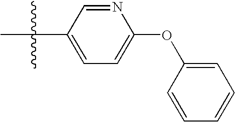 |
| 2612 | 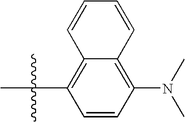 |
| 2613 | 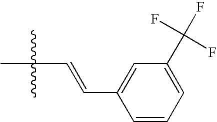 |
| 2614 | 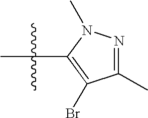 |
| 2615 | 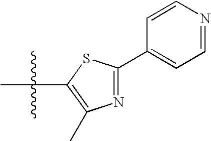 |
| 2616 | 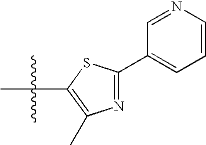 |
| 2617 | 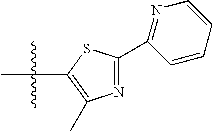 |
| 2618 | 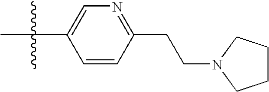 |
| 2619 | 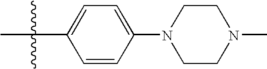 |
| 2620 | 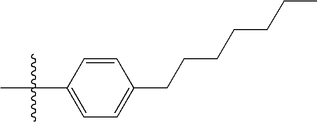 |
| 2621 | 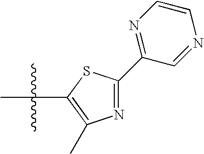 |
| 2622 | 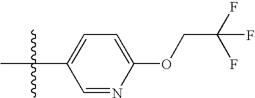 |
| 2623 | 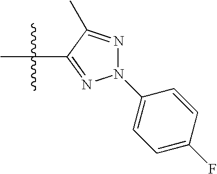 |
| 2624 | 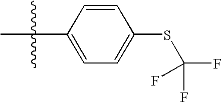 |
| 2625 | 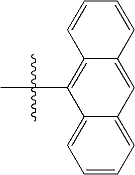 |
| 2626 | 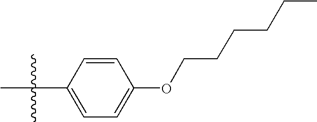 |
| 2627 | 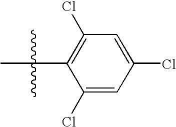 |
| 2628 | 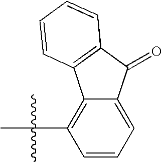 |
| 2629 | 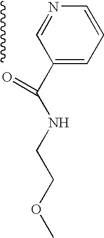 |
| 2630 | 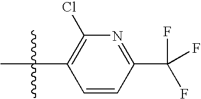 |
| 2631 | 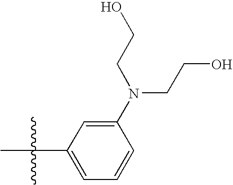 |
| 2632 | 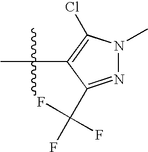 |
| 2633 | 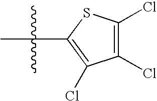 |
| 2634 | 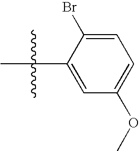 |
| 2635 | 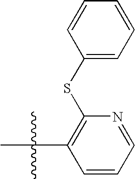 |
| 2636 | 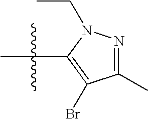 |
| 2637 | 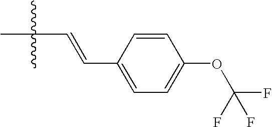 |
| 2638 | 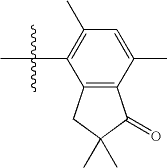 |
| 2639 | 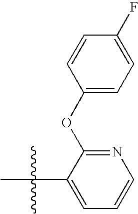 |
| 2640 | 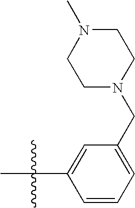 |
| 2641 | 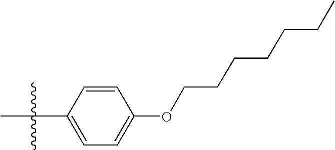 |
| 2642 | 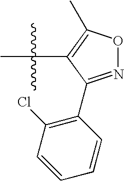 |
| 2643 | 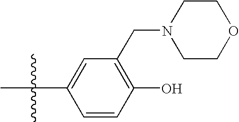 |
| 2644 | 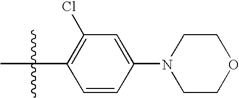 |
| 2645 | 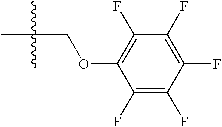 |
| 2646 | 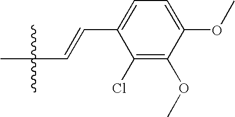 |
| 2647 | 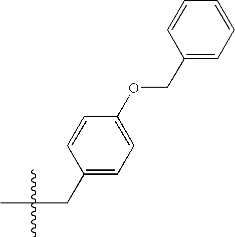 |
| 2648 | 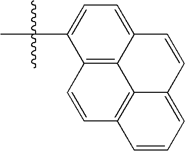 |
| 2649 | 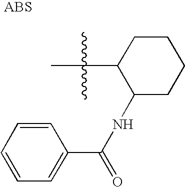 |
| 2650 | 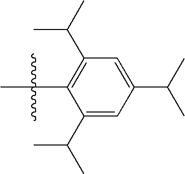 |
| 2651 | 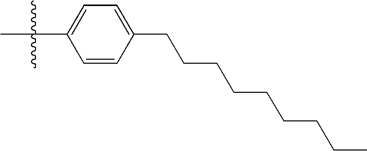 |
| 2652 | 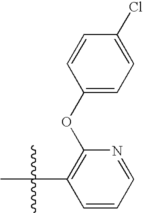 |
| 2653 | 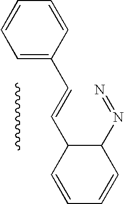 |
| 2654 | 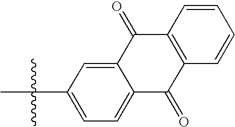 |
| 2655 | 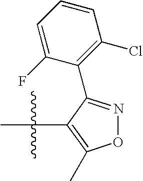 |
| 2656 | 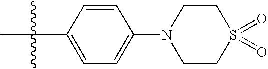 |
| 2657 | 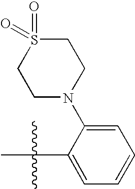 |
| 2658 | 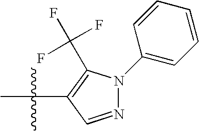 |
| 2659 | 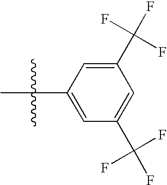 |
| 2660 | 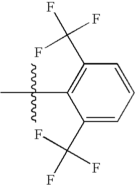 |
| 2661 | 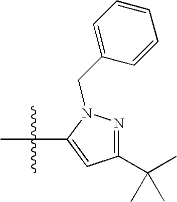 |
| 2662 | 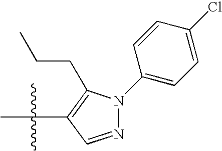 |
| 2663 | 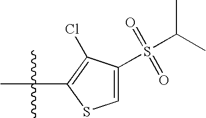 |
| 2664 | 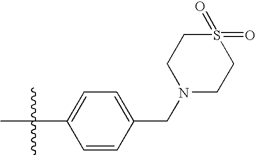 |
| 2665 | 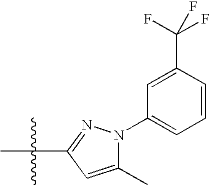 |
| 2666 | 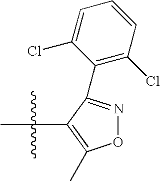 |
| 2667 | 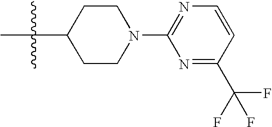 |
| 2668 | 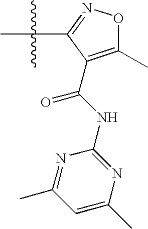 |
| 2669 | 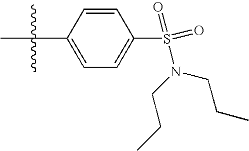 |
| 2670 | 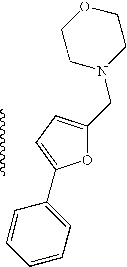 |
| 2671 | 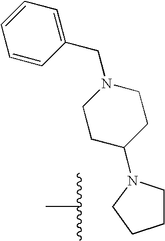 |
| 2672 | 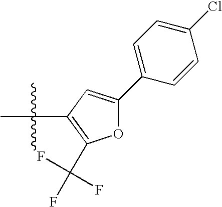 |
| 2673 | 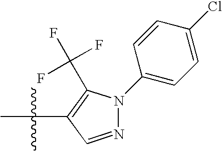 |
| 2674 | 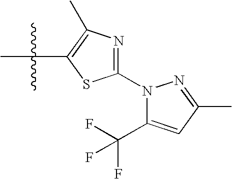 |
| 2675 | 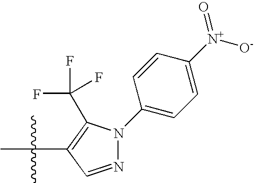 |
| 2676 | 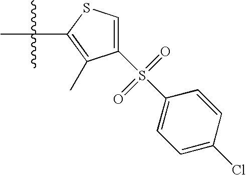 |
| 2677 | 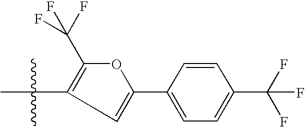 |
| 2678 | 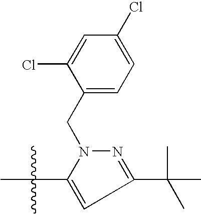 |

TABLE 6

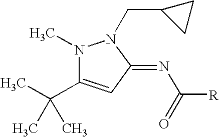

| Compound | |
|---|---|
| No. | R |
| 3001 | 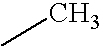 |
| 3002 | 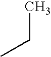 |
| 3003 | 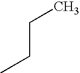 |
| 3004 | 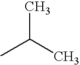 |
| 3005 | 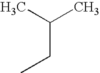 |
| 3006 | 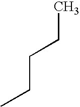 |
| 3007 | 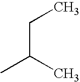 |
| 3008 | 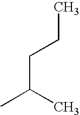 |
| 3009 | 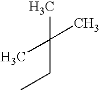 |
| 3010 | 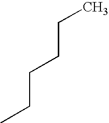 |
| 3011 | 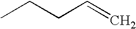 |
| 3012 | 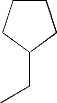 |
| 3013 | 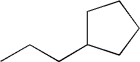 |
| 3014 | 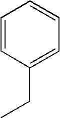 |
| 3015 | 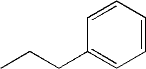 |
| 3016 | 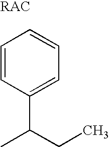 |
| 3017 | 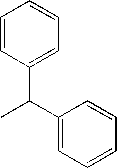 |
| 3018 | 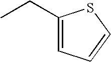 |
| 3019 | 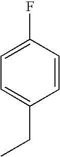 |
| 3020 | 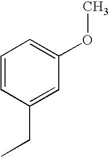 |
| 3021 | 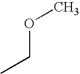 |
| 3022 | 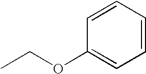 |
| 3023 | 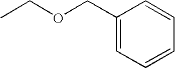 |
| 3024 | 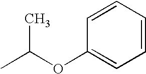 |
| 3025 | 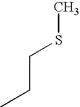 |
| 3026 | 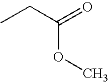 |
| 3027 | 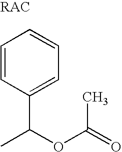 |
| 3028 | 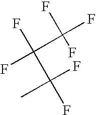 |
| 3029 | 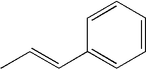 |
| 3030 | 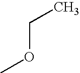 |
| 3031 | 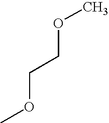 |
| 3032 | 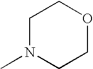 |
| 3033 | 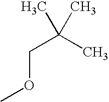 |
| 3034 | 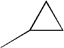 |
| 3035 | 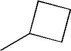 |
| 3036 | 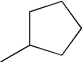 |
| 3037 | 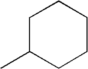 |
| 3038 | 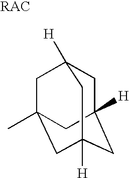 |
| 3039 | 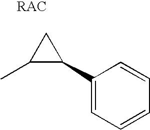 |
| 3040 | 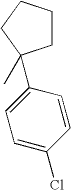 |
| 3041 | 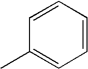 |
| 3042 | 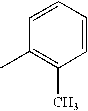 |
| 3043 | 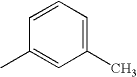 |
| 3044 | 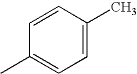 |
| 3045 | 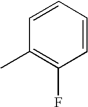 |
| 3046 | 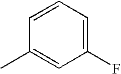 |
| 3047 | 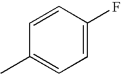 |
| 3048 | 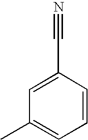 |
| 3049 | 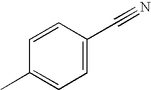 |
| 3050 | 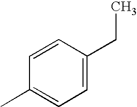 |
| 3051 | 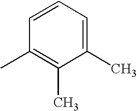 |
| 3052 | 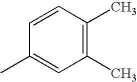 |
| 3053 | 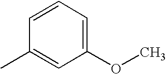 |
| 3054 | 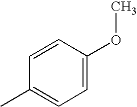 |
| 3055 | 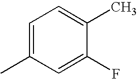 |
| 3056 | 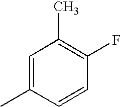 |
| 3057 | 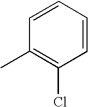 |
| 3058 | 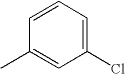 |
| 3059 | 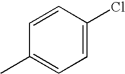 |
| 3060 | 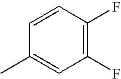 |
| 3061 | 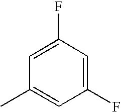 |
| 3062 | 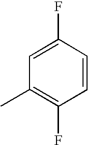 |
| 3063 | 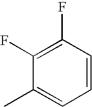 |
| 3064 | 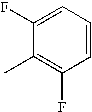 |
| 3065 | 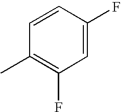 |
| 3066 | 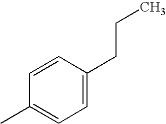 |
| 3067 | 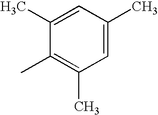 |
| 3068 | 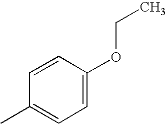 |
| 3069 | 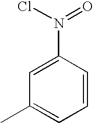 |
| 3070 | 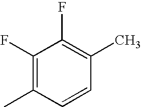 |
| 3071 | 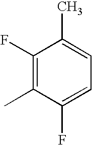 |
| 3072 | 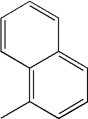 |
| 3073 | 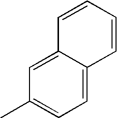 |
| 3074 | 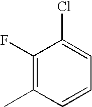 |
| 3075 | 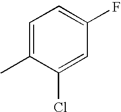 |
| 3076 | 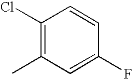 |
| 3077 | 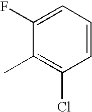 |
| 3078 | 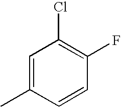 |
| 3079 | 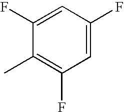 |
| 3080 | 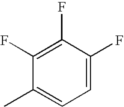 |
| 3081 | 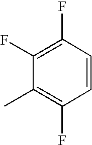 |
| 3082 | 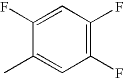 |
| 3083 | 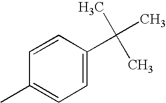 |
| 3084 | 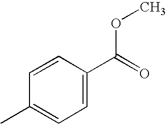 |
| 3085 | 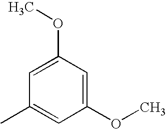 |
| 3086 | 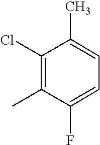 |
| 3087 | 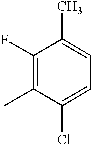 |
| 3088 | 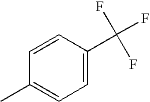 |
| 3089 | 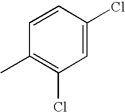 |
| 3090 | 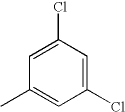 |
| 3091 | 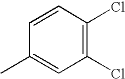 |
| 3092 | 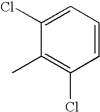 |
| 3093 | 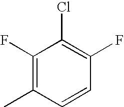 |
| 3094 | 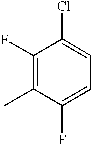 |
| 3095 | 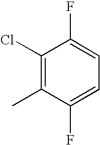 |
| 3096 | 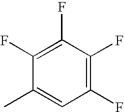 |
| 3097 | 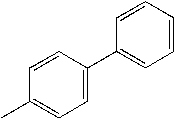 |
| 3098 | 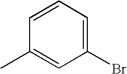 |
| 3099 | 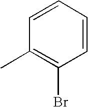 |
| 3100 | 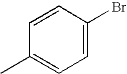 |
| 3101 | 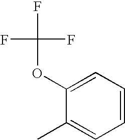 |
| 3102 | 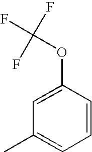 |
| 3103 | 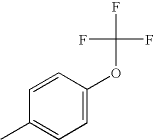 |
| 3104 | 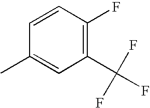 |
| 3105 | 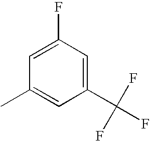 |
| 3106 | 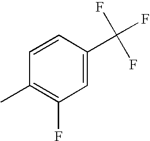 |
| 3107 | 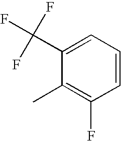 |
| 3108 | 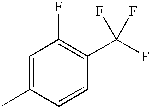 |
| 3109 | 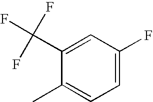 |
| 3110 | 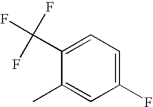 |
| 3111 | 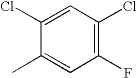 |
| 3112 | 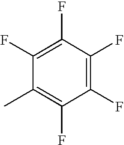 |
| 3113 | 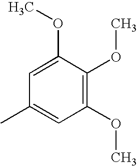 |
| 3114 | 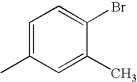 |
| 3115 | 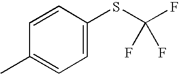 |
| 3116 | 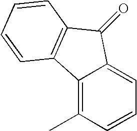 |
| 3117 | 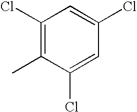 |
| 3118 | 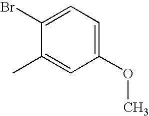 |
| 3119 | 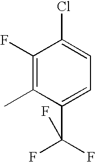 |
| 3120 | 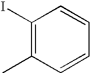 |
| 3121 | 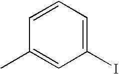 |
| 3122 | 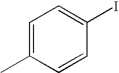 |
| 3123 | 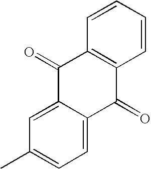 |
| 3124 | 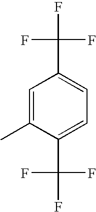 |
| 3125 | 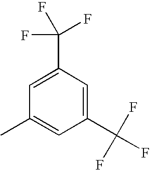 |
| 3126 | 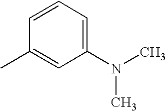 |
| 3127 | 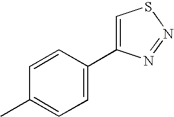 |
| 3128 | 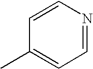 |
| 3129 | 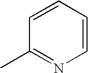 |
| 3130 | 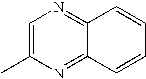 |
| 3131 | 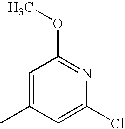 |
| 3132 | 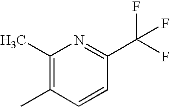 |
| 3133 | 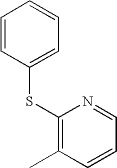 |
| 3134 | 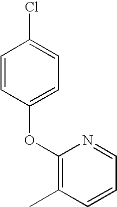 |
| 3135 | 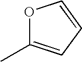 |
| 3136 | 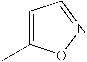 |
| 3137 | 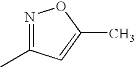 |
| 3138 | 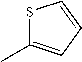 |
| 3139 | 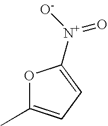 |
| 3140 | 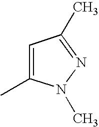 |
| 3141 | 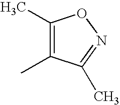 |
| 3142 | 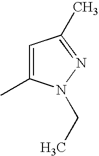 |
| 3143 | 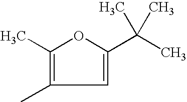 |
| 3144 | 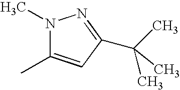 |
| 3145 | 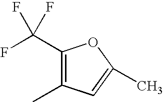 |
| 3146 | 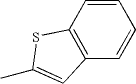 |
| 3147 | 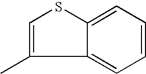 |
| 3148 | 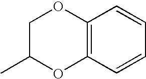 |
| 3149 | 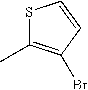 |
| 3150 | 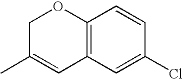 |
| 3151 | 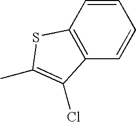 |
| 3152 | 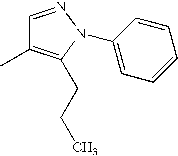 |
| 3153 | 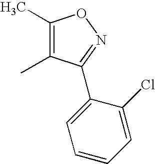 |
| 3154 | 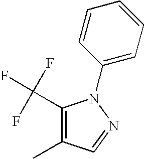 |
| 3155 | 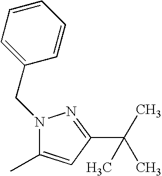 |
| 3156 | 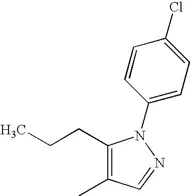 |
| 3157 | 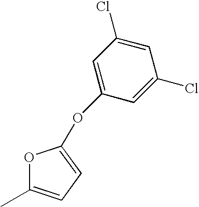 |
| 3158 | 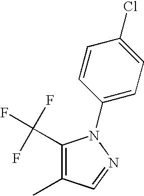 |

TABLE 7

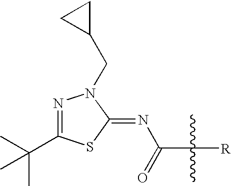

| Compound | |
|---|---|
| No. | R |
| 3159 | 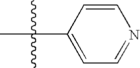 |
| 3160 | 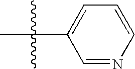 |
| 3161 | 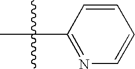 |
| 3162 | 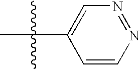 |
| 3163 | 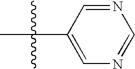 |
| 3164 | 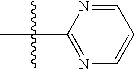 |
| 3165 | 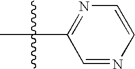 |
| 3166 | 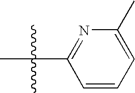 |
| 3167 | 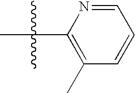 |
| 3168 | 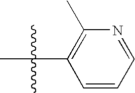 |
| 3169 | 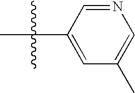 |
| 3170 | 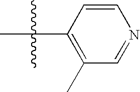 |
| 3171 | 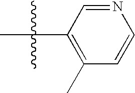 |
| 3172 | 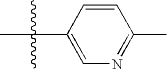 |
| 3173 | 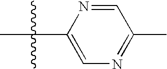 |
| 3174 | 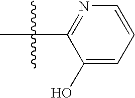 |
| 3175 | 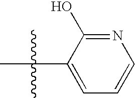 |
| 3176 | 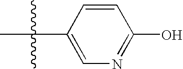 |
| 3177 | 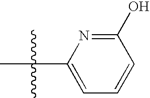 |
| 3178 | 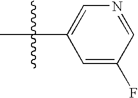 |
| 3179 | 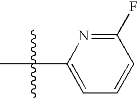 |
| 3180 | 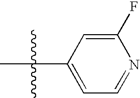 |
| 3181 | 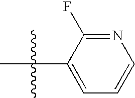 |
| 3182 | 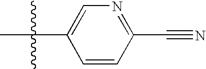 |
| 3183 | 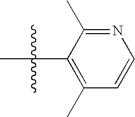 |
| 3184 | 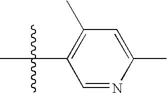 |
| 3185 | 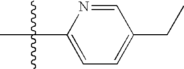 |
| 3186 | 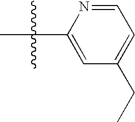 |
| 3187 | 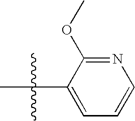 |
| 3188 | 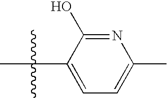 |
| 3189 | 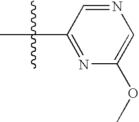 |
| 3190 | 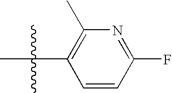 |
| 3191 | 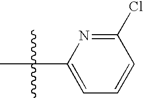 |
| 3192 | 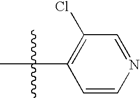 |
| 3193 | 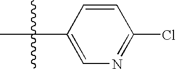 |
| 3194 | 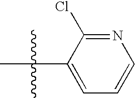 |
| 3195 | 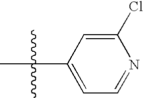 |
| 3196 | 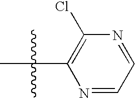 |
| 3197 | 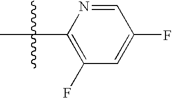 |
| 3198 | 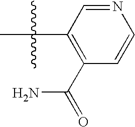 |
| 3199 | 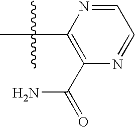 |
| 3200 | 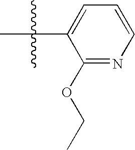 |
| 3201 | 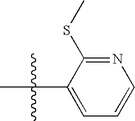 |
| 3202 | 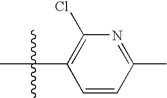 |
| 3203 | 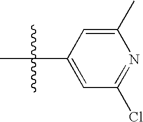 |
| 3204 | 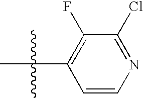 |
| 3205 | 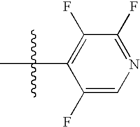 |
| 3206 | 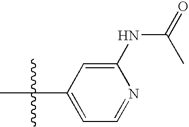 |
| 3207 | 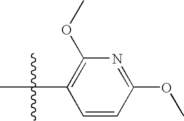 |
| 3208 | 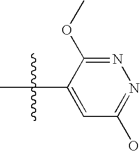 |
| 3209 | 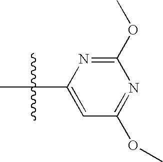 |
| 3210 | 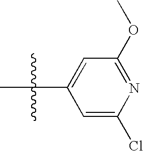 |
| 3211 | 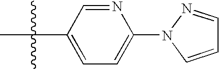 |
| 3212 | 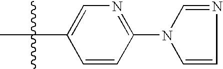 |
| 3213 | 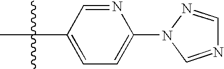 |
| 3214 | 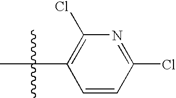 |
| 3215 | 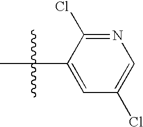 |
| 3216 | 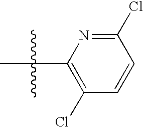 |
| 3217 | 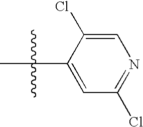 |
| 3218 | 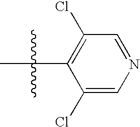 |
| 3219 | 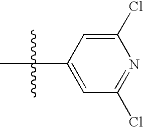 |
| 3220 | 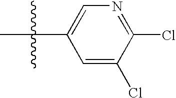 |
| 3221 | 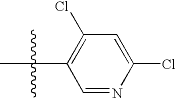 |
| 3222 | 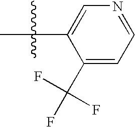 |
| 3223 | 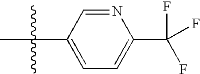 |
| 3224 | 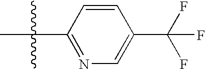 |
| 3225 | 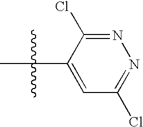 |
| 3226 | 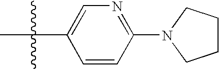 |
| 3227 | 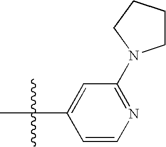 |
| 3228 | 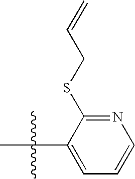 |
| 3229 | 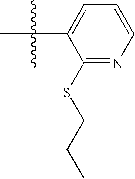 |
| 3230 | 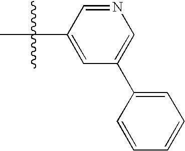 |
| 3231 | 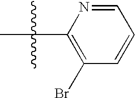 |
| 3232 | 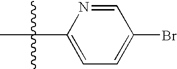 |
| 3233 | 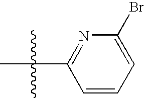 |
| 3234 | 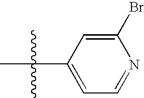 |
| 3235 | 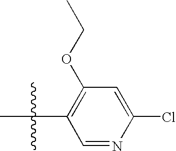 |
| 3236 | 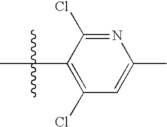 |
| 3237 | 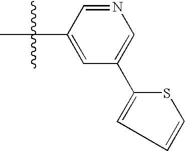 |
| 3238 | 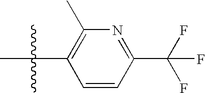 |
| 3239 | 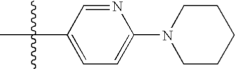 |
| 3240 | 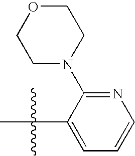 |
| 3241 | 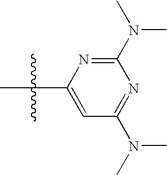 |
| 3242 | 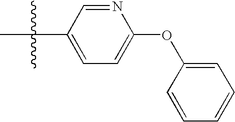 |
| 3243 | 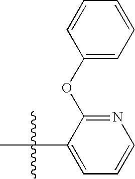 |
| 3244 | 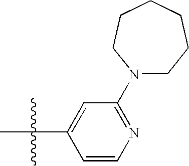 |
| 3245 | 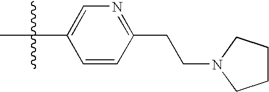 |
| 3246 | 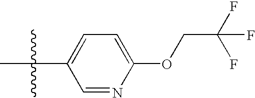 |
| 3247 | 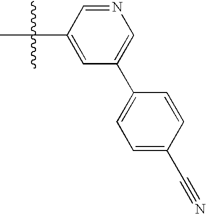 |
| 3248 | 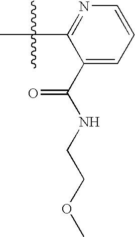 |
| 3249 | 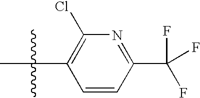 |
| 3250 | 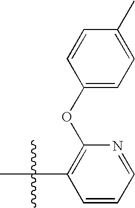 |
| 3251 | 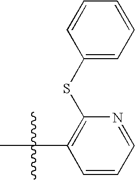 |
| 3252 | 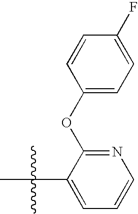 |
| 3253 | 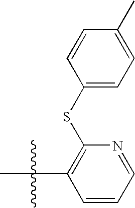 |
| 3254 | 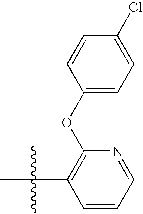 |
| 3255 | 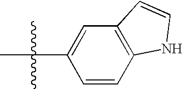 |
| 3256 | 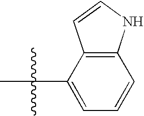 |
| 3257 | 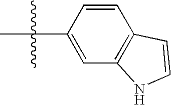 |
| 3258 | 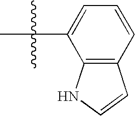 |
| 3259 | 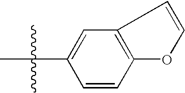 |
| 3260 | 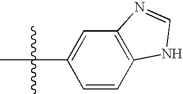 |
| 3261 | 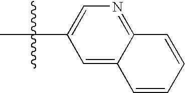 |
| 3262 | 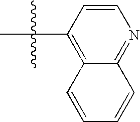 |
| 3263 | 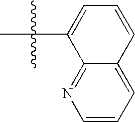 |
| 3264 | 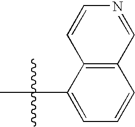 |
| 3265 | 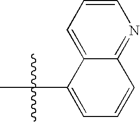 |
| 3266 | 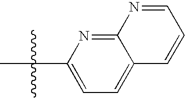 |
| 3267 | 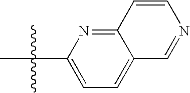 |
| 3268 | 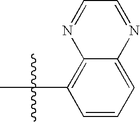 |
| 3269 | 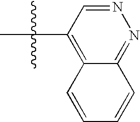 |
| 3270 | 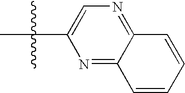 |
| 3271 | 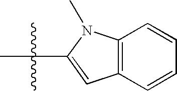 |
| 3272 | 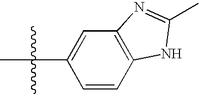 |
| 3273 | 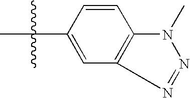 |
| 3274 | 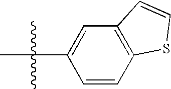 |
| 3275 | 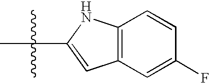 |
| 3276 | 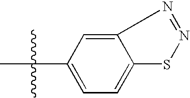 |
| 3277 | 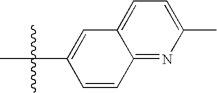 |
| 3278 | 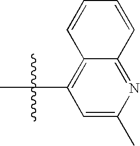 |
| 3279 | 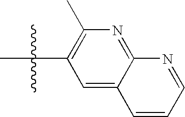 |
| 3280 | 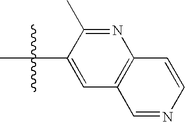 |
| 3281 | 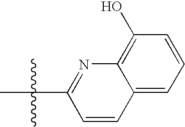 |
| 3282 | 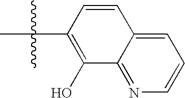 |
| 3283 | 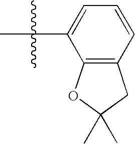 |
| 3284 | 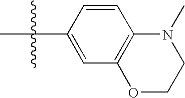 |
| 3285 | 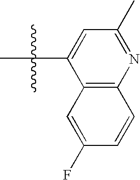 |
| 3286 | 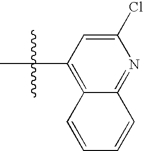 |
| 3287 | 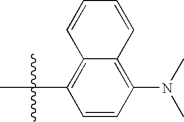 |
| 3288 | 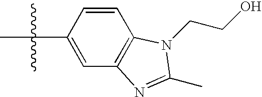 |
| 3289 | 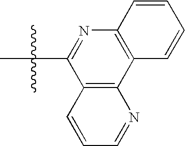 |
| 3290 | 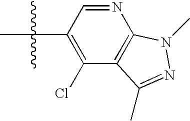 |
| 3291 | 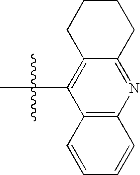 |
| 3292 | 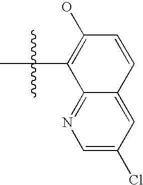 |
| 3293 | 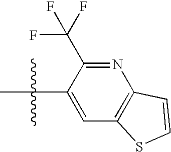 |
| 3294 | 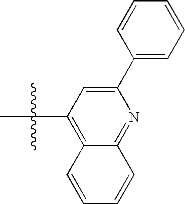 |
| 3295 | 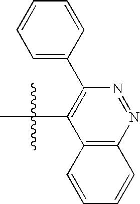 |
| 3296 | 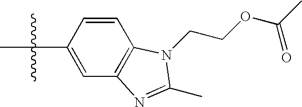 |
| 3297 | 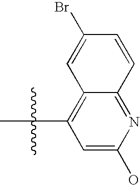 |
| 3298 | 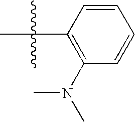 |
| 3299 | 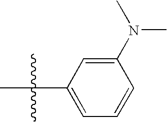 |
| 3300 | 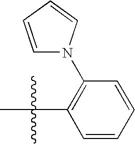 |
| 3301 | 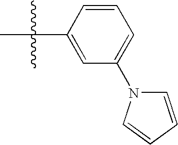 |
| 3302 | 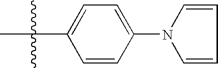 |
| 3303 | 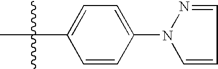 |
| 3304 | 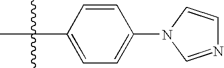 |
| 3305 | 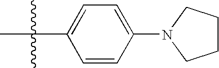 |
| 3306 | 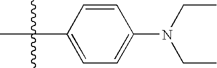 |
| 3307 | 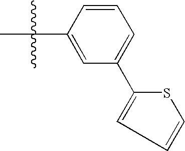 |
| 3308 | 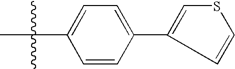 |
| 3309 | 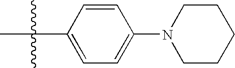 |
| 3310 | 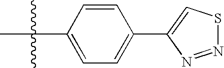 |
| 3311 | 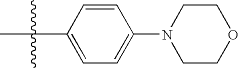 |
| 3312 | 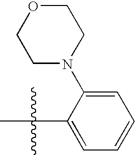 |
| 3313 | 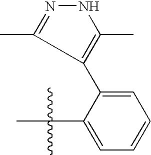 |
| 3314 | 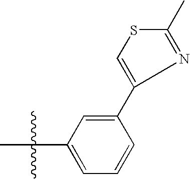 |
| 3315 | 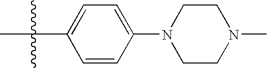 |
| 3316 | 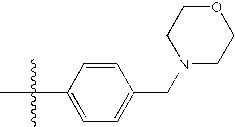 |
| 3317 | 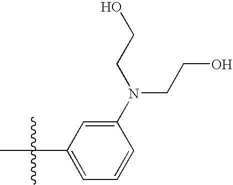 |
| 3318 | 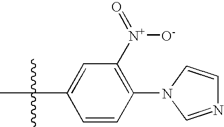 |
| 3319 | 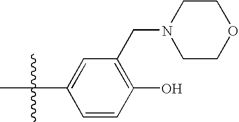 |
| 3320 | 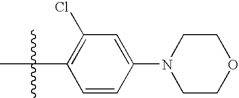 |
| 3321 | 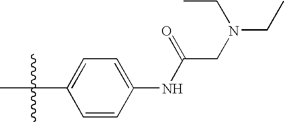 |
| 3322 | 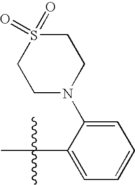 |
| 3323 | 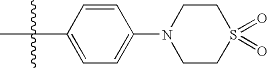 |
| 3324 | 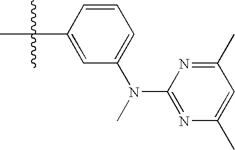 |
| 3325 | 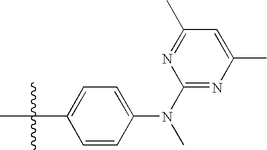 |
| 3326 | 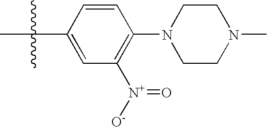 |
| 3327 | 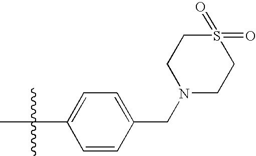 |

TABLE 8

| Compound | | | |
|---|---|---|---|
| No. | ¹H-NMR | Mass | m.p. (° C.) |
| 1 | ¹H NMR (200 MHz, CHLOROFORM-d) d ppm 0.44-0.53 (m, 2H) 0.61-0.71 (m, | ESI (Pos) | 90-91 |
| | 2H) 1.30-1.47 (m, 1H) 4.25 (d, J = 7.31 Hz, 2H) 6.82-6.92 (m, 1H) | 245 (M + H)⁺ | |
| | 7.73-7.895 (m, 2H) 8.44-8.51 (m, 1H) | | |
| 2 | ¹H NMR (200 MHz, CHLOROFORM-d) d ppm 0.42-0.55 (m, 2H) 0.62-0.78 (m, | ESI (Pos) | 105.5-106.5 |
| | 2H) 1.40-1.60 (m, 1H) 4.24 (d, J = 7.47 Hz, 2H) 6.56-6.69 (m, 1H) | 321 (M + H)⁺ | |
| | 7.45-7.75 (m, 4H) 8.38-8.59 (m, 3H) | | |
| 3 | ¹H NMR (200 MHz, CHLOROFORM-d) d ppm 0.42-0.76 (m, 4H) 1.39-1.56 (m, | ESI (Pos) | — |
| | 1H) 4.22 (d, J = 7.0 Hz, 2H) 6.53-6.65 (m, 1H) 7.06-7.22 (m, 1H) | 379 (M + H)⁺ | |
| | 7.52-7.81 (m, 3H) 8.17-8.27 (m, 1H) 8.32-8.43 (m, 1H) 8.58-8.65 (m, 1H) | | |
| 4 | ¹H NMR (200 MHz, CHLOROFORM-d) d ppm 0.53-0.73 (m, 2H) 1.23-1.53 (m, | ESI (Pos) | — |
| | 1H) 4.14 (d, J = 7.47 Hz, 2H) 6.53-6.68 (m, 1H) 7.20-7.46 (m, 3H) | 337 (M + H)⁺ | |
| | 7.49-7.64 (m, 1H) 7.64-7.77 (m, 1H) 7.85-8.03 (m, 1H) 8.22-8.37 (m, 1H) | | |
| 5 | ¹H NMR (200 MHz, CHLOROFORM-d) d ppm 3.90 (s, 3H) 6.56-6.70 (m, 1H) | ESI (Pos) | 146-147 |
| | 7.18-7.31 (m, 1H) 7.58-7.71 (m, 3H) 8.17-8.29 (m, 1H) 8.37-8.48 (m, 1H) | 299 (M + H)⁺ | |
| 6 | ¹H NMR (200 MHz, CHLOROFORM-d) d ppm 1.49 (t, J = 7.3 Hz, 3H) 4.40 (q, J = 7.3 Hz, | ESI (Pos) | 93-95 |
| | 2H) 6.60-6.71 (m, 1H) 7.18-7.31 (m, 1H) 7.56-7.69 (m, 3H) | 313 (M + H)⁺ | |
| | 8.16-8.27 (m, 1H) 8.39-8.48 (m, 1H) | | |
| 7 | ¹H NMR (200 MHz, CHLOROFORM-d) d ppm 0.97 (t, J = 7.3 Hz, 3H) 1.41 (dt, | ESI (Pos) | 51-52 |
| | J = 14.8, 7.3 Hz, 2H) 1.77-1.97 (m, 2H) 4.34 (t, J = 7.5 Hz, 2H) 6.55-6.70 (m, 1H) | 341 (M + H)⁺ | |
| | 7.17-7.31 (m, 1H) 7.55-7.70 (m, 3H) 8.15-8.28 (m, 1H) 8.41-8.50 (m, 1H) | | |
| 8 | ¹H NMR (200 MHz, CHLOROFORM-d) d ppm 1.47 (d, J = 6.6 Hz, 6H) | ESI (Pos) | 86-88 |
| | 5.84-6.06 (m, 1H) 6.64-6.77 (m, 1H) 7.18-7.32 (m, 1H) 7.53-7.78 (m, 2H) | 327 (M + H)⁺ | |
| | 8.14-8.45 (m, 2H) | | |
| 9 | ¹H NMR (200 MHz, CHLOROFORM-d) d ppm 0.38-0.74 (m, 4H) 1.32-1.53 (m, | ESI (Pos) | 58.5-59 |
| | 1H) 4.22 (d, J = 7.03 Hz, 2H) 6.61-6.72 (m, 1H) 7.18-7.31 (m, 1H) | 339 (M + H)⁺ | |
| | 7.54-7.76 (m, 3H) 8.13-8.27 (m, 1H) 8.37-8.48 (m, 1H) | | |
| 10 | ¹H NMR (200 MHz, CHLOROFORM-d) d ppm 0.36-0.51 (m, 2H) 0.60-0.74 (m, | ESI (Pos) | 53.5-54.5 |
| | 2H) 1.29-1.53 (m, 1H) 4.17 (d, J = 7.47 Hz, 2H) 6.58-6.74 (m, 1H) | 322 (M + H)⁺ | |
| | 7.16-7.41 (m, 2H) 7.55-7.82 (m, 3H) 8.33-8.45 (m, 1H) | | |
| 11 | ¹H NMR (300 MHz, CHLOROFORM-d) d ppm 0.35-0.75 (m, 4H) 1.18-1.42 (m, | ESI (Pos) | 88-89 |
| | 1H) 4.12 (d, J = 7.31 Hz, 2H) 6.61-6.71 (m, 1H) 7.19-7.42 (m, 2H) | 339 (M + H)⁺ | |
| | 7.58-7.85 (m, 3H) 8.30-8.39 (m, 1H) | | |
| 12 | ¹H NMR (600 MHz, CHLOROFORM-d) d ppm 0.44-0.72 (m, 4H) 0.74-0.90 (m, | ESI (Pos) | 93-94 |
| | 2H) 1.84-2.05 (m, 1H) 3.57-3.85 (m, 2H) 6.43-6.57 (m, 1H) 7.22-7.38 (m, | 353 (M + H)⁺ | |
| | 1H) 7.64-7.85 (m, 3H) 8.42-8.55 (m, 1H) 8.70-8.83 (m, 10H) | | |
| 13 | ¹H NMR (200 MHz, CHLOROFORM-d) d ppm −0.00-0.53 (m, 4H) 0.54-0.77 (m, | ESI (Pos) | 56-58 |
| | 1H) 1.81 (q, J = 7.0 Hz, 2H) 4.43 (t, J = 7.0 Hz, 2H) 6.58-6.69 (m, 1H) | 353 (M + H)⁺ | |
| | 7.18-7.31 (m, 1H) 7.55-7.71 (m, 3H) 8.14-8.27 (m, 1H) 8.40-8.51 (m, 1H) | | |
| 14 | ¹H NMR (200 MHz, CHLOROFORM-d) d ppm 1.12 (t, J = 7.03 Hz, 3H) 3.44 (q, | ESI (Pos) | 104.5-105.5 |
| | J = 7.03 Hz, 2H) 3.84 (t, J = 4.83 Hz, 2H) 4.54 (t, J = 4.83 Hz, 2H) 6.56-6.69 (m, 1H) | 357 (M + H)⁺ | |
| | 7.18-7.31 (m, 1H) 7.57-7.77 (m, 3H) 8.13-8.26 (m, 1H) 8.41-8.50 (m, 1H) | | |
| 15 | ¹H NMR (200 MHz, CHLOROFORM-d) d ppm 0.41-0.77 (m, 4H) 1.32-1.51 (m, | ESI (Pos) | 124-126 |
| | 1H) 4.19 (d, J = 7.5 Hz, 2H) 7.20-7.31 (m, 1H) 7.57-7.71 (m, 2H) | 439 (M + Na)⁺ | |
| | 7.81-7.87 (m, 1H) 8.14-8.26 (m, 1H) 8.35-8.43 (m, 1H) | | |
| 16 | ¹H NMR (200 MHz, CHLOROFORM-d) d ppm 3.21 (t, J = 7.3 Hz, 2H) 4.54 (t, J = 7.3 Hz, | ESI (Pos) | 117-118 |
| | 2H) 6.43-6.55 (m, 1H) 7.10-7.36 (m, 7H) 7.54-7.73 (m, 2H) | 389 (M + H)⁺ | |
| | 8.19-8.32 (m, 1H) 8.45-8.57 (m, 1H) | | |
| 17 | ¹H NMR (200 MHz, CHLOROFORM-d) d ppm 0.59-0.73 (m, 2H) 1.26-1.46 (m, | ESI (Pos) | 88.5-89 |
| | 1H) 4.13 (d, J = 7.47 Hz, 2H) 6.62-6.74 (m, 1H) 7.02-7.17 (m, 1H) | 339 (M + H)⁺ | |
| | 7.39-7.50 (m, 1H) 7.58-7.77 (m, 3H) 8.30-8.41 (m, 1H) | | |
| 18 | ¹H NMR (200 MHz, CHLOROFORM-d) d ppm 0.40-0.56 (m, 2H) 0.56-0.75 (m, | ESI (Pos) | 127-127.5 |
| | 2H) 1.34-1.56 (m, 1H) 4.22 (d, J = 7.03 Hz, 2H) 6.59-6.74 (m, 1H) | 339 (M + H)⁺ | |
| | 7.10-7.28 (m, 1H) 7.53-7.79 (m, 3H) 8.29-8.53 (m, 2H) | | |
| 19 | ¹H NMR (200 MHz, CHLOROFORM-d) d ppm 0.39-0.51 (m, 2H) 0.60-0.72 (m, | ESI (Pos) | — |
| | 2H) 1.33-1.55 (m, 1H) 3.94 (s, 3H) 4.16 (d, J = 7.03 Hz, 2H) 6.55-6.66 (m, 1H) | 351 (M + H)⁺ | |
| | 6.95-7.05 (m, 1H) 7.52-7.72 (m, 3H) 8.09-8.14 (m, 1H) 8.36-8.46 (m, 1H) | | |
| 20 | ¹H NMR (200 MHz, CHLOROFORM-d) d ppm 0.36-0.72 (m, 4H) 1.34-1.58 (m, | ESI (Pos) | — |
| | 1H) 3.80 (s, 3H) 3.85 (s, 3H) 4.15 (d, J = 7.5 Hz, 2H) 6.48-6.61 (m, 1H) | 313 (M + H)⁺ | |
| | 6.84-6.93 (m, 2H) 7.40-7.46 (m, 1H) 7.46-7.59 (m, 1H) 7.60-7.69 (m, 1H) | | |
| | 8.27-8.38 (m, 1H) | | |
| 21 | ¹H NMR (200 MHz, CHLOROFORM-d) d ppm 0.38-0.74 (m, 4H) 1.34-1.54 (m, | ESI (Pos) | — |
| | 1H) 4.20 (d, J = 7.0 Hz, 2H) 6.65-6.77 (m, 1H) 7.51-7.62 (m, 1H) | 304 (M + H)⁺ | |
| | 7.63-7.80 (m, 3H) 7.84-7.91 (m, 1H) 8.09-8.17 (m, 1H) 8.49-8.59 (m, 1H) | | |
| | 8.81-8.90 (m, 1H) 8.94-9.01 (m, 1H) | | |
| 22 | ¹H NMR (200 MHz, CHLOROFORM-d) d ppm 0.40-0.77 (m, 4H) 1.30-1.48 (m, | ESI (Pos) | — |
| | 1H) 2.38 (s, 3H) 4.23 (d, J = 7.0 Hz, 2H) 6.79-6.91 (m, 1H) 7.19-7.31 (m, 1H) | 353 (M + H)⁺ | |
| | 7.55-7.69 (m, 2H) 7.75-7.84 (m, 1H) 8.05-8.18 (m, 1H) | | |
| 23 | ¹H NMR (200 MHz, CHLOROFORM-d) d ppm 0.36-0.51 (m, 2H) 0.56-0.71 (m, | ESI (Pos) | 81-83 |
| | 2H) 1.32-1.52 (m, 1H) 2.36 (s, 3H) 4.18 (d, J = 7.0 Hz, 2H) 6.48-6.56 (m, 1H) | 353 (M + H)⁺ | |
| | 7.18-7.30 (m, 1H) 7.56-7.67 (m, 2H) 8.14-8.31 (m, 2H) | | |
| 24 | ¹H NMR (200 MHz, CHLOROFORM-d) d ppm 0.37-0.73 (m, 4H) 1.30-1.55 (m, | ESI (Pos) | 118-120 |
| | 1H) 2.25 (s, 3H) 4.21 (d, J = 7.5 Hz, 2H) 7.17-7.31 (m, 1H) 7.44-7.69 (m, 2H) | 353 (M + H)⁺ | |
| | 8.10-8.27 (m, 1H) 8.35-8.48 (m, 1H) | | |
| 25 | ¹H NMR (200 MHz, CHLOROFORM-d) d ppm 0.49-0.62 (m, 4H) 1.20-1.40 (m, | ESI (Pos) | 96-98 |
| | 1H) 2.61 (s, 3H) 4.50 (d, J = 7.0 Hz, 2H) 6.54 (d, J = 7.0 Hz, 1H) 7.19-7.30 (m, 1H) | 353 (M + H)⁺ | |
| | 7.45-7.67 (m, 2H) 8.11-8.31 (m, 2H) | | |
| 26 | ¹H NMR (600 MHz, CHLOROFORM-d) d ppm 0.39-0.45 (m, 2H) 0.67-0.73 (m, | APCI (Pos) | 93-93.5 |
| | 2H) 1.31-1.39 (m, 1H) 4.10 (d, J = 7.34 Hz, 2H) 7.47 (t, J = 7.79 Hz, 1H) 7.57 (t, | 389 (M + H)⁺ | |
| | J = 7.57 Hz, 1H) 7.65 (dd, J = 9.40, 2.06 Hz, 1H) 7.68 (d, J = 7.79 Hz, 1H) 7.76 (d, | | |
| | J = 7.79 Hz, 1H) 8.02 (s, 1H) 8.27 (d, J = 9.17 Hz, 1H) | | |
| 27 | ¹H NMR (600 MHz, CHLOROFORM-d) d ppm 0.46-0.54 (m, 2H) 0.72-0.80 (m, | APCI (Pos) | 84-85 |
| | 2H) 1.44-1.51 (m, 1H) 4.22 (d, J = 7.34 Hz, 2H) 7.56 (t, J = 7.79 Hz, 1H) | 389 (M + H)⁺ | |
| | 7.64 (dd, J = 9.63, 2.29 Hz, 1H) 7.73 (d, J = 7.79 Hz, 1H) 8.02 (s, 1H) 8.33 (d, J = 9.63 Hz, | | |
| | 1H) 8.41 (d, J = 7.79 Hz, 1H) 8.51 (s, 1H) | | |
| 28 | ¹H NMR (600 MHz, CHLOROFORM-d) d ppm −1.19-−1.12 (m, 2H) | ESI (Pos) | — |
| | −0.78-−0.72 (m, 2H) −0.14-−0.04 (m, 1H) 3.46 (d, J = 9.2 Hz, 2H) 6.92-6.97 (m, 1H) | 407 (M + H)⁺ | |
| | 7.29-7.34 (m, 1H) 7.75-7.82 (m, 1H) 8.35-8.43 (m, 2H) 8.60-8.64 (m, 1H) | | |
| 29 | ¹H NMR (200 MHz, CHLOROFORM-d) d ppm 0.40-0.77 (m, 4H) 1.37-1.52 (m, | ESI (Pos) | 85-86 |
| | 1H) 4.22 (d, J = 7.5 Hz, 2H) 6.68-6.77 (m, 1H) 7.21-7.33 (m, 1H) | 407 (M + H)⁺ | |
| | 7.61-7.74 (m, 1H) 7.78-7.88 (m, 1H) 8.16-8.30 (m, 1H) 8.65-8.73 (m, 1H) | | |
| 30 | ¹H NMR (200 MHz, CHLOROFORM-d) d ppm 0.40-0.77 (m, 4H) 1.30-1.48 (m, | ESI (Pos) | — |
| | 1H) 2.38 (s, 3H) 4.23 (d, J = 7.0 Hz, 2H) 6.79-6.91 (m, 1H) 7.19-731 (m, 1H) | 353 (M + H)⁺ | |
| | 7.55-7.69 (m, 2H) 7.75-7.84 (m, 1H) 8.05-8.18 (m, 1H) | | |
| 31 | ¹H NMR (600 MHz, CHLOROFORM-d) d ppm 0.42-0.53 (m, 2H) 0.66-0.79 (m, | APCI (Pos) | 109-110 |
| | 2H) 1.37-1.48 (m, 1H) 4.20 (d, J = 7.34 Hz, 2H) 7.18-7.23 (m, 1H) | 407 (M + H)⁺ | |
| | 7.65-770 (m, 2H) 8.03 (s, 1H) 8.36 (dd, J = 6.65, 2.52 Hz, 1H) 8.39 (d, J = 9.63 Hz, 1H) | | |
| 32 | ¹H NMR (500 MHz, CHLOROFORM-d) d ppm 0.36-0.70 (m, 4H) 1.26-1.37 (m, | ESI (Pos) | — |
| | 1H) 4.30 (d, J = 6.9 Hz, 2H) 6.97-7.03 (m, 1H) 7.17-7.22 (m, 1H) | 407 (M + H)⁺ | |
| | 7.22-7.26 (m, 1H) 7.54-7.59 (m, 1H) 7.59-7.64 (m, 1H) 7.66-7.72 (m, 1H) | | |
| 33 | ¹H NMR (200 MHz, CHLOROFORM-d) d ppm 0.38-0.74 (m, 4H) 1.30-1.49 (m, | ESI (Pos) | 91-93 |
| | 1H) 4.17 (d, J = 7.0 Hz, 2H) 6.61-6.68 (m, 1H) 7.22-7.32 (m, 1H) | 395 (M + Na)⁺ | |
| | 7.59-7.72 (m, 2H) 8.15-8.26 (m, 1H) 8.51-8.56 (m, 1H) | | |
| 34 | ¹H NMR (200 MHz, CHLOROFORM-d) d ppm 0.40-0.79 (m, 4H) 1.33-1.52 (m, | ESI (Pos) | 117-119 |
| | 1H) 4.20 (d, J = 7.5 Hz, 2H) 7.18-7.30 (m, 1H) 7.51-7.71 (m, 2H) | 373 (M + H)⁺ | |
| | 7.72-7.79 (m, 1H) 8.13-8.26 (m, 1H) 8.39-8.51 (m, 1H) | | |
| 35 | ¹H NMR (200 MHz, CHLOROFORM-d) d ppm 0.48-0.67 (m, 4H) 1.35-1.55 (m, | ESI (Pos) | 81-83 |
| | 1H) 4.67 (d, J = 7.5 Hz, 2H) 6.70-6.76 (m, 1H) 7.20-7.31 (m, 1H) | 373 (M + H)⁺ | |
| | 7.42-7.54 (m, 1H) 7.59-7.70 (m, 1H) 8.12-8.23 (m, 1H) 8.25-8.35 (m, 1H) | | |
| 36 | ¹H NMR (200 MHz, CHLOROFORM-d) d ppm 0.38-0.83 (m, 4H) 1.32-1.51 (m, | ESI (Pos) | 93-94 |
| | 1H) 4.19 (d, J = 7.5 Hz, 2H) 7.21-7.33 (m, 1H) 7.59-7.74 (m, 2H) | 407 (M + H)⁺ | |
| | 7.99-8.07 (m, 1H) 8.15-8.28 (m, 1H) 8.30-8.40 (m, 1H) | | |
| 37 | ¹H NMR (200 MHz, CHLOROFORM-d) d ppm 0.41-0.54 (m, 2H) 0.64-0.80 (m, | ESI (Pos) | 89-91 |
| | 2H) 1.31-1.51 (m, 1H) 4.23 (d, J = 7.5 Hz, 2H) 7.48-7.78 (m, 3H) | 357 (M + H)⁺ | |
| | 8.13-8.27 (m, 1H) 8.49-8.63 (m, 1H) | | |
| 38 | ¹H NMR (200 MHz, CHLOROFORM-d) d ppm 0.43-0.57 (m, 2H) 0.60-0.77 (m, | ESI (Pos) | 115-116 |
| | 2H) 1.39-1.58 (m, 1H) 4.31 (d, J = 7.0 Hz, 2H) 1.19-7.32 (m, 1H) | 415 (M + H)⁺ | |
| | 7.35-7.55 (m, 5H) 7.57-7.71 (m, 1H) 7.83-7.98 (m, 2H) 8.15-8.30 (m, 1H) | | |
| | 8.46-8.61 (m, 1H) | | |
| 39 | ¹H NMR (200 MHz, CHLOROFORM-d) d ppm 0.44-0.54 (m, 4H) 1.28-1.51 (m, | ESI (Pos) | 126-128 |
| | 1H) 4.02 (s, 3H) 4.46 (d, J = 7.5 Hz, 2H) 6.03-6.09 (m, 1H) 7.17-7.29 (m, 1H) | 369 (M + H)⁺ | |
| | 7.52-7.66 (m, 2H) 7.99-8.09 (m, 1H) 8.12-8.25 (m, 1H) | | |
| 40 | ¹H NMR (200 MHz, CHLOROFORM-d) d ppm 0.32-0.54 (m, 4H) 1.41-1.58 (m, | ESI (Pos) | — |
| | 1H) 2.73 (s, 6H) 4.49 (d, J = 6.6 Hz, 2H) 6.33-6.42 (m, 1H) 7.18-7.29 (m, 1H) | 382 (M + H)⁺ | |
| | 7.49-7.67 (m, 2H) 8.06-8.25 (m, 2H) | | |
| 41 | ¹H NMR (200 MHz, CHLOROPORM-d) d ppm 0.53-0.63 (m, 4H) 1.12-1.37 (m, | ESI (Pos) | 135-137 |
| | 1H) 2.78 (s, 3H) 4.61 (d, J = 7.0 Hz, 2H) 7.25 (d, 1H) 7.56-7.73 (m, 2H) | 431 (M + H)⁺ | |
| | 8.05-8.26 (m, 2H) | | |
| 42 | ¹H NMR (200 MHz, CHLOROFORM-d) d ppm 0.44-0.83 (m, 4H) 1.27-1.45 (m, | ESI (Pos) | 69-71 |
| | 1H) 4.16 (d, J = 7.0 Hz, 2H) 7.19-7.42 (m, 2H) 7.61-7.71 (m, 2H) | 397 (M + Na)⁺ | |
| | 8.13-8.24 (m, 1H) | | |
| 43 | ¹H NMR (200 MHz, CHLOROFORM-d) d ppm 2.23 (s, 3H) 2.43 (s, 3H) 3.81 (s, 3H) | ESI (Pos) | 117.5-118 |
| | 3.83 (s, 3H) 3.89 (s, 3H) 6.22-6.27 (m, 1H) 6.40-6.53 (m, 2H) | 301 (M + H)⁺ | |
| | 7.93-8.01 (m, 2H) | | |
| 44 | ¹H NMR (200 MHz, CHLOROFORM-d) d ppm 2.27 (s, 3H) 2.46 (s, 3H) 2.63 (s, 3H) | ESI (Pos) | — |
| | 4.97-5.30 (m, 4H) 5.89-6.12 (m, 1H) 6.27 (s, 1H) 7.11-7.32 (m, 3H) | 281 (M + H)⁺ | |
| | 7.86-7.95 (m, 1H) 8.02 (s, 1H) | | |
| 45 | ¹H NMR (200 MHz, CHLOROFORM-d) d ppm 0.50-0.60 (m, 4H) 1.15-1.37 (m, | ESI (Pos) | 114-115 |
| | 1H) 2.30 (s, 3H) 2.56 (s, 3H) 4.46 (d, J = 7.0 Hz, 2H) 6.34-6.44 (m, 1H) | 367 (M + H)⁺ | |
| | 7.16-7.28 (m, 1H) 7.54-7.67 (m, 1H) 8.09-8.24 (m, 2H) | | |
| 46 | ¹H NMR (200 MHz, CHLOROFORM-d) d ppm 0.52-0.62 (m, 4H) 1.14-1.30 (m, | ESI (Pos) | 121-122 |
| | 1H) 2.43 (s, 3H) 2.83 (s, 3H) 4.63 (d, J = 6.6 Hz, 2H) 7.18-7.29 (m, 1H) | 445 (M + H)⁺ | |
| | 7.56-7.68 (m, 1H) 8.09-8.22 (m, 1H) 8.28 (s, 1H) | | |
| 47 | ¹H NMR (200 MHz, CHLOROFORM-d) d ppm 0.48-0.73 (m, 4H) 1.23-1.49 (m, | ESI (Pos) 411 | 125-126 |
| | 1H) 4.71 (d, J = 7.0 Hz, 2H) 7.20-7.45 (m, 2H) 7.62-7.90 (m, 5H) | (M + Na)⁺ | |
| | 8.03-8.26 (m, 2H) | | |
| 48 | ¹H NMR (200 MHz, CHLOROFORM-d) d ppm 0.42-0.76 (m, 4H) 1.33-1.52 (m, | ESI (Pos) 389 | — |
| | 1H) 4.23 (d, J = 7.0 Hz, 2H) 7.06-7.13 (m, 1H) 7.26-7.37 (m, 1H) | (M + H)⁺ | |
| | 7.55-7.82 (m, 5H) 8.21-8.39 (m, 2H) | | |
| 49 | ¹H NMR (200 MHz, CHLOROFORM-d) d ppm 0.27-0.55 (m, 4H) 1.22-1.44 (m, | ESI (Pos) 340 | — |
| | 1H) 4.19 (d, J = 7.0 Hz, 2H) 6.91-6.99 (m, 1H) 7.18-7.32 (m, 1H) | (M + H)⁺ | |
| | 7.51-7.82 (m, 2H) 8.34-8.43 (m, 2H) | | |
| 50 | ¹H NMR (200 MHz, CHLOROFORM-d) d ppm 0.28-0.40 (2H, m), 0.57-0.69 (2H, | ESI (Pos) 356 | — |
| | m), 1.21-1.39 (1H, m), 3.98 (2H, d, J = 7.47 Hz), 6.54-6.64 (2H, m), | (M + H)+ | |
| | 7.45-7.73 (4H, m), 7.76-7.84 (1H, m), 8.38-8.46 (1H, m) | | |
| 51 | ¹H NMR (200 MHz, CHLOROFORM-d)d ppm 0.42-0.69 (m, 4H) 1.19-1.33 (m, 1H) | ESI (Pos) 342 | — |
| | 1.37 (s, 9H) 4.12 (d, J = 7.0 Hz, 2H) 7.17 (s, 1H) 7.18-7.28 (m, 1H) | (M + H)+ | |
| | 7.57-7.68 (m, 1H) 8.12-8.23 (m, 1H) | | |
| 52 | ¹H NMR (200 MHz, CHLOROFORM-d) d ppm 0.56-0.69 (m, 4H) 1.20-1.37 (m, | ESI (Pos) 388 | — |
| | 1H) 1.42 (d, J = 6.6 Hz, 6H) 3.13-3.31 (m, 1H) 4.24 (d, J = 7.0 Hz, 2H) | (M + H)⁺ | |
| | 7.26-7.39 (m, 1H) 7.67-7.80 (m, 1H) 8.27-8.40 (m, 1H) | | |
| 53 | ¹H NMR (200 MHz, CHLOROFORM-d) d ppm 0.41-0.73 (m, 4H) 1.17-1.41 (m, | ESI (Pos) 367 | — |
| | 1H) 1.36 (s, 9H) 4.17 (d, J = 7.0 Hz, 2H) 7.17 (s, 1H) 7.46-7.57 (m, 1H) | (M + H)⁺ | |
| | 7.65-7.73 (m, 1H) 8.38-8.46 (m, 1H) 8.48-8.57 (m, 1H) | | |
| 54 | ¹H NMR (200 MHz, CHLOROFORM-d) d ppm 0.42-0.70 (m, 4H) 1.21-1.32 (m, | ESI (Pos) 385 | — |
| | 1H) 1.36 (s, 9H) 4.13 (d, J = 7.0 Hz, 2H) 7.11-7.23 (m, 1H) 7.19 (s, 1H) | (M + H)⁺ | |
| | 7.56-7.67 (m, 1H) 8.29-8.37 (m, 1H) | | |
| 55 | ¹H NMR (200 MHz, CHLOROFORM-d) d ppm 0.38-0.69 (m, 4H) 1.20-1.34 (m, | ESI (Pos) 397 | 64-65 |
| | 1H) 1.35 (s, 9H) 3.93 (s, 3H) 4.09 (d, J = 7.5 Hz, 2H) 6.95-7.03 (m, 1H) 7.23 (s, | (M + H)⁺ | |
| | 1H) 7.54-7.62 (m, 1H) 8.05-8.12 (m, 1H) | | |
| 56 | ¹H NMR (200 MHz, CHLOROFORM-d) d ppm 0.41-0.55 (m, 2H) 0.67-0.81 (m, | ESI (Pos) 401 | 85-87 |
| | 2H) 1.20-1.35 (m, 1H) 1.43 (s, 9H) 4.01 (d, J = 7.0 Hz, 2H) 7.16-7.32 (m, 1H) | (M + H)⁺ | |
| | 7.56-7.70 (m, 1H) 7.85 (s, 1H) 8.16-8.29 (m, 1H) | | |
| 57 | ¹H NMR (200 MHz, CHLOROFORM-d) d ppm 0.44-0.58 (m, 2H) 0.68-0.83 (m, | ESI (Pos) 383 | 54-55 |
| | 2H) 1.21-1.38 (m, 1H) 1.43 (s, 9H) 4.05 (d, J = 7.5 Hz, 2H) 7.52 (t, J = 7.5 Hz, 1H) | (M + H)⁺ | |
| | 7.69 (d, J = 7.5 Hz, 1H) 7.86 (s, 1H) 8.46 (d, J = 7.5 Hz, 1H) 8.57 (s, 1H) | | |
| 58 | ¹H NMR (200 MHz, CHLOROFORM-d) d ppm 0.67-0.80 (m, 2H) 1.19-1.38 (m, | ESI (Pos) 401 | 63-65 |
| | 1H) 1.43 (s, 9H) 4.02 (d, J = 7.0 Hz, 2H) 7.09-7.28 (m, 1H) 7.55-7.68 (m, 1H) | (M + H)⁺ | |
| | 7.88 (s, 1H) 8.33-8.43 (m, 1H) | | |
| 59 | ¹H NMR (200 MHz, CHLOROFORM-d) d ppm 0.40-0.53 (m, 52H) 0.64-0.80 (m, | ESI (Pos) 413 | 95-97 |
| | 2H) 1.18-1.36 (m, 1H) 1.42 (s, 9H) 3.94 (s, 3H) 3.97 (d, J = 7.5 Hz, 2H) | (M + H)⁺ | |
| | 6.95-7.05 (m, 1H) 7.52-7.63 (m, 1H) 7.91 (s, 1H) 8.11-8.17 (m, 1H) | | |

TABLE 9

| Compound No. | H-NMR | m.p. (° C.) |
|---|---|---|
| 60 | 1H NMR (200 MHz, CHLOROFORM-D) d ppm 0.33-0.84 (m, 4H) 1.14-1.50 (m, 1H) | 96-97 |
| | 4.15 (d, J = 7.5 Hz, 2H) 6.76 (d, J = 4.8 Hz, 1H) 7.12-7.39 (m, 2H) | |
| | 7.63-7.75 (m, 1H) 8.26-8.39 (m, 1H) | |
| 61 | 1H NMR (200 MHz, CHLOROFORM-D) d ppm 1.16 (t, J = 7.0 Hz, 3H), 3.49 (q, J = 7.0 Hz, | 199-201 |
| | 2H), 3.81 (t, J = 4.8 Hz, 2H), 4.47 (t, J = 4.8 Hz, 2H), 6.70 (d, J = 4.4 Hz, | |
| | 1H), 7.17-7.36 (m, 2H), 7.62-7.76 (m, 1H), 8.25-8.37 (m, 1H) | |
| 62 | 1H NMR (200 MHz, CHLOROFORM-D) d ppm 1.41 (t, J = 7.0 Hz, 3H), 2.38 (s, 3H), | 48-50 |
| | 4.32 (q, J = 7.0 Hz, 2H), 6.36 (s, 1H), 7.27 (t, J = 7.7 Hz, 1H), 7.68 (t, | |
| | J = 7.7 Hz, 1H), 8.33 (t, J = 7.7 Hz, 1H) | |
| 63 | 1H NMR (200 MHz, CHLOROFORM-D) d ppm 0.38-0.73 (m, 4H) 1.13-1.42 (m, 1H) | 109-111 |
| | 2.41 (s, 3H) 4.20 (d, J = 7.0 Hz, 2H) 6.38 (s, 1H) 7.21-7.33 (m, 1H) | |
| | 7.63-7.74 (m, 1H) 8.26-8.36 (m, 1H) | |
| 64 | 1H NMR (200 MHz, CHLOROFORM-D) d ppm 2.33 (s, 3H), 3.81 (s, 3H), 6.71 (s, 1H), | 173-174 |
| | 7.32-7.45 (m, 1H), 8.51-8.59 (m, 1H), 8.65-8.74 (m, 1H), | |
| | 9.51-9.59 (m, 4H) | |
| 65 | 1H NMR (200 MHz, CHLOROFORM-D) d ppm 1.47 (t, J = 7.0 Hz, 3H), 2.35 (s, 3H), | 90-92 |
| | 4.35 (q, J = 7.0 Hz, 2H), 6.78 (s, 1H), 7.29 (t, J = 7.7 Hz, 1H), 7.69 (t, | |
| | J = 7.7 Hz, 1H), 8.30 (t, J = 7.7 Hz, 1H) | |
| 66 | 1H NMR (200 MHz, CHLOROFORM-D) d ppm 0.38-0.77 (m, 4H), 1.18-1.43 (m, 1H), | 115-117 |
| | 2.35 (s, 3H), 4.11 (d, J = 7.5 Hz, 2H), 6.84 (s, 1H), 7.31-7.42 (m, 1H), | |
| | 8.47-8.56 (m, 1H), 8.62-8.74 (m, 1H), 9.48-9.54 (m, 1H) | |
| 67 | 1H NMR (200 MHz, CHLOROFORM-D) d ppm 0.39-0.76 (m, 4H) 1.18-1.42 (m, 1H) | 129-130 |
| | 2.32-2.39 (m, 3H) 4.08 (d, J = 7.0 Hz, 2H) 6.83-6.89 (m, 1H) | |
| | 7.19-7.34 (m, 1H) 7.60-7.75 (m, 1H) 8.22-8.37 (m, 1H) | |
| 68 | 1H NMR (200 MHz, CHLOROFORM-D) d ppm 2.26 (s, 6H) 3.82 (s, 3H) 7.55 (t, | 110-111 |
| | J = 7.9 Hz, 1H) 7.71 (d, J = 7.9 Hz, 1H) 8.50 (d, J = 7.9 Hz, 1H) 8.61 (s, 1H) | |
| 69 | 1H NMR (200 MHz, CHLOROFORM-D) d ppm 2.26 (s, 6H) 3.78 (s, 3H) | 182-183 |
| | 7.21-7.33 (m, 1H) 7.62-7.72 (m, 1H) 8.28-8.39 (m, 1H) | |
| 70 | 1H NMR (200 MHz, CHLOROFORM-D) d ppm 1.39 (t, J = 7.0 Hz, 3H) 2.26 (s, 3H) | 198-200 |
| | 2.26 (s, 3H) 4.30 (q, J = 7.0 Hz, 2H) 7.16-7.35 (m, 1H) 7.55-7.76 (m, 1H) | |
| | 8.14-8.47 (m, 1H) | |
| 71 | 1H NMR (300 MHz, CHLOROFORM-D) d ppm 0.47-0.61 (m, 4H) 1.22-1.33 (m, 1H) | 150-152 |
| | 1.26 (t, J = 7.5 Hz, 3H) 2.24 (s, 3H) 2.27 (s, 3H) 3.11 (q, J = 7.5 Hz, 2H) | |
| | 4.14 (d, J = 6.8 Hz, 2H) 7.19-7.27 (m, 2H) 7.30-7.37 (m, 1H) 7.97-8.02 | |
| 72 | 1H NMR (200 MHz, CHLOROFORM-D) d ppm 0.53-0.65 (m, 4H), 1.18-1.41 (m, 1H), | 197-199 |
| | 2.26 (s, 3H), 2.29 (s, 3H), 4.21 (d, J = 7.0 Hz, 2H), 7.54 (t, J = 7.9 Hz, | |
| | 1H), 7.70 (d, J = 7.9 Hz, 1H), 8.44 (d, J = 7.9 Hz, 1H), 8.56 (s, 1H) | |
| 73 | 1H NMR (300 MHz, CHLOROFORM-D) d ppm 0.46-0.60 (m, 4H) 1.21-1.37 (m, 1H) | 146-148 |
| | 2.23 (s, 3H) 2.26 (s, 3H) 2.34 (s, 3H) 2.69 (s, 3H) 4.15 (d, J = 7.0 Hz, 2H) | |
| | 7.02-7.08 (m, 2H) 8.03-8.08 (m, 1H) | |
| 74 | 1H NMR (300 MHz, CHLOROFORM-D) d ppm 0.48-0.60 (m, 4H), 1.22-1.33 (m, 1H), | 125-127 |
| | 2.26 (s, 3H), 2.28 (s, 3H), 4.16 (d, J = 7.0 Hz, 2H), 7.18-7.25 (m, 1H), | |
| | 7.29-7.38 (m, 1H), 7.59-7.66 (m, 1H), 7.87-7.94 (m, 1H) | |
| 75 | 1H NMR (300 MHz, CHLOROFORM-D) d ppm 0.46-0.62 (m, 4H), 1.20-1.35 (m, 1H), | 125-127 |
| | 2.25 (s, 3H), 2.28 (s, 3H), 4.15 (d, J = 6.8 Hz, 2H), 7.23-7.34 (m, 2H), | |
| | 7.37-7.44 (m, 1H), 7.90-8.00 (m, 1H) | |
| 76 | 1H NMR (300 MHz, CHLOROFORM-D) d ppm 0.43-0.58 (m, 4H), 1.18-1.29 (m, 1H), | 114-116 |
| | 2.25 (s, 3H), 2.28 (s, 3H), 4.12 (d, J = 7.0 Hz, 2H), 7.43-7.52 (m, 1H), | |
| | 7.52-7.61 (m, 1H), 7.67-7.74 (m, 1H), 7.81-7.87 (m, 1H) | |
| 77 | 1H NMR (300 MHz, CHLOROFORM-D) d ppm 0.52-0.64 (m, 4H), 1.21-1.36 (m, 1H), | 159-160 |
| | 2.26 (s, 3H), 2.29 (s, 3H), 4.20 (d, J = 7.0 Hz, 2H), 7.26-7.35 (m, 1H), | |
| | 7.55-7.61 (m, 1H), 8.17-8.22 (m, 1H), 8.40-8.43 (m, 1H) | |
| 78 | 1H NMR (300 MHz, CHLOROFORM-D) d ppm 0.47-0.62 (m, 4H), 1.21-1.35 (m, 1H), | 130-132 |
| | 2.25 (s, 3H), 2.28 (s, 3H), 4.18 (d, J = 6.8 Hz, 2H), 7.69-7.06 (m, 1H), | |
| | 7.33-7.40 (m, 1H), 7.88-7.99 (m, 2H) | |
| 79 | 1H NMR (300 MHz, CHLOROFORM-D) d ppm 0.53-0.61 (m, 4H), 1.22-1.32 (m, 1H), | 109-111 |
| | 2.26 (s, 3H), 2.29 (s, 3H), 4.18 (d, J = 7.1 Hz, 2H), 7.06-7.16 (m, 1H), | |
| | 7.41-7.50 (m, 1H), 7.94-8.03 (m, 1H) | |
| 80 | 1H NMR (300 MHz, CHLOROFORM-D) d ppm 0.46-0.62 (m, 4H), 1.21-1.32 (m, 1H), | 139-140 |
| | 2.26 (s, 3H), 2.28 (s, 3H), 4.16 (d, J = 7.0 Hz, 2H), 6.97-7.07 (m, 1H), | |
| | 7.32-7.41 (m, 1H), 7.63-7.72 (m, 1H) | |
| 81 | 1H NMR (300 MHz, CHLOROFORM-D) d ppm 0.46-0.61 (m, 4H), 1.21-1.31 (m, 1H), | 57-58 |
| | 2.26 (s, 3H), 2.29 (s, 3H), 4.15 (d, J = 7.0 Hz, 2H), 7.24-7.31 (m, 1H), | |
| | 7.41-7.46 (m, 1H), 7.90-7.97 (m, 1H) | |
| 82 | 1H NMR (200 MHz, CHLOROFORM-D) d ppm 0.47-0.61 (m, 4H) 1.14-1.38 (m, 1H) | 113-115 |
| | 2.26 (s, 3H) 2.29 (s, 3H) 4.15 (d, J = 7.0 Hz, 2H) 7.22-7.37 (m, 2H) | |
| | 7.90-7.94 (m, 1H) | |
| 83 | 1H NMR (300 MHz, CHLOROFORM-D) d ppm 0.52-0.59 (m, 4H), 1.18-1.32 (m, 1H), | 154-155 |
| | 2.26 (s, 3H), 2.29 (s, 3H), 4.17 (d, J = 6.8 Hz, 2H), 7.26-7.35 (m, 2H), | |
| | 7.96-8.06 (m, 1H) | |
| 84 | 1H NMR (200 MHz, CHLOROFORM-D) d ppm 0.48-0.60 (m, 4H), 1.19-1.34 (m, 1H), | 144-146 |
| | 2.25 (s, 3H), 2.28 (s, 3H), 4.16 (d, J = 7.0 Hz, 2H), 6.98-7.11 (m, 1H) | |
| | 7.32-7.41 (m, 1H) 7.94-8.05 (m, 1H) | |
| 85 | 1H NMR (300 MHz, CHLOROFORM-D) d ppm 0.46-0.63 (m, 4H) 1.19-1.33 (m, 1H) | 161-163 |
| | 2.26 (s, 3H) 2.29 (s, 3H) 4.16 (d, J = 7.0 Hz, 2H) 7.21-7.29 (m, 1H) | |
| | 7.85-7.94 (m, 1H) | |
| 86 | 1H NMR (300 MHz, CHLOROFORM-D) d ppm 0.46-0.62 (m, 4H), 1.26 (s, 1H), | 59-60 |
| | 2.26 (s, 3H), 2.29 (s, 3H), 4.15 (d, J = 7.0 Hz, 2H), 7.44-7.51 (m, 1H), | |
| | 7.78-7.85 (m, 1H) | |
| 87 | 1H NMR (200 MHz, CHLOROFORM-D) d ppm 0.44-0.58 (m, 4H), 1.14-1.33 (m, 1H), | |
| | 2.25 (s, 3H), 2.28 (s, 3H), 4.15 (d, J = 7.0 Hz, 2H), 7.28-7.48 (m, 3H), | |
| | 8.06 (dd, J = 7.5, 1.8 Hz, 1H) | |
| 88 | 1H NMR (300 MHz, CHLOROFORM-D) d ppm 0.52-0.60 (m, 4H), 1.20-1.35 (m, 1H), | 124-126 |
| | 2.24 (s, 3H), 2.27 (s, 3H), 3.81 (s, 3H), 3.87 (s, 3H), 4.14 (d, J = 7.0 Hz, | |
| | 2H), 6.88-6.97 (m, 2H), 7.56-7.61 (m, 1H) | |
| 89 | 1H NMR (300 MHz, CHLOROFORM-D) d ppm 0.47-0.62 (m, 4H), 1.20-1.36 (m, 1H), | 82-84 |
| | 2.25 (s, 3H), 2.28 (s, 3H), 2.64-2.67 (m, 3H), 4.16 (d, J = 6.9 Hz, 2H), | |
| | 6.95-7.05 (m, 1H), 7.11-7.20 (m, 1H), 7.77-7.86 (m, 1H) | |
| 90 | 1H NMR (300 MHz, CHLOROFORM-D) d ppm 0.46-0.61 (m, 4H), 1.20-1.34 (m, 1H), | 124-125 |
| | 2.24 (s, 3H), 2.28 (s, 3H), 2.69 (s, 3H), 4.14 (d, J = 6.8 Hz, 2H), | |
| | 7.18-7.23 (m, 2H), 8.03-8.10 (m, 1H) | |
| 91 | 1H NMR (300 MHz, CHLOROFORM-D) d ppm 0.47-0.60 (m, 4H) 1.20-1.34 (m, 1H) | 90-92 |
| | 2.24 (s, 3H) 2.27 (s, 3H) 2.35 (s, 3H) 4.16 (d, J = 7.0 Hz, 2H) | |
| | 7.11-7.17 (m, 1H) 7.45-7.47 (m, 1H) 7.85-7.90 (m, 1H) | |
| 92 | 1H NMR (300 MHz, CHLOROFORM-D) d ppm 0.54-0.61 (m, 4H), 1.20-1.32 (m, 1H), | 122-123 |
| | 2.27 (s, 3H), 2.30 (s, 3H), 4.19 (d, J = 7.1 Hz, 2H), 7.16-7.25 (m, 1H), | |
| | 7.62-7.69 (m, 1H), 8.41-8.48 (m, 1H) | |
| 93 | 1H NMR (300 MHz, CHLOROFORM-D) d ppm 0.42-0.59 (m, 4H), 1.17-1.28 (m, 1H), | 131-133 |
| | 2.26 (s, 3H), 2.29 (s, 3H), 4.12 (d, J = 7.0 Hz, 2H), 7.10-7.19 (m, 1H), | |
| | 7.51-7.59 (m, 1H), 7.66-7.74 (m, 1H) | |
| 94 | 1H NMR (300 MHz, CHLOROFORM-D) d ppm 0.40-0.55 (m, 4H), 1.13-1.24 (m, 1H), | 111-113 |
| | 2.26 (s, 3H), 2.28 (s, 3H), 4.07 (d, J = 7.0 Hz, 2H), 7.23-7.31 (m, 1H), | |
| | 7.35-7.48 (m, 2H) | |
| 95 | 1H NMR (300 MHz, CHLOROFORM-D) d ppm 0.43-0.59 (m, 4H), 1.15-1.30 (m, 1H), | 143-144 |
| | 2.26 (s, 3H), 2.28 (s, 3H), 4.12 (d, J = 7.1 Hz, 2H), 7.20-7.30 (m, 1H), | |
| | 7.37-7.45 (m, 1H), 7.88-7.97 (m, 1H) | |
| 96 | 1H NMR (300 MHz, CHLOROFORM-D) d ppm 0.51-0.61 (m, 4H), 1.20-1.34 (m, 1H), | 107-109 |
| | 2.24 (s, 3H), 2.27 (s, 3H), 3.89 (s, 3H), 4.14 (d, J = 7.0 Hz, 2H), | |
| | 6.86-6.94 (m, 1H), 7.02-7.11 (m, 1H), 7.66-7.74 (m, 1H) | |
| 97 | 1H NMR (300 MHz, CHLOROFORM-D) d ppm 0.52-0.59 (m, 4H), 1.19-1.34 (m, 1H), | 121-123 |
| | 2.24 (s, 3H), 2.27 (s, 3H), 3.89 (s, 3H), 4.14 (d, J = 7.0 Hz, 2H), | |
| | 6.86-6.93 (m, 1H), 7.27-7.35 (m, 1H), 7.91-7.96 (m, 1H) | |
| 98 | 1H NMR (300 MHz, CHLOROFORM-D) d ppm 0.46-0.54 (m, 4H), 0.87 (t, J = 7.5 Hz, | 67-69 |
| | 6H), 1.11-1.22 (m, 1H), 1.46-1.78 (m, 4H), 2.19 (s, 3H), 2.23 (s, 3H), | |
| | 2.25-2.35 (m, 1H), 4.06 (d, J = 7.0 Hz, 2H) | |
| 99 | 1H NMR (300 MHz, CHLOROFORM-D) d ppm 0.48-0.56 (m, 4H) 0.91 (s, 9H) | 42-43 |
| | 0.97 (d, J = 6.4 Hz, 3H) 1.07-1.37 (m, 4H) 2.19 (s, 3H) 2.21-2.55 (m, 2H) | |
| | 2.23 (s, 3H) 4.05 (d, J = 7.2 Hz, 2H) | |
| 100 | 1H NMR (300 MHz, CHLOROFORM-D) d ppm 0.45-0.57 (m, 4H) 1.09-1.29 (m, 4H) | 108-109 |
| | 1.44-1.68 (m, 3H) 1.74-1.89 (m, 2H) 2.19 (s, 3H) 2.23 (s, 3H) | |
| | 2.27-2.44 (m, 1H) 2.45-2.52 (m, 2H) 4.04 (d, J = 7.0 Hz, 2H) | |
| 101 | 1H NMR (300 MHz, CHLOROFORM-D) d ppm 0.51-0.58 (m, 4H), 1.11-1.22 (m, 1H), | 92-94 |
| | 2.29 (s, 3H), 2.32 (s, 3H), 4.13 (d, J = 7.0 Hz, 2H) | |
| 102 | 1H NMR (300 MHz, CHLOROFORM-D) d ppm 0.35-0.51 (m, 4H) 0.90 (t, J = 7.3 Hz, 3H) | 108-110 |
| | 1.01-1.16 (m, 1H) 1.79-2.31 (m, 2H) 2.17 (s, 3H) 2.20 (s, 3H) | |
| | 3.59 (t, J = 7.7 Hz, 1H) 4.00 (d, J = 7.2 Hz, 2H) 7.13-7.20 (m, 1H) | |
| | 7.22-7.30 (m, 2H) 7.33-7.44 (m, 2H) | |
| 103 | 1H NMR (200 MHz, CHLOROFORM-D) d ppm 2.25 (s, 3H) 2.28 (s, 3H) 3.33 (s, 3H) | 64-66 |
| | 3.81 (t, J = 5.3 Hz, 2H) 4.42 (t, J = 5.3 Hz, 2H) 7.54 (t, J = 7.5 Hz, 1H) | |
| | 7.71 (d, J = 7.5 Hz, 1H) 8.46 (d, J = 7.5 Hz, 1H) 8.56 (s, 1H) | |
| 104 | 1H NMR (200 MHz, CHLOROFORM-D) d ppm 2.25 (s, 3H) 2.28 (s, 3H) 3.31 (s, 3H) | 182-183 |
| | 3.79 (t, J = 5.3 Hz, 2H) 4.37 (t, J = 5.3 Hz, 2H) 7.09-7.38 (m, 1H) | |
| | 7.53-7.82 (m, 1H) 8.16-8.39 (m, 1H) | |
| 105 | 1H NMR (200 MHz, CHLOROFORM-D) d ppm 2.01-2.19 (m, 2H), 2.26 (s, 6H), | 104-106 |
| | 3.33 (s, 3H), 3.43 (t, J = 5.7 Hz, 2H), 4.31 (t, J = 5.7 Hz, 2H), | |
| | 7.20-7.33 (m, 1H), 7.60-7.75 (m, 1H), 8.25-8.40 (m, 1H) | |
| 106 | 1H NMR (200 MHz, CHLOROFORM-D) d ppm 1.15 (t, J = 7.0 Hz, 3H) 2.26 (s, 3H) | 138-140 |
| | 2.29 (s, 3H) 3.48 (q, J = 7.0 Hz, 2H) 3.84 (t, J = 5.5 Hz, 2H) 4.42 (t, J = 5.5 Hz, | |
| | 2H) 7.54 (t, J = 7.5 Hz, 1H) 7.71 (d, J = 7.5 Hz, 1H) 8.46 (d, J = 7.5 Hz, 1 | |
| 107 | 1H NMR (200 MHz, CHLOROFORM-D) d ppm 1.13 (t, J = 6.7 Hz, 3H) 2.26 (s, 3H) | 147-148 |
| | 2.29 (s, 3H) 3.45 (q, J = 6.7 Hz, 2H) 3.82 (t, J = 5.3 Hz, 2H) 4.37 (t, J = 5.3 Hz, | |
| | 2H) 7.10-7.37 (m, 1H) 7.51-7.81 (m, 1H) 8.06-8.42 (m, 1H) | |
| 108 | 1H NMR (300 MHz, CHLOROFORM-D) d ppm 1.25 (t, J = 7.6 Hz, 3H), 1.40 (t, J = 7.1 Hz, | 115-117 |
| | 3H), 2.27 (s, 3H), 2.67 (q, J = 7.6, 0.5 Hz, 2H), 4.30 (q, J = 7.1 Hz, 2H), | |
| | 7.22-7.30 (m, 1H), 7.63-7.70 (m, 1H), 8.27-8.35 (m, 1H) | |
| 109 | 1H NMR (300 MHz, CHLOROFORM-D) d ppm 0.50-0.60 (m, 4H), 1.18-1.36 (m, 1H), | 164-166 |
| | 1.27 (t, J = 7.5 Hz, 3H), 2.30 (s, 3H), 2.68 (q, J = 7.5 Hz, 2H), 4.19 (d, | |
| | J = 7.1 Hz, 2H), 7.27 (t, J = 7.0 Hz, 1H), 7.67 (t, J = 7.0 Hz, 1H), 8.29 (t, | |
| 110 | 1H NMR (200 MHz, CHLOROFORM-D) d ppm 1.29 (d, J = 6.6 Hz, 6H) 2.27 (s, 3H) | 89-91 |
| | 3.08-3.28 (m, 1H) 3.78 (s, 3H) 7.21-7.33 (m, 1H) 7.62-7.72 (m, 1H) | |
| | 7.21-7.33 (m, 1H) | |
| 111 | 1H NMR (200 MHz, CHLOROFORM-D) d ppm 1.29 (d, J = 7.0 Hz, 6H) 1.40 (t, J = 7.0 Hz, | 64-66 |
| | 3H) 2.28 (s, 3H) 3.02-3.31 (m, 1H) 4.30 (q, J = 7.0 Hz, 2H) | |
| | 7.20-7.32 (m, 1H) 7.61-7.71 (m, 1H) 8.26-8.36 (m, 1H) | |
| 112 | 1H NMR (200 MHz, CHLOROFORM-D) d ppm 0.52-0.69 (m, 4H) 1.09-1.42 (m, 1H) | 89-91 |
| | 1.30 (d, J = 7.0 Hz, 6H) 2.32 (s, 3H) 3.06-3.31 (m, 1H) 4.22 (d, J = 7.0 Hz, | |
| | 2H) 7.55 (t, J = 7.5 Hz, 1H) 7.70 (d, J = 7.5 Hz, 1H) 8.44 (d, J = 7.5 Hz, 1H) | |
| 113 | 1H NMR (200 MHz, CHLOROFORM-D) d ppm 0.52-0.63 (m, 4H) 1.15-1.38 (m, 1H) | 64-66 |
| | 1.30 (d, J = 7.0 Hz, 6H) 2.32 (s, 3H) 3.09-3.27 (m, 1H) 4.19 (d, J = 7.0 Hz, | |
| | 2H) 7.19-7.34 (m, 1H) 7.58-7.73 (m, 1H) 8.20-8.36 (m, 1H) | |
| 114 | 1H NMR (200 MHz, CHLOROFORM-D) d ppm 1.29 (d, J = 7.0 Hz, 6H) 2.30 (s, 3H) | 132-134 |
| | 3.07-3.26 (m, 1H) 3.32 (s, 3H) 3.79 (t, J = 5.3 Hz, 2H) 4.37 (t, J = 5.3 Hz, | |
| | 2H) 7.20-7.32 (m, 1H) 7.61-7.72 (m, 1H) 8.23-8.34 (m, 1H) | |
| 115 | 1H NMR (200 MHz, CHLOROFORM-D) d ppm 1.12 (dd, J = 7.0 Hz, 3H) 1.29 (d, J = 6.6 Hz, | 96-97 |
| | 6H) 2.31 (s, 3H) 3.09-3.26 (m, 1H) 3.45 (q, J = 7.0 Hz, 2H) 3.82 (t, | |
| | J = 5.3 Hz, 2H) 4.38 (t, J = 5.3 Hz, 2H) 7.20-7.32 (m, 1H) 7.62-7.72 (m, 1H) | |
| | 8.23-8.34 (m, 1H) | |
| 116 | 1H NMR (200 MHz, CHLOROFORM-D) d ppm 1.42 (s, 9H), 2.38 (s, 3H), 2.71 (s, 3H), | 173-175 |
| | 3.75 (s, 3H), 7.17-7.35 (m, 3H), 8.08-8.19 (m, 1H) | |
| 117 | 1H NMR (200 MHz, CHLOROFORM-D) d ppm 1.43 (s, 9H) 2.39 (s, 3H) 3.71 (s, 3H) | 74-76 |
| | 7.40-7.62 (m, 2H) 7.67-7.75 (m, 1H) 7.84-7.92 (m, 1H) | |
| 118 | 1H NMR (200 MHz, CHLOROFORM-D) d ppm 1.43 (s, 9H), 2.40 (s, 3H), 3.81 (s, 3H), | 140-142 |
| | 7.43-7.80 (m, 2H), 8.38-8.68 (m, 2H) | |
| 119 | 1H NMR (200 MHz, CHLOROFORM-D) d ppm 1.43 (s, 9H) 2.39 (s, 3H) 3.76 (s, 3H) | 119-121 |
| | 7.20-7.36 (m, 2H) 7.37-7.48 (m, 1H) 7.91-8.04 (m, 1H) | |
| 120 | 1H NMR (200 MHz, CHLOROFORM-D) d ppm 1.43 (s, 9H) 2.39 (s, 3H) 3.76 (s, 3H) | 182-184 |
| | 7.15-7.39 (m, 1H) 7.58-7.67 (m, 1H) 7.90-7.99 (m, 1H) | |
| 121 | 1H NMR (200 MHz, CHLOROFORM-D) d ppm 1.42 (s, 9H), 2.39 (s, 3H), 3.79 (s, 3H), | 173-175 |
| | 7.20-7.43 (m, 1H), 7.54-7.63 (m, 1H), 8.17-8.29 (m, 1H), | |
| | 8.42-8.52 (m, 1H) | |
| 122 | 1H NMR (200 MHz, CHLOROFORM-D) d ppm 1.43 (s, 9H) 2.39 (s, 3H) 3.79 (s, 3H) | 83-85 |
| | 6.98-7.10 (m, 1H) 7.31-7.43 (m, 1H) 7.91-7.99 (m, 2H) | |
| 123 | 1H NMR (200 MHz, CHLOROFORM-D) d ppm 1.43 (s, 9H), 2.39 (s, 3H), 3.74 (s, 3H), | |
| | 7.22-7.51 (m, 3H), 8.06-8.17 (m, 1H) | |
| 124 | 1H NMR (200 MHz, CHLOROFORM-D) d ppm 1.42 (s, 9H), 2.38 (s, 3H), 3.00 (s, 6H), | 189-190 |
| | 3.79 (s, 3H), 6.81-6.91 (m, 1H), 7.24-7.35 (m, 1H), 7.70-7.80 (m, | |
| 125 | 1H NMR (200 MHz, CHLOROFORM-D) d ppm 1.44 (s, 9H), 2.42 (s, 3H), 3.82 (s, 3H), | 131-133 |
| | 7.47-7.59 (m, 1H), 7.67-7.78 (m, 1H), 8.46-8.55 (m, 1H), | |
| | 8.62-8.68 (m, 1H) | |
| 126 | 1H NMR (200 MHz, CHLOROFORM-D) d ppm 1.43 (s, 9H) 2.40 (s, 3H) 3.83 (s, 3H) | 192-193 |
| | 3.94 (s, 3H) 7.50 (t, J = 7.9 Hz, 1H) 8.07-8.21 (m, 1H) 8.44-8.58 (m, 1H) | |
| | 8.99 (t, J = 1.5 Hz, 1H) | |
| 127 | 1H NMR (200 MHz, CHLOROFORM-D) d ppm 1.44 (s, 9H) 2.41 (s, 3H) 3.84 (s, 3H) | 250-252 |
| | 7.55 (t, J = 7.9 Hz, 1H) 8.20 (d, J = 7.9 Hz, 1H) 8.55 (d, J = 7.9 Hz, 1H) 9.07 | |
| 128 | 1H NMR (200 MHz, CHLOROFORM-D) d ppm 1.42 (s, 9H), 2.40 (s, 3H), 3.77 (s, 3H), | 176-178 |
| | 7.04-7.28 (m, 2H), 7.88 (t, J = 6.8 Hz, 1H) | |
| 129 | 1H NMR (200 MHz, CHLOROFORM-D) d ppm 1.43 (s, 9H) 2.40 (s, 3H) 3.78 (s, 3H) | 203-205 |
| | 7.05-7.17 (m, 1H) 7.39-7.52 (m, 1H) 7.95-8.07 (m, 1H) | |
| 130 | 1H NMR (200 MHz, CHLOROFORM-D) d ppm 1.43 (s, 9H) 2.40 (s, 3H) 3.76 (s, 3H) | 125-126 |
| | 6.95-7.09 (m, 1H) 7.31-7.43 (m, 1H) 7.68-7.78 (m, 1H) | |
| 131 | 1H NMR (200 MHz, CHLOROFORM-D) d ppm 1.43 (s, 9H) 2.40 (s, 3H) 3.76 (s, 3H) | 161-162 |
| | 6.88-7.02 (m, 1H) 7.52-7.63 (m, 1H) 7.65-7.75 (m, 1H) | |
| 132 | 1H NMR (200 MHz, CHLOROFORM-D) d ppm 1.42 (s, 9H) 2.40 (s, 3H) 3.77 (s, 3H) | 144-145 |
| | 6.93-7.06 (m, 1H) 7.43-7.54 (m, 1H) 8.23-8.32 (m, 1H) | |
| 133 | 1H NMR (200 MHz, CHLOROFORM-D) d ppm 1.43 (s, 9H) 2.40 (s, 3H) 3.76 (s, 3H) | 171-173 |
| | 7.21-7.39 (m, 2H) 7.96-8.01 (m, 1H) | |
| 134 | 1H NMR (200 MHz, CHLOROFORM-d) d ppm 1.43 (s, 9H) 2.39 (s, 3H) 3.72 (s, 3H) | 143-145 |
| | 6.72-6.94 (m, 1H) 6.98-7.21 (m, 1H) | |
| 135 | 1H NMR (200 MHz, CHLOROFORM-D) d ppm 1.43 (s, 9H) 2.40 (s, 3H) 3.76 (s, 3H) | 145-146 |
| | 7.19-7.31 (m, 1H) 7.88-8.02 (m, 1H) | |
| 136 | 1H NMR (200 MHz, CHLOROFORM-D) d ppm 1.43 (s, 9H) 2.40 (s, 3H) 3.76 (s, 3H) | 205-207 |
| | 7.44-7.51 (m, 1H), 7.83-7.92 (m, 1H) | |
| 137 | 1H NMR (200 MHz, CHLOROFORM-D) d ppm 1.44 (s, 9H), 2.45 (s, 3H), 3.46 (s, 3H), | 138-140 |
| | 3.80 (s, 3H), 3.85 (s, 3H), 6.46-6.60 (m, 2H), 7.30 (s, 1H) | |
| 138 | 1H NMR (200 MHz, CHLOROFORM-D) d ppm 1.43 (s, 9H) 2.40 (s, 3H) 2.66 (s, 3H) | 145-146 |
| | 3.76 (s, 3H) 6.92-7.06 (m, 1H) 7.10-7.21 (m, 1H) 7.81-7.92 (m, 1H) | |
| 139 | 1H NMR (200 MHz, CHLOROFORM-D) d ppm 1.43 (s, 9H) 2.39 (s, 3H) 2.66 (s, 3H) | 161-162 |
| | 3.73 (s, 3H) 7.09-7.20 (m, 1H) 7.35-7.44 (m, 1H) 7.77-7.85 (m, 1H) | |
| 140 | 1H NMR (200 MHz, CHLOROFORM-D) d ppm 1.43 (s, 9H) 2.39 (s, 3H) 2.68 (s, 3H) | 143-144 |
| | 3.73 (s, 3H) 7.01-7.12 (m, 1H) 7.54-7.62 (m, 1H) 7.79-7.87 (m, 1H) | |
| 141 | 1H NMR (200 MHz, CHLOROFORM-D) d ppm 1.43 (s, 9H), 2.40 (s, 3H), 3.78 (s, 3H), | 176-178 |
| | 7.21-7.32 (m, 1H), 7.66 (t, J = 6.4 Hz, 1H), 8.31 (t, J = 6.6 Hz, 1H) | |
| 142 | 1H NMR (200 MHz, CHLOROFORM-D) d ppm 1.43 (s, 9H), 2.41 (s, 3H), 3.78 (s, 3H), | 140-142 |
| | 7.15-7.29 (m, 1H), 7.60-7.70 (m, 1H), 8.46 (dd, J = 6.6, 1.8 Hz 1H) | |
| 143 | 1H NMR (200 MHz CHLOROFORM-D) d ppm 1.43 (s, 9H) 2.40 (s, 3H) 3.72 (s, 3H) | 83-85 |
| | 7.05-7.23 (m, 1H) 7.55-7.65 (m, 1H) 7.65-7.76 (m, 1H) | |
| 144 | 1H NMR (200 MHz, CHLOROFORM-D) d ppm 1.43 (s, 9H) 2.24 (s, 3H) 2.38 (s, 3H) | 140-142 |
| | 3.71 (s, 3H) 6.73-6.86 (m, 1H) 6.98-7.20 (m, 1H) | |
| 145 | 1H NMR (200 MHz, CHLOROFORM-D) d ppm 1.44 (s, 9H) 2.39 (s, 3H) 3.67 (s, 3H) | 129-130 |
| | 7.31-7.64 (m, 2H) | |
| 146 | 1H NMR (200 MHz, CHLOROFORM-D) d ppm 1.41 (s, 9H) 2.38 (s, 3H) 3.74 (s, 3H) | 177-179 |
| | 3.90 (s, 3H) 6.85-6.94 (m, 1H) 7.26-7.36 (m, 1H) 7.96-8.02 (m, 1H) | |
| 147 | 1H NMR (200 MHz, CHLOROFORM-D) d ppm 1.41 (s, 9H) 2.38 (s, 3H) 3.74 (s, 3H) | 117-119 |
| | 3.91 (s, 3H) 6.89-7.02 (m, 2H) 7.96-8.05 (m, 1H) | |
| 148 | 1H NMR (200 MHz, CHLOROFORM-D) d ppm 1.46 (s, 9H), 2.46 (s, 3H), 3.85 (s, 3H), | 202-203 |
| | 7.92-8.03 (m, 1H), 8.75 (d, J = 5.7 Hz, 1H), 9.18 (d, J = 7.9 Hz, 1H). | |
| 149 | 1H NMR (200 MHz, CHLOROFORM-D) d ppm 1.42 (s, 9H) 2.41 (s, 3H) 3.74 (s, 3H) | 180-182 |
| 150 | 1H NMR (200 MHz, CHLOROFORM-D) d ppm 1.28 (t, J = 7.7 Hz, 3H), 1.37 (t, J = 7.3 Hz, | |
| | 3H), 1.43 (s, 9H), 2.40 (s, 3H), 3.14 (q, J = 7.7 Hz, 2H), 4.28 (q, | |
| | J = 7.3 Hz, 2H) 7.17-7.38 (m, 3H), 7.98-8.08 (m, 1H) | |
| 151 | 1H NMR (200 MHz, CHLOROFORM-D) d ppm 1.34 (t, J = 7.0 Hz, 3H) 1.43 (s, 9H) | 74-76 |
| | 2.40 (s, 3H) 4.26 (q, J = 7.0 Hz, 2H) 7.40-7.62 (m, 2H) 7.67-7.75 (m, 1H) | |
| | 7.84-7.92 (m, 1H) | |
| 152 | 1H NMR (200 MHz, CHLOROFORM-D) d ppm 1.36-1.52 (m, 12H), 2.42 (s, 3H), | 158-160 |
| | 4.35 (q, J = 7.0 Hz, 2H), 7.38-7.85 (m, 2H), 8.32-8.70 (m, 2H) | |
| 153 | 1H NMR (200 MHz, CHLOROFORM-D) d ppm 1.35 (t, J = 7.3 Hz, 3H) 1.42 (s, 9H) | 104-105 |
| | 2.40 (s, 3H) 4.30 (q, J = 7.3 Hz, 2H) 7.23-7.48 (m, 3H) 8.06-8.15 (m, 1 | |
| 154 | 1H NMR (200 MHz, CHLOROFORM-D) d ppm 1.39 (t, J = 7.0 Hz, 3H) 1.42 (s, 9H) | 105-106 |
| | 2.41 (s, 3H) 4.31 (q, J = 7.0 Hz, 2H) 6.92-7.06 (m, 1H) 7.42-7.54 (m, 1H) | |
| | 8.20-8.29 (m, 1H) | |
| 155 | 1H NMR (200 MHz, CHLOROFORM-D) d ppm 1.38 (t, J = 7.2 Hz, 3H) 1.43 (s, 9H) | 115-116 |
| | 2.41 (s, 3H) 4.30 (q, J = 7.2 Hz, 2H) 6.92-7.02 (m, 1H) 7.52-7.63 (m, 1H) | |
| | 7.63-7.74 (m, 1H) | |
| 156 | 1H NMR (200 MHz, CHLOROFORM-D) d ppm 1.38 (t, J = 7.0 Hz, 3H) 1.43 (s, 9H) | 99-100 |
| | 2.41 (s, 3H) 4.30 (q, J = 7.0 Hz, 2H) 6.98-7.11 (m, 1H) 7.32-7.42 (m, 1H) | |
| | 7.97-8.09 (m, 1H) | |
| 157 | 1H NMR (200 MHz, CHLOROFORM-D) d ppm 1.39 (t, J = 7.2 Hz, 3H) 1.42 (s, 9H) | 87-89 |
| | 2.41 (s, 3H) 4.30 (q, J = 7.2 Hz, 2H) 6.80-6.94 (m, 1H) 7.60-7.72 (m, 1H) | |
| | 8.38-8.47 (m, 1H) | |
| 158 | 1H NMR (300 MHz, CHLOROFORM-D) d ppm 1.38 (t, J = 7.2 Hz, 3H) 1.43 (s, 9H) | 127-128 |
| | 2.42 (s, 3H) 4.30 (q, J = 7.2 Hz, 2H) 7.23-7.31 (m, 1H) 7.31-7.38 (m, 1H) | |
| | 7.94-7.99 (m, 1H) | |
| 159 | 1H NMR (200 MHz, CHLOROFORM-D) d ppm 1.38 (t, J = 7.2 Hz, 3H) 1.43 (s, 9H) | 107-108 |
| | 2.42 (s, 3H) 4.29 (q, J = 7.2 Hz, 2H) 7.16-7.33 (m, 1H), 7.86-8.00 (m, 1 | |
| 160 | 1H NMR (300 MHz, CHLOROFORM-D) d ppm 1.38 (t, J = 7.1 Hz, 3H), 1.43 (s, 9H), | 112-114 |
| | 2.42 (s, 3H), 4.29 (q, J = 7.1 Hz, 2H), 7.45-7.50 (m, 1H), 7.83-7.89 (m, | |
| 161 | 1H NMR (200 MHz, CHLOROFORM-D) d ppm 1.39 (t, J = 7.0 Hz, 3H) 1.43 (s, 9H) | 108-109 |
| | 2.41 (s, 3H) 2.67 (s, 3H) 4.30 (q, J = 7.0 Hz, 2H) 6.92-7.06 (m, 1H) | |
| | 7.10-7.21 (m, 1H) 7.80-7.91 (m, 1H) | |
| 162 | 1H NMR (200 MHz, CHLOROFORM-D) d ppm 1.38 (t, J = 7.0 Hz, 3H), 1.42 (s, 9H), | 125-126 |
| | 2.40 (s, 3H), 2.70 (s, 3H), 4.29 (q, J = 7.0 Hz, 2H), 7.15-7.24 (m, 2H), | |
| | 8.06-8.13 (m, 1H) | |
| 163 | 1H NMR (200 MHz, CHLOROFORM-D) d ppm 1.37 (t, J = 7.3 Hz, 3H), 1.42 (s, 9H), | 100-102 |
| | 2.34 (s, 3H), 2.40 (s, 3H), 4.30 (q, J = 7.3 Hz, 2H), 7.09-7.17 (m, 1H), | |
| | 7.44-7.49 (m, 1H), 7.86-7.93 (m, 1H) | |
| 164 | 1H NMR (200 MHz, CHLOROFORM-D) d ppm 1.34 (t, J = 7.2 Hz, 3H) 1.43 (s, 9H) | 103-104 |
| | 2.41 (s, 3H) 4.27 (q, J = 7.2 Hz, 2H) 7.07-7.21 (m, 1H) 7.54-7.64 (m, 1H) | |
| | 7.65-7.76 (m, 1H) | |
| 165 | 1H NMR (200 MHz, CHLOROFORM-D) d ppm 1.39 (t, J = 7.0 Hz, 3H) 1.43 (s, 9H) | 97-99 |
| | 2.42 (s, 3H) 4.32 (q, J = 7.0 Hz, 2H) 7.20-7.32 (m, 1H) 7.61-7.72 (m, 1H) | |
| | 8.25-8.36 (m, 1H) | |
| 166 | 1H NMR (200 MHz, CHLOROFORM-D) d ppm 1.39 (t, J = 7.2 Hz, 3H) 1.42 (s, 9H) | 113-115 |
| | 2.41 (s, 3H) 4.30 (q, J = 7.2 Hz, 2H) 6.80-6.94 (m, 1H) 7.60-7.72 (m, 1H) | |
| | 8.38-8.47 (m, 1H) | |
| 167 | 1H NMR (200 MHz, CHLOROFORM-D) d ppm 1.37 (t, J = 7.3 Hz, 3H) 1.41 (s, 9H) | 108-109 |
| | 2.40 (s, 3H) 3.90 (s, 3H) 4.28 (q, J = 7.3 Hz, 2H) 6.85-6.94 (m, 1H) | |
| | 7.26-7.36 (m, 1H) 7.94-7.99 (m, 1H) | |
| 168 | 1H NMR (200 MHz, CHLOROFORM-D) d ppm 1.37 (t, J = 7.0 Hz, 3H), 1.41 (s, 9H), | |
| | 2.39 (s, 3H), 3.91 (s, 3H), 4.27 (q, J = 7.0 Hz, 2H) 6.91-6.99 (m, 2H), | |
| | 7.96-8.03 (m, 1H) | |
| 169 | 1H NMR (200 MHz, CHLOROFORM-D) d ppm 1.42 (t, J = 7.2 Hz, 3H) 1.43 (s, 9H) | |
| | 2.42 (s, 3H) 4.35 (q, J = 7.2 Hz, 2H) 7.37 (dd, J = 7.9, 5.3 Hz, 1H) 8.52 (d, | |
| | J = 7.9 Hz, 1H) 8.67 (d, J = 5.3 Hz, 1H) 9.53 (s, 1H) | |
| 170 | 1H NMR (300 MHz, CHLOROFORM-D) d ppm 1.36 (t, J = 7.2 Hz, 3H) 1.44 (s, 9H) | 110-112 |
| | 2.42 (s, 3H) 4.28 (q, J = 7.2 Hz, 2H) 7.59 (d, J = 5.3 Hz, 1H) 8.78 (d, J = 5.3 Hz, | |
| | 1H) 9.25 (s, 1H) | |
| 171 | 1H NMR (300 MHz, CHLOROFORM-D) d ppm 1.14-1.35 (m, 2H) 1.30 (t, J = 7.1 Hz, 3H) | 149-150 |
| | 1.38 (s, 9H) 1.47-1.69 (m, 4H) 1.77-1.90 (m, 2H) 2.31-2.45 (m, 1H) | |
| | 2.35 (s, 3H) 2.48-2.53 (m, 2H) 4.18 (q, J = 7.1 Hz, 2H) | |
| 172 | 1H NMR (300 MHz, CHLOROFORM-D) d ppm 1.34 (t, J = 7.1 Hz, 3H), 1.42 (s, 9H), | 113-115 |
| | 2.42 (s, 3H) 4.27 (q, J = 7.1 Hz, 2H) | |
| 173 | 1H NMR (200 MHz, CHLOROFORM-d) d ppm 1.32-1.46 (m, 12H) 2.42 (s, 3H) | 63-65 |
| | 2.76 (s, 3H) 4.30 (q, J = 7.3 Hz, 2H) 7.26-7.35 (m, 1H) 7.47-7.57 (m, 1H) | |
| | 8.37-8.43 (m, 1H) | |
| 174 | 1H NMR (200 MHz, CHLOROFORM-d) d ppm 1.37 (t, J = 7.3 Hz, 3H) 1.43 (s, 9H) | 95-97 |
| | 2.41 (s, 3H) 4.29 (q, J = 7.3 Hz, 2H) 6.33-7.16 (m, 2H) 7.46-7.56 (m, 1H) | |
| | 8.17-8.21 (m, 1H) | |
| 175 | 1H NMR (200 MHz, CHLOROFORM-D) d ppm 1.06 (t, J = 7.5 Hz, 3H), 1.43 (s, 9H), | 172-174 |
| | 1.73-1.96 (m, 2H), 2.41 (s, 3H) 4.24 (t, J = 7.9 Hz, 2H), 7.54 (t, J = 7.7 Hz, | |
| | 1H), 7.65-7.77 (d, J = 7.7 Hz, 1H), 8.46 (d, J = 7.7 Hz, 1H), 8.60 (s, 1 | |
| 176 | 1H NMR (200 MHz, CHLOROFORM-D) d ppm 1.43 (s, 9H), 1.78 (d, J = 6.6 Hz, 6H), | 135-137 |
| | 2.41 (s, 3H), 4.48-4.87 (m, 1H), 7.55 (t, J = 7.7 Hz, 1H), 7.71 (d, J = 7.7 Hz, | |
| | 1H), 8.46 (d, J = 7.7 Hz, 1H), 8.57 (s, 1H) | |
| 177 | 1H NMR (200 MHz, CHLOROFORM-D) d ppm 1.00 (d, J = 7.0 Hz, 6H) 1.44 (s, 9H) | |
| | 2.23-2.50 (m, 1H) 2.39 (s, 3H) 4.11 (d, J = 7.5 Hz, 2H) 7.37 (dd, J = 7.9, | |
| | 5.3 Hz, 1H) 8.51 (d, J = 7.9 Hz, 1H) 8.67 (d, J = 5.3 Hz, 1H) 9.51 (s, 1H) | |
| 178 | 1H NMR (200 MHz, CHLOROFORM-D) d ppm 0.52-0.67 (m, 4H), 1.17-1.36 (m, 1H), | 106-107 |
| | 1.44 (s, 9H), 2.44 (s, 3H), 4.24 (d, J = 7.0 Hz, 2H), 7.35-7.49 (m, 3H), | |
| | 8.23-8.35 (m, 2H) | |
| 179 | 1H NMR (300 MHz, CHLOROFORM-D) d ppm 0.56-0.63 (m, 4H), 1.22-1.29 (m, 1H), | 132-134 |
| | 1.43 (s, 9H), 2.42 (s, 3H), 2.44 (s, 3H), 4.24 (d, J = 6.8 Hz, 2H), | |
| | 7.27-7.35 (m, 2H), 8.06-8.13 (m, 2H) | |
| 180 | 1H NMR (300 MHz, CHLOROFORM-D) d ppm 0.48-0.62 (m, 4H), 1.19-1.31 (m, 1H), | |
| | 1.44 (s, 9H), 2.43 (s, 3H), 2.71 (s, 3H), 4.19 (d, J = 6.8 Hz, 2H), | |
| | 7.18-7.34 (m, 3H), 8.05-8.13 (m, 1H) | |
| 181 | 1H NMR (200 MHz, CHLOROFORM-D) d ppm 0.46-0.62 (m, 4H) 1.10-1.36 (m, 1H) | 120-122 |
| | 1.27 (t, J = 7.5 Hz, 3H) 1.44 (s, 9H) 2.43 (s, 3H) 3.12 (q, J = 7.5 Hz, 2H) | |
| | 4.17 (d, J = 6.6 Hz, 2H) 7.14-7.39 (m, 3H) 7.93-8.01 (m, 1H) | |
| 182 | 1H NMR (200 MHz, CHLOROFORM-D) d ppm 0.25-0.69 (m, 4H) 1.06-1.34 (m, 1H) | 168-169 |
| | 1.27 (d, J = 7.0 Hz, 6H) 1.43 (s, 9H) 2.42 (s, 3H) 3.86-4.09 (m, 1H) | |
| | 4.15 (d, J = 7.0 Hz, 2H) 7.08-7.49 (m, 3H) 7.69-7.90 (m, 1H) | |
| 183 | 1H NMR (200 MHz, CHLOROFORM-D) d ppm 0.39-0.62 (m, 4H) 1.07-1.31 (m, 1H) | 90-91 |
| | 1.43 (s, 9H) 2.42 (s, 3H) 4.13 (d, J = 7.0 Hz, 2H) 7.39-7.62 (m, 2H) | |
| | 7.68 (d, J = 7.0 Hz, 1H) 7.82 (d, J = 7.0 Hz, 1H) | |
| 184 | 1H NMR (200 MHz, CHLOROFORM-D) d ppm 0.54-0.64 (m, 4H) 1.16-1.35 (m, 1H) | 93-94 |
| | 1.45 (s, 9H) 2.46 (s, 3H) 4.25 (d, J = 7.0 Hz, 2H) 7.54 (t, J = 7.7 Hz, 1H) | |
| | 7.70 (d, J = 7.7 Hz, 1H) 8.44 (d, J = 7.7 Hz, 1H) 8.56 (s, 1H) | |
| 185 | 1H NMR (300 MHz, CHLOROFORM-D) d ppm 0.53-0.62 (m, 4H), 1.18-1.29 (m, 1H), | 182-183 |
| | 1.43 (s, 9H), 2.44 (s, 3H), 4.21 (d, J = 7.0 Hz, 2H), 7.04-7.22 (m, 2H), | |
| | 7.35-7.43 (m, 1H), 8.06-8.16 (m, J = 7.8, 1H) | |
| 186 | 1H NMR (300 MHz, CHLOROFORM-D) d ppm 0.51-0.59 (m, 4H), 1.17-1.30 (m, 1H), | |
| | 1.44 (s, 9H), 2.44 (s, 3H), 4.19 (d, J = 6.8 Hz, 2H), 7.26-7.31 (m, 2H), | |
| | 7.38-7.45 (m, 1H), 7.92-7.96 (m, 1H) | |
| 187 | 1H NMR (200 MHz, CHLOROFORM-D) d ppm 0.55-0.63 (m, 4H), 1.18-1.34 (m, 1H), | |
| | 1.44 (s, 9H), 2.45 (s, 3H), 4.23 (d, J = 7.0 Hz, 2H), 7.30-7.46 (m, 2H), | |
| | 8.10-8.27 (m, 2H) | |
| 188 | 1H NMR (300 MHz, CHLOROFORM-D) d ppm 0.50-0.63 (m, 4H) 1.16-1.35 (m, 1H) | 94-95 |
| | 1.44 (s, 9H) 2.44 (s, 3H) 4.22 (d, J = 7.0 Hz, 2H) 7.39 (d, J = 8.9 Hz, 2H) | |
| | 8.21 (d, J = 8.9 Hz, 2H) | |
| 189 | 1H NMR (300 MHz, CHLOROFORM-D) d ppm 0.51-0.58 (m, 4H), 1.16-1.28 (m, 1H), | |
| | 1.44 (s, 9H), 2.44 (s, 3H), 4.19 (d, J = 7.0 Hz, 2H), 7.21 (t, J = 7.3 Hz, | |
| | 1H), 7.33 (t, J = 7.3 Hz, 1H), 7.62 (d, J = 7.3 Hz, 1H), 7.89 (d, J = 7.3 Hz, 1 | |
| 190 | 1H NMR (200 MHz, CHLOROFORM-D) d ppm 0.50-0.63 (m, 4H), 1.17-1.34 (m, 1H), | |
| | 1.44 (s, 9H), 2.44 (s, 3H), 4.19 (d, J = 7.0 Hz, 2H), 7.14-7.38 (m, 2H), | |
| | 7.58-7.66 (m, 1H), 7.85-7.93 (m, 1H) | |
| 191 | 1H NMR (300 MHz, CHLOROFORM-D) d ppm 0.48-0.61 (m, 4H), 1.15-1.33 (m, 1H), | |
| | 1.44 (s, 9H), 2.44 (s, 3H), 4.21 (d, J = 7.0 Hz, 2H), 7.01-7.08 (m, 1H), | |
| | 7.24-7.42 (m, 1H), 7.87-7.99 (m, 2H) | |
| 192 | 1H NMR (200 MHz, CHLOROFORM-D) d ppm 0.53-0.65 (m, 4H) 1.15-1.36 (m, 1H) | 114-115 |
| | 1.44 (s, 9H) 2.45 (s, 3H) 4.23 (d, J = 7.0 Hz, 2H) 7.16 (t, J = 7.9 Hz, 1H) | |
| | 7.73-7.81 (m, 1H) 8.18-8.26 (m, 1H) 8.62 (t, J = 1.5 Hz, 1H) | |
| 193 | 1H NMR (300 MHz, CHLOROFORM-D) d ppm 0.54-0.62 (m, 4H), 1.20-1.32 (m, 1H), | |
| | 1.43 (s, 9H), 2.43 (s, 3H), 3.86 (s, 3H), 4.21 (d, J = 6.8 Hz, 2H), | |
| | 6.93 (d, J = 9.0 Hz, 2H), 8.24 (d, J = 9.0 Hz, 2H) | |
| 194 | 1H NMR (300 MHz, CHLOROFORM-D) d ppm 0.53-0.67 (m, 4H), 1.17-1.34 (m, 1H), | 108-109 |
| | 1.44 (s, 9H), 2.44 (s, 3H), 3.88 (s, 3H), 4.23 (d, J = 7.0 Hz, 2H), | |
| | 6.99-7.05 (m, 1H), 7.30-7.37 (m, 1H), 7.84-7.92 (m, 2H) | |
| 195 | 1H NMR (300 MHz, CHLOROFORM-D) d ppm 0.53-0.61 (m, 4H), 1.17-1.30 (m, 1H), | |
| | 1.42 (s, 9H), 2.42 (s, 3H), 3.91 (s, 3H), 4.17 (d, J = 7.0 Hz, 2H), | |
| | 6.93-7.01 (m, 2H), 7.33-7.42 (m, 1H), 7.96 (dd, J = 7.8, 1.8 Hz, 1H) | |
| 196 | 1H NMR (300 MHz, CHLOROFORM-D) d ppm 0.55-0.63 (m, 4H), 1.17-1.31 (m, 1H), | 106-108 |
| | 1.44 (s, 9H), 2.45 (s, 3H), 4.23 (d, J = 7.0 Hz, 2H), 7.24-7.32 (m, 1H), | |
| | 7.45 (t, J = 7.9 Hz, 1H), 8.12-8.22 (m, 2H) | |
| 197 | 1H NMR (200 MHz, CHLOROFORM-D) d ppm 0.48-0.59 (m, 4H), 1.11-1.33 (m, 1H), | |
| | 1.44 (s, 9H), 2.44 (s, 3H), 4.18 (d, J = 6.6 Hz, 2H), 7.28-7.49 (m, 3H), | |
| | 8.01-8.10 (m, 1H) | |
| 198 | 1H NMR (300 MHz, CHLOROFORM-D) d ppm 0.56-0.63 (m, 4H), 1.20-1.30 (m, 1H), | 109-111 |
| | 1.44 (s, 9H), 2.43-2.48 (m, 3H), 4.24 (d, J = 7.0 Hz, 2H), 5.94 (tt, | |
| | J = 53.2, 3.0 Hz, 1H), 7.26-7.32 (m, 1H), 7.39-7.48 (m, 1H), 8.13-8.21 | |
| 199 | 1H NMR (200 MHz, CHLOROFORM-D) d ppm 0.36-0.74 (m, 4H) 1.18-1.38 (m, 1H) | 178-180 |
| | 1.45 (s, 9H) 2.45 (s, 3H) 4.14 (d, J = 6.6 Hz, 2H) 6.66-7.05 (m, 2H) | |
| | 7.24-7.51 (m, 1H) 8.10-8.19 (m, 1H) 13.16 (s, 1H) | |
| 200 | 1H NMR (300 MHz, CHLOROFORM-D) d ppm 0.52-0.63 (m, 4H) 1.20-1.35 (m, 1H) | 158-159 |
| | 1.43 (s, 9H) 2.42 (s, 3H) 2.93 (s, 3H) 4.18 (d, J = 6.8 Hz, 2H) | |
| | 6.57-6.69 (m, 2H) 7.28-7.36 (m, 1H) 8.34 (dd, J = 7.9, 1.7 Hz, 1H) 8.53-8.70 (m, 1 | |
| 201 | 1H NMR (200 MHz, CHLOROFORM-D) d ppm 0.55-0.70 (m, 4H), 1.17-1.35 (m, 1H), | 128-130 |
| | 1.46 (s, 9H), 2.48 (s, 3H), 3.22 (s, 6H), 4.36 (d, J = 7.0 Hz, 2H), | |
| | 7.57 (t, J = 7.9 Hz, 1H), 8.13 (d, J = 7.9 Hz, 1H), 8.35 (d, J = 7.9 Hz, 1H), 8.54 (s, | |
| 202 | 1H NMR (300 MHz, CHLOROFORM-D) d ppm 0.51-0.67 (m, 4H) 1.14-1.31 (m, 1H) | 89-90 |
| | 1.19 (t, J = 7.1 Hz, 6H) 1.43 (s, 9H) 2.43 (s, 3H) 3.41 (q, J = 7.1 Hz, 4H) | |
| | 4.22 (d, J = 7.0 Hz, 2H) 6.79 (dd, J = 8.2, 2.8 Hz, 1H) 7.26 (t, J = 7.6 Hz, 1H) | |
| | 7.59 (d, J = 7.6 Hz, 1H) 7.67-7.73 (m, 1H) | |
| 203 | 1H NMR (200 MHz, CHLOROFORM-D) d ppm 0.51-0.64 (m, 4H), 1.20 (t, J = 7.0 Hz, | 134-136 |
| | 6H), 1.14-1.34 (m, 1H), 1.42 (s, 9H), 2.41 (s, 3H), 3.41 (q, J = 7.0 Hz, 4H), | |
| | 4.19 (d, J = 7.0 Hz, 2H), 6.66 (d, J = 9.2 Hz, 2H), 8.15 (d, J = 9.2 Hz, 2H) | |
| 204 | 1H NMR (200 MHz, CHLOROFORM-D) d ppm 0.51-0.67 (m, 4H) 1.19-1.36 (m, 1H) | 118-120 |
| | 1.43 (s, 9H) 1.96-2.09 (m, 4H) 2.43 (s, 3H) 3.29-3.41 (m, 4H) 4.23 (d, | |
| | J = 7.0 Hz, 2H) 6.67 (dd, J = 8.1, 2.4 Hz, 1H) 7.27 (t, J = 8.1 Hz, 1H) | |
| | 7.52-7.57 (m, 1H) 7.61 (d, J = 7.5 Hz, 1H) | |
| 205 | 1H NMR (300 MHz, CHLOROFORM-D) d ppm 0.53-0.67 (m, 4H) 1.19-1.36 (m, 1H) | 89-90 |
| | 1.44 (s, 9H) 1.50-1.80 (m, 5H) 2.45 (s, 3H) 3.20-3.29 (m, 4H) 4.24 (d, | |
| | J = 7.0 Hz, 2H) 7.07 (dd, J = 8.2, 2.4 Hz, 1H) 7.31 (t, J = 8.2 Hz, 1H) 7.77 (d, | |
| | J = 8.2 Hz, 1H) 7.90-7.96 (m, 1H) | |
| 206 | 1H NMR (300 MHz, CHLOROFORM-D) d ppm 0.48-0.60 (m, 4H) 1.15-1.33 (m, 1H) | |
| | 1.44 (s, 9H) 2.43 (s, 3H) 2.99-3.12 (m, 4H) 3.76-3.86 (m, 4H) 4.15 (d, | |
| | J = 6.8 Hz, 2H) 6.92-7.03 (m, 2H) 7.27-7.35 (m, 1H) 7.72 (d, J = 7.6 Hz, 1 | |
| 207 | 1H NMR (200 MHz, CHLOROFORM-D) d ppm 0.52-0.68 (m, 4H) 1.17-1.36 (m, 1H) | 114-115 |
| | 1.44 (s, 9H) 2.44 (s, 3H) 3.17-3.30 (m, 4H) 3.83-3.95 (m, 4H) 4.23 (d, | |
| | J = 7.0 Hz, 2H) 7.03 (dd, J = 7.9, 2.2 Hz, 1H) 7.33 (t, J = 7.9 Hz, 1H) 7.82 (d, | |
| | J = 7.5 Hz, 1H) 7.86-7.94 (m, 1H) | |
| 208 | 1H NMR (300 MHz, CHLOROFORM-D) d ppm 0.50-0.69 (m, 4H) 1.15-1.36 (m, 1H) | 126-128 |
| | 1.43 (s, 9H) 2.38 (s, 3H) 2.44 (s, 3H) 2.55-2.71 (m, 4H) 3.21-3.39 (m, | |
| | 4H) 4.23 (d, J = 6.8 Hz, 2H) 7.06 (d, J = 7.3 Hz, 1H) 7.32 (t, J = 7.3 Hz, 1H) | |
| | 7.79 (d, J = 7.3 Hz, 1H) 7.91 (s, 1H) | |
| 209 | 1H NMR (200 MHz, CHLOROFORM-D) d ppm 0.56-0.69 (m, 4H) 1.14-1.38 (m, 1H) | 87-89 |
| | 1.45 (s, 9H) 2.47 (s, 3H) 4.28 (d, J = 7.0 Hz, 2H) 7.60 (t, J = 7.5 Hz, 1H) | |
| | 8.29 (d, J = 7.5 Hz, 1H) 8.57 (d, J = 7.5 Hz, 1H) 9.10 (s, 1H) | |
| 210 | 1H NMR (200 MHz, CHLOROFORM-D) d ppm 0.55-0.66 (m, 4H) 1.09-1.37 (m, 1H) | 125-126 |
| | 1.45 (s, 9H) 2.46 (s, 3H) 4.24 (d, J = 7.0 Hz, 2H) 7.71 (d, J = 8.8 Hz, 2H) | |
| | 8.35 (d, J = 8.8 Hz, 2H) | |
| 211 | 1H NMR (200 MHz, CHLOROFORM-D) d ppm 0.39-0.83 (m, 4H) 1.09-1.38 (m, 1H) | |
| | 1.45 (s, 9H) 2.46 (s, 3H) 4.25 (d, J = 7.0 Hz, 2H) 7.54 (t, J = 7.7 Hz, 1H) | |
| | 7.65-7.86 (m, 1H) 8.43-8.59 (m, 2H) | |
| 212 | 1H NMR (200 MHz, CHLOROFORM-D) d ppm 0.47-0.80 (m, 4H) 1.13-1.39 (m, 1H) | 76-78 |
| | 1.45 (s, 9H) 2.46 (s, 3H) 4.26 (d, J = 6.6 Hz, 2H) 7.53 (t, J = 7.7 Hz, 1H) | |
| | 8.03 (d, J = 7.7 Hz, 1H) 8.44 (d, J = 7.7 Hz, 1H) 8.63 (s, 1H) | |
| 213 | 1H NMR (200 MHz, CHLOROFORM-D) d ppm 0.55-0.64 (m, 4H), 1.16-1.34 (m, 1H), | 122-123 |
| | 1.44 (s, 9H), 2.46 (s, 3H), 3.94 (s, 3H), 4.25 (d, J = 7.0 Hz, 2H), | |
| | 8.09 (d, J = 8.8 Hz, 2H), 8.32 (d, J = 8.8 Hz, 2H) | |
| 214 | 1H NMR (200 MHz, CHLOROFORM-D) d ppm 0.57-0.65 (m, 4H), 1.19-1.33 (m, 1H), | 196-197 |
| | 1.45 (s, 9H), 2.46 (s, 3H), 4.26 (d, J = 7.0 Hz, 2H), 8.16 (d, J = 8.8 Hz, | |
| | 2H), 8.36 (d, J = 8.8 Hz, 2H) | |
| 215 | 1H NMR (300 MHz, CHLOROFORM-D) d ppm 0.51-0.60 (m, 4H), 1.18-1.28 (m, 1H), | 97-98 |
| | 1.44 (s, 9H), 2.32 (s, 3H), 2.43 (s, 3H), 2.51 (s, 3H), 4.16 (d, J = 7.0 Hz, | |
| | 2H), 7.07-7.15 (m, 1H), 7.16-7.21 (m, 1H), 7.67-7.74 (m, 1H) | |
| 216 | 1H NMR (300 MHz, CHLOROFORM-D) d ppm 0.54 (s, 4H), 1.19-1.30 (m, 1H), | 100-101 |
| | 1.43 (s, 9H), 2.34 (s, 3H), 2.42 (s, 3H), 2.69 (s, 1H), 4.18 (d, J = 6.8 Hz, | |
| | 2H), 7.00-7.08 (m, 2H), 8.02-8.06 (m, 1H) | |
| 217 | 1H NMR (300 MHz, CHLOROFORM-D) d ppm 0.51-0.60 (m, 4H), 1.17-1.32 (m, 1H), | 106-107 |
| | 1.43 (s, 9H), 2.35 (s, 3H), 2.43 (s, 3H), 2.65 (s, 3H), 4.19 (d, J = 6.8 Hz, | |
| | 2H), 7.07-7.14 (m, 2H), 7.89 (s, 1H) | |
| 218 | 1H NMR (200 MHz, CHLOROFORM-D) d ppm 0.38-0.57 (m, 4H) 1.07-1.32 (m, 1H) | 98-100 |
| | 1.45 (s, 9H) 2.29 (s, 6H) 2.43 (s, 3H) 4.11 (d, J = 6.6 Hz, 2H) 6.95-7.18 | |
| 219 | 1H NMR (200 MHz, CHLOROFORM-D) d ppm 0.35-0.75 (m, 4H) 1.02-1.37 (m, 1H) | 91-92 |
| | 1.44 (s, 9H) 2.45 (s, 3H) 2.76 (s, 3H) 4.20 (d, J = 7.0 Hz, 2H) 7.31 (d, | |
| | J = 7.9 Hz, 1H) 7.52 (d, J = 7.9 Hz, 1H) 8.41 (s, 1H) | |
| 220 | 1H NMR (200 MHz, CHLOROFORM-D) d ppm 0.30-0.73 (m, 4H) 0.99-1.35 (m, 1H) | 159-161 |
| | 1.45 (s, 9H) 2.46 (s, 3H) 4.16 (d, J = 7.0 Hz, 2H) 7.72 (d, J = 8.4 Hz, 1H) | |
| | 7.84 (d, J = 8.4 Hz, 1H) 8.15 (s, 1H) | |
| 221 | 1H NMR (200 MHz, CHLOROFORM-D) d ppm 0.50-0.67 (m, 4H) 1.08-1.34 (m, 1H) | 170-172 |
| | 1.44 (s, 9H) 2.45 (s, 3H) 4.20 (d, J = 7.0 Hz, 2H) 6.98-7.12 (m, 2H) | |
| | 7.71-7.87 (m, 1H) | |
| 222 | 1H NMR (200 MHz, CHLOROFORM-D) d ppm 0.47-0.69 (m, 4H) 1.08-1.36 (m, 1H) | |
| | 1.44 (s, 9H) 2.45 (s, 3H) 4.21 (d, J = 7.0 Hz, 2H) 6.98-7.35 (m, 2H) | |
| | 7.76-7.93 (m, 1H) | |
| 223 | 1H NMR (300 MHz, CHLOROFORM-D) d ppm 0.44-0.62 (m, 4H), 1.13-1.29 (m, 1H), | 123-124 |
| | 1.44 (s, 9H), 2.44 (s, 3H), 4.13 (d, J = 6.8 Hz, 2H), 6.90 (t, J = 7.8 Hz, | |
| | 2H), 7.20-7.32 (m, 1H) | |
| 224 | 1H NMR (300 MHz, CHLOROFORM-D) d ppm 0.53-0.61 (m, 4H), 1.18-1.27 (m, 1H), | 111-112 |
| | 1.43 (s, 9H), 2.44 (s, 3H), 4.20 (d, J = 7.1 Hz, 2H), 6.79-6.93 (m, 2H), | |
| | 8.09-8.21 (m, 1H) | |
| 225 | 1H NMR (300 MHz, CHLOROFORM-D) d ppm 0.53-0.63 (m, 4H), 1.19-1.30 (m, 1H), | |
| | 1.44 (s, 9H), 2.45 (s, 3H), 4.21 (d, J = 7.1 Hz, 2H), 7.06-7.16 (m, 1H), | |
| | 7.40-7.50 (m, 1H), 7.93-8.02 (m, 1H) | |
| 226 | 1H NMR (300 MHz, CHLOROFORM-D) d ppm 0.53-0.61 (m, 4H), 1.16-1.29 (m, 1H), | 108-110 |
| | 1.43 (s, 9H), 2.45 (s, 3H), 4.20 (d, J = 7.0 Hz, 2H), 7.09-7.21 (m, 2H), | |
| | 8.02-8.12 (m, 1H) | |
| 227 | 1H NMR (300 MHz, CHLOROFORM-D) d ppm 0.52-0.62 (m, 4H), 1.15-1.30 (m, 1H), | 121-131 |
| | 1.43 (s, 9H), 2.45 (s, 3H), 4.20 (d, J = 7.1 Hz, 2H), 7.24-7.35 (m, 2H), | |
| | 8.00 (t, J = 8.0 Hz, 1H) | |
| 228 | 1H NMR (300 MHz, CHLOROFORM-D) d ppm 0.48-0.62 (m, 4H), 1.17-1.29 (m, 1H), | 92-94 |
| | 1.44 (s, 9H), 2.45 (s, 3H), 4.19 (d, J = 6.8 Hz, 2H), 6.89-7.00 (m, 1H), | |
| | 7.53-7.67 (m, 2H) | |
| 229 | 1H NMR (300 MHz, CHLOROFORM-D) d ppm 0.49-0.61 (m, 4H), 1.18-1.29 (m, 1H), | 126-128 |
| | 1.44 (s, 9H), 2.44 (s, 3H), 4.19 (d, J = 7.0 Hz, 2H), 6.09-7.10 (m, 1H) | |
| | 7.33-7.40 (m, 1H) 7.94-8.03 (m, 1H) | |
| 230 | 1H NMR (300 MHz, CHLOROFORM-D) d ppm 0.54-0.61 (m, 4H), 1.17-1.28 (m, 1H), | 113-114 |
| | 1.44 (s, 9H), 2.45 (s, 3H), 4.20 (d, J = 7.0 Hz, 2H), 6.93-7.04 (m, 1H), | |
| | 7.43-7.52 (m, 1H), 8.19-8.26 (m, 1H) | |
| 231 | 1H NMR (200 MHz, CHLOROFORM-D) d ppm 0.52-0.65 (m, 4H) 1.06-1.35 (m, 1H) | 95-96 |
| | 1.44 (s, 9H) 2.45 (s, 3H) 4.20 (d, J = 7.0 Hz, 2H) 6.79-6.93 (m, 1H) | |
| | 7.59-7.74 (m, 1H) 8.36-8.45 (m, 1H) | |
| 232 | 1H NMR (300 MHz, CHLOROFORM-D) d ppm 0.48-0.63 (m, 4H), 1.16-1.29 (m, 1H), | 129-131 |
| | 1.44 (s, 9H), 2.45 (s, 3H), 4.19 (d, J = 7.0 Hz, 2H), 7.23-7.28 (m, 1H), | |
| | 7.30-7.37 (m, 1H), 7.90-7.95 (m, 1H) | |
| 233 | 1H NMR (300 MHz, CHLOROFORM-D) d ppm 0.55-0.63 (m, 4H), 1.30 (s, 1H), | |
| | 1.44 (s, 9H), 2.46 (s, 3H), 4.23 (d, J = 7.0 Hz, 2H), 7.43 (t, J = 1.9 Hz, 1H), | |
| | 8.12 (d, J = 1.9 Hz, 2H) | |
| 234 | 1H NMR (200 MHz, CHLOROFORM-D) d ppm 0.52-0.64 (m, 4H) 1.14-1.34 (m, 1H) | 83-85 |
| | 1.43 (s, 9H) 2.43 (s, 3H) 3.80 (s, 3H) 3.87 (s, 3H) 4.19 (d, J = 6.6 Hz, 2H) | |
| | 6.86-6.98 (m, 2H) 7.52-7.59 (m, 1H) | |
| 235 | 1H NMR (200 MHz, CHLOROFORM-D) d ppm 0.49-0.59 (m, 4H) 1.15-1.32 (m, 1H) | 93-94 |
| | 1.43 (s, 9H) 2.43 (s, 3H) 3.88 (s, 3H) 3.95 (s, 1H) 4.17 (d, J = 6.6 Hz, 2H) | |
| | 6.96 (d, J = 7.5 Hz, 1H) 7.07 (t, J = 7.5 Hz, 1H) 7.46 (d, J = 7.5 Hz, 1H) | |
| 236 | 1H NMR (200 MHz, CHLOROFORM-D) d ppm 0.49-0.62 (m, 4H), 1.19-1.32 (m, 1H), | |
| | 1.41 (s, 9H), 2.41 (s, 3H), 3.85 (s, 3H), 3.90 (s, 3H), 4.16 (d, J = 7.9 Hz, | |
| | 2H), 6.46-6.56 (m, 2H), 8.06-8.14 (m, 1H) | |
| 237 | 1H NMR (300 MHz, CHLOROFORM-D) d ppm 0.51-0.59 (m, 4H) 1.17-1.33 (m, 1H) | 116-118 |
| | 1.42 (s, 9H) 1.42 (t, J = 7.0 Hz, 3H) 1.46 (t, J = 7.0 Hz, 3H) 2.40 (s, 3H) | |
| | 4.07 (q, J = 7.0 Hz, 2H) 4.11-4.20 (m, 4H) 6.46-6.51 (m, 2H) 8.02-8.07 | |
| 238 | 1H NMR (300 MHz, CHLOROFORM-D) d ppm 0.51-0.57 (m, 4H) 1.17-1.31 (m, 1H) | 97-99 |
| | 1.39 (t, J = 7.0 Hz, 6H) 1.43 (s, 9H) 2.42 (s, 3H) 4.03 (q, J = 7.0 Hz, 2H) | |
| | 4.11 (q, J = 7.0 Hz, 2H) 4.17 (d, J = 6.8 Hz, 2H) 6.88-6.91 (m, 2H) 7.47- | |
| 239 | 1H NMR (300 MHz, CHLOROFORM-D) d ppm 0.50-0.65 (m, 4H), 1.17-1.29 (m, 1H), | 117-119 |
| | 1.43 (s, 9H), 2.35 (s, 3H), 2.44 (s, 3H), 4.21 (d, J = 7.0 Hz, 2H), | |
| | 6.92-7.02 (m, 1H), 7.13-7.22 (m, 1H), 7.85-7.92 (m, 1H) | |
| 240 | 1H NMR (200 MHz, CHLOROFORM-D) d ppm 0.48-0.62 (m, 4H) 1.16-1.35 (m, 1H) | 94-96 |
| | 1.44 (s, 9H) 2.44 (s, 3H) 2.57 (d, J = 2.2 Hz, 3H) 4.17 (d, J = 7.0 Hz, 2H) | |
| | 6.98-7.13 (m, 1H) 7.09-7.24 (m, 1H) 7.79 (d, J = 7.5 Hz, 1H) | |
| 241 | 1H NMR (200 MHz, CHLOROFORM-D) d ppm 0.48-0.64 (m, 4H) 1.18-1.37 (m, 1H) | 91-93 |
| | 1.44 (s, 9H) 2.44 (s, 3H) 2.66 (s, 3H) 4.19 (d, J = 6.6 Hz, 2H) | |
| | 6.92-7.06 (m, 1H) 7.09-7.21 (m, 1H) 7.75-7.85 (m, 1H) | |
| 242 | 1H NMR (300 MHz, CHLOROFORM-D) d ppm 0.47-0.62 (m, 4H), 1.19-1.30 (m, 1H), | 108-109 |
| | 1.44 (s, 9H), 2.43 (s, 3H), 2.69 (s, 3H), 4.18 (d, J = 6.8 Hz, 2H), | |
| | 7.17-7.23 (m, 2H), 8.06 (d, J = 8.9 Hz, 1H) | |
| 243 | 1H NMR (200 MHz, CHLOROFORM-D) d ppm 0.47-0.62 (m, 4H) 1.11-1.34 (m, 1H) | 88-90 |
| | 1.44 (s, 9H) 2.44 (s, 3H) 2.65 (s, 3H) 4.16 (d, J = 6.6 Hz, 2H) 7.14 (t, | |
| | J = 7.5 Hz, 1H) 7.39 (d, J = 7.5 Hz, 1H) 7.76 (d, J = 7.5 Hz, 1H) | |
| 244 | 1H NMR (300 MHz, CHLOROFORM-D) d ppm 0.46-0.54 (m, 4H), 1.08-1.22 (m, 1H), | 159-160 |
| | 1.45 (s, 9H), 2.31 (s, 3H), 2.44 (s, 3H), 4.11 (d, J = 6.8 Hz, 2H), | |
| | 7.04-7.24 (m, 3H) | |
| 245 | 1H NMR (300 MHz, CHLOROFORM-D) d ppm 0.47-0.62 (m, 4H), 1.16-1.28 (m, 1H), | 89-90 |
| | 1.44 (s, 9H), 2.44 (s, 3H), 2.67 (s, 3H), 4.15 (d, J = 6.8 Hz, 2H), | |
| | 7.02-7.11 (m, 1H), 7.54-7.61 (m, 1H), 7.74-7.81 (m, 1H) | |
| 246 | 1H NMR (300 MHz, CHLOROFORM-D) d ppm 0.51-0.58 (m, 4H), 1.18-1.29 (m, 1H), | 82-84 |
| | 1.43 (s, 9H), 2.34 (s, 3H), 2.43 (s, 3H), 4.19 (d, J = 7.0 Hz, 2H), | |
| | 7.10-7.16 (m, 1H), 7.44-7.48 (m, 1H), 7.83-7.90 (m, 1H) | |
| 247 | 1H NMR (300 MHz, CHLOROFORM-D) d ppm 0.50-0.59 (m, 4H), 1.17-1.27 (m, 1H), | 142-144 |
| | 1.44 (s, 9H), 2.43 (s, 3H), 2.46 (s, 3H), 4.15 (d, J = 6.8 Hz, 2H), | |
| | 7.17-7.27 (m, 2H), 7.46-7.53 (m, 1H) | |
| 248 | 1H NMR (300 MHz, CHLOROFORM-D) d ppm 0.46-0.54 (m, 4H), 1.10-1.23 (m, 1H), | 89-90 |
| | 1.45 (s, 9H), 2.31 (s, 3H), 2.44 (s, 3H), 4.12 (d, J = 6.8 Hz, 2H), | |
| | 7.00-7.15 (m, 2H), 7.34-7.41 (m, 1H) | |
| 249 | 1H NMR (300 MHz, CHLOROFORM-D) d ppm 0.48-0.59 (m, 4H), 1.15-1.29 (m, 1H), | 149-151 |
| | 1.44 (s, 9H), 2.43 (s, 3H), 2.51 (s, 3H), 4.15 (d, J = 6.8 Hz, 2H), | |
| | 7.20-7.29 (m, 2H), 7.35-7.42 (m, 1H) | |
| 250 | 1H NMR (200 MHz, CHLOROFORM-D) d ppm 0.51-0.64 (m, 4H) 1.11-1.33 (m, 1H) | |
| | 1.44 (s, 9H) 2.46 (s, 3H) 4.21 (d, J = 7.0 Hz, 2H) 7.26 (t, J = 7.0 Hz, 1H) | |
| | 7.58-7.72 (m, 1H) 8.20-8.34 (m, 1H) | |
| 251 | 1H NMR (200 MHz, CHLOROFORM-D) d ppm 0.47-0.67 (m, 4H) 1.08-1.39 (m, 1H) | 113-115 |
| | 1.44 (s, 9H) 2.46 (s, 3H) 4.22 (d, J = 7.0 Hz, 2H) 7.20 (t, J = 8.8 Hz, 1H) | |
| | 7.56-7.72 (m, 1H) 8.43 (dd, J = 6.8, 2.4 Hz, 1H) | |
| 252 | 1H NMR (300 MHz, CHLOROFORM-D) d ppm 0.55-0.63 (m, 4H), 1.17-1.31 (m, 1H), | 123-124 |
| | 1.44 (s, 9H), 2.46 (s, 3H), 4.23 (d, J = 7.0 Hz, 2H), 7.18-7.28 (m, 1H), | |
| | 8.40-8.47 (m, 1H), 8.57 (dd, J = 7.3, 1.9 Hz, 1H) | |
| 253 | 1H NMR (300 MHz, CHLOROFORM-D) d ppm 0.49-0.69 (m, 4H), 1.17-1.34 (m, 1H), | 100-102 |
| | 1.45 (s, 9H), 2.47 (s, 3H), 4.24 (d, J = 7.0 Hz, 2H), 7.36-7.43 (m, 1H), | |
| | 8.08-8.15 (m, 1H), 8.35 (s, 1H) | |
| 254 | 1H NMR (200 MHz, CHLOROFORM-D) d ppm 0.37-0.70 (m, 4H) 0.99-1.33 (m, 1H) | 110-112 |
| | 1.44 (s, 9H) 2.44 (s, 3H) 4.15 (d, J = 6.6 Hz, 2H) 7.07-7.20 (m, 1H) | |
| | 7.48-7.58 (m, 1H) 7.64-7.75 (m, 1H) | |
| 255 | 1H NMR (200 MHz, CHLOROFORM-D) d ppm 0.33-0.78 (m, 4H) 0.99-1.35 (m, 1H) | |
| | 1.42 (s, 9H) 2.42 (s, 3H) 4.13 (d, J = 7.0 Hz, 2H) 7.08-7.57 (m, 2H) | |
| | 7.76-8.02 (m, 1H) | |
| 256 | 1H NMR (200 MHz, CHLOROFORM-D) d ppm 0.42-0.55 (m, 4H) 1.03-1.30 (m, 1H) | 128-130 |
| | 1.44 (s, 9H) 2.43 (s, 3H) 4.10 (d, J = 7.0 Hz, 2H) 7.19-7.51 (m, 3H) | |
| 257 | 1H NMR (300 MHz, CHLOROFORM-D) d ppm 0.50-0.64 (m, 4H), 1.16-1.30 (m, 1H), | 100-102 |
| | 1.45 (s, 9H), 2.46 (s, 3H), 4.19 (d, J = 7.0 Hz, 2H), 7.49-7.57 (m, 2H), | |
| | 8.24-8.27 (m, 1H) | |
| 258 | 1H NMR (200 MHz, CHLOROFORM-D) d ppm 0.24-0.80 (m, 4H) 1.04-1.34 (m, 1H) | 93-94 |
| | 1.45 (s, 9H) 2.45 (s, 3H) 4.15 (d, J = 7.0 Hz, 2H) 7.37 (t, J = 7.9 Hz, 1H) | |
| | 7.70 (d, J = 7.9 Hz, 1H) 7.88 (d, J = 7.9 Hz, 1H) | |
| 259 | 1H NMR (200 MHz, CHLOROFORM-D) d ppm 0.51-0.68 (m, 4H) 1.14-1.37 (m, 1H) | 122-124 |
| | 1.45 (s, 9H) 2.46 (s, 3H) 4.23 (d, J = 7.0 Hz, 2H) 7.51-7.59 (m, 1H) | |
| | 8.29-8.38 (m, 1H) 8.60-8.65 (m, 1H) | |
| 260 | 1H NMR (200 MHz, CHLOROFORM-D) d ppm 0.47-0.63 (m, 4H), 0.88 (s, 9H), | 88-89 |
| | 1.14-1.45 (m, 1H), 1.49 (s, 6H), 1.78 (s, 2H), 2.45 (s, 3H), 4.23 (d, | |
| | J = 7.0 Hz, 2H), 7.20 (t, J = 9.2 Hz, 1H), 7.60-7.71 (m, 1H), 8.39-8.51 (m, | |
| 261 | 1H NMR (200 MHz, CHLOROFORM-D) d ppm 0.49-0.62 (m, 4H) 1.14-1.38 (m, 1H) | 95-96 |
| | 1.43 (s, 9H) 2.42 (s, 3H) 2.74 (s, 3H) 3.84 (s, 3H) 4.18 (d, J = 7.0 Hz, 2H) | |
| | 6.70-6.82 (m, 2H) 8.16-8.24 (m, 1H) | |
| 262 | 1H NMR (300 MHz, CHLOROFORM-D) d ppm 0.53-0.60 (m, 4H), 1.18-1.29 (m, 1H), | 87-89 |
| | 1.43 (s, 9H), 2.43 (s, 3H), 3.89 (s, 3H), 4.17 (d, J = 6.8 Hz, 2H), | |
| | 6.85-6.94 (m, 1H) 7.00-7.11 (m, 1H) 7.63-7.71 (m, 1H) | |
| 263 | 1H NMR (300 MHz, CHLOROFORM-D) d ppm 0.46-0.61 (m, 4H), 1.11-1.24 (m, 1H), | 119-121 |
| | 1.43 (s, 9H), 2.42 (s, 3H), 3.82 (s, 3H), 4.11 (d, J = 7.1 Hz, 2H), | |
| | 6.66-6.74 (m, 2H), 7.16-7.28 (m, 1H) | |
| 264 | 1H NMR (200 MHz, CHLOROFORM-D) d ppm 0.51-0.62 (m, 4H) 1.13-1.33 (m, 1H) | 163-165 |
| | 1.42 (s, 9H) 2.42 (s, 3H) 3.90 (s, 3H) 4.16 (d, J = 7.0 Hz, 2H) | |
| | 6.89-7.01 (m, 2H) 7.88-7.97 (m, 1H) | |
| 265 | 1H NMR (200 MHz, CHLOROFORM-D) d ppm 0.52-0.63 (m, 4H) 1.09-1.34 (m, 1H) | 98-100 |
| | 1.43 (s, 9H) 2.43 (s, 3H) 3.89 (s, 3H) 4.17 (d, J = 6.6 Hz, 2H) | |
| | 6.84-8.93 (m, 1H) 7.25-7.37 (m, 1H) 7.88-7.94 (m, 1H) | |
| 266 | 1H NMR (200 MHz, CHLOROFORM-D) d ppm 0.28-0.71 (m, 4H) 1.07-1.34 (m, 1H) | 169-170 |
| | 1.44 (s, 9H) 2.44 (s, 3H) 4.17 (d, J = 6.6 Hz, 2H) 6.74 (t, J = 76.0 Hz, 1H) | |
| | 7.06-7.14 (m, 1H) 7.46-7.55 (m, 1H) 8.14-8.19 (m, 1H) | |
| 267 | 1H NMR (200 MHz, CHLOROFORM-D) d ppm 0.48-0.66 (m, 4H) 1.07-1.34 (m, 1H) | 175-177 |
| | 1.43 (s, 9H) 2.44 (s, 3H) 3.95 (s, 3H) 4.18 (d, J = 7.0 Hz, 2H) 7.02 (d, | |
| | J = 9.2 Hz, 1H) 7.60 (d, J = 9.2 Hz, 1H) 8.27 (s, 1H) | |
| 268 | 1H NMR (200 MHz, CHLOROFORM-D) d ppm 0.50-0.65 (m, 4H) 1.10-1.35 (m, 1H) | 140-142 |
| | 1.40-1.53 (m, 3H) 1.44 (s, 9H) 2.44 (s, 3H) 4.11-4.30 (m, 2H) 4.21 (d, | |
| | J = 7.0 Hz, 2H) 7.00 (d, J = 8.8 Hz, 1H) 7.56 (d, J = 8.8 Hz, 1H) 8.20 (s, 1H) | |
| 269 | 1H NMR (200 MHz, CHLOROFORM-D) d ppm 0.48-0.64 (m, 4H) 1.12-1.34 (m, 1H) | 106-108 |
| | 1.39 (d, J = 6.2 Hz, 6H) 1.43 (s, 9H) 2.43 (s, 3H) 4.15 (d, J = 6.6 Hz, 2H) | |
| | 4.55-4.77 (m, 1H) 7.00 (d, J = 8.8 Hz, 1H) 7.55 (d, J = 8.8 Hz, 1H) 8.12 (s, | |
| 270 | 1H NMR (200 MHz, CHLOROFORM-D) d ppm 0.46-0.66 (m, 4H) 1.06-1.34 (m, 1H) | 108-110 |
| | 1.44 (s, 9H) 2.44 (s, 3H) 3.42 (s, 3H) 3.76-3.86 (m, 2H) 4.18 (d, J = 6.6 Hz, | |
| | 2H) 4.22-4.35 (m, 2H) 7.08 (d, J = 8.8 Hz, 1H) 7.58 (d, J = 8.8 Hz, 1H) | |
| 271 | 1H NMR (200 MHz, CHLOROFORM-D) d ppm 0.50-0.66 (m, 4H) 1.13-1.34 (m, 1H) | 131-132 |
| | 1.44 (s, 9H) 2.44 (s, 3H) 3.41 (s, 3H) 3.49 (t, J = 5.5 Hz, 2H) 3.65 (t, | |
| | J = 5.5 Hz, 2H) 4.20 (d, J = 7.0 Hz, 2H) 6.73 (d, J = 8.8 Hz, 1H) 7.47 (d, J = 8.8 Hz, | |
| | 1H) 8.61 (s, 1H) 9.09-9.27 (m, 1H) | |
| 272 | 1H NMR (200 MHz, CHLOROFORM-D) d ppm 0.52-0.65 (m, 4H) 1.16-1.36 (m, 1H) | 131-133 |
| | 1.44 (s, 9H) 2.44 (s, 3H) 3.49 (t, J = 5.5 Hz, 2H) 3.88 (t, J = 5.5 Hz, 2H) | |
| | 4.21 (d, J = 6.6 Hz, 2H) 6.77 (d, J = 8.8 Hz, 1H) 7.42-7.53 (m, 1H) 8.64 (s, | |
| 273 | 1H NMR (200 MHz, CHLOROFORM-D) d ppm 0.50-0.66 (m, 4H) 1.13-1.34 (m, 1H) | 131-132 |
| | 1.44 (s, 9H) 2.44 (s, 3H) 3.41 (s, 3H) 3.49 (t, J = 5.5 Hz, 2H) 3.65 (t, | |
| | J = 5.5 Hz, 2H) 4.20 (d, J = 7.0 Hz, 2H) 6.73 (d, J = 8.8 Hz, 1H) 7.47 (d, J = 8.8 Hz, | |
| | 1H) 8.61 (s, 1H) 9.09-9.27 (m, 1H) | |
| 274 | 1H NMR (200 MHz, CHLOROFORM-D) d ppm 0.49-0.64 (m, 4H) 1.12 (t, J = 7.2 Hz, 3H) | 101-103 |
| | 1.18-1.37 (m, 1H) 1.43 (s, 9H) 2.44 (s, 3H) 2.87 (s, 3H) 3.29 (q, | |
| | J = 7.2 Hz, 2H) 4.15 (d, J = 7.0 Hz, 2H) 6.90 (d, J = 8.4 Hz, 1H) 7.43 (d, J = 8.4 Hz, | |
| | 1H) 7.92 (s, 1H) | |
| 275 | 1H NMR (200 MHz, CHLOROFORM-D) d ppm 0.52-0.66 (m, 4H) 1.08-1.33 (m, 1H) | 100-102 |
| | 1.43 (s, 9H) 2.29 (qn, J = 7.5 Hz, 2H) 2.43 (s, 3H) 3.96 (t, J = 7.5 Hz, 4H) | |
| | 4.16 (d, J = 6.6 Hz, 2H) 6.48 (d, J = 9.2 Hz, 1H) 7.43 (d, J = 9.2 Hz, 1H) 7.98 | |
| 276 | 1H NMR (200 MHz, CHLOROFORM-D) d ppm 0.48-0.62 (m, 4H) 1.09 (t, J = 7.0 Hz, 6H) | 131-133 |
| | 1.15-1.33 (m, 1H) 1.44 (s, 9H) 2.43 (s, 3H) 3.27 (q, J = 7.0 Hz, 4H) | |
| | 4.14 (d, J = 7.0 Hz, 2H) 6.94 (d, J = 8.4 Hz, 1H) 7.43 (d, J = 8.4 Hz, 1H) 7.89 | |
| 277 | 1H NMR (200 MHz, CHLOROFORM-D) d ppm 0.51-0.64 (m, 4H) 1.11-1.31 (m, 1H) | 126-128 |
| | 1.44 (s, 9H) 2.44 (s, 3H) 2.91 (s, 6H) 4.16 (d, J = 7.0 Hz, 2H) 6.90 (d, | |
| | J = 8.8 Hz, 1H) 7.45 (dd, J = 8.8, 2.0 Hz, 1H) 8.00 (d, J = 2.0 Hz, 1H) | |
| 278 | 1H NMR (200 MHz, CHLOROFORM-D) d ppm 0.51-0.64 (m, 4H), 1.10-1.32 (m, 1H), | |
| | 1.43 (s, 9H), 1.83-1.97 (m, 4H), 2.43 (s, 3H), 3.22-3.38 (m, 4H), | |
| | 4.15 (d, J = 6.6 Hz, 2H), 6.75 (d, J = 8.8 Hz, 1H), 7.41 (dd, J = 8.8, 2.6 Hz, 1H), | |
| | 7.91 (d, J = 2.6 Hz, 1H) | |
| 279 | 1H NMR (200 MHz, CHLOROFORM-D) d ppm 0.48-0.62 (m, 4H), 1.15-1.34 (m, 1H), | 138-140 |
| | 1.44 (s, 9H), 1.49-1.73 (m, 6H), 2.43 (s, 3H), 3.07-3.19 (m, 4H), | |
| | 4.15 (d, J = 7.0 Hz, 2H), 6.97 (d, J = 8.8 Hz, 1H), 7.45 (dd, J = 8.8, 2.2 Hz, 1H), | |
| | 7.96 (d, J = 2.2 Hz, 1H) | |
| 280 | 1H NMR (200 MHz, CHLOROFORM-D) d ppm 0.48-0.65 (m, 4H) 1.09-1.35 (m, 1H) | 86-87 |
| | 1.45 (s, 9H) 2.44 (s, 3H) 3.05-3.22 (m, 4H) 3.75-3.91 (m, 4H) 4.16 (d, | |
| | J = 6.6 Hz, 2H) 6.98 (d, J = 8.8 Hz, 1H) 7.51 (dd, J = 8.8, 1.8 Hz, 1H) 8.03 (d, | |
| 281 | 1H NMR (200 MHz, CHLOROFORM-D) d ppm 0.43-0.88 (m, 4H) 1.08-1.39 (m, 1H) | 195-197 |
| | 1.45 (s, 9H) 2.46 (s, 3H) 4.23 (d, J = 7.0 Hz, 2H) 7.03-7.55 (m, 1H) | |
| | 8.27-8.77 (m, 2H) | |
| 282 | 1H NMR (300 MHz, CHLOROFORM-D) d ppm 0.51-0.64 (m, 4H), 1.16-1.31 (m, 1H), | 82-84 |
| | 1.44 (s, 9H), 2.45 (s, 3H), 4.21 (d, J = 7.0 Hz, 2H), 6.93-7.03 (m, 1H), | |
| | 7.83-7.93 (m, 1H) | |
| 283 | 1H NMR (300 MHz, CHLOROFORM-D) d ppm 0.54-0.60 (m, 4H), 1.17-1.29 (m, 1H), | 78-79 |
| | 1.43 (s, 9H), 2.45 (s, 3H), 4.20 (d, J = 7.0 Hz, 2H), 6.88-7.01 (m, 1H) | |
| | 7.94-8.03 (m, 1H) | |
| 284 | 1H NMR (300 MHz, CHLOROFORM-D) d ppm 0.47-0.63 (m, 4H), 1.16-1.30 (m, 1H), | 112-114 |
| | 1.44 (s, 9H), 2.45 (s, 3H), 4.18 (d, J = 7.0 Hz, 2H), 7.20-7.29 (m, 1H), | |
| | 7.83-7.94 (m, 2H) | |
| 285 | 1H NMR (200 MHz, CHLOROFORM-D) d ppm 0.49-0.67 (m, 4H) 1.08-1.35 (m, 1H) | 76-77 |
| | 1.44 (s, 9H) 2.45 (s, 3H) 4.21 (d, J = 7.0 Hz, 2H) 6.93-7.07 (m, 1H) | |
| | 7.94-8.12 (m, 1H) | |
| 286 | 1H NMR (300 MHz, CHLOROFORM-D) d ppm 0.52-0.60 (m, 4H), 1.18-1.34 (m, 1H), | 88-89 |
| | 1.43 (s, 9H), 2.42 (s, 3H), 3.89 (s, 3H), 3.90 (s, 3H), 3.99 (s, 3H), | |
| | 4.18 (d, J = 6.8 Hz, 2H), 6.71 (d, J = 8.9 Hz, 1H), 7.78 (d, J = 8.9 Hz, 1H) | |
| 287 | 1H NMR (300 MHz, CHLOROFORM-D) d ppm 0.50-0.63 (m, 4H), 1.17-1.28 (m, 1H), | 122-124 |
| | 1.43 (s, 9H), 2.33 (d, J = 2.0 Hz, 3H), 2.44 (s, 3H), 4.21 (d, J = 7.0 Hz, | |
| | 2H), 6.91-6.99 (m, 1H), 7.71-7.81 (m, 1H) | |
| 288 | 1H NMR (200 MHz, CHLOROFORM-D) d ppm 0.42-0.69 (m, 4H) 0.94-1.34 (m, 1H) | 109-111 |
| | 1.45 (s, 9H) 2.45 (s, 3H) 4.12 (d, J = 7.0 Hz, 2H) 6.92-7.05 (m, 1H) | |
| | 7.40-7.68 (m, 1H) | |
| 289 | 1H NMR (200 MHz, CHLOROFORM-D) d ppm 0.37-0.58 (m, 4H) 1.06-1.26 (m, 1H) | 182-183 |
| | 1.45 (s, 9H) 2.44 (s, 3H) 4.09 (d, J = 7.0 Hz, 2H) 7.34-7.52 (m, 2H) | |
| 290 | 1H NMR (200 MHz, CHLOROFORM-D) d ppm 0.51-0.67 (m, 4H), 1.15-1.32 (m, 1H), | |
| | 1.45 (s, 9H), 2.45 (s, 3H), 4.28 (d, J = 7.0 Hz, 2H), 7.28-7.39 (m, 1H), | |
| | 7.72-7.84 (m, 1H), 8.30 (d, J = 7.7 Hz, 1H), 8.75 (d, J = 7.7 Hz, 1H) | |
| 291 | 1H NMR (200 MHz, CHLOROFORM-D) d ppm 0.59 (d, J = 6.6 Hz, 4H), 1.17-1.38 (m, | |
| | 1H), 1.45 (s, 9H), 2.46 (s, 3H), 4.24 (d, J = 7.0 Hz, 2H), 7.30-7.43 (m, 1H), | |
| | 8.43-8.55 (m, 1H), 8.63-8.71 (m, 1H), 9.46-9.52 (m, 1H) | |
| 292 | 1H NMR (200 MHz, CHLOROFORM-D) d ppm 0.53-0.64 (m, 4H), 1.18-1.31 (m, 1H), | |
| | 1.46 (s, 9H), 2.47 (s, 3H), 4.26 (d, J = 7.0 Hz, 2H), 8.58-8.64 (m, 1H), | |
| | 8.68-8.74 (m, 1H), 9.49-9.54 (m, 1H) | |
| 293 | 1H NMR (300 MHz, CHLOROFORM-D) d ppm 0.48-0.56 (m, 4H), 1.10-1.28 (m, 1H), | 88-89 |
| | 1.44 (s, 9H), 2.43 (s, 3H), 2.54 (s, 3H), 4.19 (d, J = 6.8 Hz, 2H), | |
| | 7.16-7.22 (m, 1H), 7.51-7.55 (m, 1H), 8.46-8.51 (m, 1H) | |
| 294 | 1H NMR (200 MHz, CHLOROFORM-D) d ppm 0.52-0.64 (d, 4H), 1.08-1.38 (m, 1H), | |
| | 1.44 (s, 9H), 2.42 (s, 3H), 2.46 (s, 3H), 4.24 (d, J = 7.0 Hz, 2H), | |
| | 7.31-7.47 (m, 1H), 7.67-7.94 (m, 1H), 8.35-8.50 (m, 1H) | |
| 295 | 1H NMR (200 MHz, CHLOROFORM-D) d ppm 0.42-0.66 (m, 4H), 1.14-1.34 (m, 1H), | |
| | 1.44 (s, 9H), 2.45 (s, 3H), 2.97 (s, 3H), 4.18 (d, J = 6.6 Hz, 2H), | |
| | 7.24-7.46 (m, 1H), 7.64-7.93 (m, 1H), 8.41-8.55 (m, 1H) | |
| 296 | 1H NMR (300 MHz, CHLOROFORM-D) d ppm 0.52-0.62 (m, 4H) 1.16-1.31 (m, 1H) | 228-230 |
| | 1.44 (s, 9H) 2.45 (s, 3H) 2.62 (s, 3H) 4.22 (d, J = 7.0 Hz, 2H) 7.22 (d, | |
| | J = 7.9 Hz, 1H) 8.38 (dd, J = 7.9, 2.2 Hz, 1H) 9.38 (d, J = 2.2 Hz, 1H) | |
| 297 | 1H NMR (300 MHz, CHLOROFORM-D) d ppm 0.49-0.67 (m, 4H), 1.15-1.33 (m, 1H), | 104-106 |
| | 1.29 (t, J = 7.6 Hz, 3H), 1.44 (s, 9H), 2.45 (s, 3H), 2.73 (q, J = 7.6 Hz, | |
| | 2H), 4.29 (d, J = 7.0 Hz, 2H), 7.18 (d, J = 5.0 Hz, 1H), 8.15 (s, 1H), 8.62 | |
| 298 | 1H NMR (200 MHz, CHLOROFORM-D) d ppm 0.43-0.62 (m, 4H) 1.05-1.35 (m, 1H) | 74-76 |
| | 1.45 (s, 9H) 2.45 (s, 3H) 4.16 (d, J = 7.0 Hz, 2H) 7.58 (d, J = 4.8 Hz, 1H) | |
| | 8.77 (d, J = 4.8 Hz, 1H) 9.20 (s, 1H) | |
| 299 | 1H NMR (300 MHz, CHLOROFORM-D) d ppm 0.40-0.58 (m, 4H), 1.07-1.29 (m, 1H), | 156-157 |
| | 1.46 (s, 9H), 2.29 (s, 3H), 2.45 (s, 3H), 2.52 (s, 3H), 4.11 (d, J = 6.8 Hz, | |
| | 2H), 6.96 (d, J = 5.1 Hz, 1H), 8.31 (d, J = 5.1 Hz, 1H) | |
| 300 | 1H NMR (200 MHz, CHLOROFORM-D) d ppm 0.50-0.66 (m, 4H), 1.09-1.38 (m, 1H), | 83-84 |
| | 1.44 (s, 9H), 2.46 (s, 3H), 4.24 (d, J = 22.0 Hz, 2H), 7.20-7.34 (m, 1H), | |
| | 8.21-8.32 (m, 1H), 8.45-8.59 (m, 1H) | |
| 301 | 1H NMR (200 MHz, CHLOROFORM-D) d ppm 0.49-0.63 (m, 4H) 1.10-1.34 (m, 1H) | 124-125 |
| | 1.45 (s, 9H) 2.45 (s, 3H) 4.20 (d, J = 7.0 Hz, 2H) 7.28 (dd, J = 7.5, 4.8 Hz, 1H) | |
| | 8.25 (dd, J = 7.5, 2.2 Hz, 1H) 8.41 (dd, J = 4.8, 2.2 Hz, 1H) | |
| 302 | 1H NMR (200 MHz, CHLOROFORM-D) d ppm 0.46-0.65 (m, 4H) 1.14-1.35 (m, 1H) | |
| | 1.44 (s, 9H) 2.45 (s, 3H) 4.20 (d, J = 7.0 Hz, 2H) 7.36 (d, J = 5.3 Hz, 1H) | |
| | 8.47 (d, J = 5.3 Hz, 1H) 9.21 (s, 1H) | |
| 303 | 1H NMR (200 MHz, CHLOROFORM-D) d ppm 0.52-0.67 (m, 4H) 1.13-1.37 (m, 1H) | 152-154 |
| | 1.45 (s, 9H) 2.46 (s, 3H) 4.23 (d, J = 6.6 Hz, 2H) 8.60 (dd, J = 2.6, 1.3 Hz, 1H) | |
| | 8.73 (d, J = 2.6 Hz, 1H) 9.36 (d, J = 1.3 Hz, 1H) | |
| 304 | 1H NMR (200 MHz, CHLOROFORM-D) d ppm 0.46-0.58 (m, 4H) 1.08-1.34 (m, 1H) | 141-142 |
| | 1.45 (s, 9H) 2.44 (s, 3H) 4.19 (d, J = 7.0 Hz, 2H) 7.17 (dd, J = 7.9, 4.8 Hz, 1H) | |
| | 7.93 (d, J = 7.9 Hz, 1H) 8.58 (d, J = 4.8 Hz, 1H) | |
| 305 | 1H NMR (200 MHz, CHLOROFORM-D) d ppm 0.47-0.61 (m, 4H) 1.09-1.34 (m, 1H) | 115-116 |
| | 1.45 (s, 9H) 2.46 (s, 3H) 4.17 (d, J = 7.0 Hz, 2H) 8.37 (d, J = 2.6 Hz, 1H) | |
| | 8.52 (d, J = 2.6 Hz, 1H) | |
| 306 | 1H NMR (200 MHz, CHLOROFORM-D) d ppm 0.48-0.61 (m, 4H) 1.06-1.31 (m, 1H) | 143-145 |
| | 1.44 (s, 9H) 2.44 (s, 3H) 4.15 (d, J = 7.0 Hz, 2H) 7.26 (d, J = 8.4 Hz, 1H) | |
| | 7.67 (d, J = 8.4 Hz, 1H) | |
| 307 | 1H NMR (200 MHz, CHLOROFORM-D) d ppm 0.44-0.66 (m, 4H) 1.07-1.37 (m, 1H) | 110-112 |
| | 1.45 (s, 9H) 2.47 (s, 3H) 4.19 (d, J = 7.0 Hz, 2H) 7.77 (s, 1H) 8.41 (s, 1 | |
| 308 | 1H NMR (200 MHz, CHLOROFORM-D) d ppm 0.40-0.64 (m, 4H) 0.98-1.32 (m, 1H) | 260-261 |
| | 1.46 (s, 9H) 2.46 (s, 3H) 4.11 (d, J = 7.0 Hz, 2H) 8.48 (s, 2H) | |
| 309 | 1H NMR (300 MHz, CHLOROFORM-D) d ppm 0.48-0.64 (m, 4H), 1.17-1.29 (m, 1H), | 113-134 |
| | 1.45 (s, 9H), 2.47 (s, 3H), 4.20 (d, J = 6.8 Hz, 2H), 8.23 (d, J = 2.6 Hz, | |
| | 1H), 8.36 (d, J = 2.6 Hz, 1H) | |
| 310 | 1H NMR (200 MHz, CHLOROFORM-D) d ppm 0.49-0.63 (m, 4H) 1.15-1.34 (m, 1H) | 102-104 |
| | 1.43 (s, 9H) 1.44 (t, J = 7.0 Hz, 3H) 2.43 (s, 3H) 4.18 (d, J = 7.0 Hz, 2H) | |
| | 4.53 (q, J = 7.0 Hz, 2H) 6.90 (dd, J = 7.5, 4.8 Hz, 1H) 8.19 (dd, J = 4.8, 2.2 Hz, | |
| | 1H) 8.28 (dd, J = 7.5, 2.2 Hz, 1H) | |
| 311 | 1H NMR (200 MHz, CHLOROFORM-D) d ppm 0.49-0.60 (m, 4H), 1.22-1.35 (m, 1H), | 151-153 |
| | 1.40 (d, J = 6.2 Hz, 6H), 1.43 (s, 9H), 2.42 (s, 3H), 4.17 (d, J = 7.0 Hz, | |
| | 2H), 5.35-5.55 (m, 1H), 6.80-6.92 (m, 1H), 8.13-8.26 (m, 2H) | |
| 312 | 1H NMR (300 MHz, CHLOROFORM-D) d ppm 0.53-0.61 (m, 4H), 1.20-1.31 (m, 1H), | 138-139 |
| | 1.43 (s, 9H), 2.45 (s, 3H), 2.52 (s, 3H), 4.29 (d, J = 6.8 Hz, 2H), | |
| | 7.01-7.10 (m, 1H), 8.46-8.53 (m, 2H) | |
| 313 | 1H NMR (200 MHz, CHLOROFORM-D) d ppm 5.11-5.22 (m, 4H) 5.82-6.07 (m, 1H) | 111-114 |
| | 6.16 (s, 9H) 7.15 (s, 3H) 8.85 (d, J = 7.0 Hz, 2H) 11.79 (dd, J = 7.5, 4.8 Hz, | |
| | 1H) 11.83-11.96 (m, 3H) 12.04-12.19 (m, 2H) 12.92 (dd, J = 4.8, 2.2 Hz, 1H) | |
| | 13.14 (dd, J = 7.5, 2.2 Hz, 1H) | |
| 314 | 1H NMR (200 MHz, CHLOROFORM-D) d ppm 0.48-0.66 (m, 4H) 1.07-1.33 (m, 1H) | 124-126 |
| | 1.43 (s, 9H) 2.45 (s, 3H) 4.08 (s, 3H) 4.14 (s, 3H) 4.16 (d, J = 6.6 Hz, 2H) | |
| | 7.39 (s, 1H) | |
| 315 | 1H NMR (200 MHz, CHLOROFORM-D) d ppm 0.49-0.63 (m, 4H), 1.14-1.31 (m, 1H), | 97-98 |
| | 1.43 (s, 9H), 1.82-1.94 (m, 4H), 2.43 (s, 3H), 3.38-3.51 (m, 4H), | |
| | 4.13 (d, J = 6.6 Hz, 2H), 6.58 (dd, J = 7.5, 4.8 Hz, 1H), 7.89 (dd, J = 7.5, 2.2 Hz, | |
| | 1H), 8.17 (dd, J = 4.8, 2.2 Hz, 2H) | |
| 316 | 1H NMR (200 MHz, CHLOROFORM-D) d ppm 0.34-0.78 (m, 4H) 1.03-1.38 (m, 1H) | 67-69 |
| | 1.44 (s, 9H) 2.45 (s, 3H) 2.89 (s, 3H) 4.18 (d, J = 7.0 Hz, 2H) | |
| | 6.73-6.81 (m, 1H) 8.45-8.57 (m, 1H) | |
| 317 | 1H NMR (200 MHz, CHLOROFORM-D) d ppm 0.52-0.66 (m, 4H), 1.12-1.34 (m, 1H), | 127-128 |
| | 1.44 (s, 9H), 2.46 (s, 3H), 3.98 (s, 3H), 4.22 (d, J = 7.0 Hz, 2H), | |
| | 7.43 (d, J = 0.9 Hz, 1H), 7.65 (d, J = 0.9 Hz, 1H) | |
| 318 | 1H NMR (300 MHz, CHLOROFORM-D) d ppm 0.53-0.60 (m, 4H), 0.85 (t, J = 7.3 Hz, | 104-105 |
| | 3H), 1.20-1.31 (m, 1H), 1.43 (s, 9H), 1.52-1.70 (m, 2H), 2.42 (s, 3H), | |
| | 3.07-3.16 (m, 2H), 4.17 (d, J = 6.8 Hz, 2H), 7.39-7.54 (m, 5H), 8.18 | |
| 319 | 1H NMR (200 MHz, CHLOROFORM-D) d ppm 0.54-0.70 (m, 4H) 1.07-1.31 (m, 1H) | 129-130 |
| | 1.44 (s, 9H) 2.47 (s, 3H) 4.21 (d, J = 7.0 Hz, 2H) 7.19 (d, J = 4.0 Hz, 1H) | |
| | 7.33 (d, J = 4.0 Hz, 1H) | |
| 320 | 1H NMR (200 MHz, CHLOROFORM-D) d ppm 0.55-0.68 (m, 4H) 1.16-1.39 (m, 1H) | 139-141 |
| | 1.46 (s, 9H) 2.48 (s, 3H) 4.27 (d, J = 7.0 Hz, 2H) 7.84 (d, J = 9.7 Hz, 1H) | |
| | 8.33 (d, J = 9.7 Hz, 1H) 8.77 (s, 1H) | |
| 321 | 1H NMR (200 MHz, CHLOROFORM-D) d ppm 0.49-0.71 (m, 4H) 1.17-1.40 (m, 1H) | 109-110 |
| | 1.47 (s, 9H) 2.47 (s, 3H) 4.25 (d, J = 7.0 Hz, 2H) 7.65 (dd, J = 8.1, 7.3 Hz, 1H) | |
| | 8.05 (d, J = 8.1 Hz, 1H) 8.56 (d, J = 6.2 Hz, 1H) 8.62 (dd, J = 7.3, 1.5 Hz, 1H) | |
| | 9.14 (d, J = 6.2 Hz, 1H) 9.26 (s, 1H) | |
| 322 | 1H NMR (200 MHz, CHLOROFORM-D) d ppm 0.46-0.67 (m, 4H) 1.03-1.38 (m, 1H) | 193-195 |
| | 1.45 (s, 9H) 2.44 (s, 3H) 4.15 (d, J = 7.0 Hz, 2H) 7.38 (dd, J = 8.1, 4.2 Hz, 1H) | |
| | 7.56 (dd, J = 8.1, 7.0 Hz, 1H) 7.84 (dd, J = 8.1, 1.8 Hz, 1H) 8.06 (dd, | |
| | J = 7.0, 1.8 Hz, 1H) 8.15 (dd, J = 8.1, 2.0 Hz, 1H) 9.04 (dd, J = 4.2, 2.0 Hz, 1 | |
| 323 | 1H NMR (200 MHz, CHLOROFORM-D) d ppm 0.47-0.68 (m, 4H), 1.12-1.39 (m, 1H) | 124-126 |
| | 1.47 (s, 9H) 2.48 (s, 3H) 4.23 (d, J = 7.0 Hz, 2H) 7.57 (ddd, J = 8.4, 6.8, 1.5 Hz, | |
| | 1H) 7.71 (ddd, J = 8.4, 6.8, 1.5 Hz, 1H) 8.01 (d, J = 4.4 Hz, 1H) 8.13 (d, | |
| | J = 7.5 Hz, 1H) 9.00 (d, J = 4.4 Hz, 1H) 9.05 (dd, J = 7.5, 1.1 Hz, 1H) | |
| 324 | 1H NMR (300 MHz, CHLOROFORM-D) d ppm 0.47-0.54 (m, 4H), 1.12-1.24 (m, 1H), | |
| | 1.48 (s, 9H), 2.46 (s, 3H), 4.22 (d, J = 7.9 Hz, 2H), 7.52-7.87 (m, 4H), | |
| | 8.56-8.72 (m, 2H) | |
| 325 | 1H NMR (200 MHz, CHLOROFORM-D) d ppm 0.43-0.75 (m, 4H), 1.15-1.41 (m, 1H), | 140-141 |
| | 1.50 (s, 9H), 2.54 (s, 3H), 4.13-4.50 (m, 2H), 8.02-8.27 (m, 2H), | |
| | 8.72-8.92 (m, 1H), 9.54-9.75 (m, 1H), 9.90-10.21 (m, 1H) | |
| 326 | 1H NMR (200 MHz, CHLOROFORM-D) d ppm 0.46-0.72 (m, 4H) 1.12-1.38 (m, 1H) | 201-203 |
| | 1.48 (s, 9H) 2.49 (s, 3H) 4.23 (d, J = 7.0 Hz, 2H) 7.57 (t, J = 8.4 Hz, 1H) | |
| | 7.72 (t, J = 8.4 Hz, 1H) 7.98 (s, 1H) 8.04 (d, J = 8.4 Hz, 1H) 9.00 (d, J = 8.4 | |
| 327 | 1H NMR (200 MHz, CHLOROFORM-D) d ppm 0.38-0.59 (m, 4H) 1.01-1.28 (m, 1H) | 172-174 |
| | 1.46 (s, 9H) 2.44 (s, 3H) 4.10 (d, J = 7.0 Hz, 2H) 7.56 (d, J = 9.2 Hz, 1H) | |
| | 7.65 (d, J = 9.2 Hz, 1H) 8.09 (d, J = 2.6 Hz, 1H) 8.80 (d, J = 2.6 Hz, 1H) | |
| 328 | 1H NMR (200 MHz, CHLOROFORM-D) d ppm 0.36-0.65 (m, 4H) 1.06-1.36 (m, 1H) | 147-149 |
| | 1.46 (s, 9H) 2.46 (s, 3H) 4.18 (d, J = 7.0 Hz, 2H) 7.69 (d, J = 5.3 Hz, 1H) | |
| | 7.92 (d, J = 5.3 Hz, 1H) 8.69 (s, 1H) | |
| 329 | 1H NMR (200 MHz, CHLOROFORM-D) d ppm 1.38 (s, 9H) 2.07 (s, 3H) 3.15 (t, | |
| | J = 7.0 Hz, 2H) 4.46 (t, J = 7.0 Hz, 2H) 7.10-7.19 (m, 2H) 7.22-7.36 (m, 3H) | |
| | 7.40 (dd, J = 7.9, 4.8 Hz, 1H) 8.56 (d, J = 7.9 Hz, 1H) 8.69, (d, J = 4.8 Hz, 1 | |
| 330 | 1H NMR (200 MHz, CHLOROFORM-D) d ppm 0.45-0.61 (m, 4H) 1.02-1.22 (m, 1H) | 102-104 |
| | 1.24 (s, 9H) 1.39 (s, 9H) 2.39 (s, 3H) 4.10 (d, J = 7.0 Hz, 2H) | |
| 331 | 1H NMR (300 MHz, CHLOROFORM-D) d ppm 0.49-0.60 (m, 4H), 1.09-1.20 (m, 1H), | 189-191 |
| | 1.39 (s, 9H), 1.83-2.02 (m, 2H), 2.12-2.42 (m, 4H), 2.39 (s, 1H), | |
| | 3.22-3.35 (m, 1H), 4.08 (d, J = 6.8 Hz, 2H) | |
| 332 | 1H NMR (200 MHz, CHLOROFORM-D) d ppm 0.39-0.65 (m, 4H) 1.02-2.07 (m, 12H) | 141-142 |
| | 1.39 (s, 9H) 2.38 (s, 3H) 4.08 (d, J = 7.0 Hz, 2H) | |
| 333 | 1H NMR (200 MHz, CHLOROFORM-D) d ppm 0.52-0.64 (m, 4H), 1.07-1.36 (m, 1H), | |
| | 1.44 (s, 9H), 2.47 (s, 3H), 4.15 (d, J = 7.0 Hz, 2H) | |
| 334 | 1H NMR (200 MHz, CHLOROFORM-D) d ppm 0.49-0.63 (m, 4H) 1.05-1.29 (m, 1H) | 93-94 |
| | 1.43 (s, 9H) 2.46 (s, 3H) 4.14 (d, J = 7.0 Hz, 2H) 6.21 (tt, J = 53.2, 5.7 Hz, | |
| 335 | 1H NMR (300 MHz, CHLOROFORM-D) d ppm 0.46-0.60 (m, 4H), 1.08-1.22 (m, 1H), | 102-103 |
| | 1.41 (s, 9H), 2.41 (s, 3H), 2.47-2.64 (m, 2H), 2.68-2.76 (m, 2H), | |
| | 4.08 (m, J = 6.8 Hz, 2H) | |
| 336 | 1H NMR (300 MHz, CHLOROFORM-D) d ppm 0.53-0.59 (m, 4H), 1.08-1.20 (m, 1H), | 147-148 |
| | 1.43 (s, 9H), 2.47 (s, 3H), 4.15 (d, J = 7.0 Hz, 2H) | |
| 337 | 1H NMR (200 MHz, CHLOROFORM-D) d ppm 0.46-0.59 (m, 4H) 1.05-1.36 (m, 2H) | 110-112 |
| | 1.37 (s, 9H) 1.60-1.74 (m, 1H) 2.07-2.20 (m, 1H) 2.38 (s, 3H) | |
| | 2.49-2.65 (m, 1H) 4.07 (d, J = 6.6 Hz, 2H) 7.08-7.32 (m, 5H) | |
| 338 | 1H NMR (300 MHz, CHLOROFORM-D) d ppm 1.42 (s, 9H), 2.44 (s, 3H), 3.29 (s, 3H), | 69-71 |
| | 3.70 (t, J = 5.2 Hz, 2H), 4.34 (t, J = 5.1 Hz, 2H) | |
| 339 | 1H NMR (300 MHz, CHLOROFORM-D) d ppm 0.35-0.51 (m, 4H), 1.01-1.16 (m, 1H), | 92-94 |
| | 1.37 (s, 9H), 2.36 (s, 3H), 3.76 (s, 2H), 4.01 (d, J = 7.0 Hz, 2H), | |
| | 7.14-7.22 (m, 1H), 7.22-7.38 (m, 4H) | |
| 340 | 1H NMR (300 MHz, CHLOROFORM-D) d ppm 0.43-0.54 (m, 4H), 1.04-1.18 (m, 1H), | 157-159 |
| | 1.38 (s, 9H), 2.38 (s, 3H), 3.97 (s, 2H), 4.07 (d, J = 7.0 Hz, 2H), | |
| | 6.91-6.94 (m, 2H), 7.13-7.17 (m, 1H) | |
| 341 | 1H NMR (300 MHz, CHLOROFORM-D) d ppm 0.21-0.54 (m, 4H), 0.97-1.08 (m, 1H), | 147-148 |
| | 1.36 (s, 9H), 2.22 (s, 3H), 2.35 (s, 3H), 3.92 (dd, J = 14.2, 6.5 Hz, 1H), | |
| | 4.08 (dd, J = 14.2, 7.5 Hz, 1H), 6.14 (s, 1H), 7.23-7.36 (m, 3H), 7.50 | |
| 342 | 1H NMR (300 MHz, CHLOROFORM-D) d ppm 0.32-0.47 (m, 4H), 0.99-1.10 (m, 1H), | 89-91 |
| | 1.37 (s, 9H), 2.36 (s, 3H), 3.75 (s, 6H), 3.78 (s, 2H), 3.96 (d, J = 7.0 Hz, | |
| | 2H), 6.69-6.88 (m, 3H) | |
| 343 | 1H NMR (200 MHz, CHLOROFORM-D) d ppm 1.42 (s, 9H), 2.37 (s, 3H), | |
| | 4.92-5.00 (m, 2H), 5.02-5.30 (m, 2H), 5.89-6.11 (m, 1H), 7.35-7.47 (m, 3H), | |
| | 8.26-8.35 (m, 2H) | |
| 344 | 1H NMR (200 MHz, CHLOROFORM-D) d ppm 1.44 (s, 9H) 2.44 (s, 3H) 3.34 (s, 3H) | 172-173 |
| | 3.81 (t, J = 5.5 Hz, 2H) 4.44 (t, J = 5.5 Hz, 2H) 7.54 (t, J = 7.5 Hz, 3H) | |
| | 7.71 (d, J = 7.5 Hz, 1H) 8.45 (d, J = 7.5 Hz, 1H) 8.56 (s, 1H) | |
| 345 | 1H NMR (300 MHz, CHLOROFORM-D) d ppm 1.43 (s, 9H), 2.44 (s, 3H), 3.34 (s, 3H), | 89-91 |
| | 3.81 (t, J = 5.4 Hz, 2H), 4.42 (t, J = 5.4 Hz, 2H), 7.26-7.33 (m, 1H), | |
| | 7.55-7.61 (m, 1H), 8.18-8.23 (m, 1H), 8.40-8.44 (m, 1H) | |
| 346 | 1H NMR (200 MHz, CHLOROFORM-D) d ppm 1.43 (s, 9H) 2.43 (s, 3H) 3.34 (s, 3H) | 119-121 |
| | 3.80 (t, J = 5.5 Hz, 2H) 4.41 (t, J = 5.5 Hz, 2H) 7.16 (t, J = 7.9 Hz, 1H) | |
| | 7.78 (d, J = 7.9 Hz, 1H) 8.23 (d, J = 7.9 Hz, 1H) 8.62 (s, 1H) | |
| 347 | 1H NMR (200 MHz, CHLOROFORM-D) d ppm 1.42 (s, 9H), 2.42 (s, 3H), 3.31 (s, 3H), | 68-69 |
| | 3.77 (t, J = 5.5 Hz, 2H), 4.37 (t, J = 5.5 Hz, 2H), 7.07-7.21 (m, 2H), | |
| | 8.02-8.14 (m, 1H) | |
| 348 | 1H NMR (200 MHz, CHLOROFORM-D) d ppm 1.43 (s, 9H), 2.43 (s, 3H), 3.32 (s, 3H), | 95-97 |
| | 3.78 (t, J = 5.3 Hz, 2H), 4.38 (t, J = 5.3 Hz, 2H), 6.92-7.04 (m, 1H), | |
| | 7.43-7.53 (m, 1H), 8.18-8.26 (m, 1H) | |
| 349 | 1H NMR (200 MHz, CHLOROFORM-D) d ppm 1.43 (s, 9H) 2.44 (s, 3H) 3.31 (s, 3H) | 161-163 |
| | 3.75 (t, J = 5.3 Hz, 2H) 4.37 (t, J = 5.3 Hz, 2H) 7.43-7.51 (m, 1H) 7.78- | |
| 350 | 1H NMR (200 MHz, CHLOROFORM-D) d ppm 1.43 (s, 9H) 2.43 (s, 3H) 3.34 (s, 3H) | 114-116 |
| | 3.80 (t, J = 5.5 Hz, 2H) 4.42 (t, J = 5.5 Hz, 2H) 5.39-6.34 (m, 1H) | |
| | 7.18-7.36 (m, 1H) 7.38-7.50 (m, 1H) 8.01-8.32 (m, 2H) | |
| 351 | 1H NMR (200 MHz, CHLOROFORM-D) d ppm 1.43 (s, 9H) 2.43 (s, 3H) 2.66 (s, 3H) | 106-107 |
| | 3.32 (s, 3H) 3.77 (t, J = 5.5 Hz, 2H) 4.37 (t, J = 5.5 Hz, 2H) 6.91-7.07 (m, | |
| | 1H) 7.08-7.23 (m, 1H) 7.72-7.90 (m, 1H) | |
| 352 | 1H NMR (200 MHz, CHLOROFORM-D) d ppm 1.14 (t, J = 7.0 Hz, 3H) 1.43 (s, 9H) | 91-92 |
| | 2.46 (s, 3H) 3.47 (q, J = 7.0 Hz, 2H) 3.84 (t, J = 5.5 Hz, 2H) 4.44 (t, J = 5.5 Hz, | |
| | 2H) 7.54 (t, J = 7.7 Hz, 1H) 7.71 (d, J = 7.7 Hz, 1H) 8.46 (d, J = 7.7 Hz, 1 | |
| 353 | 1H NMR (200 MHz, CHLOROFORM-D) d ppm 1.43 (s, 9H) 2.42 (s, 3H) 3.93 (s, 1H) | 147-149 |
| | 4.08 (t, J = 5.3 Hz, 2H) 4.48 (t, J = 5.3 Hz, 2H) 7.55 (t, J = 7.9 Hz, 1H) | |
| | 7.72 (d, J = 7.9 Hz, 1H) 8.42 (d, J = 7.9 Hz, 1H) 8.51 (s, 1H) | |
| 354 | 1H NMR (200 MHz, CHLOROFORM-D) d ppm 1.44 (s, 9H) 2.45 (s, 3H) 3.19 (t, | 76-78 |
| | J = 6.4 Hz, 2H) 4.37 (t, J = 6.4 Hz, 2H) 7.55 (t, J = 7.7 Hz, 1H) 7.71 (d, J = 7.7 Hz, | |
| | 1H) 8.46 (d, J = 7.7 Hz, 1H) 8.56 (s, 1H) | |
| 355 | 1H NMR (200 MHz, CHLOROFORM-d) d ppm 1.42 (s, 9H) 2.16-2.33 (m, 2H) | 129-130 |
| | 3.82-3.93 (m, 2H) 4.32-4.44 (m, 2H) 7.37-7.50 (m, 1H) 7.63-7.77 (m, 3H) | |
| | 7.79-7.88 (m, 2H) 8.35-8.44 (m, 1H) 8.51-8.55 (m, 1H) | |
| 356 | 1H NMR (200 MHz, CHLOROFORM-D) d ppm 0.53-0.71 (m, 4H) 1.16-1.45 (m, 1H) | 115-116 |
| | 1.37 (t, J = 7.0 Hz, 3H) 2.79 (s, 3H) 4.34 (q, J = 7.0 Hz, 2H) 4.30 (d, J = 7.9 Hz, | |
| | 2H) 7.58 (t, J = 7.7 Hz, 1H) 7.75 (d, J = 7.7 Hz, 1H) 8.45 (d, J = 7.7 Hz, 1 | |
| 357 | 1H NMR (200 MHz, CHLOROFORM-D) d ppm 0.63-1.01 (m, 4H) 1.16-1.41 (m, 1H) | 291-293 |
| | 2.34-2.73 (m, 3H) 3.53-3.87 (m, 2H) 7.01-7.24 (m, 1H) 7.30-7.59 (m, | |
| | 1H) 7.91-8.29 (m, 2H) | |
| 358 | 1H NMR (200 MHz, CHLOROFORM-D) d ppm 0.52-0.71 (m, 4H) 1.17-1.43 (m, 1H) | 212-214 |
| | 1.25 (d, J = 6.6 Hz, 6H) 2.78 (s, 3H) 4.10-4.35 (m, 1H) 4.29 (d, J = 7.0 Hz, | |
| | 2H) 5.47-5.65 (m, 1H) 7.58 (t, J = 7.7 Hz, 1H) 7.76 (d, J = 7.7 Hz, 1H) | |
| | 8.44 (d, J = 7.7 Hz, 1H) 8.55 (s, 1H) | |
| 359 | 1H NMR (200 MHz, CHLOROFORM-D) d ppm 0.54-0.70 (m, 4H) 1.17-1.45 (m, 1H) | 138-140 |
| | 2.49 (s, 3H) 3.13 (s, 6H) 4.25 (d, J = 7.0 Hz, 2H) 7.57 (t, J = 7.7 Hz, 1H) | |
| | 7.74 (d, J = 7.7 Hz, 1H) 8.44 (d, J = 7.7 Hz, 1H) 8.56 (s, 1H) | |
| 360 | 1H NMR (200 MHz, CHLOROFORM-D) d ppm 0.54-0.71 (m, 4H) 1.19-1.45 (m, 1H) | 125-127 |
| | 1.89-2.06 (m, 4H) 2.59 (s, 3H) 3.53-3.74 (m, 4H) 4.27 (d, J = 7.0 Hz, 2H) | |
| | 7.57 (t, J = 7.7 Hz, 1H) 7.74 (d, J = 7.7 Hz, 1H) 8.44 (d, J = 7.7 Hz, 1H) | |
| 361 | 1H NMR (200 MHz, CHLOROFORM-D) d ppm 0.54-0.71 (m, 4H) 1.14-1.46 (m, 1H) | 156-158 |
| | 2.43-2.57 (m, 4H) 2.58 (t, J = 6.2 Hz, 2H) 2.78 (s, 3H) 3.51 (q, J = 6.2 Hz, | |
| | 2H) 3.67-3.83 (m, 4H) 4.30 (d, J = 7.0 Hz, 2H) 6.31-6.49 (m, 1H) | |
| | 7.59 (t, J = 7.7 Hz, 1H) 7.76 (d, J = 7.7 Hz, 1H) 8.45 (d, J = 7.7 Hz, 1H) 8.55 (s, 1 | |
| 362 | 1H NMR (200 MHz, CHLOROFORM-d) d ppm 1.35 (6H, d, J = 5.27 Hz), 3.47-3.72 (1H, | |
| | m), 3.67 (3H, s), 7.84-7.95 (1H, m), 8.52-8.61 (1H, m), | |
| | 8.79-8.86 (1H, m), 9.49-9.58 (1H, m) | |
| 363 | 1H NMR (200 MHz, CHLOROFORM-D) d ppm 0.52-0.63 (m, 2H), 0.81-0.96 (m, 2H), | 139-141 |
| | 1.21-1.51 (m, 1H), 1.36 (d, J = 7.0 Hz, 6H), 3.45-3.67 (m, 1H), | |
| | 4.41 (d, J = 7.5 Hz, 2H), 8.25 (s 1H), 9.18 (s, 1H), 9.43 (s, 1H) | |
| 364 | 1H NMR (300 MHz, CHLOROFORM-D) d ppm 1.42 (t, J = 7.1 Hz, 3H), 2.36 (s, 3H), | 158-160 |
| | 4.33 (q, J = 7.0 Hz, 2H), 7.24-7.33 (m, 1H), 7.66-7.74 (m, 1H), 8.27- | |
| 365 | 1H NMR (300 MHz, CHLOROFORM-D) d ppm 0.49-0.67 (m, 4H) 1.20-1.38 (m, 1H) | 130-132 |
| | 2.39 (s, 3H) 4.21 (d, J = 7.0 Hz, 2H) 7.29 (t, J = 7.7 Hz, 1H) 7.70 (t, | |
| | J = 7.7 Hz, 1H) 8.29 (t, J = 7.7 Hz, 1H) | |
| 366 | 1H NMR (200 MHz, CHLOROFORM-D) d ppm 0.13-0.48 (m, 4H), 0.98-1.18 (m, 1H), | 161-162 |
| | 2.15 (s, 3H), 4.07 (d, J = 7.0 Hz, 2H), 7.27-7.41 (m, 2H), | |
| | 7.47-7.63 (m, 4H), 7.72 (d, J = 7.5 Hz, 1H), 8.48 (d, J = 7.5 Hz, 1H), 8.59 (s, 1H) | |
| 367 | 1H NMR (200 MHz, CHLOROFORM-D) d ppm 2.04 (s, 3H), 2.22 (s, 3H), 2.65 (s, 3H), | |
| | 4.39-4.47 (m, 2H), 5.10-5.30 (m, 2H), 5.79-6.01 (m, 1H), | |
| | 7.11-7.33 (m, 3H), 7.96-8.05 (m, 1H) | |
| 368 | 1H NMR (200 MHz, CHLOROFORM-D) d ppm 4.66-4.83 (m, 2H), 4.89-5.26 (m, 2H), | 141-143 |
| | 5.86-6.07 (m, 1H), 7.10-7.56 (m, 13H), 8.31-8.42 (m, 2H) | |
| 369 | 1H NMR (600 MHz, CHLOROFORM-d) d ppm 0.43-0.48 (m, 2H) 0.56-0.61 (m, 2H) | |
| | 1.13-1.20 (m, 1H) 1.26 (d, J = 7.3 Hz, 6H) 2.13 (s, 3H) 2.90-2.96 (m, | |
| | 1H) 3.70 (d, J = 7.3 Hz, 2H) 7.21-7.25 (m, 1H) 7.61-7.65 (m, 1H) 8.17- | |
| 370 | 1H NMR (200 MHz, CHLOROFORM-d) d ppm 0.42-0.68 (m, 4H) 1.14-1.36 (m, 1H) | 65-67 |
| | 1.31 (d, J = 7.0 Hz, 6H) 2.14 (s, 3H) 2.86-3.05 (m, 1H) 3.74 (d, J = 7.0 Hz, | |
| | 2H) 7.44-7.57 (m, 1H) 7.64-7.73 (m, 1H) 8.37-8.44 (m, 1H) 8.47- | |
| 371 | 1H NMR (300 MHz, CHLOROFORM-d) d ppm 0.43-0.64 (m, 4H) 1.08-1.24 (m, 1H) | 76-78 |
| | 1.29 (d, J = 7.0 Hz, 6H) 2.14 (s, 3H) 2.86-3.00 (m, 1H) 3.71 (d, J = 7.1 Hz, | |
| | 2H) 7.10-7.22 (m, 1H) 7.57-7.68 (m, 1H) 8.30-8.39 (m, 1H) | |
| 372 | 1H NMR (200 MHz, CHLOROFORM-d) d ppm 0.41-0.65 (m, 4H) 1.09-1.31 (m, 1H) | 118-120 |
| | 1.20 (d, J = 7.0 Hz, 6H) 2.11 (s, 3H) 2.80-2.97 (m, 1H) 3.68 (d, J = 7.0 Hz, | |
| | 2H) 3.90 (s, 3H) 6.93-7.01 (m, 1H) 7.53-7.61 (m, 1H) 8.03-8.07 | |
| 373 | 1H NMR (200 MHz, CHLOROFORM-D) d ppm 0.50-0.65 (m, 4H) 1.35-1.54 (m, 1H) | 115-117 |
| | 2.75 (s, 3H) 4.42 (d, J = 7.0 Hz, 2H) 7.20-7.54 (m, 6H) 7.70 (d, J = 7.0 Hz, | |
| | 1H) 8.21 (d, J = 7.9 Hz, 1H) | |
| 374 | 1H NMR (200 MHz, CHLOROFORM-D) d ppm 0.52-0.74 (m, 4H), 1.33-1.55 (m, 1H), | 156-158 |
| | 3.03 (s, 6H), 4.45 (d, J = 7.0 Hz, 2H), 6.86-6.97 (m, 1H), | |
| | 7.25-7.51 (m, 4H), 7.67-7.80 (m, 3H) | |
| 375 | 1H NMR (200 MHz, CHLOROFORM-D) d ppm 0.52-0.66 (m, 4H), 1.34-1.53 (m, 1H), | 181-183 |
| | 2.98 (s, 3H), 4.43 (d, J = 7.5 Hz, 2H), 7.18-7.57 (m, 4H), 7.73 (d, | |
| | J = 7.0 Hz, 1H), 8.42-8.51 (m, 1H), 8.54-8.62 (m, 1H) | |
| 376 | 1H NMR (200 MHz, CHLOROFORM-D) d ppm 0.53-0.65 (m, 4H) 1.34-1.52 (m, 1H) | 160-161 |
| | 2.36 (s, 3H) 2.41 (s, 3H) 2.74 (s, 3H) 4.38 (d, J = 7.0 Hz, 2H) | |
| | 7.17-7.42 (m, 3H) 7.19 (s, 1H) 7.45 (s, 1H) 8.19 (d, J = 7.9 Hz, 1H) | |
| 377 | 1H NMR (200 MHz, CHLOROFORM-D) d ppm 0.55-0.65 (m, 4H), 1.32-1.50 (m, 1H), | 143-145 |
| | 2.74 (s, 3H), 4.39 (d, J = 7.0 Hz, 2H), 7.12-7.46 (m, 6H), 8.17-8.25 | |
| 378 | 1H NMR (300 MHz, CHLOROFORM-D) d ppm 0.50-0.59 (m, 2H), 0.62-0.72 (m, 2H), | 146-148 |
| | 1.45-1.61 (m, 1H), 2.75 (s, 3H), 4.98 (d, J = 7.1 Hz, 2H), | |
| | 7.17-7.48 (m, 5H), 7.60 (d, J = 7.6 Hz, 1H), 8.22 (d, J = 7.8 Hz, 1H) | |
| 379 | 1H NMR (300 MHz, CHLOROFORM-D) d ppm 0.49-0.69 (m, 4H), 1.19-1.35 (m, 1H), | 133-134 |
| | 2.74 (s, 3H), 2.86 (s, 3H), 4.78 (d, J = 6.5 Hz, 2H), 7.15-7.40 (m, 5H), | |
| | 7.51-7.59 (m, 1H), 8.19 (d, J = 6.2 Hz, 1H) | |
| 380 | 1H NMR (300 MHz, CHLOROFORM-D) d ppm 0.50-0.65 (m, 4H), 1.40-1.52 (m, 1H), | 146-148 |
| | 2.74 (s, 3H), 4.57 (d, J = 8.5 Hz, 2H), 6.92-7.03 (m, 1H), | |
| | 7.19-7.43 (m, 4H), 8.22 (d, J = 7.5 Hz, 1H) | |
| 381 | 1H NMR (200 MHz, CHLOROFORM-D) d ppm 0.52-0.65 (m, 4H) 1.18-1.42 (m, 1H) | 159-160 |
| | 1.79-2.04 (m, 4H) 2.56-2.70 (m, 4H) 4.15 (d, J = 7.0 Hz, 2H) 7.55 (t, | |
| | J = 7.7 Hz, 1H) 7.71 (d, J = 7.7 Hz, 1H) 8.46 (d, J = 7.7 Hz, 1H) 8.57 (s, 1H) | |
| 382 | 1H NMR (200 MHz, CHLOROFORM-d) d ppm 0.56-0.69 (m, 4H) 1.28-1.50 (m, 1H) | 156-158 |
| | 4.42 (d, J = 7.5 Hz, 2H) 7.24-7.47 (m, 2H) 7.62-7.82 (m, 2H) | |
| | 8.29-8.42 (m, 1H) 8.49-8.57 (m, 1H) | |

TABLE 10

| Compound | |
|---|---|
| No. | m.p. (° C.) or NMR |
| 383 | 178.5-180.5 |
| 384 | 141.5-142.5 |
| 385 | 1H NMR (200 MHz, CHLOROFORM-D) d ppm 1.35 (s, |
| | 9 H), 2.25 (s, 3 H), 3.43 (s, 3 H), 7.38- |
| | 7.52 (m, 3 H), 7.94-8.02 (m, 2 H) |
| 386 | 173-174.5 |
| 387 | 190-192 |
| 388 | 1H NMR (200 MHz, CHLOROFORM-D) d ppm 0.29-0.51 |
| | (m, 4 H), 0.89-1.12 (m, 1 H), 1.36 (s, |
| | 9 H), 2.29 (s, 3 H), 3.84 (d, J = 7.0 Hz, 2 |
| | H), 7.37-7.54 (m, 3 H), 7.91-8.01 (m, 2 H) |
| 389 | 198-199.5 |
| 390 | 121-124 |
| 391 | 122-123 |
| 392 | 125-127 |
| 393 | 1H NMR (200 MHz, CHLOROFORM-D) d ppm 0.29-0.54 |
| | (m, 4 H), 0.99-1.19 (m, 1 H), 1.36 (s, |
| | 9 H), 2.31 (s, 3 H), 3.85 (d, J = 6.6 Hz, 2 |
| | H), 7.29-7.50 (m, 3 H), 8.15-8.26 (m, 1 H) |
| 394 | 126-128 |
| 395 | 159-160 |
| 396 | 140.5-141.5 |
| 397 | 132-134 |
| 398 | 100-102 |
| 399 | 104-105 |
| 400 | 130-131 |
| 401 | 149-151 |
| 402 | 132-135 |
| 403 | 110-112 |
| 404 | 150-152 |
| 405 | 69-72 |
| 406 | 127-129 |
| 407 | 124-126 |
| 408 | 126.5-128 |
| 409 | 136-138 |
| 410 | 136-138 |
| 411 | 129-131 |
| 412 | 1H NMR (200 MHz, CHLOROFORM-D) d ppm 0.31-0.53 |
| | (m, 4 H), 0.94-1.11 (m, 1 H), 1.36 (s, |
| | 9 H), 2.30 (s, 3 H), 3.84 (d, J = 7.0 Hz, |
| | 2 H), 7.11-7.23 (m, 1 H), 7.31-7.44 (m, 2 H), |
| | 7.87-7.95 (m, 2 H) |
| 413 | 132.5-134 |
| 414 | 1H NMR (200 MHz, CHLOROFORM-D) d ppm 0.29-0.58 |
| | (m, 4 H), 0.96-1.13 (m, 1 H), 1.36 (s, |
| | 9 H), 2.30 (s, 3 H), 2.67 (s, 3 H), 7.00-7.12 |
| | (m, 1 H), 7.16-7.27 (m, 1 H), 7.74-7.84 |
| 415 | |
| 416 | |
| 417 | 124-125.5 |
| 418 | 1H NMR (200 MHz, CHLOROFORM-D) d ppm 0.30-0.44 |
| | (m, 4 H), 0.90-1.11 (m, 1 H), 1.38 (s, |
| | 9 H), 2.37 (s, 3 H), 3.80 (s, 3 H), 3.84 (d, |
| | J = 7.0 Hz, 2 H), 6.70-6.84 (m, 2 H), 7.87- |
| 419 | 158-160 |
| 420 | 97-99 |
| 421 | 86-88 |
| 422 | 145.5-147 |
| 423 | 146-147.5 |
| 424 | 85-87 |
| 425 | 145-146 |
| 426 | 156-158.5 |
| 427 | 207.5-208.5 |
| 428 | 1H NMR (200 MHz, CHLOROFORM-D) d ppm 1.35 (s, |
| | 9 H), 2.15 (s, 3 H), 5.21 (s, 2 H), 7.10- |
| | 7.18 (m, 1 H), 7.23-7.57 (m, 6 H), 7.84-7.92 (m, 2 H) |
| 429 | 94-95 |
| 430 | 186.5-188 |
| 431 | 273-274.5 |
| 432 | 139-142 |
| 433 | 171-173 |
| 434 | 1H NMR (200 MHz, CHLOROFORM-D) d ppm 1.20 (t, |
| | J = 7.3 Hz, 3 H), 1.36 (s, 9 H), 2.18 (s, 3 |
| | H), 4.13 (q, J = 7.0 Hz, 2 H), 4.68 (s, |
| | 2 H), 7.36-7.53 (m, 3 H), 7.88-7.95 (m, 2 H) |
| 435 | 163.5-165 |
| 436 | 103-104 |
| 437 | 75-79 |
| 438 | 194-195 |
| 439 | 68-69 |
| 440 | 111-112 |
| 441 | 74-78 |
| 442 | 1H NMR (200 MHz, CHLOROFORM-D) d ppm 0.19-0.43 |
| | (m, 4 H), 0.91-1.12 (m, 1 H), 2.14 (s, |
| | 3 H), 2.74 (t, J = 6.2 Hz, 4 H), 3.77 (t, |
| | J = 6.2 Hz, 2 H), 3.80 (d, J = 7.0 Hz, 2 H), 7.45- |
| | 7.66 (m, 3 H), 7.82-8.01 (m, 2 H), 8.28-8.38 |
| | (m, 2 H), 8.85-8.93 (m, 1 H) |
| 443 | 135-136.5 |
| 444 | 144-146 |
| 445 | 170-172 |
| 446 | 1H NMR (200 MHz, CHLOROFORM-D) d ppm 4.63-4.75 |
| | (m, 2 H), 5.05-5.27 (m, 2 H), 5.65-5.96 (m, |
| | 1 H), 7.10-7.64 (m, 8 H), 7.88-8.06 (m, 2 H) |
| 447 | 174-175.5 |

TABLE 11

| Compound No. | 1H-NMR | MASS | m.p. (° C.) |
|---|---|---|---|
| 448 | 1H NMR (200 MHz, CHLOROFORM-d) δ ppm 1.75-2.17 (m, 6 H) 2.31 | ESI(Pos) 370 (M + H)⁺ | 137-133 |
| | (s, 3 H) 3.59 (s, 3 H) 4.36 (d, J = 7.0 Hz, 2 H) 6.99 (s, 1 H) 7.13-7.29 (m, 1 | | |
| | H) 7.51-7.63 (m, 1 H) 8.11-8.24 (m, 1 H) | | |
| 449 | 1H NMR (200 MHz, CHLOROFORM-d) δ ppm −0.03-0.12 (m, 2 H) 0.38- | ESI(Pos) 370 (M + H)⁺ | 97-98 |
| | 0.55 (m, 2 H) 0.56-0.75 (m, 1 H) 1.63 (q, J = 7.0 Hz, 2 H) 2.32 (s, 3 H) | | |
| | 3.64 (s, 3 M) 4.37 (t, J = 7.0 Hz, 2 H) 6.98 (s, 1 H) 7.12-7.25 (m, 1 H) 8.09- | | |
| | 8.26 (m, 1 H) | | |
| 450 | 1H NMR (200 MHz, CHLOROFORM-d) δ ppm 1.88-2.31 (m, 4 H). 2.35 | ESI(Pos) 412 (M + H)⁺ | amorphous |
| | (s, 3 H), 3.63 (s, 3 H) 4.39 (t, J = 7.0 Hz, 2 H), 7.00 (s, 1 H), 7.13-7.29 (m, | | |
| | 1 H), 7.52-7.64 (m, 5 H), 8.09-8.23 (m, 1 H) | | |
| 451 | 1H NMR (200 MHz, CHLOROFORM-d) δ ppm 2.33 (s, 3 H) 3.30 (s, 3 H) | ESI(Pos) 360 (M + H)⁺ | 144-145 |
| | 3.63-3.74 (m, 5 H) 4.45 (t, J = 4.6 Hz, 2 H) 6.95 (s, 1 H) 7.15-7.25 (m, 1 | | |
| | H) 7.51-7.62 (m, 1 H) 8.11-8.22 (m, 1 H) | | |
| 452 | 1H NMR (200 MHz, CHLOROFORM-d) δ ppm 1.05-1.22 (m, 3 H) 2.33 | ESI(Pos) 374 (M + H)⁺ | 136-137 |
| | (s, 3 H) 3.33-3.53 (m, 2 H) 3.72 (s, 3 H) 3.63-3.81 (m, 2 H) 4.36-4.53 | | |
| | (m, 2 H) 6.96 (s, 1 H) 7.10-7.25 (m, 1 H) 7.49-7.62 (m, 1 H) 8.07-8.25 | | |
| | (m, 1 H) | | |
| 453 | 1H NMR (200 MHz, CHLOROFORM-d) δ ppm 1.89-2.17 (m, 2 H) 2.33 | ESI(Pos) 374 (M + H)⁺ | 88-90 |
| | (s, 3 H) 3.32 (s, 3 H) 3.38 (t, J = 5.7 Hz, 2 H) 3.65 (s, 3 H) 4.38 (t, J = 6.8 Hz, | | |
| | 2 H) 6.98 (s, 1 H) 7.13-7.25 (m, 1 H) 7.51-7.62 (m, 1 H) 8.12-8.23 (m, | | |
| | 1 H) | | |
| 454 | 1H NMR (200 MHz, CHLOROFORM-d) δ ppm 1.08 (d, J = 6.2 Hz, 6 H) | ESI(Pos) 388 (M + H)⁺ | 88-90 |
| | 2.33 (s, 3 H) 3.43-3.59 (m, 1 H) 3.73 (t, J = 4.4 Hz, 2 H) 3.73 (s, 3 H) 4.45 | | |
| | (t, J = 4.4 Hz, 2 H) 6.96 (s, 1 H) 7.14-7.25 (m, 1 H) 7.50-7.61 (m, 1 H) | | |
| | 8.11-8.22 (m, 1 H) | | |
| 455 | 1H NMR (200 MHz, CHLOROFORM-d) δ ppm 2.34 (s, 3H) 2.51-2.71 (m, | ESI(Pos) 420 (M + H)⁺ | amorphous |
| | 1 H) 3.02-3.32 (m, 3 H) 3.62 (m, 1H) 3.65 (s, 3H) 4.38-4.54 (m, 1 H) | | |
| | 5.04-5.27 (m, 1 H) 7.04 (s, 1H) 7.25 (m, 1H) 7.55-7.67 (m, 1 H) 8.04- | | |
| | 8.16 (m, 1 H) | | |
| 456 | 1H NMR (600 MHz, CHLOROFORM-d) δ ppm 2.58-2.61 (m, 3 H), 5.15 | ESI(Pos) 470 (M + H)⁺ | amorphous |
| | (d, 1 H, J = 10.55 Hz), 5.53-5.55 (m, 2 H) 5.65-5.74 (m, 1 H), 5.74-5.78 | | |
| | (m, 2 H), 7.07-7.10 (m, 1 H), 7.16-7.18 (m, 1 H), 7.32-7.36 (m, 2 H), | | |
| | 7.38-7.43 (m, 1 H); 7.71-7.76 (m, 1 H), 8.15-8.19 (m, 1 H) | | |
| 457 | 1H NMR (200 MHz, CHLOROFORM-d) δ ppm 0.46-0.64 (m, 4 h), 0.96-1.17 | ESI(Pos) 236 (M + H)⁺ | 122-124 |
| | (m, 1 H), 1.42 (s, 9 H), 3.81 (s, 3 H), 4.17 (d, J = 7.0 Hz, 2 H), 6.60 (s, 1 H), 8.78 (s, 1 H) | | |
| 458 | 1H NMR (200 MHz, CHLOROFORM-d) δ ppm 0.51-0.63 (m, 4 H) 0.93- | ESI(Pos) 304 (M + H)⁺ | 122-124 |
| | 1.14 (m, 1 H) 1.43 (s, 9 H) 3.90 (s, 3 H) 4.21 (d, J = 7.0 Hz, 2 H) 6.94 (s, 1 | | |
| | H) | | |
| 459 | 1H NMR (200 MHz, CHLOROFORM-d) δ ppm 0.41-0.83 (m, 4 H) 1.00- | ESI(Pos) 354 (M + H)⁺ | 113-114 |
| | 1.18 (m, 1 H) 1.26 (d, J = 7.0 Hz, 6 H) 1.44 (s, 9 H) 3.79 (s, 3 H) 3.79- | | |
| | 3.96 (m, 1 H) 4.20 (d, J = 6.6 Hz, 2 H) 7.06 (s, 1 H) 7.08-7.35 (m, 4 H) 7.57- | | |
| | 7.65 (m, 1 H) | | |
| 460 | 1H NMR (200 MHz, CHLOROFORM-d) δ ppm 0.39-0.82 (m, 4 H) 0.97- | ESI(Pos) 380 (M + H)⁺ | 83-85 |
| | 1.18 (m, 1 H) 1.43 (s, 9 H) 3.81 (s, 3 H) 4.18 (d, J = 7.0 Hz, 2 H) 7.02 (s, 1 | | |
| | H) 7.24-7.79 (m, 4 H) | | |
| 461 | 1H NMR (200 MHz, CHLOROFORM-d) δ ppm 0.51-0.89 (m, 4 H) 1.00- | ESI(Pos) 380 (M + H)⁺ | 97-99 |
| | 1.29 (m, 1 H) 1.45 (s, 9 H) 3.85 (s, 3 H) 4.30 (d, J = 7.0 Hz, 2 H) 7.10 (s, 1 | | |
| | H) 7.40-7.55 (m, 1 H) 7.58-7.68 (m, 1 H) 8.35-8.47 (m, 1 H) 8.48- | | |
| | 8.58 (m, 1 H) | | |
| 462 | 1H NMR (200 MHz, CHLOROFORM-d) δ ppm 0.52-0.65 (m, 4 H) 1.02- | ESI(Pos) 428 (M + H)⁺ | 110-111 |
| | 1.23 (m, 1 H) 1.44 (s, 9 H) 3.84 (s, 3 H) 4.29 (d J = 6.6 Hz, 2 H) 5.81- | | |
| | 6.22 (m, 1 H) 7.10 (s, 1 H) 7.16-7.29 (m, 1 H) 7.32-7.44 (m, 1 H) 8.07- | | |
| | 8.20 (m, 2 H) | | |
| 463 | 1H NMR (200 MHz, CHLOROFORM-d) δ ppm 0.49-0.61 (m, 4 H) 1.01- | ESI(Pos) 380 (M + H)⁺ | 158-159 |
| | 1.17 (m, 1 H) 1.45 (s, 9 H) 3.85 (s, 3 H) 4.22 (d, J = 6.6 Hz, 2 H) 7.08 (s, 1 | | |
| | H) 710-7.22 (m, 1 H) 7.33-7.41 (m, 1 H) 7.47-7.55 (m, 1 H) | | |
| 464 | 1H NMR (200 MHz, CHLOROFORM-d) δ ppm 0.54-0.61 (m, 4 H) 1.02- | ESI(Pos) 408 (M + H)⁺ | 127-128 |
| | 1.18 (m, 1 H) 1.44 (s, 9 H) 3.85 (s, 3 H) 4.26 (d, J = 7.0 Hz, 2 H) 6.94- | | |
| | 7.05 (m, 1 H) 7.07 (s, 1 H) 7.46-7.56 (m, 1 H) 7.83-7.93 (m, 1 H) | | |
| 465 | 1H NMR (200 MHz, CHLOROFORM-d) δ ppm 0.47-0.62 (m, 4 H) 1.00- | ESI(Pos) 404 (M + H)⁺ | 153-155 |
| | 1.19 (m, 1 H) 1.44 (s, 9 H) 2.43 (s, 3 H) 3.82 (s, 3 H) 4.20 (d, J = 7.0 Hz, 2 | | |
| | H) 7.08 (s, 3 H) 7.11-7.19 (m, 2 H) 7.29-7.40 (m, 1 H) | | |
| 466 | 1H NMR (200 MHz, CHLOROFORM-d) δ ppm 0.49-0.60 (m, 4 H) 1.01- | ESI(Pos) 452 (M + H)⁺ | 147-148 |
| | 1.17 (m, 1 H) 1.45 (s, 9 H) 2.48 (s, 3 H) 3.82 (s, 3 H) 4.22 (d, J = 7.0 Hz, 2 H) | | |
| | 7.06 (s, 1 H) 7.08-7.30 (m, 3 H) | | |
| 467 | 1H NMR (200 MHz, CHLOROFORM-d) δ ppm 0.43-0.65 (m, 4 H) 0.98- | ESI(Pos) 380 (M + H)⁺ | 127-128 |
| | 1.21 (m, 1 H) 1.45 (s, 9 H) 3.82 (s, 3H) 4.19 (d, J = 6.6 Hz, 2 H) 7.03-7.15 | | |
| | (m, 2 H) 7.24-7.33 (m, 1 H) 7.49-7.57 (m, 1 H) | | |
| 468 | 1H NMR (200 MHz, CHLOROFORM-d) δ ppm 0.46-0.65 (m, 4 H) 1.00- | ESI(Pos) 404 (M + H)⁺ | 138-139 |
| | 1.17 (m, 1 H) 1.45 (s, 9 H) 2.60 (s, 3 H) 3.83 (s, 3 H) 4.21 (d, J = 6.2 Hz, 2 H) | | |
| | 6.96-7.09 (m, 1 H) 7.06 (s, 1 H) 7.45-7.60 (m, 2 H) | | |
| 469 | 1H NMR (200 MHz, CHLOROFORM-d) δ ppm 0.43-0.64 (m, 4 H) 0.96- | ESI(Pos) 452 (M + H)⁺ | 138-140 |
| | 1.17 (m, 1 H) 1.45 (s, 9 H) 2.63 (s, 3 H) 3.82 (s, 3 H) 4.20 (d, J = 7.0 Hz, 2 H) | | |
| | 6.79-6.90 (m, 1 H) 7.05 (s, 1 H) 7.53-7.61 (m, 1 H) 7.73-7.80 (m, 1 H) | | |
| 470 | 1H NMR (200 MHz, CHLOROFORM-d) δ ppm 0.43-0.65 (m, 4 H) 0.98- | ESI(Pos) 394 (M + H)⁺ | 90-91 |
| | 1.16 (m, 1 H) 1.45 (s, 9 H) 2.59-2.66 (m, 3 H) 3.83 (s, 3 H) 4.20 (d, | | |
| | J = 7.0 Hz, 2 H) 7.06 (s, 1 H) 7.17-7.28 (m, 1 H) 7.51-7.58 (m, 1 H) 7.68- | | |
| | 7.77 (m, 1 H) | | |
| 471 | 1H NMR (200 MHz, CHLOROFORM-d) δ ppm 0.50-0.60 (m, 4 H) 1.02- | ESI(Pos) 404 (M + H)⁺ | 153-154 |
| | 1.19 (m, 1 H) 1.43 (s, 9 H) 2.31 (s, 3 H) 3.81 (s, 3 H) 4.24 (d, J = 7.0 Hz, 2 | | |
| | H) 7.03-7.11 (m, 1 H) 7.09 (s, 1 H) 7.36-7.40 (m, 1 H) 7.59-7.67 (m, 1 | | |
| | H) | | |
| 472 | 1H NMR (200 MHz, CHLOROFORM-d) δ ppm 0.50-0.62 (m, 4 H) 0.97- | ESI(Pos) 376 (M + H)⁺ | 128-129 |
| | 1.20 (s, 1 M) 1.43 (s, 9 H) 3.80 (s, 3 H) 3.85 (s, 3 H) 4.19 (d, J = 6.6 Hz, 2 | | |
| | H) 6.84-6.93 (m, 2 H) 7.13 (s, 1 H) 7.60-7.69 (m, 1 H) | | |
| 473 | 1H NMR (200 MHz, CHLOROFORM-d) δ ppm 0.51-0.63 (m, 4 H) 1.02- | ESI(Pos) 380 (M + H)⁺ | 164-165 |
| | 1.18 (m, 1 H) 1.45 (s, 9 H) 3.85 (s, 3 H) 4.24 (d, J = 7.03 Hz, 2 H) 7.08 (s, | | |
| | 1 H) 7.13-7.21 (m, 1 H) 7.24-7.32 (m, 1 H) 7.66-7.75 (m, J = 2.64 Hz, 1 | | |
| | H) | | |
| 474 | 1H NMR (200 MHz, CHLOROFORM-d) δ ppm 0.51-0.65 (m, 4 H) 1.01- | ESI(Pos) 398 (M + H)⁺ | 104-106 |
| | 1.19 (m, 1 H) 1.45 (s, 9 H) 3.86 (s, 3 H) 4.26 (d, J = 7.0 H) 7.07 (s, 1 H) | | |
| | 7.13-7.25 (m, 1 H) 7.49-7.63 (m, 1 H) 8.07-8.22 (m, 1 H) | | |
| 475 | 1H NMR (200 MHz, CHLOROFORM-d) δ ppm 0.53-0.65 (m, 4 H) 1.03- | ESI(Pos) 398 (M + H)⁺ | 124-126 |
| | 1.18 (m, 1 H) 1.45 (s, 9 H) 3.86 (s, 3 H) 4.27 (d, J = 7.0 Hz, 2 H) 7.06-7.19 | | |
| | (m, 1 H) 7.10 (s, 1 H) 7.50-7.60 (m, 1 H) 8.27-8.35 (m, 1 H) | | |
| 476 | 1H NMR (200 MHz, CHLOROFORM-d) δ ppm 0.47-0.62 (m, 4 H) 1.00- | ESI(Pos) 414 (M + H)⁺ | 145-148 |
| | 1.16 (m, 1 H) 1.45 (s, 9 H) 3.85 (s, 3 H) 4.21 (d, J = 7.0 Hz, 2 H) 7.07 (s, 1 H) | | |
| | 7.27-7.37 (m, 1 H) 7.57-7.64 (m, 1 H) 7.69-7.77 (m, 1 H) | | |
| 477 | 1H NMR (200 MHz, CHLOROFORM-d) δ ppm 0.50-0.66 (m, 4 H) 1.02- | ESI(Pos) 393 (M + H)⁺ | 156-157 |
| | 1.17 (m, 1 H) 1.45 (s, 9 H) 2.68 (s, 3 H) 3.84 (s, 3 H) 4.24 (d, J = 6.6 Hz, 2 | | |
| | H) 7.06 (s, 1 H) 7.21-7.29 (m, 1 H) 7.40-7.47 (m, 1 H) 8.13-8.17 (m, 1 | | |
| | H) | | |
| 478 | 1H NMR (200 MHz, CHLOROFORM-d) δ ppm 0.51-0.65 (m, 4 H) 0.98- | ESI(Pos) 410 (M + H)⁺ | 99-101 |
| | 1.19 (m, 1 H) 1.43 (s, 9 H) 3.82 (s, 3 H) 3.80 (s, 3 H) 4.20 (d, J = 7.0 Hz, 2 | | |
| | H) 6.91-7.01 (m, 1 H) 7.12 (s, 1 H) 7.46-7.55 (m, 1 H) 7.94-8.01 (m, 1 | | |
| | H) | | |
| 479 | 1H NMR (200 MHz, CHLOROFORM-d) δ ppm 0.45-0.66 (m, 4 H) 1.00- | ESI(Pos) 344 (M + H)⁺ | 161-162 |
| | 1.20 (m, 1 H) 1.44 (s, 9 H) 2.58 (s, 3 H) 3.82 (s, 3 H) 4.22 (d, J = 7.0 Hz, 2 H) | | |
| | 6.82-6.98 (m, 1 H) 7.03-7.15 (m, 2 H) 7.50-7.61 (m, 1 H) | | |
| 480 | 1H NMR (200 MHz, CHLOROFORM-d) δ ppm 0.53-0.63 (m, 4 H) 1.03- | ESI(Pos) 360 (M + H)⁺ | 135-136 |
| | 1.25 (m, 1 H) 1.43 (s, 9 H) 2.40 (s, 3 H) 3.82 (s, 3 H) 4.28 (d, J = 7.0 Hz, 2 | | |
| | H) 7.17-7.29 (m, 1 H) 7.97-8.06 (m, 1 H) 8.19-8.24 (m, 1 H) | | |
| 481 | 1H NMR (200 MHz, CHLOROFORM-d) δ ppm 1.43 (s, 9 H) 1.82-2.08 | ESI(Pos) 394 (M + H)⁺ | 152.5-154 |
| | (m, 8 H) 2.56-2.75 (m, 1 H) 3.73 (s, 3 H) 4.34 (d, J = 7.03 Hz, 2 H) 7.08 (s, | | |
| | 1 H) 7.17 (t, J = 7.91 Hz, 1 H) 7.38 (dd, J = 7.91, 1.76 Hz, 1 H) 7.55 (dd, | | |
| | J = 7.47, 1.76 Hz, 1 H) | | |
| 482 | 1H NMR (200 MHz, CHLOROFORM-d) δ ppm 1.42 (s, 9 H) 1.83-2.11 | ESI(Pos) 412 (M + H)⁺ | amorphous |
| | (m, 8 H) 2.55-2.77 (m, 1 H) 3.74 (s, 3 H) 4.38 (d, J = 7.0 Hz, 2 H) 7.07 (s, 1 | | |
| | H) 7.15-7.26 (m, 1 H) 7.52-7.62 (m, 1 H) 8.12-8.25 (m, 1 H) | | |
| 483 | 1H NMR (600 MHz, CHLOROFORM-d) δ ppm 1.39-1.46 (m, 9 H) 1.82- | ESI(Pos) 408 (M + H)⁺ | 133-134 |
| | 1.92 (m, 4 H) 1.97-2.06 (m, 2 H) 2.59-2.68 (m, 1 H) 2.64 (s, 3 H) 3.72 | | |
| | (s, 3 H) 4.31 (s, 2 H) 7.04 (s, 1 H) 7.20-7.24 (m, J = 8.25 Hz, 1 H) 7.52- | | |
| | 7.59 (m, 1 H) 7.72-7.80 (m, 1 H) | | |
| 484 | 1H NMR (200 MHz, CHLOROFORM-d) δ ppm 1.44 (s, 8 H) 1.48-1.80 | ESI(Pos) 408 (M + H)⁺ | 168-169 |
| | (m, J = 11.43 Hz, 8 H) 2.13-2.43 (m, 1 H) 3.76 (s, 3 H) 4.23 (4 J = 7.91 Hz, | | |
| | 2 H) 7.09 (s, 1 H) 7.16 (t, J = 7.91 Hz, 1 H) 7.34-7.41 (m, 1 H) 7.53 (dd, | | |
| | J = 7.69, 1.54 Hz, 1 H) | | |
| 485 | 1H NMR (200 MHz, CHLOROFORM-d) δ ppm 1.37-1.80 (m, 8 H) 1.39- | ESI(Pos) 426 (M + H)⁺ | 140.5-141.5 |
| | 1.49 (m, 9 H) 2.18-2.40 (m, 1 H) 3.77 (s, 3 H) 4.27 (d, J = 7.47 Hz, 2 H) | | |
| | 7.08 (s, 1 H) 7.20 (t, J = 7.69 Hz, 1 H) 7.50-7.63 (m, 1 H) 8.06-8.24 (m, 1 | | |
| | H) | | |
| 486 | 1H NMR (200 MHz, CHLOROFORM-d) δ ppm 1.11 (t, J = 7.0 Hz, 3 H) 1.44 | ESI(Pos) 398 (M + H)⁺ | 145-146 |
| | (s, 9 H) 3.41 (q, J = 7.0 Hz, 2 H) 3.69 (t, J = 4.7 Hz, 2 H) 3.88 (s, 3 H) 4.43 (t, | | |
| | J = 4.7 Hz, 2 H) 7.03 (s, 1 H) 7.11-7.21 (m, 1 H) 7.33-7.41 (m, 1 H) 7.51- | | |
| | 7.58 (m, 1 H) | | |
| 487 | 1H NMR (600 MHz, CHLOROFORM-d) δ ppm 1.10 (t, J = 8.9 Hz, 3 H) | ESI(Pos) 422 (M + H)⁺ | 166-167 |
| | 1.42 (s, 9 H) 2.42 (s, 3 H) 3.39 (q, J = 6.9 Hz, 2 H) 3.69 (t, J = 4.6 Hz, 2 H) | | |
| | 3.85 (s, 3 H) 4.42 (br. s., 2 H) 7.02 (s, 1 H) 7.11-7.17 (m, 2 H) 7.35-7.39 | | |
| | (m, 1 H) | | |
| 488 | 1H NMR (200 MHz, CHLOROFORM-d) δ ppm 1.12 (t, J = 7.0 Hz, 3 H) | ESI(Pos) 416 (M + H)⁺ | 95-96 |
| | 1.43 (s, 9 H) 3.43 (q, J = 7.0 Hz, 2 H) 3.73 (t, J = 4.8 Hz, 2 H) 3.89 (s, 3 H) | | |
| | 4.47 (t, J = 4.8 Hz, 2 H) 7.02 (s, 1 H) 7.14-7.25 (m, 1 H) 7.51-7.61 (m, 1 | | |
| | H) 8.10-8.23 (m, 1 H) | | |
| 489 | 1H NMR (600 MHz, CHLOROFORM-d) δ ppm 1.11 (t, J = 6.88 Hz, 3 H) | ESI(Pos) 412 (M + H)⁺ | 132-133 |
| | 1.43 (s, 9 H) 2.63 (s, 3 H) 3.41 (q, J = 6.88 Hz, 2 H) 3.67 (t, J = 4.81 Hz, 2 H) | | |
| | 3.87 (s, 3 H) 4.40-4.47 (m, 2 H) 7.01 (s, 1 H) 7.22 (dd, J = 7.79 Hz, 1 H) | | |
| | 7.54 (d, J = 7.79 Hz, 1 H) 7.75 (d, J = 7.79 Hz, 1 H) | | |
| 490 | 1H NMR (200 MHz, CHLOROFORM-d) δ ppm 1.12 (t, J = 7.0 Hz, 3 H) | ESI(Pos) 378 (M + H)⁺ | 127-128 |
| | 1.43 (s, 9 H) 2.59 (s, 3 H) 3.42 (q, J = 7.0 Hz, 2 H) 3.68 (t, J = 4.6 Hz, 2 H) | | |
| | 3.86 (s, 3 H) 4.42 (t, J = 4.8 Hz, 2 H) 7.02 (s, 1 H) 7.04-7.14 (m, 1 H) 7.25- | | |
| | 7.33 (m, 1 H) 7.54-7.60 (m, 1 H) | | |
| 491 | 1H NMR (200 MHz, CHLOROFORM-d) δ ppm 1.12 (t, J = 6.8 Hz, 3 H) | ESI(Pos) 422 (M + H)⁺ | 125-126 |
| | 1.43 (s, 9 H) 2.57-2.65 (m, 3 H) 3.41 (q, J = 6.8 Hz, 2 H) 3.68 (t, J = 4.8 Hz, | | |
| | 2 H) 4.42 (t, J = 4.8 Hz, 2 H) 6.95-7.06 (m, 1 H) 7.02 (s, 1 H) 7.43-7.53 | | |
| | (m, 1 H) 7.55-7.64 (m, 1 H) | | |
| 492 | 1H NMR (600 MHz, CHLOROFORM-d) δ ppm 1.05 (d, J = 6.0 Hz, 6 H), | ESI(Pos) 412 (M + H)⁺ | amorphous |
| | 1.42 (s, 9 H) 3.41-3.50 (m, 1 H), 3.64-3.71 (m, 2 H), 3.88 (s, 3 H), 4.37- | | |
| | 4.45 (m, 2 H), 7.02 (s, 1 H), 7.13-7.18 (m, 1 H), 7.34-7.38 (m, 1 H), | | |
| | 7.51-7.56 (m, 1 H) | | |
| 493 | 1H NMR (200 MHz, CHLOROFORM-d) δ ppm 1.6 (d, J = 6.2 Hz, 6 H) | ESI(Pos) 430 (M + H)⁺ | amorphous |
| | 1.43 (s, 9 H) 3.42-3.57 (m, 1 H) 3.73 (t, J = 4.8 Hz, 2 H) 3.90 (s, 3 H) 4.46 | | |
| | (t, J = 4.8 Hz, 2 H) 7.03 (s, 1 H) 7.13-7.25 (m, 1 H) 7.51-7.62 (m, 1 H) | | |
| | 8.12-8.24 (m, 1 H) | | |
| 494 | 1H NMR (600 MHz, CHLOROFORM-d) δ ppm 1.06 (d, J = 6.4 Hz, 6 H), | ESI(Pos) 426 (M + H)⁺ | amorphous |
| | 1.43 (s, 9 H), 2.63 (s, 3 H), 3.44-3.50 (m, 1 H), 3.66 (t, J = 4.8 Hz, 2 H), | | |
| | 3.88 (s, 3 H), 4.40 (br. s., 2 H), 7.02 (s, 1 H), 7.19-7.25 (m, 1 H), 7.53- | | |
| | 7.56 (m, 1 H), 7.74-7.79 (m, 1 H) | | |
| 495 | 1H NMR (200 MHz, CHLOROFORM-d) δ ppm 0.48-0.62 (m, 4 H) 1.03- | ESI(Pos) 412 (M + H)⁺ | amorphous |
| | 1.22 (m, 1 H) 1.29 (t, J = 6.8 Hz, 3 H) 1.48 (s, 9 H) 4.28 (q, J = 68 Hz, 2 H) | | |
| | 4.25 (d, J = 7.0 Hz, 2 H) 7.14 (s, 1 H) 7.17-7.29 (m, 1 H) 7.54-7.67 (m, 1 | | |
| | H) 8.12-8.24 (m, 1 H) | | |
| 496 | 1H NMR (200 MHz, CHLOROFORM-d) δ ppm 0.43-0.69 (m, 4 H) 0.99- | ESI(Pos) 412 (M + H)⁺ | 149-150 |
| | 1.21 (m, 1 H) 1.52 (s, 9 H) 2.15 (s, 3 H) 3.85 (s, 3 H) 4.14 (d, J = 7.0 Hz, 2 | | |
| | H) 7.14-7.25 (m, 1 H) 7.49-7.60 (m, 1 H) 7.96-8.09 (m, 1 H) | | |
| 497 | 1H NMR (200 MHz, CHLOROFORM-d) d ppm 0.96-1.07 (m, 2 H) 1.12- | ESI(Pos) 404 (M + H)⁺ | 168.5-169.5 |
| | 1.24 (m, 2 H) 1.70-1.86 (m, 1 H) 3.52 (s, 3 H) 6.92 (s, 1 H) 7.02-7.13 (m, | | |
| | 1 H) 7.40-7.63 (m, 6 H) 7.91-8.05 (m, 1 H) | | |
| 498 | 1H NMR (600 MHz, CHLOROFORM-d) d ppm 3.93 (3 H, s), 7.06-7.10 (1 | ESI(Pos) 446 (M + H)⁺ | 169.5-170 |
| | H, m), 7.30-7.37 (5 H, m), 7.46-7.50 (1 H, m), 7.71-7.75 (1 H, m) | | |
| 499 | 1H NMR (600 MHz, CHLOROFORM-d) d ppm 3.39 (3 H, s), 3.78 (2 H, s), | ESI(Pos) 361 (M + H)⁺ | 128.5-130 |
| | 4.47 (2 H, s), 7.32-7.35 (1 H, m), 7.39-7.44 (2 H, m), 7.48-7.51 (2 H, | | |
| | m), 7.79-7.83 (1 H, m), 8.13-8.17 (1 H, m), 8.18-8.22 (1 H, m) | | |
| 500 | 1H NMR (600 MHz, CHLOROFORM-d) d ppm 0.52-0.56 (2 H, m), 0.83- | ESI(Pos) 376 (M + H)⁺ | amorphous |
| | 0.87 (2 H, m), 0.98-1.06 (1 H, m), 1.43 (9 H, s), 3.87 (3 H, s), 4.16 (2 H, d, | | |
| | J = 6.88 Hz), 6.92-6.96 (1 H, m), 6.99 (1 H, s), 7.59-7.64 (2 H, m) | | |
| 501 | 1H NMR (600 MHz, CHLOROFORM-d) ppm 1.42 (9 H, s), 3.89 (3 H, s), | ESI(Pos) 429 (M + H)⁺ | amorphous |
| | 4.22 (2 H, d, J = 6.88 Hz), 6.77 (1 H, s), 6.93 (1 H, s), 7.54 (1 H, dd J = 8.94, | | |
| | 2.52 Hz), 7.61 (1 H, d, J = 2.29 Hz), 7.91 (1 H, d, J = 8.71 Hz) | | |
| 502 | 1H NMR (600 MHz, CHLOROFORM-d) d ppm 0.28-0.37 (2 H, m), 0.51- | ESI(Pos) 361 (M + H)⁺ | 147.5-149.5 |
| | 0.58 (2 H, m), 1.01-1.09 (1 H, m), 1.43 (9 H, s), 3.83 (3 H, s), 4.04 (2 H, s), | | |
| | 6.91-6.95 (2 H, m), 7.36 (2 H, s), 7.36-7.40 (2 H, m), 10.47 (1 H, s) | | |
| 503 | 1H NMR (200 MHz, CHLOROFORM-d) δ ppm 0.34-0.57 (m, 4 H) 0.89- | ESI(Pos) 416 (M + H)⁺ | 121-122 |
| | 1.07 (m, 1 H) 1.37 (s, 9 H) 3.74 (s, 3 H) 4.03 (d, J = 7.0 Hz, 2 H) 6.18 (s, 1 | | |
| | H) 7.48-7.60 (m, 1 H) 7.62-7.72 (m, 1 H) 8.10-8.19 (m, 1 H) 8.19- | | |
| | 8.26 (m, 1 H) | | |
| 504 | 1H NMR (200 MHz, CHLOROFORM-d) d ppm 0.47-0.66 (m, 4 H) 1.35- | ESI(Pos) 402 (M + H)⁺ | amorphous |
| | 1.44 (m, 1 H) 1.43 (s, 9 H) 4.28 (d, J = 7.0 Hz, 2 H) 7.24-7.35 (m, 1 H) 7.65- | | |
| | 7.77 (m, 1 H) 8.25-8.37 (m, 1 H) | | |
| 505 | 1H NMR (200 MHz, CHLOROFORM-d) d ppm 0.50-0.68 (m, 4 H) 1.36- | ESI(Pos) 384 (M + H)⁺ | amorphous |
| | 1.52 (m, 1 H) 1.44 (s, 9 H) 4.32 (d, J = 7.5 Hz, 2 H) 7.57 (t, J = 7.7 Hz, 1 H) | | |
| | 7.75 (d, J = 7.7 Hz, 1 H) 8.47 (d, J = 7.7 Hz, 1 H) 8.57 (s, 1 H) | | |
| 506 | 1H NMR (200 MHz, CHLOROFORM-d) d ppm 0.43-0.68 (m, 4 H) 1.30- | ESI(Pos) 398 (M + H)⁺ | 63-65 |
| | 1.52 (m, 10 H) 2.75 (s, 3 H) 4.28 (d, J = 7.5 Hz, 2 H) 7.29-7.41 (m, 1 H) | | |
| | 7.49-7.65 (m, 1 H) 8.37-8.47 (m, 1 H) | | |
| 507 | 1H NMR (200 MHz, CHLOROFORM-d) d ppm 0.41-0.65 (m, 4 H) 1.29- | ESI(Pos) 402 (M + H)⁺ | 76-78 |
| | 1.50 (m, 1 H) 1.43 (s, 9 H) 4.23 (d, J = 7.5 Hz, 2 H) 7.20-7.33 (m, 1 H) 7.40- | | |
| | 7.50 (m, 1 H) 7.94-8.05 (m, 1 H) | | |
| 508 | 1H NMR (200 MHz, CHLOROFORM-d) d ppm 0.47-0.67 (m, 4 H) 1.33- | ESI(Pos) 402 (M + H)⁺ | 47-49 |
| | 1.56 (m, 1 H) 1.44 (s, 9 H) 4.29 (d, J = 7.0 Hz, 2 H) 7.16-7.31 (m, 1 H) 7.64- | | |
| | 7.78 (m, 1 H) 8.39-8.49 (m, 1 H) | | |
| 509 | 1H NMR (200 MHz, CHLOROFORM-d) d ppm 0.44-0.66 (m, 4 H) 1.30- | ESI(Pos) 414 (M + H)⁺ | 82-83 |
| | 1.52 (m, 1 H) 1.42 (s, 9 H) 3.97 (s, 3 H) 4.26 (d, J = 7.5 Hz, 2 H) 7.01-7.10 | | |
| | (m, 1 H) 7.61-7.70 (m, 1 H) 8.27-833 (m, 1 H) | | |
| 510 | 1H NMR (200 MHz, CHLOROFORM-d) d ppm 0.41-0.62 (m, 4 H) 1.29- | ESI(Pos) 367 (M + H)⁺ | 89-90 |
| | 1.51 (m, 1 H) 1.47 (s, 9 H) 4.30 (d, J = 7.5 Hz, 2 H) 7.52-7.89) (m, 4 H) 8.59- | | |
| | 8.68 (m, 2 H) | | |
| 511 | 1H NMR (200 MHz, CHLOROFORM-d) d ppm 0.43-0.67 (m, 4 H) 1.28- | ESI(Pos) 351 (M + H)⁺ | 62-63 |
| | 1.51 (m, 1 H) 1.44 (s, 9 H) 4.27 (d, J = 7.5 Hz, 2 H) 7.33-7.43 (m, 1 H) 8.46- | | |
| | 8.57 (m, 1 H) 9.20-9.33 (m, 1 H) | | |
| 512 | 1H NMR (200 MHz, CHLOROFORM-d) d ppm 0.44-0.65 (m, 4 H) 1.31- | ESI(Pos) 347 (M + H)⁺ | 94-95 |
| | 1.50 (m, 1 H) 1.42 (s, 9 H) 3.98 (s, 3 H) 4.26 (d, J = 7.0 Hz, 2 H) 6.90 (d, | | |
| | J = 5.7 Hz, 1 H) 8.54 (d, J = 5.7 Hz, 1 H) 9.22 (s 1 H) | | |
| 513 | 1H NMR (200 MHz, CHLOROFORM-d) d ppm 0.40-0.66 (m, 4 H) 1.29- | ESI(Pos) 361 (M + H)⁺ | amorphous |
| | 1.50 (m, 1 H) 1.42 (s, 9 H) 1.50 (t, J = 7.0 Hz, 3 H) 4.24 (q, J = 7.0 Hz, 2 H) | | |
| | 4.25 (d, J = 7.0 Hz, 2 H) 6.87 (d, J = 6.2 Hz, 1 H) 8.49 (d, J = 6.2 Hz, 1 H) 9.17 | | |
| | (s, 1 H) | | |
| 514 | 1H NMR (200 MHz, CHLOROFORM-d) d ppm 0.41-0.67 (m, 4 H) 1.23- | ESI(Pos) 386 (M + H)⁺ | 108-110 |
| | 1.35 (m, 1 H) 1.41 (s, 9 H) 3.82 (d, J = 7.5 Hz, 2 H) 7.20-7.31 (m, 1 H) 7.63- | | |
| | 7.74 (m, 1 H) 8.16-8.27 (m, 1 H) | | |
| 515 | 1H NMR (200 MHz, CHLOROFORM-d) d ppm 0.38-0.70 (m, 4 H) 1.18- | ESI(Pos) 386 (M + H)⁺ | 61-63 |
| | 1.38 (m, 1 H) 1.41 (s, 9 H) 3.83 (d, J = 7.5 Hz, 2 H) 7.13-7.30 (m, 1 H) 7.59- | | |
| | 7.75 (m, 1 H) 8.29-8.43 (m, 1 H) | | |

TABLE 12

| Compound No. | MASS |
|---|---|
| 1001 | APCI (Pos) 247 (M + H) |
| 1002 | APCI (Pos) 233 (M + H) |
| 1003 | APCI (Pos) 247 (M + H) |
| 1004 | APCI (Pos) 239 (M + H) |
| 1005 | APCI (Pos) 259 (M + H) |
| 1006 | APCI (Pos) 273 (M + H) |
| 1007 | APCI (Pos) 287 (M + H) |
| 1008 | APCI (Pos) 281 (M + H) |
| 1009 | APCI (Pos) 291 (M + H) |
| 1010 | APCI (Pos) 295 (M + H) |
| 1011 | APCI (Pos) 343 (M + H) |
| 1012 | APCI (Pos) 353 (M + H) |
| 1013 | APCI (Pos) 273 (M + H) |
| 1014 | APCI (Pos) 283 (M + H) |
| 1015 | APCI (Pos) 285 (M + H) |
| 1016 | APCI (Pos) 273 (M + H) |
| 1017 | APCI (Pos) 279 (M + H) |
| 1018 | APCI (Pos) 245 (M + H) |
| 1019 | APCI (Pos) 259 (M + H) |
| 1020 | APCI (Pos) 307 (M + H) |
| 1021 | APCI (Pos) 293 (M + H) |
| 1022 | APCI (Pos) 293 (M + H) |
| 1023 | APCI (Pos) 289 (M + H) |
| 1024 | APCI (Pos) 289 (M + H) |
| 1025 | APCI (Pos) 253 (M + H) |
| 1026 | APCI (Pos) 303 (M + H) |
| 1027 | APCI (Pos) 303 (M + H) |
| 1028 | APCI (Pos) 341 (M + H) |
| 1029 | APCI (Pos) 267 (M + H) |
| 1030 | APCI (Pos) 267 (M + H) |
| 1031 | APCI (Pos) 267 (M + H) |
| 1032 | APCI (Pos) 281 (M + H) |
| 1033 | APCI (Pos) 281 (M + H) |
| 1034 | APCI (Pos) 295 (M + H) |
| 1035 | APCI (Pos) 295 (M + H) |
| 1036 | APCI (Pos) 321 (M + H) |
| 1037 | APCI (Pos) 335 (M + H) |
| 1038 | APCI (Pos) 271 (M + H) |
| 1039 | APCI (Pos) 271 (M + H) |
| 1040 | APCI (Pos) 271 (M + H) |
| 1041 | APCI (Pos) 287 (M + H) |
| 1042 | APCI (Pos) 287 (M + H) |
| 1043 | APCI (Pos) 267 (M + H) |
| 1044 | APCI (Pos) 331 (M + H) |
| 1045 | APCI (Pos) 331 (M + H) |
| 1046 | APCI (Pos) 331 (M + H) |
| 1047 | APCI (Pos) 379 (M + H) |
| 1048 | APCI (Pos) 379 (M + H) |
| 1049 | APCI (Pos) 283 (M + H) |
| 1050 | APCI (Pos) 283 (M + H) |
| 1051 | APCI (Pos) 283 (M + H) |
| 1052 | APCI (Pos) 297 (M + H) |
| 1053 | APCI (Pos) 297 (M + H) |
| 1054 | APCI (Pos) 337 (M + H) |
| 1055 | APCI (Pos) 337 (M + H) |
| 1056 | APCI (Pos) 345 (M + H) |
| 1057 | APCI (Pos) 345 (M + H) |
| 1058 | APCI (Pos) 345 (M + H) |
| 1059 | APCI (Pos) 369 (M + H) |
| 1060 | APCI (Pos) 369 (M + H) |
| 1061 | APCI (Pos) 369 (M + H) |
| 1062 | APCI (Pos) 343 (M + H) |
| 1063 | APCI (Pos) 329 (M + H) |
| 1064 | APCI (Pos) 329 (M + H) |
| 1065 | APCI (Pos) 359 (M + H) |
| 1066 | APCI (Pos) 352 (M + H) |
| 1067 | APCI (Pos) 344 (M + H) |
| 1068 | APCI (Pos) 338 (M + H) |
| 1069 | APCI (Pos) 278 (M + H) |
| 1070 | APCI (Pos) 278 (M + H) |
| 1071 | APCI (Pos) 278 (M + H) |
| 1072 | APCI (Pos) 349 (M + H) |
| 1073 | APCI (Pos) 331 (M + H) |
| 1074 | APCI (Pos) 331 (M + H) |
| 1075 | APCI (Pos) 281 (M + H) |
| 1076 | APCI (Pos) 281 (M + H) |
| 1077 | APCI (Pos) 281 (M + H) |
| 1078 | APCI (Pos) 281 (M + H) |
| 1079 | APCI (Pos) 335 (M + H) |
| 1080 | APCI (Pos) 335 (M + H) |
| 1081 | APCI (Pos) 365 (M + H) |
| 1082 | APCI (Pos) 399 (M + H) |
| 1083 | APCI (Pos) 399 (M + H) |
| 1084 | APCI (Pos) 399 (M + H) |
| 1085 | APCI (Pos) 289 (M + H) |
| 1086 | APCI (Pos) 289 (M + H) |
| 1087 | APCI (Pos) 289 (M + H) |
| 1088 | APCI (Pos) 289 (M + H) |
| 1089 | APCI (Pos) 289 (M + H) |
| 1090 | APCI (Pos) 289 (M + H) |
| 1091 | APCI (Pos) 305 (M + H) |
| 1092 | APCI (Pos) 305 (M + H) |
| 1093 | APCI (Pos) 305 (M + H) |
| 1094 | APCI (Pos) 305 (M + H) |
| 1095 | APCI (Pos) 285 (M + H) |
| 1096 | APCI (Pos) 305 (M + H) |
| 1097 | APCI (Pos) 321 (M + H) |
| 1098 | APCI (Pos) 321 (M + H) |
| 1099 | APCI (Pos) 321 (M + H) |
| 1100 | APCI (Pos) 321 (M + H) |
| 1101 | APCI (Pos) 348 (M + H) |
| 1102 | APCI (Pos) 349 (M + H) |
| 1103 | APCI (Pos) 349 (M + H) |
| 1104 | APCI (Pos) 349 (M + H) |
| 1105 | APCI (Pos) 349 (M + H) |
| 1106 | APCI (Pos) 349 (M + H) |
| 1107 | APCI (Pos) 349 (M + H) |
| 1108 | APCI (Pos) 365 (M + H) |
| 1109 | APCI (Pos) 365 (M + H) |
| 1110 | APCI (Pos) 365 (M + H) |
| 1111 | APCI (Pos) 365 (M + H) |
| 1112 | APCI (Pos) 397 (M + H) |
| 1113 | APCI (Pos) 397 (M + H) |
| 1114 | APCI (Pos) 313 (M + H) |
| 1115 | APCI (Pos) 313 (M + H) |
| 1116 | APCI (Pos) 313 (M + H) |
| 1117 | APCI (Pos) 313 (M + H) |
| 1118 | APCI (Pos) 313 (M + H) |
| 1119 | APCI (Pos) 341 (M + H) |
| 1120 | APCI (Pos) 341 (M + H) |
| 1121 | APCI (Pos) 341 (M + H) |
| 1122 | APCI (Pos) 341 (M + H) |
| 1123 | APCI (Pos) 285 (M + H) |
| 1124 | APCI (Pos) 285 (M + H) |
| 1125 | APCI (Pos) 285 (M + H) |
| 1126 | APCI (Pos) 285 (M + H) |
| 1127 | APCI (Pos) 301 (M + H) |
| 1128 | APCI (Pos) 301 (M + H) |
| 1129 | APCI (Pos) 301 (M + H) |
| 1130 | APCI (Pos) 301 (M + H) |
| 1131 | APCI (Pos) 345 (M + H) |
| 1132 | APCI (Pos) 345 (M + H) |
| 1133 | APCI (Pos) 345 (M + H) |
| 1134 | APCI (Pos) 345 (M + H) |
| 1135 | APCI (Pos) 345 (M + H) |
| 1136 | APCI (Pos) 345 (M + H) |
| 1137 | APCI (Pos) 393 (M + H) |
| 1138 | APCI (Pos) 393 (M + H) |
| 1139 | APCI (Pos) 393 (M + H) |
| 1140 | APCI (Pos) 339 (M + H) |
| 1141 | APCI (Pos) 339 (M + H) |
| 1142 | APCI (Pos) 339 (M + H) |
| 1143 | APCI (Pos) 407 (M + H) |
| 1144 | APCI (Pos) 338 (M + H) |
| 1145 | APCI (Pos) 338 (M + H) |
| 1146 | APCI (Pos) 355 (M + H) |
| 1147 | APCI (Pos) 355 (M + H) |
| 1148 | APCI (Pos) 355 (M + H) |
| 1149 | APCI (Pos) 338 (M + H) |
| 1150 | APCI (Pos) 297 (M + H) |
| 1151 | APCI (Pos) 297 (M + H) |
| 1152 | APCI (Pos) 297 (M + H) |
| 1153 | APCI (Pos) 339 (M + H) |
| 1154 | APCI (Pos) 351 (M + H) |
| 1155 | APCI (Pos) 301 (M + H) |
| 1156 | APCI (Pos) 301 (M + H) |
| 1157 | APCI (Pos) 301 (M + H) |
| 1158 | APCI (Pos) 317 (M + H) |
| 1159 | APCI (Pos) 317 (M + H) |
| 1160 | APCI (Pos) 317 (M + H) |
| 1161 | APCI (Pos) 361 (M + H) |
| 1162 | APCI (Pos) 387 (M + H) |
| 1163 | APCI (Pos) 409 (M + H) |
| 1164 | APCI (Pos) 286 (M + H) |
| 1165 | APCI (Pos) 286 (M + H) |
| 1166 | APCI (Pos) 308 (M + H) |
| 1167 | APCI (Pos) 285 (M + H) |
| 1168 | APCI (Pos) 285 (M + H) |
| 1169 | APCI (Pos) 307 (M + H) |
| 1170 | APCI (Pos) 307 (M + H) |
| 1171 | APCI (Pos) 307 (M + H) |
| 1172 | APCI (Pos) 307 (M + H) |
| 1173 | APCI (Pos) 307 (M + H) |
| 1174 | APCI (Pos) 323 (M + H) |
| 1175 | APCI (Pos) 323 (M + H) |
| 1176 | APCI (Pos) 323 (M + H) |
| 1177 | APCI (Pos) 323 (M + H) |
| 1178 | APCI (Pos) 323 (M + H) |
| 1179 | APCI (Pos) 339 (M + H) |
| 1180 | APCI (Pos) 355 (M + H) |
| 1181 | APCI (Pos) 355 (M + H) |
| 1182 | APCI (Pos) 367 (M + H) |
| 1183 | APCI (Pos) 343 (M + H) |
| 1184 | APCI (Pos) 343 (M + H) |
| 1185 | ESI (Pos) 343 (M + H) |
| 1186 | APCI (Pos) 311 (M + H) |
| 1187 | APCI (Pos) 303 (M + H) |
| 1188 | APCI (Pos) 303 (M + H) |
| 1189 | APCI (Pos) 319 (M + H) |
| 1190 | APCI (Pos) 319 (M + H) |
| 1191 | APCI (Pos) 357 (M + H) |
| 1192 | APCI (Pos) 357 (M + H) |
| 1193 | APCI (Pos) 373 (M + H) |
| 1194 | APCI (Pos) 373 (M + H) |
| 1195 | APCI (Pos) 327 (M + H) |
| 1196 | APCI (Pos) 337 (M + H) |
| 1197 | APCI (Pos) 338 (M + H) |
| 1198 | APCI (Pos) 247 (M + H) |
| 1199 | APCI (Pos) 311 (M + H) |
| 1200 | APCI (Pos) 323 (M + H) |
| 1201 | APCI (Pos) 333 (M + H) |
| 1202 | APCI (Pos) 311 (M + H) |
| 1203 | APCI (Pos) 311 (M + H) |
| 1204 | APCI (Pos) 325 (M + H) |
| 1205 | APCI (Pos) 295 (M + H) |
| 1206 | APCI (Pos) 311 (M + H) |
| 1207 | APCI (Pos) 268 (M + H) |
| 1208 | APCI (Pos) 264 (M + H) |
| 1209 | APCI (Pos) 279 (M + H) |
| 1210 | APCI (Pos) 322 (M + H) |
| 1211 | APCI (Pos) 322 (M + H) |
| 1212 | APCI (Pos) 322 (M + H) |
| 1213 | APCI (Pos) 289 (M + H) |
| 1214 | APCI (Pos) 304 (M + H) |
| 1215 | APCI (Pos) 304 (M + H) |
| 1216 | APCI (Pos) 304 (M + H) |
| 1217 | APCI (Pos) 304 (M + H) |
| 1218 | APCI (Pos) 304 (M + H) |
| 1219 | APCI (Pos) 338 (M + H) |
| 1220 | APCI (Pos) 378 (M + H) |
| 1221 | APCI (Pos) 310 (M + H) |
| 1222 | APCI (Pos) 310 (M + H) |
| 1223 | APCI (Pos) 311 (M + H) |
| 1224 | APCI (Pos) 311 (M + H) |
| 1225 | APCI (Pos) 327 (M + H) |
| 1226 | APCI (Pos) 327 (M + H) |
| 1227 | APCI (Pos) 311 (M + H) |
| 1228 | APCI (Pos) 311 (M + H) |
| 1229 | APCI (Pos) 311 (M + H) |
| 1230 | APCI (Pos) 311 (M + H) |
| 1231 | APCI (Pos) 312 (M + H) |
| 1232 | APCI (Pos) 328 (M + H) |
| 1233 | APCI (Pos) 328 (M + H) |
| 1234 | ESI (Neg) 322 (M − H) |
| 1235 | APCI (Pos) 355 (M + H) |
| 1236 | APCI (Pos) 389 (M + H) |
| 1237 | APCI (Pos) 437 (M + H) |
| 1238 | APCI (Pos) 356 (M + H) |
| 1239 | APCI (Pos) 471 (M + H) |
| 1240 | APCI (Pos) 325 (M + H) |
| 1241 | APCI (Pos) 325 (M + H) |
| 1242 | APCI (Pos) 325 (M + H) |
| 1243 | APCI (Pos) 368 (M + H) |
| 1244 | APCI (Pos) 394 (M + H) |
| 1245 | APCI (Pos) 396 (M + H) |
| 1246 | APCI (Pos) 408 (M + H) |
| 1247 | APCI (Pos) 408 (M + H) |
| 1248 | APCI (Pos) 410 (M + H) |
| 1249 | APCI (Pos) 410 (M + H) |
| 1250 | APCI (Pos) 368 (M + H) |
| 1251 | APCI (Pos) 387 (M + H) |
| 1252 | APCI (Pos) 401 (M + H) |
| 1253 | APCI (Pos) 417 (M + H) |
| 1254 | APCI (Pos) 417 (M + H) |
| 1255 | APCI (Pos) 421 (M + H) |
| 1256 | APCI (Pos) 432 (M + H) |
| 1257 | APCI (Pos) 432 (M + H) |
| 1258 | APCI (Pos) 446 (M + H) |
| 1259 | APCI (Pos) 341 (M + H) |
| 1260 | APCI (Pos) 341 (M + H) |
| 1261 | APCI (Pos) 341 (M + H) |
| 1262 | APCI (Pos) 355 (M + H) |
| 1263 | APCI (Pos) 383 (M + H) |
| 1264 | APCI (Pos) 392 (M + H) |
| 1265 | APCI (Pos) 403 (M + H) |
| 1266 | APCI (Pos) 404 (M + H) |
| 1267 | APCI (Pos) 409 (M + H) |
| 1268 | APCI (Pos) 424 (M + H) |
| 1269 | APCI (Pos) 389 (M + H) |
| 1270 | APCI (Pos) 405 (M + H) |
| 1271 | APCI (Pos) 361 (M + H) |
| 1272 | APCI (Pos) 361 (M + H) |
| 1273 | APCI (Pos) 405 (M + H) |
| 1274 | APCI (Pos) 405 (M + H) |
| 1275 | APCI (Pos) 405 (M + H) |
| 1276 | APCI (Pos) 372 (M + H) |
| 1277 | APCI (Pos) 384 (M + H) |
| 1278 | APCI (Pos) 371 (M + H) |
| 1279 | APCI (Pos) 357 (M + H) |
| 1280 | APCI (Pos) 405 (M + H) |
| 1281 | APCI (Pos) 325 (M + H) |
| 1282 | APCI (Pos) 325 (M + H) |
| 1283 | APCI (Pos) 325 (M + H) |
| 1284 | APCI (Pos) 325 (M + H) |
| 1285 | APCI (Pos) 325 (M + H) |
| 1286 | APCI (Pos) 339 (M + H) |
| 1287 | APCI (Pos) 339 (M + H) |
| 1288 | APCI (Pos) 339 (M + H) |
| 1289 | APCI (Pos) 387 (M + H) |
| 1290 | APCI (Pos) 387 (M + H) |
| 1291 | APCI (Pos) 405 (M + H) |
| 1292 | APCI (Pos) 417 (M + H) |
| 1293 | APCI (Pos) 345 (M + H) |
| 1294 | APCI (Pos) 356 (M + H) |
| 1295 | APCI (Pos) 356 (M + H) |
| 1296 | APCI (Pos) 388 (M + H) |
| 1297 | APCI (Pos) 388 (M + H) |
| 1298 | APCI (Pos) 388 (M + H) |
| 1299 | APCI (Pos) 326 (M + H) |
| 1300 | APCI (Pos) 326 (M + H) |
| 1301 | APCI (Pos) 326 (M + H) |
| 1302 | APCI (Pos) 342 (M + H) |
| 1303 | APCI (Pos) 342 (M + H) |
| 1304 | APCI (Pos) 405 (M + H) |
| 1305 | APCI (Pos) 405 (M + H) |
| 1306 | APCI (Pos) 410 (M + H) |
| 1307 | APCI (Pos) 418 (M + H) |
| 1308 | APCI (Pos) 438 (M + H) |
| 1309 | APCI (Pos) 404 (M + H) |
| 1310 | APCI (Pos) 343 (M + H) |
| 1311 | APCI (Pos) 405 (M + H) |
| 1312 | APCI (Pos) 419 (M + H) |
| 1313 | APCI (Pos) 415 (M + H) |
| 1314 | APCI (Pos) 431 (M + H) |
| 1315 | APCI (Pos) 489 (M + H) |
| 1316 | APCI (Pos) 339 (M + H) |
| 1317 | APCI (Pos) 339 (M + H) |
| 1318 | APCI (Pos) 381 (M + H) |
| 1319 | APCI (Pos) 393 (M + H) |
| 1320 | APCI (Pos) 424 (M + H) |
| 1321 | APCI (Pos) 419 (M + H) |
| 1322 | APCI (Pos) 515 (M + H) |
| 1323 | APCI (Pos) 391 (M + H) |
| 1324 | APCI (Pos) 435 (M + H) |
| 1325 | APCI (Pos) 439 (M + H) |
| 1326 | APCI (Pos) 448 (M + H) |
| 1327 | APCI (Pos) 463 (M + H) |
| 1328 | APCI (Pos) 489 (M + H) |
| 1329 | APCI (Pos) 500 (M + H) |
| 1330 | APCI (Pos) 339 (M + H) |
| 1331 | APCI (Pos) 339 (M + H) |
| 1332 | APCI (Pos) 339 (M + H) |
| 1333 | APCI (Pos) 353 (M + H) |
| 1334 | APCI (Pos) 353 (M + H) |
| 1335 | APCI (Pos) 353 (M + H) |
| 1336 | APCI (Pos) 353 (M + H) |
| 1337 | APCI (Pos) 353 (M + H) |
| 1338 | APCI (Pos) 367 (M + H) |
| 1339 | APCI (Pos) 367 (M + H) |
| 1340 | APCI (Pos) 367 (M + H) |
| 1341 | APCI (Pos) 361 (M + H) |
| 1342 | APCI (Pos) 393 (M + H) |
| 1343 | APCI (Pos) 393 (M + H) |
| 1344 | APCI (Pos) 393 (M + H) |
| 1345 | APCI (Pos) 457 (M + H) |
| 1346 | APCI (Pos) 526 (M + H) |
| 1347 | APCI (Pos) 415 (M + H) |
| 1348 | APCI (Pos) 401 (M + H) |
| 1349 | APCI (Pos) 401 (M + H) |
| 1350 | APCI (Pos) 415 (M + H) |
| 1351 | APCI (Pos) 419 (M + H) |
| 1352 | APCI (Pos) 429 (M + H) |
| 1353 | APCI (Pos) 455 (M + H) |
| 1354 | APCI (Pos) 339 (M + H) |
| 1355 | APCI (Pos) 359 (M + H) |
| 1356 | APCI (Pos) 359 (M + H) |
| 1357 | APCI (Pos) 359 (M + H) |
| 1358 | APCI (Pos) 373 (M + H) |
| 1359 | APCI (Pos) 373 (M + H) |
| 1360 | APCI (Pos) 403 (M + H) |
| 1361 | APCI (Pos) 403 (M + H) |
| 1362 | APCI (Pos) 417 (M + H) |
| 1363 | APCI (Pos) 340 (M + H) |
| 1364 | APCI (Pos) 340 (M + H) |
| 1365 | APCI (Pos) 394 (M + H) |
| 1366 | APCI (Pos) 402 (M + H) |
| 1367 | APCI (Pos) 402 (M + H) |
| 1368 | APCI (Pos) 420 (M + H) |
| 1369 | APCI (Pos) 436 (M + H) |
| 1370 | APCI (Pos) 454 (M + H) |
| 1371 | APCI (Pos) 470 (M + H) |
| 1372 | APCI (Pos) 419 (M + H) |
| 1373 | APCI (Pos) 419 (M + H) |
| 1374 | APCI (Pos) 420 (M + H) |
| 1375 | APCI (Pos) 460 (M + H) |
| 1376 | APCI (Pos) 356 (M + H) |
| 1377 | APCI (Pos) 410 (M + H) |
| 1378 | APCI (Pos) 396 (M + H) |
| 1379 | APCI (Pos) 402 (M + H) |
| 1380 | APCI (Pos) 340 (M + H) |
| 1381 | APCI (Pos) 429 (M + H) |
| 1382 | APCI (Pos) 378 (M + H) |
| 1383 | APCI (Pos) 434 (M + H) |
| 1384 | APCI (Pos) 448 (M + H) |
| 1385 | APCI (Pos) 380 (M + H) |
| 1386 | APCI (Pos) 413 (M + H) |
| 1387 | APCI (Pos) 467 (M + H) |
| 1388 | APCI (Pos) 429 (M + H) |
| 1389 | APCI (Pos) 353 (M + H) |
| 1390 | APCI (Pos) 373 (M + H) |
| 1391 | APCI (Pos) 387 (M + H) |
| 1392 | APCI (Pos) 417 (M + H) |
| 1393 | APCI (Pos) 417 (M + H) |
| 1394 | APCI (Pos) 427 (M + H) |
| 1395 | APCI (Pos) 431 (M + H) |
| 1396 | APCI (Pos) 373 (M + H) |
| 1397 | APCI (Pos) 374 (M + H) |
| 1398 | APCI (Pos) 374 (M + H) |
| 1399 | APCI (Pos) 374 (M + H) |
| 1400 | APCI (Pos) 388 (M + H) |
| 1401 | APCI (Pos) 390 (M + H) |
| 1402 | APCI (Pos) 376 (M + H) |
| 1403 | APCI (Pos) 378 (M + H) |
| 1404 | APCI (Pos) 390 (M + H) |
| 1405 | APCI (Pos) 444 (M + H) |
| 1406 | APCI (Pos) 405 (M + H) |
| 1407 | APCI (Pos) 420 (M + H) |
| 1408 | APCI (Pos) 361 (M + H) |
| 1409 | APCI (Pos) 361 (M + H) |
| 1410 | APCI (Pos) 361 (M + H) |
| 1411 | APCI (Pos) 375 (M + H) |
| 1412 | APCI (Pos) 395 (M + H) |
| 1413 | APCI (Pos) 396 (M + H) |
| 1414 | APCI (Pos) 390 (M + H) |
| 1415 | APCI (Pos) 444 (M + H) |
| 1416 | APCI (Pos) 427 (M + H) |
| 1417 | APCI (Pos) 381 (M + H) |
| 1418 | APCI (Pos) 361 (M + H) |
| 1419 | APCI (Pos) 375 (M + H) |
| 1420 | APCI (Pos) 391 (M + H) |
| 1421 | APCI (Pos) 405 (M + H) |
| 1422 | APCI (Pos) 379 (M + H) |
| 1423 | APCI (Pos) 367 (M + H) |
| 1424 | APCI (Pos) 381 (M + H) |
| 1425 | APCI (Pos) 377 (M + H) |
| 1426 | APCI (Pos) 377 (M + H) |
| 1427 | APCI (Pos) 391 (M + H) |
| 1428 | APCI (Pos) 411 (M + H) |
| 1429 | APCI (Pos) 425 (M + H) |
| 1430 | APCI (Pos) 383 (M + H) |
| 1431 | APCI (Pos) 362 (M + H) |

TABLE 13

| Compound No. | MASS |
|---|---|
| 2001 | APCI: 267 (M + H)+ |
| 2002 | APCI: 267 (M + H)+ |
| 2003 | APCI: 293 (M + H)+ |
| 2004 | APCI: 297 (M + H)+ |
| 2005 | APCI: 301 (M + H)+ |
| 2006 | APCI: 301 (M + H)+ |
| 2007 | APCI: 302 (M + H)+ |
| 2008 | APCI: 302 (M + H)+ |
| 2009 | APCI: 305 (M + H)+ |
| 2010 | APCI: 306 (M + H)+ |
| 2011 | APCI: 307 (M + H)+ |
| 2012 | APCI: 307 (M + H)+ |
| 2013 | APCI: 312 (M + H)+ |
| 2014 | APCI: 315 (M + H)+ |
| 2015 | APCI: 315 (M + H)+ |
| 2016 | APCI: 315 (M + H)+ |
| 2017 | APCI: 316 (M + H)+ |
| 2018 | APCI: 317 (M + H)+ |
| 2019 | APCI: 319 (M + H)+ |
| 2020 | APCI: 319 (M + H)+ |
| 2021 | APCI: 321 (M + H)+ |
| 2022 | APCI: 322 (M + H)+ |
| 2023 | APCI: 323 (M + H)+ |
| 2024 | APCI: 323 (M + H)+ |
| 2025 | APCI: 323 (M + H)+ |
| 2026 | APCI: 323 (M + H)+ |
| 2027 | APCI: 323 (M + H)+ |
| 2028 | APCI: 327 (M + H)+ |
| 2029 | APCI: 330 (M + H)+ |
| 2030 | APCI: 331 (M + H)+ |
| 2031 | APCI: 331 (M + H)+ |
| 2032 | APCI: 332 (M + H)+ |
| 2033 | APCI: 333 (M + H)+ |
| 2034 | APCI: 335 (M + H)+ |
| 2035 | APCI: 335 (M + H)+ |
| 2036 | APCI: 337 (M + H)+ |
| 2037 | APCI: 337 (M + H)+ |
| 2038 | APCI: 337 (M + H)+ |
| 2039 | APCI: 338 (M + H)+ |
| 2040 | APCI: 338 (M + H)+ |
| 2041 | APCI: 338 (M + H)+ |
| 2042 | APCI: 338 (M + H)+ |
| 2043 | APCI: 339 (M + H)+ |
| 2044 | APCI: 339 (M + H)+ |
| 2045 | APCI: 339 (M + H)+ |
| 2046 | APCI: 339 (M + H)+ |
| 2047 | APCI: 341 (M + H)+ |
| 2048 | APCI: 341 (M + H)+ |
| 2049 | APCI: 341 (M + H)+ |
| 2050 | APCI: 347 (M + H)+ |
| 2051 | APCI: 347 (M + H)+ |
| 2052 | APCI: 351 (M + H)+ |
| 2053 | APCI: 353 (M + H)+ |
| 2054 | APCI: 353 (M + H)+ |
| 2055 | APCI: 355 (M + H)+ |
| 2056 | APCI: 355 (M + H)+ |
| 2057 | APCI: 356 (M + H)+ |
| 2058 | APCI: 356 (M + H)+ |
| 2059 | APCI: 356 (M + H)+ |
| 2060 | APCI: 356 (M + H)+ |
| 2061 | APCI: 356 (M + H)+ |
| 2062 | APCI: 357 (M + H)+ |
| 2063 | APCI: 366 (M + H)+ |
| 2064 | APCI: 366 (M + H)+ |
| 2065 | APCI: 369 (M + H)+ |
| 2066 | APCI: 371 (M + H)+ |
| 2067 | APCI: 372 (M + H)+ |
| 2068 | APCI: 372 (M + H)+ |
| 2069 | APCI: 373 (M + H)+ |
| 2070 | APCI: 375 (M + H)+ |
| 2071 | APCI: 375 (M + H)+ |
| 2072 | APCI: 379 (M + H)+ |
| 2073 | APCI: 379 (M + H)+ |
| 2074 | APCI: 379 (M + H)+ |
| 2075 | APCI: 383 (M + H)+ |
| 2076 | APCI: 383 (M + H)+ |
| 2077 | APCI: 389 (M + H)+ |
| 2078 | APCI: 395 (M + H)+ |
| 2079 | APCI: 403 (M + H)+ |
| 2080 | APCI: 406 (M + H)+ |
| 2081 | APCI: 407 (M + H)+ |
| 2082 | APCI: 412 (M + H)+ |
| 2083 | APCI: 413 (M + H)+ |
| 2084 | APCI: 427 (M + H)+ |
| 2085 | APCI: 431 (M + H)+ |
| 2086 | APCI: 269 (M + H)+ |
| 2087 | APCI: 269 (M + H)+ |
| 2088 | APCI: 283 (M + H)+ |
| 2089 | APCI: 295 (M + H)+ |
| 2090 | APCI: 295 (M + H)+ |
| 2091 | APCI: 299 (M + H)+ |
| 2092 | APCI: 304 (M + H)+ |
| 2093 | APCI: 304 (M + H)+ |
| 2094 | APCI: 304 (M + H)+ |
| 2095 | APCI: 307 (M + H)+ |
| 2096 | APCI: 308 (M + H)+ |
| 2097 | APCI: 309 (M + H)+ |
| 2098 | APCI: 309 (M + H)+ |
| 2099 | APCI: 317 (M + H)+ |
| 2100 | APCI: 317 (M + H)+ |
| 2101 | APCI: 317 (M + H)+ |
| 2102 | APCI: 317 (M + H)+ |
| 2103 | APCI: 317 (M + H)+ |
| 2104 | APCI: 318 (M + H)+ |
| 2105 | APCI: 319 (M + H)+ |
| 2106 | APCI: 321 (M + H)+ |
| 2107 | APCI: 321 (M + H)+ |
| 2108 | APCI: 323 (M + H)+ |
| 2109 | APCI: 324 (M + H)+ |
| 2110 | APCI: 325 (M + H)+ |
| 2111 | APCI: 325 (M + H)+ |
| 2112 | APCI: 325 (M + H)+ |
| 2113 | APCI: 325 (M + H)+ |
| 2114 | APCI: 325 (M + H)+ |
| 2115 | APCI: 329 (M + H)+ |
| 2116 | APCI: 331 (M + H)+ |
| 2117 | APCI: 333 (M + H)+ |
| 2118 | APCI: 333 (M + H)+ |
| 2119 | APCI: 334 (M + H)+ |
| 2120 | APCI: 335 (M + H)+ |
| 2121 | APCI: 337 (M + H)+ |
| 2122 | APCI: 337 (M + H)+ |
| 2123 | APCI: 337 (M + H)+ |
| 2124 | APCI: 339 (M + H)+ |
| 2125 | APCI: 339 (M + H)+ |
| 2126 | APCI: 340 (M + H)+ |
| 2127 | APCI: 340 (M + H)+ |
| 2128 | APCI: 340 (M + H)+ |
| 2129 | APCI: 340 (M + H)+ |
| 2130 | APCI: 341 (M + H)+ |
| 2131 | APCI: 341 (M + H)+ |
| 2132 | APCI: 341 (M + H)+ |
| 2133 | APCI: 341 (M + H)+ |
| 2134 | APCI: 343 (M + H)+ |
| 2135 | APCI: 343 (M + H)+ |
| 2136 | APCI: 349 (M + H)+ |
| 2137 | APCI: 349 (M + H)+ |
| 2138 | APCI: 355 (M + H)+ |
| 2139 | APCI: 355 (M + H)+ |
| 2140 | APCI: 357 (M + H)+ |
| 2141 | APCI: 357 (M + H)+ |
| 2142 | APCI: 357 (M + H)+ |
| 2143 | APCI: 358 (M + H)+ |
| 2144 | APCI: 358 (M + H)+ |
| 2145 | APCI: 358 (M + H)+ |
| 2146 | APCI: 358 (M + H)+ |
| 2147 | APCI: 358 (M + H)+ |
| 2148 | APCI: 359 (M + H)+ |
| 2149 | APCI: 368 (M + H)+ |
| 2150 | APCI: 368 (M + H)+ |
| 2151 | APCI: 371 (M + H)+ |
| 2152 | APCI: 373 (M + H)+ |
| 2153 | APCI: 374 (M + H)+ |
| 2154 | APCI: 375 (M + H)+ |
| 2155 | APCI: 375 (M + H)+ |
| 2156 | APCI: 377 (M + H)+ |
| 2157 | APCI: 377 (M + H)+ |
| 2158 | APCI: 379 (M + H)+ |
| 2159 | APCI: 380 (M + H)+ |
| 2160 | APCI: 381 (M + H)+ |
| 2161 | APCI: 381 (M + H)+ |
| 2162 | APCI: 385 (M + H)+ |
| 2163 | APCI: 385 (M + H)+ |
| 2164 | APCI: 391 (M + H)+ |
| 2165 | APCI: 397 (M + H)+ |
| 2166 | APCI: 405 (M + H)+ |
| 2167 | APCI: 408 (M + H)+ |
| 2168 | APCI: 414 (M + H)+ |
| 2169 | APCI: 415 (M + H)+ |
| 2170 | APCI: 429 (M + H)+ |
| 2171 | APCI: 433 (M + H)+ |
| 2172 | APCI: 283 (M + H)+ |
| 2173 | APCI: 283 (M + H)+ |
| 2174 | APCI: 297 (M + H)+ |
| 2175 | APCI: 309 (M + H)+ |
| 2176 | APCI: 313 (M + H)+ |
| 2177 | APCI: 317 (M + H)+ |
| 2178 | APCI: 317 (M + H)+ |
| 2179 | APCI: 318 (M + H)+ |
| 2180 | APCI: 318 (M + H)+ |
| 2181 | APCI: 321 (M + H)+ |
| 2182 | APCI: 322 (M + H)+ |
| 2183 | APCI: 323 (M + H)+ |
| 2184 | APCI: 323 (M + H)+ |
| 2185 | APCI: 328 (M + H)+ |
| 2186 | APCI: 331 (M + H)+ |
| 2187 | APCI: 331 (M + H)+ |
| 2188 | APCI: 331 (M + H)+ |
| 2189 | APCI: 331 (M + H)+ |
| 2190 | APCI: 332 (M + H)+ |
| 2191 | APCI: 333 (M + H)+ |
| 2192 | APCI: 335 (M + H)+ |
| 2193 | APCI: 335 (M + H)+ |
| 2194 | APCI: 337 (M + H)+ |
| 2195 | APCI: 337 (M + H)+ |
| 2196 | APCI: 338 (M + H)+ |
| 2197 | APCI: 339 (M + H)+ |
| 2198 | APCI: 339 (M + H)+ |
| 2199 | APCI: 339 (M + H)+ |
| 2200 | APCI: 339 (M + H)+ |
| 2201 | APCI: 339 (M + H)+ |
| 2202 | APCI: 339 (M + H)+ |
| 2203 | APCI: 343 (M + H)+ |
| 2204 | APCI: 345 (M + H)+ |
| 2205 | APCI: 346 (M + H)+ |
| 2206 | APCI: 347 (M + H)+ |
| 2207 | APCI: 347 (M + H)+ |
| 2208 | APCI: 348 (M + H)+ |
| 2209 | APCI: 349 (M + H)+ |
| 2210 | APCI: 351 (M + H)+ |
| 2211 | APCI: 351 (M + H)+ |
| 2212 | APCI: 353 (M + H)+ |
| 2213 | APCI: 353 (M + H)+ |
| 2214 | APCI: 353 (M + H)+ |
| 2215 | APCI: 354 (M + H)+ |
| 2216 | APCI: 354 (M + H)+ |
| 2217 | APCI: 354 (M + H)+ |
| 2218 | APCI: 354 (M + H)+ |
| 2219 | APCI: 355 (M + H)+ |
| 2220 | APCI: 355 (M + H)+ |
| 2221 | APCI: 355 (M + H)+ |
| 2222 | APCI: 355 (M + H)+ |
| 2223 | APCI: 355 (M + H)+ |
| 2224 | APCI: 355 (M + H)+ |
| 2225 | APCI: 357 (M + H)+ |
| 2226 | APCI: 357 (M + H)+ |
| 2227 | APCI: 357 (M + H)+ |
| 2228 | APCI: 363 (M + H)+ |
| 2229 | APCI: 363 (M + H)+ |
| 2230 | APCI: 363 (M + H)+ |
| 2231 | APCI: 369 (M + H)+ |
| 2232 | APCI: 369 (M + H)+ |
| 2233 | APCI: 371 (M + H)+ |
| 2234 | APCI: 371 (M + H)+ |
| 2235 | APCI: 371 (M + H)+ |
| 2236 | APCI: 372 (M + H)+ |
| 2237 | APCI: 372 (M + H)+ |
| 2238 | APCI: 372 (M + H)+ |
| 2239 | APCI: 372 (M + H)+ |
| 2240 | APCI: 373 (M + H)+ |
| 2241 | APCI: 381 (M + H)+ |
| 2242 | APCI: 381 (M + H)+ |
| 2243 | APCI: 382 (M + H)+ |
| 2244 | APCI: 382 (M + H)+ |
| 2245 | APCI: 385 (M + H)+ |
| 2246 | APCI: 387 (M + H)+ |
| 2247 | APCI: 388 (M + H)+ |
| 2248 | APCI: 388 (M + H)+ |
| 2249 | APCI: 389 (M + H)+ |
| 2250 | APCI: 389 (M + H)+ |
| 2251 | APCI: 389 (M + H)+ |
| 2252 | APCI: 391 (M + H)+ |
| 2253 | APCI: 391 (M + H)+ |
| 2254 | APCI: 393 (M + H)+ |
| 2255 | APCI: 395 (M + H)+ |
| 2256 | APCI: 395 (M + H)+ |
| 2257 | APCI: 395 (M + H)+ |
| 2258 | APCI: 399 (M + H)+ |
| 2259 | APCI: 405 (M + H)+ |
| 2260 | APCI: 411 (M + H)+ |
| 2261 | APCI: 419 (M + H)+ |
| 2262 | APCI: 423 (M + H)+ |
| 2263 | APCI: 428 (M + H)+ |
| 2264 | APCI: 429 (M + H)+ |
| 2265 | APCI: 429 (M + H)+ |
| 2266 | APCI: 443 (M + H)+ |
| 2267 | APCI: 313 (M + H)+ |
| 2268 | APCI: 313 (M + H)+ |
| 2269 | APCI: 327 (M + H)+ |
| 2270 | APCI: 339 (M + H)+ |
| 2271 | APCI: 339 (M + H)+ |
| 2272 | APCI: 343 (M + H)+ |
| 2273 | APCI: 347 (M + H)+ |
| 2274 | APCI: 347 (M + H)+ |
| 2275 | APCI: 348 (M + H)+ |
| 2276 | APCI: 348 (M + H)+ |
| 2277 | APCI: 351 (M + H)+ |
| 2278 | APCI: 352 (M + H)+ |
| 2279 | APCI: 353 (M + H)+ |
| 2280 | APCI: 353 (M + H)+ |
| 2281 | APCI: 358 (M + H)+ |
| 2282 | APCI: 361 (M + H)+ |
| 2283 | APCI: 361 (M + H)+ |
| 2284 | APCI: 361 (M + H)+ |
| 2285 | APCI: 361 (M + H)+ |
| 2286 | APCI: 361 (M + H)+ |
| 2287 | APCI: 362 (M + H)+ |
| 2288 | APCI: 363 (M + H)+ |
| 2289 | APCI: 365 (M + H)+ |
| 2290 | APCI: 365 (M + H)+ |
| 2291 | APCI: 367 (M + H)+ |
| 2292 | APCI: 367 (M + H)+ |
| 2293 | APCI: 368 (M + H)+ |
| 2294 | APCI: 369 (M + H)+ |
| 2295 | APCI: 369 (M + H)+ |
| 2296 | APCI: 369 (M + H)+ |
| 2297 | APCI: 369 (M + H)+ |
| 2298 | APCI: 369 (M + H)+ |
| 2299 | APCI: 369 (M + H)+ |
| 2300 | APCI: 373 (M + H)+ |
| 2301 | APCI: 375 (M + H)+ |
| 2302 | APCI: 376 (M + H)+ |
| 2303 | APCI: 377 (M + H)+ |
| 2304 | APCI: 377 (M + H)+ |
| 2305 | APCI: 378 (M + H)+ |
| 2306 | APCI: 379 (M + H)+ |
| 2307 | APCI: 381 (M + H)+ |
| 2308 | APCI: 381 (M + H)+ |
| 2309 | APCI: 381 (M + H)+ |
| 2310 | APCI: 381 (M + H)+ |
| 2311 | APCI: 383 (M + H)+ |
| 2312 | APCI: 383 (M + H)+ |
| 2313 | APCI: 383 (M + H)+ |
| 2314 | APCI: 384 (M + H)+ |
| 2315 | APCI: 384 (M + H)+ |
| 2316 | APCI: 384 (M + H)+ |
| 2317 | APCI: 384 (M + H)+ |
| 2318 | APCI: 385 (M + H)+ |
| 2319 | APCI: 385 (M + H)+ |
| 2320 | APCI: 385 (M + H)+ |
| 2321 | APCI: 385 (M + H)+ |
| 2322 | APCI: 385 (M + H)+ |
| 2323 | APCI: 387 (M + H)+ |
| 2324 | APCI: 387 (M + H)+ |
| 2325 | APCI: 387 (M + H)+ |
| 2326 | APCI: 393 (M + H)+ |
| 2327 | APCI: 393 (M + H)+ |
| 2328 | APCI: 393 (M + H)+ |
| 2329 | APCI: 397 (M + H)+ |
| 2330 | APCI: 397 (M + H)+ |
| 2331 | APCI: 399 (M + H)+ |
| 2332 | APCI: 399 (M + H)+ |
| 2333 | APCI: 401 (M + H)+ |
| 2334 | APCI: 401 (M + H)+ |
| 2335 | APCI: 401 (M + H)+ |
| 2336 | APCI: 401 (M + H)+ |
| 2337 | APCI: 401 (M + H)+ |
| 2338 | APCI: 402 (M + H)+ |
| 2339 | APCI: 402 (M + H)+ |
| 2340 | APCI: 402 (M + H)+ |
| 2341 | APCI: 402 (M + H)+ |
| 2342 | APCI: 402 (M + H)+ |
| 2343 | APCI: 401 (M + H)+ |
| 2344 | APCI: 403 (M + H)+ |
| 2345 | APCI: 411 (M + H)+ |
| 2346 | APCI: 412 (M + H)+ |
| 2347 | APCI: 412 (M + H)+ |
| 2348 | APCI: 417 (M + H)+ |
| 2349 | APCI: 417 (M + H)+ |
| 2350 | APCI: 418 (M + H)+ |
| 2351 | APCI: 418 (M + H)+ |
| 2352 | APCI: 419 (M + H)+ |
| 2353 | APCI: 418 (M + H)+ |
| 2354 | APCI: 419 (M + H)+ |
| 2355 | APCI: 419 (M + H)+ |
| 2356 | APCI: 419 (M + H)+ |
| 2357 | APCI: 421 (M + H)+ |
| 2358 | APCI: 421 (M + H)+ |
| 2359 | APCI: 423 (M + H)+ |
| 2360 | APCI: 425 (M + H)+ |
| 2361 | APCI: 425 (M + H)+ |
| 2362 | APCI: 425 (M + H)+ |
| 2363 | APCI: 425 (M + H)+ |
| 2364 | ESI: 429 (M + H)+ |
| 2365 | APCI: 429 (M + H)+ |
| 2366 | APCI: 429 (M + H)+ |
| 2367 | APCI: 435 (M + H)+ |
| 2368 | APCI: 441 (M + H)+ |
| 2369 | APCI: 452 (M + H)+ |
| 2370 | APCI: 453 (M + H)+ |
| 2371 | APCI: 458 (M + H)+ |
| 2372 | APCI: 459 (M + H)+ |
| 2373 | APCI: 473 (M + H)+ |
| 2374 | APCI: 477 (M + H)+ |
| 2375 | APCI: 267 (M + H)+ |
| 2376 | APCI: 281 (M + H)+ |
| 2377 | APCI: 293 (M + H)+ |
| 2378 | APCI: 295 (M + H)+ |
| 2379 | APCI: 295 (M + H)+ |
| 2380 | APCI: 297 (M + H)+ |
| 2381 | APCI: 307 (M + H)+ |
| 2382 | APCI: 309 (M + H)+ |
| 2383 | APCI: 309 (M + H)+ |
| 2384 | APCI: 309 (M + H)+ |
| 2385 | APCI: 310 (M + H)+ |
| 2386 | APCI: 319 (M + H)+ |
| 2387 | APCI: 319 (M + H)+ |
| 2388 | APCI: 320 (M + H)+ |
| 2389 | APCI: 321 (M + H)+ |
| 2390 | APCI: 323 (M + H)+ |
| 2391 | APCI: 323 (M + H)+ |
| 2392 | APCI: 323 (M + H)+ |
| 2393 | APCI: 323 (M + H)+ |
| 2394 | APCI: 325 (M + H)+ |
| 2395 | APCI: 327 (M + H)+ |
| 2396 | APCI: 330 (M + H)+ |
| 2397 | APCI: 331 (M + H)+ |
| 2398 | APCI: 331 (M + H)+ |
| 2399 | APCI: 331 (M + H)+ |
| 2400 | APCI: 332 (M + H)+ |
| 2401 | APCI: 333 (M + H)+ |
| 2402 | APCI: 333 (M + H)+ |
| 2403 | APCI: 334 (M + H)+ |
| 2404 | APCI: 335 (M + H)+ |
| 2405 | APCI: 335 (M + H)+ |
| 2406 | APCI: 336 (M + H)+ |
| 2407 | APCI: 337 (M + H)+ |
| 2408 | APCI: 338 (M + H)+ |
| 2409 | APCI: 339 (M + H)+ |
| 2410 | APCI: 339 (M + H)+ |
| 2411 | APCI: 344 (M + H)+ |
| 2412 | APCI: 344 (M + H)+ |
| 2413 | APCI: 344 (M + H)+ |
| 2414 | APCI: 345 (M + H)+ |
| 2415 | APCI: 345 (M + H)+ |
| 2416 | APCI: 346 (M + H)+ |
| 2417 | APCI: 346 (M + H)+ |
| 2418 | APCI: 346 (M + H)+ |
| 2419 | APCI: 346 (M + H)+ |
| 2420 | APCI: 347 (M + H)+ |
| 2421 | APCI: 347 (M + H)+ |
| 2422 | APCI: 347 (M + H)+ |
| 2423 | APCI: 347 (M + H)+ |
| 2424 | APCI: 347 (M + H)+ |
| 2425 | APCI: 347 (M + H)+ |
| 2426 | APCI: 348 (M + H)+ |
| 2427 | APCI: 348 (M + H)+ |
| 2428 | APCI: 349 (M + H)+ |
| 2429 | APCI: 350 (M + H)+ |
| 2430 | APCI: 351 (M + H)+ |
| 2431 | APCI: 351 (M + H)+ |
| 2432 | APCI: 351 (M + H)+ |
| 2433 | APCI: 353 (M + H)+ |
| 2434 | APCI: 354 (M + H)+ |
| 2435 | APCI: 355 (M + H)+ |
| 2436 | APCI: 355 (M + H)+ |
| 2437 | APCI: 355 (M + H)+ |
| 2438 | APCI: 357 (M + H)+ |
| 2439 | APCI: 357 (M + H)+ |
| 2440 | APCI: 357 (M + H)+ |
| 2441 | APCI: 358 (M + H)+ |
| 2442 | APCI: 359 (M + H)+ |
| 2443 | APCI: 359 (M + H)+ |
| 2444 | APCI: 359 (M + H)+ |
| 2445 | APCI: 360 (M + H)+ |
| 2446 | APCI: 360 (M + H)+ |
| 2447 | APCI: 361 (M + H)+ |
| 2448 | APCI: 361 (M + H)+ |
| 2449 | APCI: 361 (M + H)+ |
| 2450 | APCI: 361 (M + H)+ |
| 2451 | APCI: 361 (M + H)+ |
| 2452 | APCI: 364 (M + H)+ |
| 2453 | APCI: 364 (M + H)+ |
| 2454 | APCI: 364 (M + H)+ |
| 2455 | APCI: 364 (M + H)+ |
| 2456 | APCI: 365 (M + H)+ |
| 2457 | APCI: 367 (M + H)+ |
| 2458 | APCI: 368 (M + H)+ |
| 2459 | APCI: 369 (M + H)+ |
| 2460 | APCI: 369 (M + H)+ |
| 2461 | APCI: 369 (M + H)+ |
| 2462 | APCI: 369 (M + H)+ |
| 2463 | APCI: 369 (M + H)+ |
| 2464 | APCI: 369 (M + H)+ |
| 2465 | APCI: 369 (M + H)+ |
| 2466 | APCI: 371 (M + H)+ |
| 2467 | APCI: 371 (M + H)+ |
| 2468 | APCI: 371 (M + H)+ |
| 2469 | APCI: 371 (M + H)+ |
| 2470 | APCI: 372 (M + H)+ |
| 2471 | APCI: 373 (M + H)+ |
| 2472 | APCI: 373 (M + H)+ |
| 2473 | APCI: 373 (M + H)+ |
| 2474 | APCI: 373 (M + H)+ |
| 2475 | APCI: 373 (M + H)+ |
| 2476 | APCI: 373 (M + H)+ |
| 2477 | APCI: 374 (M + H)+ |
| 2478 | APCI: 374 (M + H)+ |
| 2479 | APCI: 374 (M + H)+ |
| 2480 | APCI: 375 (M + H)+ |
| 2481 | APCI: 375 (M + H)+ |
| 2482 | APCI: 376 (M + H)+ |
| 2483 | APCI: 376 (M + H)+ |
| 2484 | APCI: 377 (M + H)+ |
| 2485 | APCI: 378 (M + H)+ |
| 2486 | APCI: 378 (M + H)+ |
| 2487 | APCI: 379 (M + H)+ |
| 2488 | APCI: 379 (M + H)+ |
| 2489 | APCI: 379 (M + H)+ |
| 2490 | APCI: 380 (M + H)+ |
| 2491 | APCI: 381 (M + H)+ |
| 2492 | APCI: 381 (M + H)+ |
| 2493 | APCI: 381 (M + H)+ |
| 2494 | APCI: 381 (M + H)+ |
| 2495 | APCI: 381 (M + H)+ |
| 2496 | APCI: 381 (M + H)+ |
| 2497 | APCI: 381 (M + H)+ |
| 2498 | APCI: 382 (M + H)+ |
| 2499 | APCI: 382 (M + H)+ |
| 2500 | APCI: 382 (M + H)+ |
| 2501 | APCI: 383 (M + H)+ |
| 2502 | APCI: 383 (M + H)+ |
| 2503 | APCI: 383 (M + H)+ |
| 2504 | APCI: 383 (M + H)+ |
| 2505 | APCI: 384 (M + H)+ |
| 2506 | APCI: 385 (M + H)+ |
| 2507 | APCI: 385 (M + H)+ |
| 2508 | APCI: 385 (M + H)+ |
| 2509 | APCI: 385 (M + H)+ |
| 2510 | APCI: 386 (M + H)+ |
| 2511 | APCI: 387 (M + H)+ |
| 2512 | APCI: 387 (M + H)+ |
| 2513 | APCI: 387 (M + H)+ |
| 2514 | APCI: 387 (M + H)+ |
| 2515 | APCI: 387 (M + H)+ |
| 2516 | APCI: 387 (M + H)+ |
| 2517 | APCI: 387 (M + H)+ |
| 2518 | APCI: 388 (M + H)+ |
| 2519 | APCI: 389 (M + H)+ |
| 2520 | APCI: 389 (M + H)+ |
| 2521 | APCI: 389 (M + H)+ |
| 2522 | APCI: 389 (M + H)+ |
| 2523 | APCI: 389 (M + H)+ |
| 2524 | APCI: 389 (M + H)+ |
| 2525 | APCI: 389 (M + H)+ |
| 2526 | APCI: 389 (M + H)+ |
| 2527 | APCI: 390 (M + H)+ |
| 2528 | APCI: 390 (M + H)+ |
| 2529 | APCI: 390 (M + H)+ |
| 2530 | APCI: 393 (M + H)+ |
| 2531 | APCI: 393 (M + H)+ |
| 2532 | APCI: 395 (M + H)+ |
| 2533 | APCI: 395 (M + H)+ |
| 2534 | APCI: 395 (M + H)+ |
| 2535 | APCI: 395 (M + H)+ |
| 2536 | APCI: 395 (M + H)+ |
| 2537 | APCI: 395 (M + H)+ |
| 2538 | APCI: 396 (M + H)+ |
| 2539 | APCI: 396 (M + H)+ |
| 2540 | APCI: 397 (M + H)+ |
| 2541 | APCI: 397 (M + H)+ |
| 2542 | APCI: 397 (M + H)+ |
| 2543 | APCI: 397 (M + H)+ |
| 2544 | APCI: 397 (M + H)+ |
| 2545 | APCI: 397 (M + H)+ |
| 2546 | APCI: 397 (M + H)+ |
| 2547 | APCI: 398 (M + H)+ |
| 2548 | APCI: 398 (M + H)+ |
| 2549 | APCI: 398 (M + H)+ |
| 2550 | APCI: 398 (M + H)+ |
| 2551 | APCI: 398 (M + H)+ |
| 2552 | APCI: 398 (M + H)+ |
| 2553 | ESI: 399 (M + H)+ |
| 2554 | APCI: 399 (M + H)+ |
| 2555 | APCI: 399 (M + H)+ |
| 2556 | APCI: 399 (M + H)+ |
| 2557 | APCI: 401 (M + H)+ |
| 2558 | APCI: 401 (M + H)+ |
| 2559 | APCI: 401 (M + H)+ |
| 2560 | APCI: 401 (M + H)+ |
| 2561 | APCI: 401 (M + H)+ |
| 2562 | APCI: 403 (M + H)+ |
| 2563 | APCI: 403 (M + H)+ |
| 2564 | APCI: 403 (M + H)+ |
| 2565 | APCI: 404 (M + H)+ |
| 2566 | APCI: 404 (M + H)+ |
| 2567 | APCI: 404 (M + H)+ |
| 2568 | APCI: 404 (M + H)+ |
| 2569 | APCI: 405 (M + H)+ |
| 2570 | APCI: 405 (M + H)+ |
| 2571 | APCI: 406 (M + H)+ |
| 2572 | APCI: 408 (M + H)+ |
| 2573 | APCI: 408 (M + H)+ |
| 2574 | ESI: 409 (M + H)+ |
| 2575 | APCI: 410 (M + H)+ |
| 2576 | APCI: 410 (M + H)+ |
| 2577 | APCI: 410 (M + H)+ |
| 2578 | APCI: 411 (M + H)+ |
| 2579 | APCI: 412 (M + H)+ |
| 2580 | APCI: 412 (M + H)+ |
| 2581 | APCI: 412 (M + H)+ |
| 2582 | APCI: 412 (M + H)+ |
| 2583 | APCI: 412 (M + H)+ |
| 2584 | APCI: 413 (M + H)+ |
| 2585 | APCI: 413 (M + H)+ |
| 2586 | APCI: 413 (M + H)+ |
| 2587 | APCI: 413 (M + H)+ |
| 2588 | APCI: 413 (M + H)+ |
| 2589 | APCI: 414 (M + H)+ |
| 2590 | APCI: 415 (M + H)+ |
| 2591 | APCI: 415 (M + H)+ |
| 2592 | APCI: 415 (M + H)+ |
| 2593 | APCI: 415 (M + H)+ |
| 2594 | APCI: 415 (M + H)+ |
| 2595 | APCI: 415 (M + H)+ |
| 2596 | APCI: 415 (M + H)+ |
| 2597 | APCI: 416 (M + H)+ |
| 2598 | APCI: 417 (M + H)+ |
| 2599 | APCI: 417 (M + H)+ |
| 2600 | APCI: 418 (M + H)+ |
| 2601 | APCI: 418 (M + H)+ |
| 2602 | APCI: 419 (M + H)+ |
| 2603 | APCI: 419 (M + H)+ |
| 2604 | APCI: 419 (M + H)+ |
| 2605 | APCI: 419 (M + H)+ |
| 2606 | APCI: 419 (M + H)+ |
| 2607 | APCI: 421 (M + H)+ |
| 2608 | APCI: 421 (M + H)+ |
| 2609 | APCI: 421 (M + H)+ |
| 2610 | APCI: 421 (M + H)+ |
| 2611 | APCI: 422 (M + H)+ |
| 2612 | APCI: 422 (M + H)+ |
| 2613 | APCI: 423 (M + H)+ |
| 2614 | APCI: 425 (M + H)+ |
| 2615 | APCI: 427 (M + H)+ |
| 2616 | APCI: 427 (M + H)+ |
| 2617 | APCI: 427 (M + H)+ |
| 2618 | APCI: 427 (M + H)+ |
| 2619 | APCI: 427 (M + H)+ |
| 2620 | APCI: 427 (M + H)+ |
| 2621 | APCI: 428 (M + H)+ |
| 2622 | APCI: 428 (M + H)+ |
| 2623 | APCI: 428 (M + H)+ |
| 2624 | APCI: 429 (M + H)+ |
| 2625 | APCI: 429 (M + H)+ |
| 2626 | APCI: 429 (M + H)+ |
| 2627 | APCI: 431 (M + H)+ |
| 2628 | APCI: 431 (M + H)+ |
| 2629 | APCI: 431 (M + H)+ |
| 2630 | APCI: 432 (M + H)+ |
| 2631 | APCI: 432 (M + H)+ |
| 2632 | APCI: 435 (M + H)+ |
| 2633 | APCI: 437 (M + H)+ |
| 2634 | APCI: 437 (M + H)+ |
| 2635 | APCI: 438 (M + H)+ |
| 2636 | APCI: 439 (M + H)+ |
| 2637 | APCI: 439 (M + H)+ |
| 2638 | APCI: 339 (M + H)+ |
| 2639 | APCI: 440 (M + H)+ |
| 2640 | APCI: 441 (M + H)+ |
| 2641 | APCI: 443 (M + H)+ |
| 2642 | APCI: 444 (M + H)+ |
| 2643 | APCI: 444 (M + H)+ |
| 2644 | APCI: 448 (M + H)+ |
| 2645 | APCI: 449 (M + H)+ |
| 2646 | APCI: 449 (M + H)+ |
| 2647 | APCI: 449 (M + H)+ |
| 2648 | APCI: 453 (M + H)+ |
| 2649 | APCI: 454 (M + H)+ |
| 2650 | APCI: 455 (M + H)+ |
| 2651 | APCI: 455 (M + H)+ |
| 2652 | APCI: 456 (M + H)+ |
| 2653 | APCI: 457 (M + H)+ |
| 2654 | APCI: 459 (M + H)+ |
| 2655 | APCI: 462 (M + H)+ |
| 2656 | APCI: 462 (M + H)+ |
| 2657 | APCI: 462 (M + H)+ |
| 2658 | APCI: 463 (M + H)+ |
| 2659 | APCI: 465 (M + H)+ |
| 2660 | APCI: 465 (M + H)+ |
| 2661 | APCI: 465 (M + H)+ |
| 2662 | APCI: 471 (M + H)+ |
| 2663 | APCI: 475 (M + H)+ |
| 2664 | APCI: 476 (M + H)+ |
| 2665 | APCI: 477 (M + H)+ |
| 2666 | APCI: 478 (M + H)+ |
| 2667 | APCI: 482 (M + H)+ |
| 2668 | APCI: 483 (M + H)+ |
| 2669 | APCI: 492 (M + H)+ |
| 2670 | APCI: 494 (M + H)+ |
| 2671 | APCI: 495 (M + H)+ |
| 2672 | APCI: 497 (M + H)+ |
| 2673 | APCI: 497 (M + H)+ |
| 2674 | APCI: 498 (M + H)+ |
| 2675 | APCI: 508 (M + H)+ |
| 2676 | APCI: 523 (M + H)+ |
| 2677 | APCI: 531 (M + H)+ |
| 2678 | APCI: 533 (M + H)+ |

TABLE 14

| No. | MASS |
|---|---|
| 3001 | ESI (Pos) 250 (M + H)+ |
| 3002 | ESI (Pos) 264 (M + H)+ |
| 3003 | ESI (Pos) 278 (M + H)+ |
| 3004 | ESI (Pos) 278 (M + H)+ |
| 3005 | ESI (Pos) 292 (M + H)+ |
| 3006 | ESI (Pos) 292 (M + H)+ |
| 3007 | ESI (Pos) 292 (M + H)+ |
| 3008 | ESI (Pos) 306 (M + H)+ |
| 3009 | ESI (Pos) 306 (M + H)+ |
| 3010 | ESI (Pos) 306 (M + H)+ |
| 3011 | ESI (Pos) 290 (M + H)+ |
| 3012 | ESI (Pos) 316 (M + H)+ |
| 3013 | ESI (Pos) 332 (M + H)+ |
| 3014 | ESI (Pos) 326 (M + H)+ |
| 3015 | ESI (Pos) 340 (M + H)+ |
| 3016 | ESI (Pos) 354 (M + H)+ |
| 3017 | ESI (Pos) 402 (M + H)+ |
| 3018 | ESI (Pos) 330 (M + H)+ |
| 3019 | ESI (Pos) 342 (M + H)+ |
| 3020 | ESI (Pos) 356 (M + H)+ |
| 3021 | ESI (Pos) 280 (M + H)+ |
| 3022 | ESI (Pos) 340 (M + H)+ |
| 3023 | ESI (Pos) 356 (M + H)+ |
| 3024 | ESI (Pos) 356 (M + H)+ |
| 3025 | ESI (Pos) 310 (M + H)+ |
| 3026 | ESI (Pos) 308 (M + H)+ |
| 3027 | ESI (Pos) 384 (M + H)+ |
| 3028 | ESI (Pos) 404 (M + H)+ |
| 3029 | ESI (Pos) 338 (M + H)+ |
| 3030 | ESI (Pos) 280 (M + H)+ |
| 3031 | ESI (Pos) 310 (M + H)+ |
| 3032 | ESI (Pos) 321 (M + H)+ |
| 3033 | ESI (Pos) 322 (M + H)+ |
| 3034 | ESI (Pos) 276 (M + H)+ |
| 3035 | ESI (Pos) 290 (M + H)+ |
| 3036 | ESI (Pos) 304 (M + H)+ |
| 3037 | ESI (Pos) 318 (M + H)+ |
| 3038 | ESI (Pos) 370 (M + H)+ |
| 3039 | ESI (Pos) 352 (M + H)+ |
| 3040 | ESI (Pos) 414 (M + H)+ |
| 3041 | ESI (Pos) 312 (M + H)+ |
| 3042 | ESI (Pos) 326 (M + H)+ |
| 3043 | ESI (Pos) 326 (M + H)+ |
| 3044 | ESI (Pos) 326 (M + H)+ |
| 3045 | ESI (Pos) 330 (M + H)+ |
| 3046 | ESI (Pos) 330 (M + H)+ |
| 3047 | ESI (Pos) 330 (M + H)+ |
| 3048 | ESI (Pos) 337 (M + H)+ |
| 3049 | ESI (Pos) 337 (M + H)+ |
| 3050 | ESI (Pos) 340 (M + H)+ |
| 3051 | ESI (Pos) 340 (M + H)+ |
| 3052 | ESI (Pos) 340 (M + H)+ |
| 3053 | ESI (Pos) 342 (M + H)+ |
| 3054 | ESI (Pos) 342 (M + H)+ |
| 3055 | ESI (Pos) 344 (M + H)+ |
| 3056 | ESI (Pos) 344 (M + H)+ |
| 3057 | ESI (Pos) 346 (M + H)+ |
| 3058 | ESI (Pos) 346 (M + H)+ |
| 3059 | ESI (Pos) 346 (M + H)+ |
| 3060 | ESI (Pos) 348 (M + H)+ |
| 3061 | ESI (Pos) 348 (M + H)+ |
| 3062 | ESI (Pos) 348 (M + H)+ |
| 3063 | ESI (Pos) 348 (M + H)+ |
| 3064 | ESI (Pos) 348 (M + H)+ |
| 3065 | ESI (Pos) 348 (M + H)+ |
| 3066 | ESI (Pos) 354 (M + H)+ |
| 3067 | ESI (Pos) 354 (M + H)+ |
| 3068 | ESI (Pos) 356 (M + H)+ |
| 3069 | ESI (Pos) 357 (M + H)+ |
| 3070 | ESI (Pos) 362 (M + H)+ |
| 3071 | ESI (Pos) 362 (M + H)+ |
| 3072 | ESI (Pos) 362 (M + H)+ |
| 3073 | ESI (Pos) 362 (M + H)+ |
| 3074 | ESI (Pos) 364 (M + H)+ |
| 3075 | ESI (Pos) 364 (M + H)+ |
| 3076 | ESI (Pos) 364 (M + H)+ |
| 3077 | ESI (Pos) 364 (M + H)+ |
| 3078 | ESI (Pos) 364 (M + H)+ |
| 3079 | ESI (Pos) 366 (M + H)+ |
| 3080 | ESI (Pos) 366 (M + H)+ |
| 3081 | ESI (Pos) 366 (M + H)+ |
| 3082 | ESI (Pos) 366 (M + H)+ |
| 3083 | ESI (Pos) 368 (M + H)+ |
| 3084 | ESI (Pos) 392 (M + H)+ |
| 3085 | ESI (Pos) 372 (M + H)+ |
| 3086 | ESI (Pos) 378 (M + H)+ |
| 3087 | ESI (Pos) 378 (M + H)+ |
| 3088 | ESI (Pos) 380 (M + H)+ |
| 3089 | ESI (Pos) 380 (M + H)+ |
| 3090 | ESI (Pos) 378 (M + H)+ |
| 3091 | ESI (Pos) 380 (M + H)+ |
| 3092 | ESI (Pos) 380 (M + H)+ |
| 3093 | ESI (Pos) 382 (M + H)+ |
| 3094 | ESI (Pos) 382 (M + H)+ |
| 3095 | ESI (Pos) 382 (M + H)+ |
| 3096 | ESI (Pos) 384 (M + H)+ |
| 3097 | ESI (Pos) 388 (M + H)+ |
| 3098 | ESI (Pos) 390 (M + H)+ |
| 3099 | ESI (Pos) 390 (M + H)+ |
| 3100 | ESI (Pos) 390 (M + H)+ |
| 3101 | ESI (Pos) 396 (M + H)+ |
| 3102 | ESI (Pos) 396 (M + H)+ |
| 3103 | ESI (Pos) 396 (M + H)+ |
| 3104 | ESI (Pos) 398 (M + H)+ |
| 3105 | ESI (Pos) 398 (M + H)+ |
| 3106 | ESI (Pos) 398 (M + H)+ |
| 3107 | ESI (Pos) 398 (M + H)+ |
| 3108 | ESI (Pos) 398 (M + H)+ |
| 3109 | ESI (Pos) 398 (M + H)+ |
| 3110 | ESI (Pos) 398 (M + H)+ |
| 3111 | ESI (Pos) 400 (M + H)+ |
| 3112 | ESI (Pos) 402 (M + H)+ |
| 3113 | ESI (Pos) 402 (M + H)+ |
| 3114 | ESI (Pos) 404 (M + H)+ |
| 3115 | ESI (Pos) 412 (M + H)+ |
| 3116 | ESI (Pos) 414 (M + H)+ |
| 3117 | ESI (Pos) 414 (M + H)+ |
| 3118 | ESI (Pos) 418 (M + H)+ |
| 3119 | ESI (Pos) 432 (M + H)+ |
| 3120 | ESI (Pos) 438 (M + H)+ |
| 3121 | ESI (Pos) 438 (M + H)+ |
| 3122 | ESI (Pos) 438 (M + H)+ |
| 3123 | ESI (Pos) 442 (M + H)+ |
| 3124 | ESI (Pos) 448 (M + H)+ |
| 3125 | ESI (Pos) 448 (M + H)+ |
| 3126 | ESI (Pos) 355 (M + H)+ |
| 3127 | ESI (Pos) 396 (M + H)+ |
| 3128 | ESI (Pos) 313 (M + H)+ |
| 3129 | ESI (Pos) 313 (M + H)+ |
| 3130 | ESI (Pos) 364 (M + H)+ |
| 3131 | ESI (Pos) 377 (M + H)+ |
| 3132 | ESI (Pos) 395 (M + H)+ |
| 3133 | ESI (Pos) 421 (M + H)+ |
| 3134 | ESI (Pos) 439 (M + H)+ |
| 3135 | ESI (Pos) 302 (M + H)+ |
| 3136 | ESI (Pos) 303 (M + H)+ |
| 3137 | ESI (Pos) 317 (M + H)+ |
| 3138 | ESI (Pos) 318 (M + H)+ |
| 3139 | ESI (Pos) 347 (M + H)+ |
| 3140 | ESI (Pos) 330 (M + H)+ |
| 3141 | ESI (Pos) 331 (M + H)+ |
| 3142 | ESI (Pos) 344 (M + H)+ |
| 3143 | ESI (Pos) 372 (M + H)+ |
| 3144 | ESI (Pos) 372 (M + H)+ |
| 3145 | ESI (Pos) 384 (M + H)+ |
| 3146 | ESI (Pos) 368 (M + H)+ |
| 3147 | ESI (Pos) 368 (M + H)+ |
| 3148 | ESI (Pos) 368 (M + H)+ |
| 3149 | ESI (Pos) 398 (M + H)+ |
| 3150 | ESI (Pos) 400 (M + H)+ |
| 3151 | ESI (Pos) 403 (M + H)+ |
| 3152 | ESI (Pos) 420 (M + H)+ |
| 3153 | ESI (Pos) 427 (M + H)+ |
| 3154 | ESI (Pos) 446 (M + H)+ |
| 3155 | ESI (Pos) 448 (M + H)+ |
| 3156 | ESI (Pos) 454 (M + H)+ |
| 3157 | ESI (Pos) 462 (M + H)+ |
| 3158 | ESI (Pos) 480 (M + H)+ |

TABLE 15

| No. | MASS |
|---|---|
| 3159 | APCI: 317 (M + H)+ |
| 3160 | APCI: 317 (M + H)+ |
| 3161 | APCI: 317 (M + H)+ |
| 3162 | APCI: 318 (M + H)+ |
| 3163 | APCI: 318 (M + H)+ |
| 3164 | APCI: 318 (M + H)+ |
| 3165 | APCI: 318 (M + H)+ |
| 3166 | APCI: 331 (M + H)+ |
| 3167 | APCI: 331 (M + H)+ |
| 3168 | APCI: 331 (M + H)+ |
| 3169 | APCI: 331 (M + H)+ |
| 3170 | APCI: 331 (M + H)+ |
| 3171 | APCI: 331 (M + H)+ |
| 3172 | APCI: 331 (M + H)+ |
| 3173 | APCI: 332 (M + H)+ |
| 3174 | APCI: 333 (M + H)+ |
| 3175 | APCI: 333 (M + H)+ |
| 3176 | APCI: 333 (M + H)+ |
| 3177 | APCI: 333 (M + H)+ |
| 3178 | APCI: 335 (M + H)+ |
| 3179 | APCI: 335 (M + H)+ |
| 3180 | APCI: 335 (M + H)+ |
| 3181 | APCI: 335 (M + H)+ |
| 3182 | APCI: 342 (M + H)+ |
| 3183 | APCI: 345 (M + H)+ |
| 3184 | APCI: 345 (M + H)+ |
| 3185 | APCI: 345 (M + H)+ |
| 3186 | APCI: 345 (M + H)+ |
| 3187 | APCI: 347 (M + H)+ |
| 3188 | APCI: 347 (M + H)+ |
| 3189 | APCI: 348 (M + H)+ |
| 3190 | APCI: 349 (M + H)+ |
| 3191 | APCI: 351 (M + H)+ |
| 3192 | APCI: 351 (M + H)+ |
| 3193 | APCI: 351 (M + H)+ |
| 3194 | APCI: 351 (M + H)+ |
| 3195 | APCI: 351 (M + H)+ |
| 3196 | APCI: 352 (M + H)+ |
| 3197 | APCI: 353 (M + H)+ |
| 3198 | APCI: 360 (M + H)+ |
| 3199 | APCI: 361 (M + H)+ |
| 3200 | APCI: 361 (M + H)+ |
| 3201 | APCI: 363 (M + H)+ |
| 3202 | APCI: 365 (M + H)+ |
| 3203 | APCI: 365 (M + H)+ |
| 3204 | APCI: 369 (M + H)+ |
| 3205 | APCI: 371 (M + H)+ |
| 3206 | APCI: 374 (M + H)+ |
| 3207 | APCI: 377 (M + H)+ |
| 3208 | APCI: 378 (M + H)+ |
| 3209 | APCI: 378 (M + H)+ |
| 3210 | APCI: 381 (M + H)+ |
| 3211 | APCI: 383 (M + H)+ |
| 3212 | APCI: 383 (M + H)+ |
| 3213 | APCI: 384 (M + H)+ |
| 3214 | APCI: 385 (M + H)+ |
| 3215 | APCI: 385 (M + H)+ |
| 3216 | APCI: 385 (M + H)+ |
| 3217 | APCI: 385 (M + H)+ |
| 3218 | APCI: 385 (M + H)+ |
| 3219 | APCI: 385 (M + H)+ |
| 3220 | APCI: 385 (M + H)+ |
| 3221 | APCI: 385 (M + H)+ |
| 3222 | APCI: 385 (M + H)+ |
| 3223 | APCI: 385 (M + H)+ |
| 3224 | APCI: 386 (M + H)+ |
| 3225 | APCI: 386 (M + H)+ |
| 3226 | APCI: 386 (M + H)+ |
| 3227 | APCI: 386 (M + H)+ |
| 3228 | APCI: 389 (M + H)+ |
| 3229 | APCI: 391 (M + H)+ |
| 3230 | APCI: 393 (M + H)+ |
| 3231 | APCI: 395 (M + H)+ |
| 3232 | APCI: 395 (M + H)+ |
| 3233 | APCI: 395 (M + H)+ |
| 3234 | APCI: 395 (M + H)+ |
| 3235 | APCI: 395 (M + H)+ |
| 3236 | APCI: 399 (M + H)+ |
| 3237 | APCI: 399 (M + H)+ |
| 3238 | APCI: 399 (M + H)+ |
| 3239 | APCI: 400 (M + H)+ |
| 3240 | APCI: 403 (M + H)+ |
| 3241 | APCI: 405 (M + H)+ |
| 3242 | APCI: 409 (M + H)+ |
| 3243 | APCI: 410 (M + H)+ |
| 3244 | APCI: 415 (M + H)+ |
| 3245 | APCI: 414 (M + H)+ |
| 3246 | APCI: 415 (M + H)+ |
| 3247 | APCI: 418 (M + H)+ |
| 3248 | APCI: 418 (M + H)+ |
| 3249 | APCI: 419 (M + H)+ |
| 3250 | ESI: 423 (M + H)+ |
| 3251 | APCI: 425 (M + H)+ |
| 3252 | APCI: 427 (M + H)+ |
| 3253 | APCI: 440 (M + H)+ |
| 3254 | APCI: 443 (M + H)+ |
| 3255 | APCI: 355 (M + H)+ |
| 3256 | APCI: 355 (M + H)+ |
| 3257 | APCI: 355 (M + H)+ |
| 3258 | APCI: 355 (M + H)+ |
| 3259 | APCI: 356 (M + H)+ |
| 3260 | APCI: 356 (M + H)+ |
| 3261 | APCI: 367 (M + H)+ |
| 3262 | APCI: 367 (M + H)+ |
| 3263 | APCI: 367 (M + H)+ |
| 3264 | APCI: 367 (M + H)+ |
| 3265 | APCI: 367 (M + H)+ |
| 3266 | APCI: 368 (M + H)+ |
| 3267 | APCI: 368 (M + H)+ |
| 3268 | APCI: 368 (M + H)+ |
| 3269 | APCI: 368 (M + H)+ |
| 3270 | APCI: 368 (M + H)+ |
| 3271 | APCI: 369 (M + H)+ |
| 3272 | APCI: 370 (M + H)+ |
| 3273 | APCI: 371 (M + H)+ |
| 3274 | APCI: 372 (M + H)+ |
| 3275 | APCI: 373 (M + H)+ |
| 3276 | APCI: 374 (M + H)+ |
| 3277 | APCI: 381 (M + H)+ |
| 3278 | APCI: 381 (M + H)+ |
| 3279 | APCI: 382 (M + H)+ |
| 3280 | APCI: 382 (M + H)+ |
| 3281 | APCI: 383 (M + H)+ |
| 3282 | APCI: 383 (M + H)+ |
| 3283 | APCI: 387 (M + H)+ |
| 3284 | APCI: 387 (M + H)+ |
| 3285 | APCI: 399 (M + H)+ |
| 3286 | APCI: 401 (M + H)+ |
| 3287 | APCI: 409 (M + H)+ |
| 3288 | APCI: 414 (M + H)+ |
| 3289 | APCI: 419 (M + H)+ |
| 3290 | APCI: 419 (M + H)+ |
| 3291 | APCI: 421 (M + H)+ |
| 3292 | APCI: 435 (M + H)+ |
| 3293 | APCI: 441 (M + H)+ |
| 3294 | APCI: 443 (M + H)+ |
| 3295 | APCI: 444 (M + H)+ |
| 3296 | APCI: 456 (M + H)+ |
| 3297 | APCI: 477 (M − H)− |
| 3298 | APCI: 359 (M + H)+ |
| 3299 | APCI: 359 (M + H)+ |
| 3300 | APCI: 381 (M + H)+ |
| 3301 | APCI: 381 (M + H)+ |
| 3302 | APCI: 381 (M + H)+ |
| 3303 | APCI: 382 (M + H)+ |
| 3304 | APCI: 382 (M + H)+ |
| 3305 | APCI: 385 (M + H)+ |
| 3306 | APCI: 387 (M + H)+ |
| 3307 | APCI: 398 (M + H)+ |
| 3308 | APCI: 398 (M + H)+ |
| 3309 | APCI: 399 (M + H)+ |
| 3310 | APCI: 400 (M + H)+ |
| 3311 | APCI: 401 (M + H)+ |
| 3312 | APCI: 402 (M + H)+ |
| 3313 | APCI: 410 (M + H)+ |
| 3314 | APCI: 413 (M + H)+ |
| 3315 | APCI: 414 (M + H)+ |
| 3316 | APCI: 415 (M + H)+ |
| 3317 | APCI: 419 (M + H)+ |
| 3318 | APCI: 427 (M + H)+ |
| 3319 | APCI: 431 (M + H)+ |
| 3320 | APCI: 435 (M + H)+ |
| 3321 | APCI: 444 (M + H)+ |
| 3322 | APCI: 449 (M + H)+ |
| 3323 | APCI: 449 (M + H)+ |
| 3324 | APCI: 451 (M + H)+ |
| 3325 | APCI: 451 (M + H)+ |
| 3326 | APCI: 459 (M + H)+ |
| 3327 | APCI: 463 (M + H)+ |

### Experimental Example 1

### Human CB2 Receptor Binding Inhibition Test

First, cDNA sequence (Munro et al., Nature, 1993, 365, 61-65) encoding a human CB2 receptor was inserted in the forward direction in an animal-cell expression vector, pTARGET Vector (manufactured by Promega) at a region downstream of a CMV promoter. Host cells CHO-DHFR(−) were transfected with the obtained expression vector with the aid of Lipofectamine (manufactured by Invitrogen) to obtain cells capable of stably expressing the CB2 receptor.

The membrane fractions prepared from CHO cells capable of stably expressing the CB2 receptor were incubated together with a test compound and [³H]CP-55,940 (final concentration: nM, manufactured by Perkin Elmer) in an assay buffer (50 mM Tris-HCl buffer (pH 7.4), 2.5 mM EDTA, 5 mM MgCl₂) containing 0.2% bovine serum albumin at 25° C. for 2 hours, and thereafter, filtrated by a glass filter GF/C treated with 0.1% poly-L-lysine (manufactured by SIGMA). After the filtrate was washed with an assay buffer containing 0.1% bovine serum albumin, the radioactivity on the glass filter was measured by a liquid scintillation counter. Nonspecific binding was measured in the presence of 2.0 μM CP-55,940 (manufactured by Tocris). 50% inhibitory concentration (IC₅₀ value) of the test compound for the specific binding was obtained. The test results are shown in Table 16. As shown in the table, the tested compounds exhibited affinity for the CB2 receptor.

TABLE 16
Inhibition of binding to human CB2 receptor

| | CB2 |
|---|---|
| Compound | IC50 |
| No. | (nM) |
| 9 | 11.3 |
| 12 | 13.3 |
| 23 | 8.9 |
| 24 | 7.5 |
| 25 | 17.4 |
| 29 | 8.6 |
| 30 | 6.6 |
| 33 | 8.2 |
| 34 | 6.6 |
| 35 | 3.4 |
| 36 | 2.2 |
| 37 | 11.3 |
| 41 | 1.4 |
| 45 | 16.5 |
| 46 | 1.2 |
| 51 | 2.6 |
| 54 | 18.7 |
| 56 | 0.8 |
| 57 | 3.8 |
| 58 | 1.8 |
| 59 | 7.2 |
| 63 | 4.9 |
| 67 | 3.0 |
| 70 | 14.9 |
| 71 | 5.9 |
| 73 | 10.2 |
| 74 | 4.6 |
| 76 | 14.4 |
| 77 | 19.3 |
| 78 | 9.8 |
| 79 | 8.4 |
| 80 | 17.9 |
| 81 | 12.3 |
| 85 | 18.9 |
| 86 | 6.5 |
| 88 | 17.8 |
| 89 | 9.8 |
| 90 | 6.5 |
| 91 | 7.9 |
| 92 | 3.9 |
| 93 | 17.6 |
| 94 | 2.9 |
| 96 | 7.7 |
| 97 | 3.3 |
| 99 | 6.5 |
| 100 | 7.0 |
| 104 | 1.7 |
| 105 | 1.7 |
| 106 | 65 |
| 107 | 0.5 |
| 109 | 1.3 |
| 113 | 1.1 |
| 114 | 1.6 |
| 115 | 0.5 |
| 117 | 5.6 |
| 119 | 18.5 |
| 120 | 7.4 |
| 122 | 8.9 |
| 129 | 7.1 |
| 130 | 8.5 |
| 131 | 5.6 |
| 132 | 15.7 |
| 133 | 7.3 |
| 134 | 6.8 |
| 135 | 11.9 |
| 136 | 11.6 |
| 138 | 12.0 |
| 139 | 7.4 |
| 140 | 6.1 |
| 141 | 13.9 |
| 143 | 7.4 |
| 144 | 4.3 |
| 145 | 3.9 |
| 146 | 6.6 |
| 147 | 9.6 |
| 149 | 12.2 |
| 150 | 15.0 |
| 151 | 3.2 |
| 153 | 17.8 |
| 154 | 5.9 |
| 155 | 4.3 |
| 156 | 11.4 |
| 157 | 12.1 |
| 158 | 7.1 |
| 159 | 8.4 |
| 160 | 8.5 |
| 161 | 9.7 |
| 162 | 12.0 |
| 163 | 14.9 |
| 164 | 4.1 |
| 165 | 4.3 |
| 166 | 7.2 |
| 167 | 4.6 |
| 168 | 5.4 |
| 170 | 13.8 |
| 174 | 11.7 |
| 179 | 17.8 |
| 180 | 1.0 |
| 181 | 0.8 |
| 182 | 0.3 |
| 183 | 0.5 |
| 184 | 12.0 |
| 185 | 11.0 |
| 186 | 1.7 |
| 187 | 17.0 |
| 189 | 1.5 |
| 190 | 1.0 |
| 191 | 0.7 |
| 192 | 13.2 |
| 194 | 19.1 |
| 195 | 3.2 |
| 196 | 7.6 |
| 197 | 2.0 |
| 198 | 2.7 |
| 200 | 15.1 |
| 201 | 19.1 |
| 206 | 5.7 |
| 209 | 1.4 |
| 210 | 15.7 |
| 211 | 12.0 |
| 215 | 1.1 |
| 216 | 1.7 |
| 217 | 2.6 |
| 218 | 5.7 |
| 219 | 2.1 |
| 220 | 3.9 |
| 221 | 15.6 |
| 222 | 11.3 |
| 223 | 2.0 |
| 224 | 13.0 |
| 225 | 3.9 |
| 226 | 7.7 |
| 227 | 8.2 |
| 228 | 0.5 |
| 229 | 1.0 |
| 230 | 1.5 |
| 231 | 3.0 |
| 232 | 0.5 |
| 234 | 0.7 |
| 235 | 3.4 |
| 236 | 2.0 |
| 237 | 13.1 |
| 238 | 4.4 |
| 239 | 2.4 |
| 240 | 3.4 |
| 241 | 1.0 |
| 242 | 4.0 |
| 243 | 0.4 |
| 244 | 3.2 |
| 245 | 0.8 |
| 246 | 0.8 |
| 247 | 0.3 |
| 248 | 5.6 |
| 249 | 0.4 |
| 250 | 1.1 |
| 251 | 3.4 |
| 254 | 1.4 |
| 255 | 0.6 |
| 256 | 1.1 |
| 257 | 1.3 |
| 258 | 0.6 |
| 261 | 1.0 |
| 262 | 0.6 |
| 263 | 0.6 |
| 264 | 1.0 |
| 265 | 1.0 |
| 266 | 0.8 |
| 267 | 1.1 |
| 268 | 1.1 |
| 269 | 3.6 |
| 270 | 0.7 |
| 272 | 5.0 |
| 274 | 13.2 |
| 277 | 9.0 |
| 282 | 14.5 |
| 283 | 5.9 |
| 284 | 1.2 |
| 285 | 14.2 |
| 286 | 12.7 |
| 287 | 13.5 |
| 288 | 0.2 |
| 289 | 0.5 |
| 293 | 7.0 |
| 295 | 12.3 |
| 298 | 1.5 |
| 299 | 2.8 |
| 300 | 15.0 |
| 301 | 2.0 |
| 304 | 2.3 |
| 305 | 3.7 |
| 306 | 0.5 |
| 307 | 1.0 |
| 308 | 3.6 |
| 309 | 2.1 |
| 310 | 17.3 |
| 312 | 14.0 |
| 316 | 5.3 |
| 319 | 18.6 |
| 320 | 6.2 |
| 321 | 2.3 |
| 322 | 5.6 |
| 323 | 6.2 |
| 324 | 2.0 |
| 325 | 13.0 |
| 326 | 2.9 |
| 327 | 3.6 |
| 328 | 11.8 |
| 330 | 13.8 |
| 332 | 3.3 |
| 335 | 5.5 |
| 336 | 2.9 |
| 337 | 2.8 |
| 338 | 9.1 |
| 341 | 5.3 |
| 344 | 12.2 |
| 345 | 12.7 |
| 346 | 13.5 |
| 348 | 3.8 |
| 349 | 1.9 |
| 350 | 5.2 |
| 351 | 2.2 |
| 352 | 2.3 |
| 365 | 3.4 |
| 369 | 1.4 |
| 382 | 17.0 |
| 387 | 13.7 |
| 399 | 6.8 |
| 402 | 15.4 |
| 410 | 7.1 |
| 411 | 8.7 |
| 427 | 5.9 |
| 470 | 10.6 |
| 470 | 10.6 |
| 474 | 4.9 |
| 476 | 3.9 |
| 481 | 3.4 |
| 482 | 1.2 |
| 483 | 8.3 |
| 484 | 2.7 |
| 485 | 1.1 |
| 486 | 5.1 |
| 487 | 9.9 |
| 488 | 4.0 |
| 489 | 6.0 |
| 490 | 10.0 |
| 491 | 9.3 |
| 492 | 5.0 |
| 493 | 1.9 |
| 494 | 2.1 |
| 495 | 6.3 |
| 504 | 15.4 |
| 509 | 5.9 |
| 510 | 13.2 |
| 513 | 16.9 |
| 514 | 2.6 |

### Experimental Example 2

### Human CB1 Receptor Binding Inhibition Test

The CHO-DHFR(−) cells capable of stably expressing a CB1 receptor were prepared in the same manner as in Experimental Example 1. The binding test to the human CB1 receptor was performed to obtain 50% inhibitory concentration (IC₅₀ value) of a test compound. The test results are shown in Table 17. As shown in the table, the tested compounds exhibited affinity for the CB1 receptor.

TABLE 17
Inhibition of binding to human CB1 receptor

| | CB1 |
|---|---|
| Compound | IC50 |
| No. | (nM) |
| 56 | 294 |
| 104 | 327 |
| 107 | 18 |
| 115 | 25 |
| 180 | 395 |
| 181 | 115 |
| 182 | 22 |
| 183 | 138 |
| 186 | 271 |
| 189 | 212 |
| 190 | 53 |
| 191 | 193 |
| 206 | 460 |
| 215 | 61 |
| 223 | 168 |
| 228 | 228 |
| 229 | 263 |
| 232 | 211 |
| 235 | 315 |
| 236 | 215 |
| 237 | 200 |
| 240 | 168 |
| 243 | 73 |
| 245 | 32 |
| 246 | 319 |
| 247 | 3 |
| 249 | 2 |
| 256 | 102 |
| 258 | 19 |
| 261 | 375 |
| 262 | 435 |
| 263 | 56 |
| 264 | 181 |
| 265 | 100 |
| 286 | 435 |
| 288 | 49 |
| 289 | 136 |
| 295 | 497 |
| 299 | 28 |
| 301 | 191 |
| 304 | 372 |
| 306 | 68 |
| 308 | 336 |
| 309 | 405 |
| 323 | 362 |
| 324 | 353 |
| 327 | 55 |
| 332 | 98 |
| 486 | 211 |
| 487 | 248 |
| 490 | 291 |
| 491 | 172 |
| 492 | 265 |
| 494 | 279 |

### Experimental Example 3

### Test for GTPγS Binding Via Human CB1 Receptor

CHO cells capable of stably expressing the human CB1 receptor were prepared in the same manner as in Experimental Example 2. The membrane fractions thereof were incubated together with a test compound in an assay buffer [50 mM Tris-HCl (pH 7.4), 2.5 mM EDTA, 5 mM MgCl₂, 3 μM GDP (manufactured by SIGMA), 30 μg/ml Saponin (manufactured by SIGMA)] containing 0.2% bovine serum albumin at 30° C. for 30 minutes. Thereafter, 0.1 nM [³⁵S]GTPγS (manufactured by Perkin Elmer) was added to the buffer, incubation was performed at 30° C. for 30 minutes. The resultant solution was filtrated by a glass filter GF/C and washed. Thereafter, radioactivity on the glass filter was measured by a liquid scintillation counter. The nonspecific binding was measured in the absence of the test compound. On the condition that the maximum activity value for each of the tested compounds was regarded as 100%, an effective concentration exhibiting 50% activity (EC₅₀ value) was calculated.

The EC₅₀ values for test compounds Nos. 247 and 249 were 33 nM and 19 nM, respectively. In this way, the compounds according to the present invention showed an agonist effect on the CB1 receptor.

### Experimental Example 4

### Test for GTPγS Binding Via Human CB2 Receptor

The CHO cells capable of stably expressing the human CB2 receptor were prepared in the same manner as in Experimental Example 1. The GTPγS binding test was performed in the same manner as in Experimental Example 3 to obtain an effective concentration of the test compound exhibiting 50% activity value), on the condition that the maximum activity value for each of the test compounds was regarded as 50%.

The EC₅₀ values of test compounds Nos. 9, 184, 267 and 474 were 23.7 nM, 9.8 nM, 0.4 nM and 2.3 nM, respectively. In this way, the compounds according to the present invention exhibited an agonist effect on the CB2 receptor.

### Experimental Example 5

### Writhing Test with Acetic Acid in Mouse

This test was performed in accordance with the method of Futaki N et al. (Gen Pharmacol. 24(1):105-110 (1993). A test compound suspended in a 5% gum Arabic solution was orally administered to Jcl: ICR-series male mice (5 weeks old). One hour after the administration of the test compound, a 0.9% aqueous acetic acid solution was intraperitoneally administered. Five minutes after the administration, the number of abdomen stretch movements (pain-related behavior) were counted for 10 minutes. Only the 5% gum Arabic solution was orally administered to the control group. A pain-related behavior inhibition rate (%) was obtained based on the following equation.

$$\begin{matrix}Pain \text{-} related behavior \\ inhibition ( \% )\end{matrix} = \frac{\begin{matrix}The number of pain \text{-} \\ related behaviors counted \\ for control group\end{matrix} - \begin{matrix}The number of pain \text{-} related \\ behaviors counted for test \\ compound group\end{matrix}}{\begin{matrix}The number of pain \text{-} \\ related behaviors counted \\ for control group\end{matrix}} \times 100$$

The inhibitory rates of test compounds Nos. 59, 247, 267, 411, 474 and 510 when they were orally administered in a dose of 30 mg/kg were 44.8%, 93.0%, 59.9%, 49.0%, 61.9% and 19.6%, respectively. The compounds according to the present invention exhibited an analgesic effect.

### Experimental Example 6

### Neuropathic Pain Test in Rat

Using SD:IGS-series male rats (5 weeks old), neuropathic pain model were prepared by partially clipping the sciatic nerve of the femoral region in accordance with the method of Seltzer et al. (SeltzerZ; Pain. 43(2):205-218 (1990)). When the plantar surface of the paw on the affected side was stimulated by touching with a von Frey filament(s) (nylon fiber for use in a touch test: North Coast Medical, Inc.), the pain threshold (load (g) applied to the filament when an animal responds to touch stimulation) was measured. The test compound was suspended in a 5% gum Arabic solution and administered in a dose of 0 mg/kg, 3 mg/kg, 10 mg/kg and 30 mg/kg. One hour after the administration, pain threshold (g) was measured.

The oral administration of test compound No. 184 increased pain threshold in a dose depending manner and demonstrated improvement of pain sensitivity (FIG. 1).

### Experimental Example 7

### Test for Edema on Ear in Mouse

Using Balb/c male mice (5 weeks old), a test compound dissolved in acetone was applied in an amount of 20 μL to the inside the ear. Ten minutes after the application of the test compound, an acetone solution (20 μL) containing 0.8 μg of PMA (phorbol 12-myristate 13-acetate) was applied. Five hours later, the thickness (hyperplasia) of the ear was measured by a dial sickness gauge. Acetone alone was applied to the control group. The inhibition rate (%) of edema on the ear was calculated based on the following equation.

$$\begin{matrix}Ear edema \\ inhibition ( \% )\end{matrix} = \frac{\begin{matrix}Ear thickness of \\ PMA \text{-} treated control \\ group\end{matrix} - \begin{matrix}Ear thickness of \\ PMA \text{-} treated test \\ compound group\end{matrix}}{\begin{matrix}Ear thickness of \\ PMA \text{-} treated control \\ group\end{matrix} - \begin{matrix}Ear thickness of \\ PMA \text{-} untreated \\ control group\end{matrix}} \times 100$$

The inhibition rates of test compounds No. 184, 267 and 474 at a dose of 1 mg per murine ear, were 65%, 84% and 37%, respectively. The compounds according to the present invention exhibited an anti-edema effect.

### INDUSTRIAL APPLICABILITY

In the present invention, there is provided an imine compound having a cannabinoid receptor agonist effect. The imine compound of the present invention has a cannabinoid receptor agonist effect, and is useful as a therapeutic drug or prophylactic drug for pain and autoimmune disease.

### BRIEF DESCRIPTION OF THE DRAWING

FIG. 1 shows the results of the neuropathic pain test in rat of Experimental Example 6.

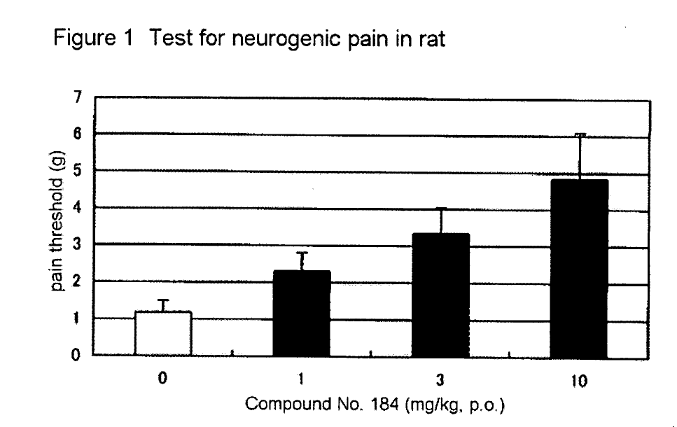

1. A cannabinoid-receptor agonist comprising an imine compound represented by Formula (I) 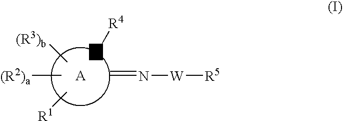
[where A represents any one of the rings represented by the following formulas (where X represents an oxygen atom or a sulfur atom, and X′ represents CH or a nitrogen atom): 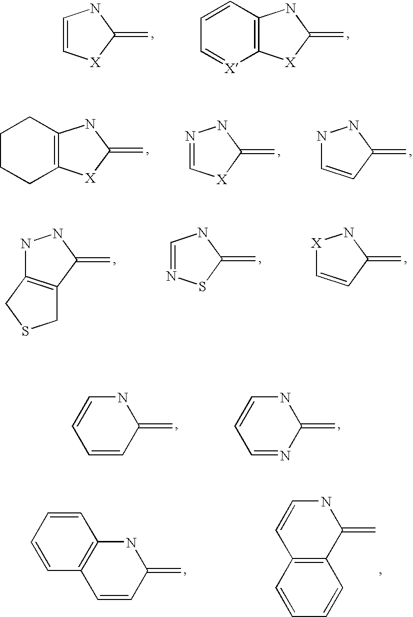
R¹ represents
a hydrogen atom;
a halogen atom;
a C₁₋₁₀ alkyl group that may be substituted with an aryl group(s) substituted with a halogen atom(s);
a C₃₋₁₀ cycloalkyl group;
a C₂₋₆ alkenyl group;
a C₁₋₆ haloalkyl group;
a C₁₋₁₀ alkoxy group;
a carboxyl group;
a C₂₋₆ alkoxycarbonyl group;
a hydroxy-C₁₋₆ alkyl group;
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group);
a group represented by Formula —CON(R⁶¹)R⁷¹ (where R⁶¹ and R⁷¹ each represent a hydrogen atom or a C₁₋₆ alkyl group that may be substituted with a cyclic amino group(s), or R⁶¹ and R⁷¹, in combination with the adjacent nitrogen atom, form a cyclic amino group); or
an aryl group that may be substituted with 1 to 3 groups selected from the group consisting of: a C₁₋₆ alkyl group, a C₁₋₆ haloalkyl group and a halogen atom,
R² and R³ each represent
a hydrogen atom;
a halogen atom;
a C₁₋₆ alkyl group;
a C₁₋₆ haloalkyl group; or
an aryl group that may be substituted with 1 to 3 groups selected from the group consisting of: a C₁₋₆ alkyl group, a C₁₋₆ haloalkyl group and a halogen atom,
R⁴ represents
a C₁₋₁₀ alkyl group;
a C₁₋₆ haloalkyl group;
a C₁₋₁₀ alkyl group or C₂₋₆ alkenyl group substituted with: a C₃₋₁₀ cycloalkyl group(s), a C₁₋₆ alkoxy group(s), a hydroxyl group(s), an amino group(s), a phthalimide group(s), a cyano group(s), an arylthio group(s), a C₂₋₆ alkoxycarbonyl group(s), a carboxyl group(s), a group(s) represented by Formula —CON(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group), or an aryl group(s) that may be substituted with a C₁₋₆ haloalkyl group(s), a C₂₋₆ alkoxycarbonyl group(s), a carboxyl group(s), or an N-piperidinocarbamoyl group(s);
a C₂₋₆ haloalkenyl group;
a C₂₋₆ alkynyl group;
a 1,1-dioxothiolanyl group; or
an aryl group,
R⁵ represents
a hydrogen atom;
a C₁₋₁₀ alkoxy group;
a C₁₋₆ alkoxy-C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, a C₂₋₆ alkoxycarbonyl group, a C₁₋₆ haloalkyl group, a C₁₋₆ alkoxy group that may be substituted with a C₁₋₆ alkoxy group(s) or an aryl group(s), a C₃₋₁₀ cycloalkoxy group that may be substituted with 1 to 2 C₁₋₆ alkyl groups, an aryl or aryloxy group that may be substituted with 1 to 5 groups selected from the group consisting of a C₁₋₆ alkyl group, a C₁₋₆ alkoxy group, a C₁₋₆ haloalkyl group, a C₁₋₆ haloalkoxy group, an aralkyloxy group, a nitro group and a halogen atom, a heterocyclic group, a phthalimide group, a C₁₋₆ alkanoyloxy group, an aralkyloxy group, a C₁₋₆ alkylthio group, an arylthio group, and a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with a C₁₋₆ alkyl group(s), a C₁₋₆ alkoxy group(s), a C₂₋₆ alkoxycarbonyl group(s), a C₁₋₆ haloalkyl group(s) or a C₁₋₆ haloalkoxy group(s);
an aralkyloxy group; or
a group represented by Formula (II) 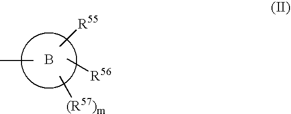
{where
B represents
a C₃₋₁₀ cycloalkyl group;
an aryl group;
a heterocyclic group;
a C₂₋₆ cyclic amino group;
a fluorenyl group;
a phthalimide group;
a 2-oxopyrrolidinyl group;
a group represented by Formula (III) 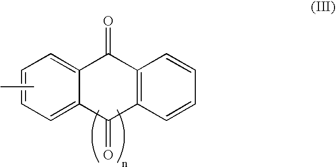
(where n represents 0 or 1); or
a group represented by Formula (IV) 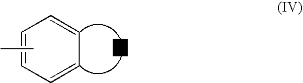
(where Y represents —(CH₂)ₚ—, —CO—CH₂—CH₂—, —CO—CH₂—CH₂—CH₂—, —O—CH₂—CH₂—, —O—CH₂—CH═CH—, or —O—(CH₂)_{q}—O—, in which p represents an integer of 2 to 4 and q represents an integer of 1 to 3);
R⁵⁵ represents
a hydrogen atom;
a halogen atom;
a C₁₋₁₀ alkyl group that may be substituted with an aryl group(s), a heterocyclic group(s) or an aryloxy group(s), each of which may be substituted with a halogen atom(s); or with a group(s) represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ alkylsulfonyl group;
an arylsulfonyl group that may be substituted with a C₁₋₆ alkyl group(s) or a halogen atom(s);
a C₁₋₆ haloalkoxy group;
a C₁₋₆ haloalkylthio group;
a C₃₋₁₀ cycloalkyl group;
a C₂₋₆ alkenyl group;
a C₂₋₆ alkenyloxy group;
a C₂₋₆ alkenylthio group;
a C₁₋₆ alkoxy-C₁₋₆alkoxy group;
an aryl group that may be substituted with 1 to 3 groups selected from the group consisting of: a C₁₋₆ alkyl group, a C₁₋₆ haloalkyl group, a halogen atom, a C₁₋₆ alkoxy group, a cyano group and a nitro group;
a heterocyclic group that may be substituted with a C₁₋₆ alkyl group(s) or a C₁₋₆ haloalkyl group(s);
an aryloxy group or arylthio group that may be substituted with a halogen atom(s) or a C₁₋₆ alkyl group(s);
a group represented by Formula —N(R⁶³)R⁷³ (where R⁶³ and R⁷³ each represent a hydrogen atom, a C₁₋₆ alkyl group, a C₁₋₆ hydroxyalkyl group, a C₁₋₆ alkoxy-C₁₋₆ alkyl group, an aryl group, a C₁₋₆ alkanoyl group, a di-C₁₋₆ alkylamino-C₂₋₆ alkanoyl group, a benzoyl group, or a heterocyclic group that may be substituted with a C₁₋₆ alkyl group(s), or R⁶³ and R⁷³, in combination with the adjacent nitrogen atom, form a cyclic amino group);
a hydroxyl group;
a cyano group;
a nitro group;
a C₁₋₆ alkanoyl group;
a C₁₋₆ alkanoyloxy group;
a C₁₋₆ alkanoyloxy-C₁₋₆ alkyl group;
a C₂₋₆ haloalkanoyl group;
a carboxyl group;
a C₂₋₆ alkoxycarbonyl group;
a C₂₋₆ cyclic amino group that may be substituted with an aryl group(s);
a group represented by Formula —CON(R⁶⁴)R⁷⁴ (where R⁶⁴ and R⁷⁴ each represent a hydrogen atom, a C₁₋₆ alkyl group, a C₁₋₆ alkoxy-C₁₋₆ alkyl group, or a heterocyclic group that may be substituted with a C₁₋₆ alkyl group(s), or R⁶⁴ and R⁷⁴, in combination with the adjacent nitrogen atom, form a cyclic amino group);
a group represented by Formula —SO₂N(R⁶²)R⁷² (where R⁶² and R⁷² each represent a hydrogen atom, or a C₁₋₆ alkyl group, or R⁶² and R⁷², in combination with the adjacent nitrogen atom, form a cyclic amino group);
a C₁₋₆ alkylsulfenyl group;
a C₁₋₆ alkylsulfonyl group that may be substituted with a halogen atom(s); or
an arylsulfonyl group that may be substituted with a halogen atom(s),
R⁵⁶ represents
a hydrogen atom;
a halogen atom;
a C₁₋₁₀ alkyl group that may be substituted with: an aryl group(s), a pyridyl group(s), a thienyl group(s) or a heterocyclic group(s), each of which may be substituted with a C₁₋₆ alkyl group(s) or a halogen atom(s);
a C₁₋₆ haloalkyl group;
a C₃₋₁₀ cycloalkyl group;
a C₁₋₁₀ alkoxy group;
a C₂₋₆ alkenyl group;
an aryl group that may be substituted with 1 to 3 groups selected from the group consisting of: a C₁₋₆ alkyl group, a halogen atom, a C₁₋₆ alkoxy group and a nitro group;
a heterocyclic group that may be substituted with a C₁₋₆ alkyl group(s);
a C₁₋₆ alkanoyl group;
a C₁₋₆ alkylsulfenyl group;
a C₁₋₆ alkylsulfonyl group;
an arylsulfonyl group that may be substituted with a halogen atom(s);
a hydroxyl group;
a cyano group; or
a nitro group,
R⁵⁷ represents
a hydrogen atom;
a C₁₋₁₀ alkyl group that may be substituted with a pyridyl group(s) or a thienyl group(s);
a C₁₋₆ haloalkyl group;
a C₃₋₁₀ cycloalkyl group;
a halogen atom;
a C₂₋₆ alkenyl group;
an aryl group that may be substituted with a halogen atom(s);
a C₁₋₁₀ alkoxy group;
a C₁₋₆ alkanoyl group; or
a C₁₋₆ alkylsulfenyl group, and
m represents an integer of 1 to 3},
a and b each represent 0 or 1, and
W represents —CO—, —CO—CO—, —CO—NH—, —CS—NH—, or —SO₂—],
or a pharmaceutically acceptable salt thereof, as an active ingredient.

2. The cannabinoid-receptor agonist according to claim 1 comprising the imine compound or the pharmaceutically acceptable salt thereof, as an active ingredient,
wherein
R¹ represents
a hydrogen atom;
a halogen atom;
a C₁₋₁₀ alkyl group that may be substituted with an aryl group(s) substituted with a halogen atom(s);
a C₃₋₁₀ cycloalkyl group;
a C₁₋₆ haloalkyl group;
a C₁₋₁₀ alkoxy group;
a carboxyl group;
a C₂₋₆ alkoxycarbonyl group;
a hydroxy-C₁₋₆ alkyl group;
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group);
a group represented by Formula —CON(R⁶¹)R⁷¹ (where R⁶¹ and R⁷¹ each represent a hydrogen atom or a C₁₋₆ alkyl group that may be substituted with a cyclic amino group(s), or R⁶¹ and R⁷¹, in combination with the adjacent nitrogen atom, form a cyclic amino group); or
an aryl group that may be substituted with 1 to 3 groups selected from the group consisting of: a C₁₋₆ alkyl group, a C₁₋₆ haloalkyl group and a halogen atom,
R⁴ represents
a C₁₋₁₀ alkyl group;
a C₁₋₆ haloalkyl group;
a C₁₋₁₀ alkyl group substituted with a C₃₋₁₀ cycloalkyl group(s), a C₁₋₆ alkoxy group(s), a hydroxyl group(s), an amino group(s), a phthalimide group(s), a cyano group(s), an arylthio group(s), a C₂₋₆ alkoxycarbonyl group(s), a carboxyl group(s), a group(s) represented by Formula —CON(R⁶²)R⁷² (where R⁶² and R⁷² each represent a hydrogen atom, or a C₁₋₆ alkyl group, or R⁶² and R⁷², in combination with the adjacent nitrogen atom, form a cyclic amino group), or an aryl group(s) that may be substituted with a C₁₋₆ haloalkyl group(s), a C₂₋₆ alkoxycarbonyl group(s), a carboxyl group(s) or an N-piperidinocarbamoyl group(s);
a C₂₋₆ alkenyl group that may be substituted with an aryl group(s);
a C₂₋₆ haloalkenyl group;
a C₂₋₆ alkynyl group;
a 1,1-dioxothiolanyl group; or
an aryl group,
R⁵ represents
a C₁₋₁₀ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, a C₂₋₆ alkoxycarbonyl group, a C₁₋₆ haloalkyl group, a C₁₋₆ alkoxy group that may be substituted with a C₁₋₆ alkoxy group(s) or an aryl group(s), a C₃₋₁₀ cycloalkoxy group that may be substituted with 1 to 2 C₁₋₆ alkyl groups, an aryl group or aryloxy group that may be substituted with 1 to 5 groups selected from the group consisting of a C₁₋₆ alkyl group, a C₁₋₆ alkoxy group, a C₁₋₆ haloalkyl group, a C₁₋₆ haloalkoxy group, an aralkyloxy group, a nitro group and a halogen atom, a heterocyclic group, a phthalimide group, a C₁₋₆ alkanoyloxy group, an aralkyloxy group, a C₁₋₆ alkylthio group, an arylthio group, and a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with a C₁₋₆ alkyl group(s), a C₁₋₆ alkoxy group(s), a C₂₋₆ alkoxycarbonyl group(s), a C₁₋₆ haloalkyl group(s) or a C₁₋₆ haloalkoxy group(s); or
a group represented by Formula (II), where
B represents
a C₃₋₁₀ cycloalkyl group, an aryl group, or a heterocyclic group,
R⁵⁵ and R⁵⁶ each represent
a hydrogen atom;
a halogen atom;
a C₁₋₁₀ alkyl group that may be substituted with: an aryl group(s), a heterocyclic group(s) or an aryloxy group(s), each of which may be substituted with a halogen atom(s); or with a group(s) represented by Formula —N(R⁶²)R⁷² (R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ alkylsulfonyl group;
an arylsulfonyl group that may be substituted with a C₁₋₆ alkyl group(s) or a halogen atom(s);
a C₁₋₆ haloalkoxy group
a C₁₋₆ haloalkylthio group;
a C₃₋₁₀ cycloalkyl group;
a C₂₋₆ alkenyl group;
a C₂₋₆ alkenyloxy group;
a C₂₋₆ alkenylthio group;
a C₁₋₆ alkoxy-C₁₋₆alkoxy group;
an aryl group that may be substituted with 1 to 3 groups selected from the group consisting of: a C₁₋₆ alkyl group, a C₁₋₆ haloalkyl group, a halogen atom, a C₁₋₆ alkoxy group, a cyano group and a nitro group;
a heterocyclic group that may be substituted with a C₁₋₆ alkyl group(s) or a C₁₋₆ haloalkyl group(s);
an aryloxy group or arylthio group that may be substituted with a halogen atom(s) or a C₁₋₆ alkyl group(s);
a group represented by Formula —N(R⁶³)R⁷³ (where R⁶³ and R⁷³ each represent a hydrogen atom, a C₁₋₆ alkyl group, a C₁₋₆ hydroxyalkyl group, a C₁₋₆ alkoxy-C₁₋₆ alkyl group, an aryl group, a C₁₋₆ alkanoyl group, a di-C₁₋₆ alkylamino-C₂₋₆ alkanoyl group, a benzoyl group or a heterocyclic group that may be substituted with a C₁₋₆ alkyl group(s), or R⁶³ and R⁷³, in combination with the adjacent nitrogen atom, form a cyclic amino group);
a hydroxyl group;
a cyano group;
a nitro group;
a C₁₋₆ alkanoyl group;
a C₁₋₆ alkanoyloxy group;
a C₁₋₆ alkanoyloxy-C₁₋₆ alkyl group;
a C₂₋₆ haloalkanoyl group;
a carboxyl group;
a C₂₋₆ alkoxycarbonyl group; or
a group represented by Formula —CON(R⁶⁴)R⁷⁴ (where R⁶⁴ and R⁷⁴ each represent a hydrogen atom, a C₁₋₆ alkyl group, a C₁₋₆ alkoxy-C₁₋₆ alkyl group, or a heterocyclic group that may be substituted with a C₁₋₆ alkyl group(s), or R⁶⁴ and R⁷⁴, in combination with the adjacent nitrogen atom, form a cyclic amino group),
R⁵⁷ represents
a hydrogen atom;
a C₁₋₁₀ alkyl group;
a C₁₋₆ haloalkyl group;
a halogen atom; or
a C₁₋₁₀ alkoxy group.

3. A cannabinoid-receptor agonist comprising an imine compound represented by Formula (I-1) 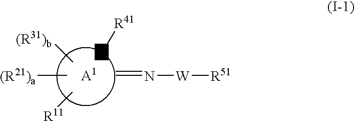
[where A¹ represents any one of the rings represented by the following formulas (where X represents an oxygen atom or a sulfur atom): 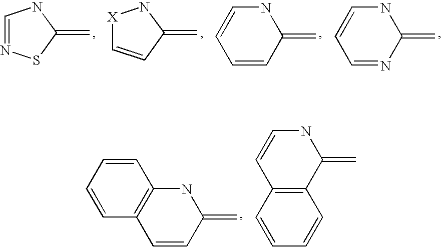
R¹¹ represents
a hydrogen atom;
a halogen atom;
a C₁₋₆ alkyl group
a C₂₋₆ alkenyl group;
a C₁₋₆ haloalkyl group;
a C₁₋₆ alkoxy group; or
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group),
R² and R³¹ each represent
a hydrogen atom;
a halogen atom; or
a C₁₋₆ alkyl group,
R⁴¹ represents
a C₁₋₁₀ alkyl group or C₂₋₆ alkenyl group that may be substituted with a halogen atom(s), a C₃₋₁₀ cycloalkyl group(s), an aryl group (s) or a C₁₋₆ alkoxy group(s),
R⁵¹ represents
a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, an aryl group or aryloxy group that may be substituted with 1 to 5 groups selected from the group consisting of a C₁₋₆ alkyl group, a C₁₋₆ alkoxy group, a C₁₋₆ haloalkyl group and a halogen atom, and a heterocyclic group;
a group represented by Formula (II-1) 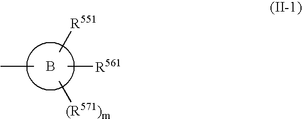
{where
B represents
a C₃₋₁₀ cycloalkyl group;
an aryl group;
a heterocyclic group;
a fluorenyl group; or
a group represented by Formula (IV-1) 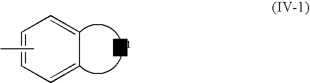
(where Y¹ represents —(CH₂)ₚ—, or —O—(CH₂)_{q}—O—, in which p represents an integer of 2 to 4, and q represents an integer of 1 to 3),
R⁵⁵¹ and R⁵⁶¹ each represent
a hydrogen atom;
a halogen atom;
a C₁₋₁₀ alkyl group that may be substituted with: an aryl group(s), a heterocyclic group(s) or an aryloxy group(s), each of which may be substituted with a halogen atom(s);
or with a group(s) represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ haloalkoxy group;
a C₃₋₁₀ cycloalkyl group;
a C₂₋₆ alkenyl group;
an aryl group that may be substituted with 1 to 3 groups selected from the group consisting of: a C₁₋₆ alkyl group, a C₁₋₆ haloalkyl group, a halogen atom, a C₁₋₆ alkoxy group, and a nitro group;
a heterocyclic group that may be substituted with a C₁₋₆ alkyl group(s);
an aryloxy group that may be substituted with a halogen atom(s);
a group represented by Formula —N(R⁶³⁴)R⁷³⁴ (where R⁶³⁴ and R⁷³⁴ each represent a hydrogen atom, a C₁₋₆ alkyl group, an aryl group, or a C₁₋₆ alkanoyl group, or R⁶³⁴ and R⁷³⁴, in combination with the adjacent nitrogen atom, form a cyclic amino group);
a hydroxyl group;
a cyano group;
a nitro group;
a C₁₋₆ alkanoyl group;
a C₂₋₆ haloalkanoyl group;
a C₁₋₆ alkylsulfonyl group; or
an arylsulfonyl group that may be substituted with a halogen atom(s),
R⁵⁷¹ represents
a hydrogen atom;
C₁₋₁₀ alkyl group;
a C₁₋₁₀ alkoxy group; or
a halogen atom, and
m represents an integer of 1 to 3}
a and b each represent 0 or 1, and
W represents CO or SO₂]
or a pharmaceutically acceptable salt thereof, as an active ingredient.

4. A cannabinoid-receptor agonist comprising the imine compound or the pharmaceutically acceptable salt thereof according to claim 3, as an active ingredient, wherein
R⁵¹ represents a C₁₋₁₀ alkyl group or C₂₋₆ alkenyl group that may be substituted with 1 to 3 groups selected from the group consisting of: a halogen atom, a C₃₋₁₀ cycloalkyl group, and an aryl group, a thienyl group and an aryloxy group, each of which may be substituted with a C₁₋₆ haloalkyl group(s) or a halogen atom(s); or
represents a group represented by Formula (II-1) where
R⁵⁵¹ represents
a hydrogen atom;
a halogen atom;
a C₁₋₁₀ alkyl group that may be substituted with an aryl group(s) that may be substituted with a halogen atom(s), a heterocyclic group(s) or an aryloxy group(s);
a C₁₋₆ haloalkyl group;
a C₁₋₁₀ alkoxy group;
a C₁₋₆ haloalkoxy group;
a C₃₋₁₀ cycloalkyl group;
an aryl group that may be substituted with 1 to 3 groups selected from the group consisting of: a C₁₋₆ alkyl group, a halogen atom, a C₁₋₆ alkoxy group and a nitro group;
a heterocyclic group that may be substituted with a C₁₋₆ alkyl group(s); an aryloxy group that may be substituted with a halogen atom(s);
a group represented by Formula —N(R⁶³¹)R⁷³¹ (where R⁶³¹ and R⁷³¹ each represent a hydrogen atom, a C₁₋₆ alkyl group, an aryl group or a C₁₋₆ alkanoyl group, or R^{63l} and R⁷³¹, in combination with the adjacent nitrogen atom, form a cyclic amino group);
a cyano group;
a C₁₋₆ alkanoyl group;
a C₂₋₆ haloalkanoyl group; or
a C₁₋₆ alkylsulfonyl group,
R⁵⁶ represents
a hydrogen atom;
a halogen atom;
a C₁₋₁₀ alkyl group;
a C₁₋₆ haloalkyl group; or
a C₁₋₁₀ alkoxy group,
R⁵⁷¹ represents
a hydrogen atom;
a halogen atom;
C₁₋₁₀ alkyl group; or
a C₁₋₁₀ alkoxy group.

5. A cannabinoid-receptor agonist comprising the imine compound or the pharmaceutically acceptable salt thereof according to claim 3, as an active ingredient, wherein
A¹ is a ring represented by Formula below:

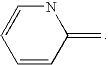

6. A cannabinoid-receptor agonist comprising the imine compound or the pharmaceutically acceptable salt thereof according to claim 5, as an active ingredient, wherein
R⁵¹ represents
a C₁₋₁₀ alkyl group that may be substituted with: a C₃₋₁₀ cycloalkyl group(s), or an aryl group(s), a thienyl group(s) or an aryloxy group(s), each of which may be substituted with a C₁₋₆ haloalkyl group(s) or a halogen atom(s); or
a group(s) represented by Formula (II-1), where
B represents
a C₃₋₁₀ cycloalkyl group, an aryl group, or a heterocyclic group,
R⁵⁵¹ represents
a halogen atom;
a C₁₋₁₀ alkyl group;
a C₁₋₆ haloalkyl group;
a C₁₋₁₀ alkoxy group;
a C₁₋₆ haloalkoxy group;
a C₃₋₁₀ cycloalkyl group;
an aryl group that may be substituted with 1 to 3 groups selected from the group consisting of a C₁₋₆ alkyl group and a halogen atom;
a heterocyclic group that may be substituted with a C₁₋₆ alkyl group(s);
an aryloxy group;
a morpholino group;
an arylamino group;
a cyano group;
a C₁₋₆ alkanoyl group;
a C₂₋₆ haloalkanoyl group; or
a C₁₋₆ alkylsulfonyl group,
R⁵⁶¹ represents
a hydrogen atom;
a halogen atom;
a C₁₋₁₀ alkyl group;
a C₁₋₆ haloalkyl group; or
a C₁₋₁₀ alkoxy group,
R⁵⁷¹ represents
a hydrogen atom;
a halogen atom;
a C₁₋₁₀ alkyl group; or
a C₁₋₁₀ alkoxy group.

7. A cannabinoid-receptor agonist comprising the imine compound or the pharmaceutically acceptable salt thereof according to claim 6, as an active ingredient, wherein
R⁵¹ represents a group represented by Formula (II-1),
B represents a phenyl group,
R⁵⁵¹ represents
a halogen atom;
a C₁₀ alkyl group
a C₁₋₆ haloalkyl group;
a C₁₋₆ alkoxy group;
a C₁₋₆ haloalkoxy group;
a C₃₋₁₀ cycloalkyl group;
an aryl group
an aryloxy group;
a morpholino group;
an arylamino group
a cyano group
a C₁₋₆ alkanoyl group;
a C₂₋₆ haloalkanoyl group; or
a C₁₋₆ alkylsulfonyl group,
R⁵⁶¹ represents
a hydrogen atom;
a halogen atom;
a C₁₋₆ haloalkyl group; or
a C₁₋₆ alkoxy group,
R⁵⁷¹ represents
a hydrogen atom;
a halogen atom;
a C₁₀ alkyl group; or
a C₁₋₁₀ alkoxy group (provided that when R⁵⁵¹, R⁵⁷¹ and R⁴¹ each are an alkyl group, the number of carbon atoms of R⁵⁵¹ and R⁵⁷¹ are 1 to 6, and the number of carbon atoms of R⁴¹ is 2 to 10), and
m represents 1.

8. The cannabinoid-receptor agonist according to claim 1, being a cannabinoid type-1 receptor agonist or a cannabinoid type-2 receptor agonist.

9. The cannabinoid-receptor agonist according to claim 1, being a therapeutic drug or prophylactic drug for pain.

10. The cannabinoid-receptor agonist according to claim 1, being a therapeutic drug or prophylactic drug for autoimmune disease.

11. An imine compound represented by Formula (I-1) 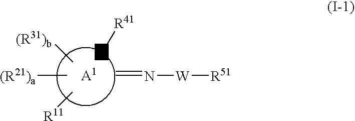
[where A¹ represents any one of the rings represented by the following formulas (where X represents an oxygen atom or a sulfur atom): 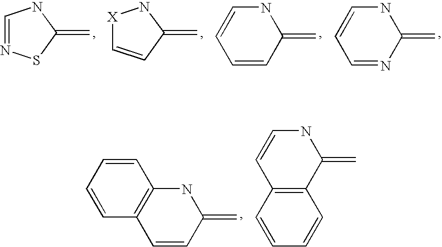
R¹¹ represents
a hydrogen atom;
a halogen atom;
a C₁₋₆ alkyl group
a C₂₋₆ alkenyl group;
a C₁₋₆ haloalkyl group;
a C₁₋₆ alkoxy group; or
a group represented by Formula —N(R⁶)R⁷ (where R⁶ and R⁷ each represent a hydrogen atom or a C₁₋₆ alkyl group, or R⁶ and R⁷, in combination with the adjacent nitrogen atom, form a cyclic amino group),
R²¹ and R³¹ each represent
a hydrogen atom;
a halogen atom; or
a C₁₋₆ alkyl group,
R⁴¹ represents
a C₁₋₁₀ alkyl group or C₂₋₆ alkenyl group that may be substituted with a halogen atom(s), a C₃₋₁₀ cycloalkyl group(s), an aryl group(s) or a C₁₋₆ alkoxy group(s),
R⁵¹ represents
a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, an aryl group or aryloxy group that may be substituted with 1 to 5 groups selected from the group consisting of a C₁₋₆ alkyl group, a C₁₋₆ alkoxy group, a C₁₋₆ haloalkyl group and a halogen atom, and a heterocyclic group;
a group represented by Formula (II-1) 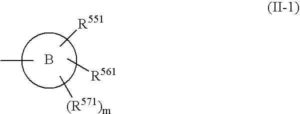
{where
B represents
a C₃₋₁₀ cycloalkyl group;
an aryl group;
a heterocyclic group;
a fluorenyl group; or
a group represented by Formula (IV-1) 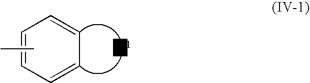
(where Y¹ represents —(CH₂)ₚ—, or —O—(CH₂)_{q}—O—, in which p represents an integer of 2 to 4, and q represents an integer of 1 to 3),
R⁵⁵¹ and R⁵⁶¹ each represent
a hydrogen atom;
a halogen atom;
a C₁₋₁₀ alkyl group that may be substituted with: an aryl group(s), a heterocyclic group(s) or an aryloxy group(s), each of which may be substituted with a halogen atom(s); or with a group(s) represented by Formula —N(R²)R⁷² (where R¹² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ haloalkoxy group a C₃₋₁₀ cycloalkyl group;
a C₂₋₆ alkenyl group;
an aryl group that may be substituted with 1 to 3 groups selected from the group consisting of: a C₁₋₆ alkyl group, a C₁₋₆ haloalkyl group, a halogen atom, a C₁₋₆ alkoxy group and a nitro group;
a heterocyclic group that may be substituted with a C₁₋₆ alkyl group(s);
an aryloxy group that may be substituted with a halogen atom(s);
a group represented by Formula —N(R⁶³⁴)R⁷³⁴ (where R⁶³⁴ and R⁷³⁴ each represent a hydrogen atom, a C₁₋₆ alkyl group, an aryl group, or a C₁₋₆ alkanoyl group, or R⁶³⁴ and R⁷³⁴, in combination with the adjacent nitrogen atom, form a cyclic amino group);
a hydroxyl group;
a cyano group;
a nitro group;
a C₁₋₆ alkanoyl group;
a C₂₋₆ haloalkanoyl group;
a C₁₋₆ alkylsulfonyl group; or
an arylsulfonyl group that may be substituted with a halogen atom(s),
R⁵⁷ represents
a hydrogen atom;
a halogen atom;
a C₁₋₁₀ alkyl group; or
a C₁₋₁₀ alkoxy group, and
m represents an integer of 1 to 3}
a and b each represent 0 or 1, and
W represents CO or SO₂],
or a pharmaceutically acceptable salt thereof.

12. The imine compound or the pharmaceutically acceptable salt thereof according to claim 11, wherein
R⁵¹ represents
a C₁₋₁₀ alkyl group or C₂₋₆ alkenyl group that may be substituted with 1 to 3 groups selected from the group consisting of: a halogen atom, a C₃₋₁₀ cycloalkyl group, and an aryl group, a thienyl group and an aryloxy group, each of which may be substituted with a C₁₋₆ haloalkyl group(s) or a halogen atom(s); or represents a group represented by Formula (II-1) where
R⁵⁵¹ represents
a hydrogen atom;
a halogen atom;
a C₁₋₁₀ alkyl group that may be substituted with an aryl group(s), a heterocyclic group(s) or an aryloxy group(s) that may be substituted with a halogen atom(s);
a C₁₋₆ haloalkyl group;
a C₁₋₁₀ alkoxy group;
a C₁₋₆ haloalkoxy group;
a C₃₋₁₀ cycloalkyl group;
an aryl group that may be substituted with 1 to 3 groups selected from the group consisting of: a C₁₋₆ alkyl group, a halogen atom, a C₁₋₆ alkoxy group and a nitro group;
a heterocyclic group that may be substituted with a C₁₋₆ alkyl group(s);
an aryloxy group that may be substituted with a halogen atom(s);
a group represented by Formula —N(R⁶³¹)R⁷³¹ (where R⁶³¹ and R⁷³¹ each represent a hydrogen atom, a C₁₋₆ alkyl group, an aryl group or a C₁₋₆ alkanoyl group, or R⁶³¹ and R⁷³¹, in combination with the adjacent nitrogen atom, form a cyclic amino group);
a cyano group;
a C₁₋₆ alkanoyl group;
a C₂₋₆ haloalkanoyl group; or
a C₁₋₆ alkylsulfonyl group,
R⁵⁶¹ represents
a hydrogen atom;
a halogen atom;
a C₁₋₁₀ alkyl group;
a C₁₋₆ haloalkyl group; or
a C₁₋₁₀ alkoxy group,
R⁵⁷¹ represents
a hydrogen atom;
a halogen atom;
a C₁₋₁₀ alkyl group; or
a C₁₋₁₀ alkoxy group.

13. The imine compound or the pharmaceutically acceptable salt thereof according to claim 11, wherein W is —CO—.

14. The imine compound or the pharmaceutically acceptable salt thereof according to claim 13, wherein
R⁴¹ represents a C₁₋₁₀ alkyl group substituted with a C₁₋₆ alkoxy group(s), or an aryl group(s); R⁵⁵¹ is a C₁₋₆ haloalkyl group; and R⁵⁶⁴ is a halogen atom.

15. The imine compound or the pharmaceutically acceptable salt thereof according to claim 13, wherein R⁴¹ represents a C₁₋₁₀ alkyl group substituted with a C₃₋₁₀ cycloalkyl group(s) or a C₁₋₆ alkoxy group(s).

16. The imine compound or the pharmaceutically acceptable salt thereof according to claim 14, wherein A¹ is a 1,2-dihydropyridine ring.

17. The imine compound or the pharmaceutically acceptable salt thereof according to claim 16, wherein
R⁵¹ represents a C₁₋₁₀ alkyl group that may be substituted with: a C₃₋₁₀ cycloalkyl group(s), or an aryl group, a thienyl group(s) or an aryloxy group(s), each of which may be substituted with a C₁₋₆ haloalkyl group(s) or a halogen atom(s); or
a group represented by Formula (II-1),
B represents a C₃₋₁₀ cycloalkyl group, an aryl group, or a heterocyclic group;
R⁵⁵¹ represents
a halogen atom;
a C₁₋₁₀ alkyl group
a C₁₋₆ haloalkyl group;
a C₁₋₁₀ alkoxy group;
a C₁₋₆ haloalkoxy group;
a C₃₋₁₀ cycloalkyl group;
an aryl group that may be substituted with 1 to 3 groups selected from the group consisting of a C₁₋₆ alkyl group and a halogen atom;
a heterocyclic group that may be substituted with a C₁₋₆ alkyl group(s);
an aryloxy group;
a morpholino group;
an arylamino group
a cyano group
a C₁₋₆ alkanoyl group;
a C₂₋₆ haloalkanoyl group; or
a C₁₋₆ alkylsulfonyl group,
R⁵⁶¹ represents
a hydrogen atom;
a halogen atom;
a C₁₋₆ alkyl group;
a C₁₋₆ haloalkyl group; or
a C₁₋₁₀ alkoxy group,
R⁵⁷¹ represents
a hydrogen atom;
a halogen atom;
a C₁₋₁₀ alkyl group; or
a C₁₋₁₀ alkoxy group.

18. The imine compound or the pharmaceutically acceptable salt thereof according to claim 17, wherein
R⁵¹ represents a group represented by Formula (II-1),
B represents a phenyl group,
R⁵⁵¹ represents
a halogen atom;
a C₁₀ alkyl group
a C₁₋₆ haloalkyl group;
a C₁₋₆ alkoxy group;
a C₁₋₆ haloalkoxy group;
a C₃₋₁₀ cycloalkyl group;
an aryl group
an aryloxy group;
a morpholino group;
an arylamino group
a cyano group
a C₁₋₆ alkanoyl group;
a C₂₋₆ haloalkanoyl group; or
a C₁₋₆ alkylsulfonyl group,
R⁵⁶¹ represents
a hydrogen atom;
a halogen atom;
a C₁₋₆ haloalkyl group; or
a C₁₋₆ alkoxy group,
R⁵⁷¹ represents
a hydrogen atom;
a halogen atom;
C₁₋₁₀ alkyl group; or
a C₁₋₁₀ alkoxy group, and
m represents 1.

19. The imine compound or the pharmaceutically acceptable salt thereof according to claim 18, wherein R⁴¹ is a C₁₋₁₀ alkyl group substituted with a C₃₋₁₀ cycloalkyl group(s).

20. The imine compound or the pharmaceutically acceptable salt thereof according to claim 19, wherein
R⁵¹ represents a phenyl group substituted with 1 to 3 groups selected from the group consisting of: a halogen atom, a C₁₋₁₀ alkyl group, a C₁₋₆ haloalkyl group, a C₁₋₁₀ alkoxy group, a cyano group, and a C₁₋₆ haloalkoxy group.

21. The imine compound or the pharmaceutically acceptable salt thereof according to claim 13, wherein the double bond made of the carbon atom and the nitrogen atom contained in the group represented by >C═N—CO— in Formula (I-1) is in (Z) configuration.

22. A cannabinoid-receptor agonist comprising an imine compound represented by Formula (I-2) 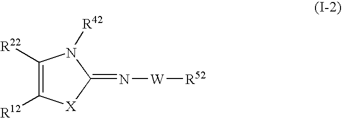
[where R¹² and R²² each represent
a hydrogen atom;
a halogen atom;
a C₁₋₁₀ alkyl group;
a C₁₋₆ haloalkyl group;
a C₁₋₆ alkoxy group;
a carboxyl group;
a C₂₋₆ alkoxycarbonyl group;
a hydroxy-C₁₋₆ alkyl group;
an aryl group that may be substituted with 1 to 3 halogen atoms; or
a group represented by Formula —CON(R⁶¹)R⁷¹ (where R⁶¹ and R⁷¹ each represent a hydrogen atom or a C₁₋₆ alkyl group that may be substituted with a cyclic amino group(s), or R⁶¹ and R⁷¹, in combination with the adjacent nitrogen atom, form a cyclic amino group), or,
R¹¹ and R²², in combination with the adjacent carbon atom, form a benzene ring, a pyridine ring or a cyclohexenyl ring that may be substituted with a C₁₋₆ alkyl group(s) or a halogen atom(s);
R¹² represents
a C₁₋₁₀ alkyl group or C₂₋₆ alkenyl group that may be substituted with: a halogen atom(s), a cyano group(s), a carboxyl group(s), a C₂₋₆ alkoxycarbonyl group(s), a C₃₋₁₀ cycloalkyl group(s), an aryl group(s) that may be substituted with a C₁₋₆ haloalkyl group(s), a C₁₋₆ haloalkoxy group(s), a C₁₋₆ haloalkylthio group(s), a carboxyl group(s), a C₂₋₆ alkoxycarbonyl group(s) or a piperidinocarbamoyl group(s), an arylthio group(s), a C₁₋₆ alkoxy group(s), or a group(s) represented by Formula —CON(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group); or
a C₂₋₆ alkynyl group,
R⁵² represents
a hydrogen atom;
a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a halogen atom, a C₃₋₁₀ cycloalkyl group, a C₂₋₆ alkoxycarbonyl group, a C₁₋₆ alkoxy group that may be substituted with a C₁₋₆ alkoxy group(s) or an aryl group(s), a C₃₋₁₀ cycloalkoxy group that may be substituted with a C₁₋₆ alkyl group(s), an aryl group or aryloxy group that may be substituted with 1 to 5 groups selected from the group consisting of a C₁₋₆ alkyl group, a C₁₋₆ haloalkyl group, a C₁₋₆ alkoxy group, an aralkyloxy group, a nitro group and a halogen atom, a heterocyclic group, a phthalimide group, a C₁₋₆ alkanoyloxy group, an aralkyloxy group, a C₁₋₆ alkylthio group, an arylthio group, and a group represented by —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with a C₁₋₆ alkyl group(s), a C₁₋₆ alkoxy group(s), C₂₋₆ alkoxycarbonyl group(s), or a C₁₋₆ haloalkyl group(s);
an aralkyloxy group;
a group represented by Formula (II-2) 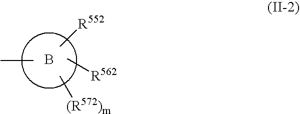
{where
B represents
a C₃₋₁₀ cycloalkyl group;
an aryl group;
a heterocyclic group;
a C₂₋₆ cyclic amino group;
a fluorenyl group;
a 2-oxopyrrolidinyl group
a group represented by Formula (III) 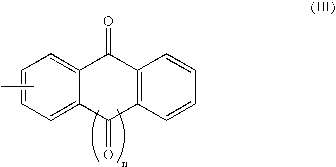
(where n represents 0 or 1); or
a group represented by Formula (IV-2) 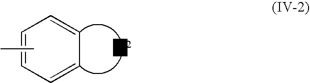
(where Y² represents —(CH₂)ₚ—, —CO—CH₂—CH₂—, —O—CH₂—CH═CH—, or —O—(CH₂)_{q}—O—, in which represents an integer of 2 to 4, and q represents an integer of 1 to 3),
R⁵⁵² represents
a hydrogen atom;
a halogen atom;
a C₁₋₁₀ alkyl group that may be substituted with an aryl group(s) that may be substituted with a halogen atom(s); with an aryloxy group(s); or with a group(s) represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ alkylsulfonyl group an arylthio group that may be substituted with a C₁₋₆ alkyl group(s) or a halogen atom(s);
an arylsulfonyl group that may be substituted with a C₁₋₆ alkyl group(s) or a halogen atom(s);
a C₁₋₆ haloalkoxy group;
a C₁₋₆ haloalkylthio group;
a C₃₋₁₀ cycloalkyl group;
a C₂₋₆ alkenyl group;
a C₁₋₆ alkoxy-C₁₋₆ alkoxy group;
an aryl group that may be substituted with 1 to 3 groups selected from the group consisting of: a C₁₋₆ alkyl group, a C₁₋₆ haloalkyl group, a halogen atom, a C₁₋₆ alkoxy group and a nitro group;
a heterocyclic group that may be substituted with a C₁₋₆ alkyl group(s) or a C₁₋₆ haloalkyl group(s);
an aryloxy group that may be substituted with a halogen atom(s);
a group represented by Formula —N(R⁶³)R⁷³ (where R⁶¹ and R⁷³ each represent a hydrogen atom, a C₁₋₆ alkyl group, a C₁₋₆ hydroxyalkyl group, a C₁₋₆ alkoxy-C₁₋₆alkyl group, an aryl group, a C₁₋₆ alkanoyl group, or R⁶³ and R⁷³, in combination with the adjacent nitrogen atom, form a cyclic amino group);
a hydroxyl group;
a cyano group;
a nitro group;
a carboxyl group;
a C₂₋₆ alkoxycarbonyl group;
a C₂₋₆ cyclic amino group that may be substituted with an aralkyl group(s) or an aryl group(s);
a group represented by Formula —CON(R⁶⁴)R⁷⁴ (where R⁶⁴ and R⁷⁴ each represent a hydrogen atom, a C₁₋₆ alkyl group, a C₁₋₆ alkoxy-C₁₋₆ alkyl group, or a heterocyclic group that may be substituted with a C₁₋₆ alkyl group(s), or R⁶⁴ and R⁷⁴, in combination with the adjacent nitrogen atom, form a cyclic amino group);
a group represented by Formula —SO₂N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
a C₁₋₆ alkylsulfonyl group that may be substituted with a halogen atom(s);
an arylsulfonyl group that may be substituted with a halogen atom(s); or
a 2-oxa-3-oxobicyclo[2.2.1]heptyl group,
R⁵⁶² represents
a hydrogen atom;
a halogen atom;
a C₁₋₁₀ alkyl group;
a C₁₋₆ haloalkyl group; or
a C₁₋₆ alkoxy group,
R⁵⁷² represents
a hydrogen atom;
a C₁₋₁₀ alkyl group
a C₁₋₆ haloalkyl group;
a halogen atom; or
a C₁₋₆ alkoxy group;
m represents an integer of 1 to 3}
X represents an oxygen atom or a sulfur atom;
W represents CO or SO₂],
or a pharmaceutically acceptable salt thereof, as an active ingredient.

23. A cannabinoid-receptor agonist comprising the imine compound or the pharmaceutically acceptable salt thereof according to claim 22, as an active ingredient, wherein
X is a sulfur atom.

24. A cannabinoid-receptor agonist comprising the imine compound or the pharmaceutically acceptable salt thereof according to claim 23, as an active ingredient, wherein
R¹² represents
a hydrogen atom, a halogen atom, a C₁₋₁₀ alkyl group, a carboxyl group, a C₂₋₆ alkoxycarbonyl group, a group represented by Formula —CON(R⁶¹)R⁷¹ (where R⁶¹ and R⁷¹ each represent a hydrogen atom, or a C₁₋₆ alkyl group that may be substituted with a cyclic amino group(s), or R⁶¹ and R⁷¹, in combination with the adjacent nitrogen atom, form a cyclic amino group), or an aryl group,
R²² represents
a hydrogen atom, a C₁₋₁₀ alkyl group, a C₁₋₆ haloalkyl group, or an aryl group, or
R¹¹ and R²², in combination with the adjacent carbon atom, form a benzene ring, a pyridine ring or a cyclohexenyl ring that may be substituted with a C₁₋₆ alkyl group(s) or a halogen atom(s).

25. A cannabinoid-receptor agonist comprising the imine compound or the pharmaceutically acceptable salt thereof according to claim 24, as an active ingredient, wherein
R⁴² represents
a C₁₋₁₀ alkyl group or C₂₋₆ alkenyl group that may be substituted with: a halogen atom(s), a cyano group(s), a carboxyl group(s), a C₂₋₆ alkoxycarbonyl group(s), a C₃₋₁₀ cycloalkyl group(s), an aryl group(s) that may be substituted with a C₁₋₆ haloalkylthio group(s), a carboxyl group(s), or a C₂₋₆ alkoxycarbonyl group(s), an arylthio group(s), a C₁₋₆ alkoxy group(s) or a group represented by Formula —CON(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group); or
a C₂₋₆ alkynyl group,
R⁵² represents
a C₁₋₆ alkoxy group;
a C₁₋₆ haloalkyl group;
a C₁₀ alkyl group or C₂₋₆ alkenyl group that may be substituted with 1 to 3 groups selected from the group consisting of: a C₃₋₁₀ cycloalkyl group, a C₂₋₆ alkoxycarbonyl group, a C₁₋₆ alkoxy group, an aryl group or aryloxy group that may be substituted with 1 to 5 groups selected from the group consisting of a C₁₋₆ alkyl group, a C₁₋₆ haloalkyl group, a C₁₋₆ alkoxy group, an aralkyloxy group, a nitro group and a halogen atom, a heterocyclic group, a C₁₋₆ alkylthio group, an arylthio group, and a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with a C₁₋₆ alkoxy group(s), C₂₋₆ alkoxycarbonyl group(s), or a C₁₋₆ haloalkyl group(s); or
a group represented by Formula (II), where
B represents
a C₃₋₁₀ cycloalkyl group;
an aryl group; or
a heterocyclic group,
R⁵⁵² represents
a hydrogen atom;
a halogen atom;
a C₁₋₁₀ alkyl group that may be substituted with an aryl group(s), or a group(s) represented by Formula —N(R⁶²)R⁷² (R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₋₁₀ alkoxy group;
a C₁₋₆ alkylthio group;
a C₁₋₆ alkylsulfonyl group;
an arylthio group;
an arylsulfonyl group that may be substituted with a halogen atom(s);
a C₁₋₆ haloalkoxy group;
a C₁₋₆ haloalkylthio group;
a C₁₋₆ alkoxy-C₁₋₆alkoxy group;
an aryl group that may be substituted with 1 to 3 groups selected from the group consisting of: a C₁₋₁₆ haloalkyl group, a halogen atom, and a nitro group;
a heterocyclic group that may be substituted with a C₁₋₆ haloalkyl group(s);
an aryloxy group that may be substituted with a halogen atom(s);
a group represented by Formula —N(R⁶³)R⁷³ (where R⁶³ and R⁷³ each represent a hydrogen atom, a C₁₋₆ alkyl group, a C₁₋₆ hydroxyalkyl group, a C₁₋₆ alkoxy-C₁₋₆ alkyl group, or a benzoyl group, or R⁶³ and R⁷³, in combination with the adjacent nitrogen atom, form a cyclic amino group);
a hydroxyl group;
a cyano group;
a nitro group;
a carboxyl group;
a C₂₋₆ alkoxycarbonyl group; or
a group represented by Formula —CON(R⁶⁴)R⁷⁴ (where R⁶⁴ and R⁷⁴ each represent a hydrogen atom, a C₁₋₆ alkyl group, a C₁₋₆ alkoxy-C₁₋₆ alkyl group or a heterocyclic group that may be substituted with a C₁₋₆ alkyl group(s), or R⁶⁴ and R⁷⁴, in combination with the adjacent nitrogen atom, form a cyclic amino group),
R⁵⁶² represents
a hydrogen atom;
a halogen atom;
a C₁₋₁₀ alkyl group;
a C₁₋₆ haloalkyl group; or
a C₁₋₁₀ alkoxy group,
R⁵⁷² represents
a hydrogen atom;
a halogen atom;
C₁₋₁₀ alkyl group; or
a C₁₋₆ alkoxy group.

26. A cannabinoid-receptor agonist comprising the imine compound or the pharmaceutically acceptable salt thereof according to claim 25, as an active ingredient, wherein
R⁵² represents a group represented by Formula (II-2) where
B represents a phenyl group or a pyridyl group;
R⁵⁵² represents
a hydrogen atom;
a halogen atom;
a C₁₋₁₀ alkyl group;
a C₁₋₆ haloalkyl group; or
a C₁₋₁₀ alkoxy group,
R⁵⁶² represents
a hydrogen atom;
a halogen atom;
a C₁₋₁₀ alkyl group; or
a C₁₋₆ alkoxy group,
R⁵⁷² represents
a hydrogen atom;
a halogen atom; or
a C₁₋₆ alkoxy group.

27. A cannabinoid-receptor agonist comprising the imine compound or the pharmaceutically acceptable salt thereof according to claim 26, as an active ingredient, wherein
R⁴² represents
a C₁₋₁₀ alkyl group that may be substituted with: a halogen atom(s), a cyano group(s), a carboxyl group(s), a C₂₋₆ alkoxycarbonyl group(s), a C₃₋₁₀ cycloalkyl group(s), an aryl group(s) that may be substituted with a C₁₋₆ haloalkylthio group(s), a carboxyl group(s) or a C₂₋₆ alkoxycarbonyl group(s), an arylthio group(s), a C₁₋₆ alkoxy group(s), or a group(s) represented by Formula —CON(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group).

28. The cannabinoid-receptor agonist according to claim 22, being a cannabinoid type-1 receptor agonist or a cannabinoid type-2 receptor agonist.

29. The cannabinoid-receptor agonist according to claim 22, being a therapeutic drug or prophylactic drug for pain.

30. The cannabinoid-receptor agonist according to claim 22, being a therapeutic drug or prophylactic drug for autoimmune disease.

31. An imine compound represented by
Formula (I-2) 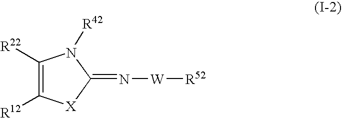
where
W is CO,
R¹ represents
a halogen atom;
a C₁₋₆ alkyl group;
a C₁₋₆ haloalkyl group;
a C₁₋₆ alkoxy group;
a carboxyl group;
a C₂₋₆ alkoxycarbonyl group;
a hydroxy-C₁₋₆ alkyl group; or
an aryl group that may be substituted with 1 to 3 halogen atoms; or
a group represented by Formula —CON(R⁶¹)R⁷¹ (where R⁶¹ and R⁷¹ each represent a hydrogen atom or a C₁₋₆ alkyl group that may be substituted with a cyclic amino group(s), or R⁶¹ and R⁷¹, in combination with the adjacent nitrogen atom, form a cyclic amino group),
R²² represents
a hydrogen atom:
a halogen atom;
a C₁₋₁₀ alkyl group;
a C₁₋₆ haloalkyl group; or
an aryl group, or
R¹² and R²², in combination with the adjacent carbon atom, form a benzene ring, a pyridine ring or a cyclohexenyl ring that may be substituted with a C₁₋₆ alkyl group(s) or a halogen atom(s),
R⁴² represents
a C₁₋₁₀ alkyl group or C₂₋₆ alkenyl group substituted with a C₃₋₁₀ cycloalkyl group(s) or a C₁₋₆ alkoxy group(s), and
X is the same as those as defined regarding R⁵², or a pharmaceutically acceptable salt thereof.

32. The imine compound or the pharmaceutically acceptable salt thereof according to claim 31, wherein X is a sulfur atom.

33. The imine compound or the pharmaceutically acceptable salt thereof according to claim 32, wherein R¹² represents a halogen atom or a C₁₋₁₀ alkyl group; and R²² represents a C₁₋₁₀ alkyl group or a C₁₋₆ haloalkyl group.

34. The imine compound or the pharmaceutically acceptable salt thereof according to claim 33, wherein
R⁵² represents
a C₁₋₆ alkoxy group;
a C₁₋₆ haloalkyl group;
a C₁₋₁₀ alkyl group or C₂₋₆ alkenyl group that may be substituted with 1 to 3 groups selected from the group consisting of: a C₃₋₁₀ cycloalkyl group, a C₂₋₆ alkoxycarbonyl group, a C₁₋₆ alkoxy group, an aryl group or aryloxy group that may be substituted with 1 to 5 groups selected from the group consisting of a C₁₋₆ alkyl group, a C₁₋₆ haloalkyl group, a C₁₋₆ alkoxy group, an aralkyloxy group, a nitro group and a halogen atom, a heterocyclic group, a C₁₋₆ alkylthio group, an arylthio group, and a group represented by Formula —N(R⁶²)R⁷² (where R⁶² and R⁷² each represent a hydrogen atom or a C₁₋₆ alkyl group, or R⁶² and R⁷², in combination with the adjacent nitrogen atom, form a cyclic amino group);
an aryloxy group that may be substituted with a C₁₋₆ alkoxy group(s), C₂₋₆ alkoxycarbonyl group(s), or a C₁₋₆ haloalkyl group(s); or
a group represented by Formula (II-2) where
B represents
a C₃₋₁₀ cycloalkyl group;
an aryl group; or
a heterocyclic group,
R⁵⁵² represents
a hydrogen atom;
a halogen atom;
a C₁₋₁₀ alkyl group that may be substituted with an aryl group(s), or a group(s) represented by Formula —N(R⁶²)R⁷² (R⁶² and R⁷² each represent a hydrogen atom or a C₁₋₆ alkyl group, or R⁶² and R⁷², in combination with the adjacent nitrogen atom, form a cyclic amino group);
a C₁₋₆ haloalkyl group;
a C₁₀ alkoxy group;
a C₁₋₆ alkylthio group;
a C₁₋₆ alkylsulfonyl group;
an arylthio group
an arylsulfonyl group that may be substituted with a halogen atom(s);
a C₁₋₆ haloalkoxy group
a C₁₋₆ haloalkylthio group;
a C₁₋₆ alkoxy-C₁₋₆alkoxy group;
an aryl group that may be substituted with 1 to 3 groups selected from the group consisting of: a C₁₋₆ haloalkyl group, a halogen atom, and a nitro group;
a heterocyclic group that may be substituted with a C₁₋₆ haloalkyl group(s);
an aryloxy group that may be substituted with a halogen atom(s);
a hydroxyl group;
a cyano group;
a nitro group;
a carboxyl group; or
a C₂₋₆ alkoxycarbonyl group,
R⁵⁶² represents
a hydrogen atom;
a halogen atom;
a C₁₋₁₀ alkyl group; or
a C₁₋₁₀ alkoxy group,
R⁵⁷² represents
a hydrogen atom;
a halogen atom;
a C₁₋₁₀ alkyl group; or
a C₁₋₁₀ alkoxy group.

35. The imine compound or the pharmaceutically acceptable salt thereof according to claim 34, wherein
R⁵² represents a group represented by Formula (II-2) where
B represents a phenyl group or a pyridyl group
R⁵⁵² represents
a hydrogen atom;
a halogen atom;
a C₁₋₁₀ alkyl group;
a C₁₋₆ haloalkyl group;
a C₁₋₁₀ alkoxy group; or
a C₁₋₆ haloalkoxy group,
R⁵⁶² represents
a hydrogen atom;
a halogen atom;
a C₁₋₁₀ alkyl group; or
a C₁₋₁₀ alkoxy group,
R⁵⁷² represents
a hydrogen atom;
a halogen atom;
a C₁₋₁₀ alkyl group; or
a C₁₋₆ alkoxy group.

36. The imine compound or the pharmaceutically acceptable salt thereof according to claim 31, wherein the double bond represented by >C═N—CO— in Formula (I-2) is in (Z) configuration.

37. A cannabinoid-receptor agonist comprising an imine compound represented by Formula (I-3) 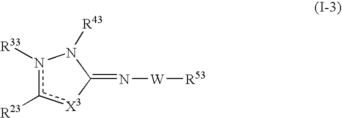
[where the broken line indicates that one of the bonds is a double bond,
X³ represents C(R¹³), S or O,
R¹³, R²³ and R³³ each represent
a hydrogen atom;
a C₁₋₁₀ alkyl group that may be substituted with aryl group(s) substituted with a halogen atom(s);
a C₁₋₆ haloalkyl group;
a C₃₋₁₀ cycloalkyl group; or
an aryl group or aralkyl group that may be substituted with 1 to 3 halogen atoms, or in the case where X³ is C(R¹³), R¹³ and R²³ together represent a group represented by —CH₂—S—CH₂—(with the proviso that, R³³ is not substituted in the case where X³ is S or O),
R⁴³ represents
a 1,1-dioxothiolanyl group; or
a C₁₋₁₀ alkyl group or C₂₋₆ alkenyl group that may be substituted with a group(s) selected from the group consisting of: a C₃₋₁₀ cycloalkyl group, a C₁₋₆ haloalkyl group and a C₁₋₆ alkoxy group; or
an aryl group,
R⁵³ represents
a hydrogen atom;
a C₁₋₁₀ alkoxy group;
a C₁₋₆ alkoxy-C₁₋₆alkoxy group;
a C₁₋₆ haloalkyl group;
a C₁₋₁₀ alkyl group or C₂₋₆ alkenyl group that may be substituted with 1 to 3 groups selected from the group consisting of: a C₁₋₆ alkoxy group, a C₃₋₁₀ cycloalkyl group, a C₂₋₆ alkoxycarbonyl group, an aryl group or aryloxy group that may be substituted with 1 to 5 groups selected from the group consisting of a C₁₋₆ alkoxy group and a halogen atom, a heterocyclic group, a C₁₋₆ alkanoyloxy group, an aralkyloxy group, and a C₁₋₆ alkylthio group;
a group represented by Formula 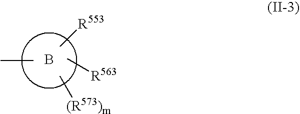
{where B represents
a C₃₋₁₀ cycloalkyl group;
an aryl group;
a heterocyclic group;
a C₂₋₆ cyclic amino group;
a group represented by Formula (III) 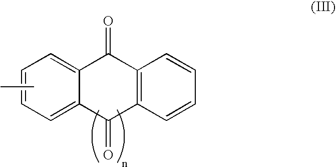
(where n represents 0 or 1); or
a group represented by Formula (IV-3) 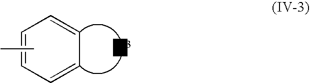
(where Y³ represents —O—CH₂—CH═CH— or —O—(CH₂)_{q}—O—, in which q represents an integer of 1 to 3),
R⁵⁵³ represents
a hydrogen atom;
a halogen atom;
an aryl group;
a C₁₋₁₀ alkyl group;
a C₁₋₆ alkanoyloxy-C₁₋₆ alkyl group;
a C₁₋₆ haloalkyl group;
a C₁₋₆ alkoxy group;
a C₁₋₆ alkylthio group;
a C₂₋₆ alkenyloxy group;
a C₂₋₆ alkenylthio group;
a C₁₋₆ haloalkoxy group;
a C₁₋₆ haloalkylthio group;
an aryl group that may be substituted with 1 to 3 halogen atoms or cyano groups;
a heterocyclic group;
an aryloxy group or arylthio group that may be substituted with a halogen atom(s) or a C₁₋₆ alkyl group(s);
a group represented by Formula —N(R⁶³³)R⁷³³ (where R⁶³³ and R⁷³³ each represent a hydrogen atom, a C₁₋₆ alkyl group, a C₁₋₆ alkanoyl group, a di-C₁₋₆ alkylamino-C₂₋₆ alkanoyl group, or a heterocyclic group that may be substituted with a C₁₋₆ alkyl group(s), or R⁶³³ and R⁷³³, in combination with the adjacent nitrogen atom, form a cyclic amino group);
a cyano group;
a nitro group; or
a C₂₋₆ alkoxycarbonyl group,
R⁵⁶³ represents
a hydrogen atom;
a halogen atom;
a C₁₋₁₀ alkyl group; or
a C₁₋₆ haloalkyl group,
R⁵⁷³ represents
a hydrogen atom;
a C₁₋₁₀ alkyl group;
a halogen atom; or
a C₁₋₁₀ alkoxy group,
m represents an integer of 1 to 3}, and
W represents —CO—, —CO—CO—, —CO—NH—, —CS—NH— or —SO₂—]
or a pharmaceutically acceptable salt thereof, as an active ingredient.

38. A cannabinoid-receptor agonist comprising the imine compound or the pharmaceutically acceptable salt thereof according to claim 37, as an active ingredient, wherein
R¹³ represents a hydrogen atom or a phenyl group; and R²³ and R³³ each represent a C₁₋₆ alkyl group, a C₁₋₆ haloalkyl group, or a C₃₋₁₀ cycloalkyl group,
R⁴³ represents
a C₁₋₁₀ alkyl group that may be substituted with a group(s) selected from the group consisting of:
a C₃₋₁₀ cycloalkyl group, a C₁₋₆ haloalkyl group and a C₁₋₆ alkoxy group,
R⁵³ represents
a C₁₋₁₀ alkoxy group;
a C₁₋₆ alkoxy-C₁₋₆alkoxy group;
a C₁₋₆ haloalkyl group;
a C₁₋₁₀ alkyl group or C₂₋₆ alkenyl group that may be substituted with 1 to 3 groups selected from the group consisting of: a C₁₋₆ alkoxy group, a C₃₋₁₀ cycloalkyl group, a C₂₋₆ alkoxycarbonyl group, an aryl group or aryloxy group that may be substituted with 1 to 5 groups selected from the group consisting of a C₁₋₆ alkoxy group and a halogen atom, a heterocyclic group, a C₁₋₆ alkanoyloxy group, an aralkyloxy group, and a C₁₋₆ alkylthio group;
a group represented by Formula (II-3);
where B represents
an aryl group, a furyl group, a thienyl group, pyrazolyl group, isoxazolyl group, pyridyl group, a group represented by Formula (III), or a group represented by Formula (IV-3).

39. A cannabinoid-receptor agonist comprising the imine compound or the pharmaceutically acceptable salt thereof according to claim 38, as an active ingredient, wherein W is —CO—.

40. A cannabinoid-receptor agonist comprising the imine compound or the pharmaceutically acceptable salt thereof according to claim 39, as an active ingredient, wherein
X³ represents C(R¹³),
R⁵³ represents
a C₁₋₁₀ alkoxy group;
a C₁₋₆ haloalkyl group;
a C₁₋₁₀ alkyl group that may be substituted with 1 to 3 groups selected from the group consisting of: a C₁₋₆ alkoxy group, a C₃₋₁₀ cycloalkyl group, an aryl group that may be substituted with 1 to 5 groups selected from the group consisting of a C₁₋₆ alkoxy group and a halogen atom; or
a group represented by Formula (II-3) where B represents a phenyl group.

41. A cannabinoid-receptor agonist comprising the imine compound or the pharmaceutically acceptable salt thereof according to claim 40, as an active ingredient, wherein
R⁵⁵³ represents
a halogen atom;
a C₁₋₁₀ alkyl group;
a C₁₋₆ haloalkyl group;
a C₁₋₁₀ alkoxy group;
a C₁₋₆ haloalkoxy group;
an aryl group that may be substituted with 1 to 3 halogen atoms;
an aryloxy group that may be substituted with a halogen atom(s);
a group represented by Formula —N(R⁶³³)R⁷³³ (where R⁶³³ and R⁷³³ each represent a hydrogen atom or a C₁₋₆ alkyl group);
a cyano group;
a nitro group; or
a C₂₋₆ alkoxycarbonyl group,
R⁵⁶³ represents
a hydrogen atom;
a halogen atom;
a C₁₋₁₀ alkyl group;
a C₁₋₆ alkoxy group; or
a C₁₋₆ haloalkyl group,
R⁵⁷³ represents
a hydrogen atom;
a C₁₋₁₀ alkyl group;
a halogen atom; or
a C₁₋₁₀ alkoxy group.

42. A cannabinoid-receptor agonist comprising the imine compound or the pharmaceutically acceptable salt thereof according to claim 39, as an active ingredient, wherein X³ is a sulfur atom; and R⁵³ is a group represented by Formula (II-3) where B is a phenyl group.

43. A cannabinoid-receptor agonist comprising the imine compound or the pharmaceutically acceptable salt thereof according to claim 42, as an active ingredient, wherein
R⁵⁵³ represents
a halogen atom;
a C₁₋₁₀ alkyl group;
a C₁₋₆ haloalkyl group; or
a C₁₋₁₀ alkoxy group;
R⁵⁶³ represents
a hydrogen atom;
a halogen atom;
a C₁₋₁₀ alkyl group;
a C₁₋₆ alkoxy group; or
a C₁₋₆ haloalkyl group,
R⁵⁷³ represents a hydrogen atom.

44. A cannabinoid-receptor agonist comprising the imine compound or the pharmaceutically acceptable salt thereof according to claim 41, as an active ingredient, where R⁴³ is a C₁₋₁₀ alkyl group substituted with a C₃₋₁₀ cycloalkyl group(s).

45. The cannabinoid-receptor agonist according to claim 37, being a cannabinoid type-1 receptor agonist or a cannabinoid type-2 receptor agonist.

46. The cannabinoid-receptor agonist according to claim 37, being a therapeutic drug or prophylactic drug for pain.

47. The cannabinoid-receptor agonist according to of claim 37, being a therapeutic drug or prophylactic drug for autoimmune disease.

48. An imine compound represented by Formula (I-3) 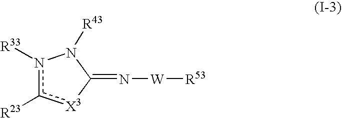
[where a broken line indicates that one of the bonds is a double bond,
X³ represents C(R¹³), S or O,
R¹³, R²³ and R³³ each represent
a hydrogen atom;
a C₁₋₁₀ alkyl group that may be substituted with aryl group(s) substituted with a halogen atom(s);
a C₁₋₆ haloalkyl group;
a C₃₋₁₀ cycloalkyl group; or
an aryl group or aralkyl group that may be substituted with 1 to 3 halogen atoms, or
in the case where X³ is C(R¹³), R¹³ and R²³ together represent a group represented by —CH₂—S—CH₂— (with the proviso that, R³³ is not substituted in the case where X³ is S or O),
R⁴³ represents
a 1,1-dioxothiolanyl group;
a C₁₋₁₀ alkyl group or C₂₋₆ alkenyl group that may be substituted with a group(s) selected from the group consisting of: a C₃₋₁₀ cycloalkyl group, a C₁₋₆ haloalkyl group and a C₁₋₆ alkoxy group; or
an aryl group,
R⁵³ represents
a hydrogen atom;
a C₁₋₁₀ alkoxy group;
a C₁₋₆ alkoxy-C₁₋₆alkoxy group;
a C₁₋₆ haloalkyl group;
a C₁₋₁₀ alkyl group or C₂₋₆ alkenyl group that may be substituted with 1 to 3 groups selected from the group consisting of: a C₁₋₆ alkoxy group, a C₃₋₁₀ cycloalkyl group, a C₂₋₆ alkoxycarbonyl group, an aryl group or aryloxy group that may be substituted with 1 to 5 groups selected from the group consisting of a C₁₋₆ alkoxy group and a halogen atom, a heterocyclic group, a C₁₋₆ alkanoyloxy group, an aralkyloxy group, and a C₁₋₆ alkylthio group;
a group represented by Formula (II-3) 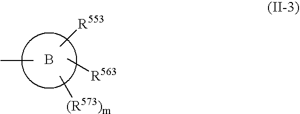
{where B represents
a C₃₋₁₀ cycloalkyl group;
an aryl group;
a heterocyclic group;
a C₂₋₆ cyclic amino group;
a group represented by Formula (III) 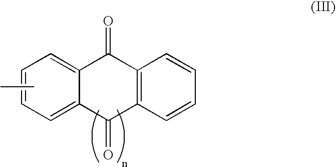
(where n represents 0 or 1); or
a group represented by Formula (IV-3) 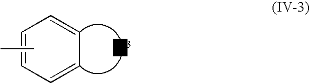
(where Y³ represents —O—CH₂—CH═CH— or —O—(CH₂)_{q}—O—, where q represents an integer of 1 to 3),
R⁵⁵³ represents
a hydrogen atom;
a halogen atom;
an aryl group;
a C₁₋₁₀ alkyl group;
a C₁₋₆ alkanoyloxy-C₁₋₆alkyl group;
a C₁₋₆ haloalkyl group;
a C₁₋₁₀ alkoxy group;
a C₁₋₆ alkylthio group;
a C₂₋₆ alkenyloxy group;
a C₂₋₆ alkenylthio group;
a C₁₋₆ haloalkoxy group;
a C₁₋₆ haloalkylthio group,
an aryl group that may be substituted with 1 to 3 halogen atoms or cyano groups;
a heterocyclic group;
an aryloxy group or arylthio group that may be substituted with a halogen atom(s) or a C₁₋₆ alkyl group(s);
a group represented by Formula —N(R⁶³³)R⁷³³ (where R⁶³³ and R⁷³³ each represent a hydrogen atom, a C₁₋₆ alkyl group, a C₁₋₆ alkanoyl group, a di-C₁₋₆ alkylamino-C₂₋₆ alkanoyl group, or a heterocyclic group that may be substituted with a C₁₋₆ alkyl group(s), or R⁶³³ and R⁷³³, in combination with the adjacent nitrogen atom, form a cyclic amino group);
a cyano group;
a nitro group; or
a C₂₋₆ alkoxycarbonyl group,
R⁵⁶³ represents
a hydrogen atom;
a halogen atom;
a C₁₋₁₀ alkyl group; or
a C₁₋₆ haloalkyl group,
R⁵⁷³ represents
a hydrogen atom;
a C₁₋₁₀ alkyl group;
a halogen atom; or
a C₁₋₁₀ alkoxy group, and
m represents an integer of 1 to 3}, and
W represents —CO—, —CO—CO—, —CO—NH—, —CS—NH— or —SO₂—]
or a pharmaceutically acceptable salt thereof.

49. The imine compound or the pharmaceutically acceptable salt thereof according to claim 48, wherein R¹³ represents a hydrogen atom or a phenyl group; and R²³ and R³³ each represent a C₁₋₆ alkyl group, a C₁₋₆ haloalkyl group or a C₃₋₁₀ cycloalkyl group,
R⁴³ represents
a C₁₋₁₀ alkyl group that may be substituted with a group(s) selected from the group consisting of:
a C₃₋₁₀ cycloalkyl group and a C₁₋₆ alkoxy group; or
a C₁₋₆ haloalkyl group,
R⁵³ represents
a C₁₋₆ alkoxy group;
a C₁₋₆ alkoxy-C₁₋₆alkoxy group;
a C₁₋₆ haloalkyl group;
a C₁₋₁₀ alkyl group or C₂₋₆ alkenyl group that may be substituted with 1 to 3 groups selected from the group consisting of: a C₁₋₆ alkoxy group, a C₃₋₁₀ cycloalkyl group, a C₂₋₆ alkoxycarbonyl group, an aryl group or aryloxy group that may be substituted with 1 to 5 groups selected from the group consisting of a C₁₋₆ alkoxy group and a halogen atom, a heterocyclic group, a C₁₋₆ alkanoyloxy group, an aralkyloxy group, and a C₁₋₆ alkylthio group; or
a group represented by Formula (II-3), where
B represents
an aryl group, a furyl group, a thienyl group, pyrazolyl group, isoxazolyl group, pyridyl group, a group represented by Formula (III), or a group represented by Formula (IV-3).

50. The imine compound or the pharmaceutically acceptable salt thereof according to claim 49, wherein W is —CO—.

51. The imine compound or the pharmaceutically acceptable salt thereof according to claim 50, wherein
X³ represents C(R¹³);
R⁵³ represents
a C₁₋₁₀ alkoxy group;
a C₁₋₆ haloalkyl group;
a C₁₋₁₀ alkyl group that may be substituted with 1 to 3 groups selected from the group consisting of: a C₁₋₆ alkoxy group, a C₃₋₁₀ cycloalkyl group, and an aryl group that may be substituted with 1 to 5 groups selected from the group consisting of a C₁₋₆ alkoxy group and a halogen atom; or
a group represented by Formula (II-3), where B represents a phenyl group.

52. The imine compound or the pharmaceutically acceptable salt thereof according to claim 51, wherein
R⁵⁵³ represents
a halogen atom;
a C₁₋₁₀ alkyl group;
a C₁₋₆ haloalkyl group;
a C₁₋₁₀ alkoxy group;
a C₁₋₆ haloalkoxy group;
an aryl group that may be substituted with 1 to 3 halogen atoms;
an aryloxy group that may be substituted with a halogen atom(s);
a group represented by Formula —N(R⁶³³)R⁷³³ (where R⁶³³ and R⁷³³ each represent a hydrogen atom or a C₁₋₆ alkyl group;
a cyano group;
a nitro group; or
a C₂₋₆ alkoxycarbonyl group,
R⁵⁶³ represents
a hydrogen atom;
a halogen atom;
a C₁₋₁₀ alkyl group; or
a C₁₋₆ haloalkyl group,
R⁵⁷³ represents
a hydrogen atom;
a C₁₋₁₀ alkyl group;
a halogen atom; or
a C₁₀ alkoxy group.

53. The imine compound or the pharmaceutically acceptable salt thereof according to claim 52, wherein R⁴³ represents a C₁₋₁₀ alkyl group substituted with a C₃₋₁₀ cycloalkyl group(s).

54. The imine compound or the pharmaceutically acceptable salt thereof according to claim 49, wherein X³ is a sulfur atom and W is —CO— or —SO₂—.

55. The imine compound or the pharmaceutically acceptable salt thereof according to claim 54, wherein
R²³ represents a C₁₋₆ alkyl group; R⁴³ represents
a C₁₋₁₀ alkyl group substituted with a C₃₋₁₀ cycloalkyl group; R⁵³ represents a group represented by Formula (II-3), where
B represents an aryl group, R⁵⁵³ represents a C₁₋₆ haloalkyl group; and R⁵⁶³ represents a hydrogen atom, a halogen atom, a C₁₋₁₀ alkyl group or a C₁₋₁₀ alkoxy group; R⁵⁷³ represents a hydrogen atom.

56. The imine compound or the pharmaceutically acceptable salt thereof according to claim 50, wherein the double bond represented by >C═N—CO— in Formula (I-3) is in (Z) configuration.
